(19) United States
(12) Patent Application Publication
(10) Pub. No.: US 2020/0040334 A1
(43) Pub. Date: Feb. 6, 2020

(54) COMPOSITIONS AND METHODS FOR GENE EDITING

(71) Applicants: ZHEJIANG UNIVERSITY, Hangzhou (CN); HEBEI UNIVERSITY OF SCIENCE AND TECHNOLOGY, Shijiazhuang (CN)

(72) Inventors: Xiao SHEN, Hangzhou (CN); Chunyu HAN, Shijiazhuang (CN)

(21) Appl. No.: 16/064,435

(22) PCT Filed: Dec. 20, 2016
(86) PCT No.: PCT/CN2016/111045

§ 371 Date: Jun. 20, 2018

(30) Foreign Application Priority Data
Dec. 21, 2015 (CN) 201510971234.5
May 23, 2016 (CN) 201610349444.5

(51) Int. Cl. C12N 15/113; C07K 14/195; C12N 15/10; C12N 15/86

(52) U.S. Cl. CPC C12N 15/113; C07K 14/195; C12N 2310/20; C12N 15/86; C12N 15/102

(57) ABSTRACT

(57) Abstract: The present application provides methods, compositions, delivery systems, ami kits for modifying a target nucleic acid using an Argonaute (Ago) and a single-stranded guide DNA. In some embodiments, methods of gene editing in a cell, such as a mammalian cell, arc provided.

### CROSS REFERENCE TO RELATED APPLICATIONS

This application claims priority benefit of Chinese Patent Application No. 201510971234.5 filed Dec. 21, 2015, and Chinese Patent Application No. 201610349444.5 filed May 25, 2016, the contents of each of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 777072000140SEQLIST.txt, date recorded: Dec. 15, 2016, size: 309 KB).

### FIELD OF THE INVENTION

The present invention relates to Argonaute-based methods, compositions, and delivery systems useful for sequence-specific modification of a target nucleic acid, including gene editing in a cell.

### BACKGROUND OF THE INVENTION

Deep understanding of genetic information and development of gene therapies require accurate and effective gene editing tools. As a rapidly developing technology field in genetic engineering, gene or genome editing employs nucleases to introduce sequence-specific modifications, such as mutations, insertions, and substitutions, etc., in a gene or genome. Current gene editing technologies typically involve three steps. First, one or more specially designed guide DNA or RNA (guide element) and an endonuclease (gene-cleaving element) bind to each other, wherein the former element guides the latter element to a specific locus in a target gene. Second, the endonuclease cleaves the specific locus in the target gene, inducing a nick or gap in each of the two strands of the target gene, thereby yielding a double-strand break (DSB). Finally, the DSB activates endogenous DNA repair machinery in the cell, which repairs the break and simultaneously introduces modifications such as mutations, insertions, and substitutions at the repair site. Most eukaryotic cells rely on two DSB repair mechanisms: Non-homologous End-joining (NHEJ) and Homology Directed Repair (HDR). NHEJ uses a series of enzymes to directly connect two broken ends in a DNA. Thus, NHEJ is an error-prone, low-fidelity repair pathway, which often introduces mutations during repair. In comparison, HDR requires a homologous sequence to serve as a template to recover a lost DNA sequence at a DSB. The requirement for a homologous template in HDR can be exploited to introduce exogenous sequences into a target locus in the genome. The mechanism of HDR is similar to homologous recombination. However, because HDR is a DSB-based repair pathway, the efficiency for introducing exogenous sequences using HDR is about three orders of magnitude higher than that using homologous recombination. Therefore, by repairing and modifying induced DSBs, NEJH and HDR enable highly efficient gene editing using a nuclease. Gene editing technology has revolutionary impacts on our understanding of gene functions, engineering of genes, and development of gene therapies.

Currently, four major families of nucleases are widely used in gene editing: Zinc finger nucleases (ZFNs), Transcription Activator-Like Effector Nucleases (TALENs), engineered meganuclease/re-engineered homing endonucleases, and the CRISPR/Cas9 system. Application of the first three nuclease families is challenged by various limitations, including requirement for special target sequences, customized design of the nuclease to accommodate different target sequences, and high off-target effects. CRISPR/Cas9 is currently the most commonly used gene editing system. An enzyme of bacterial origin, Cas9 can use an RNA guide to theoretically cleave and induce a DSB at any target locus proximal and upstream to a PAM sequence in the genome. However, as RNA is prone to secondary structure formation, the Cas9 system suffers from low efficiency in editing GC-rich genes. Additionally, as cells naturally produce a large number of RNA fragments, there remains a probability for Cas9 to bind non-specific RNA and thereby cleave non-target loci in the genome. Cas9 is also known to have relatively high tolerability for mismatch between guide RNA and target DNA. For example, up to 5-nucleotide mismatches between guide RNA and target DNA do not prevent cleavage by Cas9. Therefore, the Cas9 system is limited by its off-target effects.

Argonautes are a family of nucleic acid-binding proteins ubiquitously found in the cells of bacteria, plants, archaea and animals. Argonautes are most well-known for their role in RNA interference (RNAi). Argonaute proteins from most species bind to noncoding oligonucleotide RNAs, which serve as guide RNAs for the Argonaute proteins to recognize target mRNAs via sequence complementarity, and subsequently induce mRNA degradation or translational repression thereby inhibiting gene translation. In recent years, Argonaute from the bacterial species Thermus thermophilus (“TtAgo”) is shown to bind single-stranded DNAs (ssDNAs) and use the ssDNAs as guides to degrade DNA plasmids. This phenomenon suggests that Argonautes from some species are DNA-guided DNA endonucleases. However, TtAgo requires a high temperature (>65° C.) to carry out its endonuclease activity, and thus is not suitable as a gene editing tool.

The disclosures of all publications, patents, patent applications and published patent applications referred to herein are hereby incorporated herein by reference in their entirety.

### BRIEF SUMMARY OF THE INVENTION

The present application provides methods, compositions, delivery systems and kits for modifying a target nucleic acid using an Argonaute (Ago) protein and a short single-stranded guide DNA. The target nucleic acids may be present in vitro, or inside a cell. Exemplary modifications to the target nucleic acid include, but are not limited to, site-specific cleavage, introduction of mutations, insertion of exogenous sequences, sequence substitutions, and alteration of gene expression.

One aspect of the present application provides a method of modifying a target nucleic acid, comprising contacting the target nucleic acid with an Argonaute (Ago) protein and a single-stranded guide DNA at a temperature of about 10° C. to about 60° C. (such as about 37° C.), wherein the Ago protein and the guide DNA form a complex that specifically recognizes a target locus in the target nucleic acid, and wherein the target locus comprises a sequence that is complementary to the sequence of the guide DNA. In some embodiments, the complex cleaves the target locus. In some embodiments, wherein the target locus is a double-stranded DNA, the Ago protein induces a double-strand break in the target locus.

In some embodiments according to any one of the methods described above, the Ago protein and the guide DNA are present in a pre-formed complex. In some embodiments, the guide DNA does not dissociate from the Ago protein at a temperature lower than about 50° C.

In some embodiments according to any one of the methods described above, the sequence of the target locus comprises no more than about 3 (such as any one of 3, 2, 1, or 0) mismatches to the sequence of the guide DNA.

In some embodiments according to any one of the methods described above, the target locus has a GC content of at least about 60% (such as at least about any one of 60%, 65%, 70%, 75%, 80% or more).

In some embodiments according to any one of the methods described above, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site.

In some embodiments according to any one of the methods described above, the Ago protein is derived from an organism selected from the group consisting of Natronobacterium gregoryi, Microcystis aeruginosa, Halogeometricum pallidum, Natrialba asiatica, Natronorubrum tibetense, Natrinema pellirubrum, Halogeometricum borinquense, Thermococcus barophilus, Thermosynechococcus elongatus, Halorubrum lacusprofundi, Microcystis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolyticus, Aromatoleum aromaticum, Thermococcus onnurineus, Methanopyrus kandleri, Synechococcus elongatus, Anoxybacillus flavithermus, Exiguobacterium sp., Lyngbya sp., Clostridium butyricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the Ago protein is derived from Natronobacterium gregoryi. In some embodiments, the Ago protein is derived from Pedobacter heparinus. In some embodiments, the Ago protein is derived from Microcystis sp. In some embodiments, the Ago protein is derived from Microcystis aeruginosa.

In some embodiments according to any one of the methods described above, the Ago protein comprises an amino acid sequence having at least about 80% sequence homology (such as at least about any one of 85%, 90%, 95%, 98% or more sequence homology, or about 100% sequence identity) to a sequence selected from the group consisting of SEQ ID NOs: 1-42. In some embodiments, the Ago protein comprises an amino acid sequence having at least about 80% sequence homology (such as at least about any one of 85%, 90%, 95%, 98% or more sequence homology, or about 100% sequence identity) to SEQ ID NO: 1. In some embodiments, the Ago protein comprises an amino acid sequence having at least about 80% sequence homology (such as at least about any one of 85%, 90%, 95%, 98% or more sequence homology, or about 100% sequence identity) to SEQ ID NO: 2. In some embodiments, the Ago protein comprises an amino acid sequence having at least about 80% sequence homology (such as at least about any one of 85%, 90%, 95%, 98% or more sequence homology, or about 100% sequence identity) to SEQ ID NO: 11. In some embodiments, the Ago protein comprises an amino acid sequence having at least about 80% sequence homology (such as at least about any one of 85%, 90%, 95%, 98% or more sequence homology, or about 100% sequence identity) to SEQ ID NO: 41.

In some embodiments according to any one of the methods described above, the guide DNA is phosphorylated at the 5′ terminus.

In some embodiments according to any one of the methods described above, the guide DNA is about 10 to about 50 nucleotides (nt) long, such as about 15 nt to about 35 nt, about 20 nt to about 27 nt, or about 23 nt to 25 nt.

In some embodiments according to any one of the methods described above, the contacting is in the presence of a divalent metal ion, such as Mg²⁺. In some embodiments, the concentration of the divalent metal ion is at least about 0.1 mM.

In some embodiments according to any one of the methods described above, the target nucleic acid is an isolated DNA. In some embodiments, the target nucleic acid is present in a cell.

In some embodiments according to any one of the methods described above, wherein the target nucleic acid is present in a cell, the method comprises transfecting the cell with the guide DNA and a nucleic acid encoding the Ago protein. In some embodiments, the guide DNA and the nucleic acid encoding the Ago protein are transfected into the cell simultaneously. In some embodiments, the guide DNA is transfected into the cell prior to the nucleic acid encoding the Ago protein. In some embodiments, the cell is transfected with the guide DNA for at least two times. In some embodiments, the molar ratio of the guide DNA to the nucleic acid encoding the Ago protein is at least about 100:1. In some embodiments, the nucleic acid encoding the Ago protein is operably linked to a promoter. In some embodiments, the nucleic acid encoding the Ago protein is present in a vector, such as a viral vector. In some embodiments, the vector is an ultrapure plasmid. In some embodiments, the nucleic acid encoding the Ago protein is an mRNA. In some embodiments, the nucleic acid encoding the Ago protein is codon-optimized for the organism from which the cell is derived.

In some embodiments according to any one of the methods described above, wherein the target nucleic acid is present in a cell, the method comprises delivering a pre-formed complex comprising the Ago protein and the guide DNA into the cell. In some embodiments, the pre-formed complex is delivered into the cell by electroporation, microinjection, or mechanical deformation via a microfluidic channel. In some embodiments, the pre-formed complex is delivered into the cell via a vehicle selected from the group consisting of a cell-penetrating peptide, a virus-like particle, a nanocarrier, a liposome, a polymer, and a nanoparticle-stabilized nanocapsule. In some embodiments, the Ago protein is fused to the cell-penetrating peptide.

In some embodiments according to any one of the methods described above, wherein the target nucleic acid is present in a cell, the method comprises treating the cell with one or more antibiotics. In some embodiments, the cell is free from contamination by non-viral microorganisms, such as mycoplasma.

In some embodiments according to any one of the methods described above, wherein the target nucleic acid is present in a cell, the Ago protein comprises a nuclear localization signal (NLS).

In some embodiments according to any one of the methods described above, wherein the target nucleic acid is present in a cell, the target nucleic acid is endogenous to the cell. In some embodiments, the target nucleic acid is a genomic DNA. In some embodiments, the target nucleic acid is exogenous to the cell. In some embodiments, the target nucleic acid is a viral DNA. In some embodiments, the target nucleic acid is integrated in the genome of the cell. In some embodiments, the target nucleic acid is not integrated in the genome of the cell.

In some embodiments according to any one of the methods described above, wherein the target nucleic acid is present in a cell, the cell is a prokaryotic cell. In some embodiments, the cell is a eukaryotic cell. In some embodiments, the cell is a mammalian cell, such as a human cell. In some embodiments, the cell is a yeast cell, a fungus cell, or a plant cell. In some embodiments, the cell is derived from a cell line. In some embodiments, the cell is a primary cell. In some embodiments, the cell is an immune cell.

In some embodiments according to any one of the methods described above, the modifying comprises site-specific cleavage of the target nucleic acid.

In some embodiments according to any one of the methods described above, wherein the target nucleic acid is present in a cell, the modifying comprises introducing a mutation at the target locus selected from an insertion, a deletion, and a frameshift mutation.

In some embodiments according to any one of the methods described above, wherein the target nucleic acid is present in a cell, the method further comprises contacting the target nucleic acid with a donor DNA comprising a sequence homologous to the sequence of the target locus under a condition that allows integration of the donor DNA at the target locus. In some embodiments, the donor DNA encodes a selection marker, such as a reporter protein. In some embodiments, the method further comprises assessing the cell for expression of the selection marker. In some embodiments, the modifying comprises knocking in an exogenous sequence at the target locus, wherein the donor DNA comprises the exogenous sequence. In some embodiments, the modifying comprises introducing a substitution mutation at the target locus, wherein the donor DNA comprises the substitution mutation. In some embodiments, the substitution mutation is a single nucleotide substitution. In some embodiments, the target locus is a disease-associated locus.

In some embodiments according to any one of the methods described above, wherein the target nucleic acid is present in a cell, the method further comprises sequencing the target nucleic acid after the modifying.

In some embodiments according to any one of the methods described above, wherein the target nucleic acid is present in a cell, the modifying comprises inducing a phenotypic change to the cell. In some embodiments, the method further comprises assessing the phenotypic change to the cell. In some embodiments, the modifying comprises altering expression of the target nucleic acid. In some embodiments, the modifying comprises introducing a knockout mutation at the target locus. In some embodiments, the target locus is a disease-associated locus.

One aspect of the present application provides a composition comprising a complex comprising an Ago protein and a single-stranded guide DNA, wherein the complex is capable of specifically recognizing a target locus at a temperature of about 10° C. to about 60° C. (such as about 37° C.), and wherein the target locus comprises a sequence that is complementary to the sequence of the guide DNA. In some embodiments, the molar ratio between the guide DNA and the Ago protein is at least about 1:1 (such as about 1:1).

One aspect of the present application provides a delivery system comprising a complex comprising an Ago protein and a single-stranded guide DNA, and a vehicle suitable for intracellular delivery of the complex, wherein the complex is capable of specifically recognizing a target locus at a temperature of about 10° C. to about 60° C. (such as about 37° C.), and wherein the target locus comprises a sequence that is complementary to the sequence of the guide DNA. In some embodiments, the vehicle is selected from the group consisting of a cell-penetrating peptide, a virus-like particle, a nanocarrier, a liposome, a polymer, and a nanoparticle-stabilized nanocapsule. In some embodiments, the Ago protein is fused to the cell-penetrating peptide. In some embodiments, the molar ratio between the guide DNA and the Ago protein is at least about 1:1 (such as about 1:1).

One aspect of the present application provides a kit comprising a nucleic acid encoding an Ago protein and a single-stranded guide DNA, wherein the Ago protein and the guide DNA forms a complex that is capable of specifically recognizing a target locus at a temperature of about 10° C. to about 60° C. (such as about 37° C.), and wherein the target locus comprises a sequence that is complementary to the sequence of the guide DNA. In some embodiments, the nucleic acid encoding the Ago protein is operably linked to a promoter. In some embodiments, the nucleic acid encoding the Ago protein is present in a vector, such as a viral vector. In some embodiments, the vector is an ultrapure plasmid. In some embodiments, the nucleic acid encoding the Ago protein is an mRNA. In some embodiments, the nucleic acid encoding the Ago protein is codon-optimized for an organism of interest.

In some embodiments according to any one of the compositions, delivery systems, or kits described above, the Ago protein is capable of cleaving the target locus. In some embodiments, wherein the target locus is a double-stranded DNA, the Ago protein is capable of inducing a double-strand break in the target locus.

In some embodiments according to any one of the compositions, delivery systems, or kits described above, the guide DNA does not dissociate from the Ago protein at a temperature lower than about 50° C.

In some embodiments according to any one of the compositions, delivery systems, or kits described above, the guide DNA and the Ago protein do not naturally occur in the same organism.

In some embodiments according to any one of the compositions, delivery systems, or kits described above, the guide DNA is phosphorylated at the 5′ terminus.

In some embodiments according to any one of the compositions, delivery systems, or kits described above, the guide DNA is about 10 to about 50 nucleotides long, such as about 15 nt to about 35 nt, about 20 nt to about 27 nt, or about 23 nt to 25 nt.

In some embodiments according to any one of the compositions, delivery systems, or kits described above, the sequence of the target locus comprises no more than about 3 (such as any one of 3, 2, 1, or 0) mismatches to the sequence of the guide DNA.

In some embodiments according to any one of the compositions, delivery systems, or kits described above, the target locus has a GC content of at least about 60% (such as at least about any one of 60%, 65%, 70%, 75%, 80% or more).

In some embodiments according to any one of the compositions, delivery systems, or kits described above, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site.

In some embodiments according to any one of the compositions, delivery systems, or kits described above, the Ago protein is derived from an organism selected from the group consisting of Natronobacterium gregoryi, Microcystis aeruginosa, Halogeometricum pallidum, Natrialba asiatica, Natronorubrum tibetense, Natrinema pellirubrum, Halogeometricum borinquense, Thermococcus barophilus, Thermosynechococcus elongatus, Halorubrum lacusprofundi, Microcystis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolvticus, Aromatoleum aromaticum. Thermococcus onnurineus, Methanopyrus kandleri, Synechococcus elongatus, Anoxybacillus flavithermus, Exiguobacterium sp., Lyngbya sp., Clostridium butyricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the Ago protein is derived from Natronobacterium gregoryi. In some embodiments, the Ago protein is derived from Pedobacter heparinus. In some embodiments, the Ago protein is derived from Microcystis sp. In some embodiments, the Ago protein is derived from Microcystis aeruginosa.

In some embodiments according to any one of the compositions, delivery systems, or kits described above, the Ago protein comprises an amino acid sequence having at least about 80% sequence homology (such as at least about any one of 85%, 90%, 95%, 98% or more sequence homology, or about 100% sequence identity) to a sequence selected from the group consisting of SEQ ID NOs:1-42. In some embodiments, the Ago protein comprises an amino acid sequence having at least about 80% sequence homology (such as at least about any one of 85%, 90%, 95%, 98% or more sequence homology, or about 100% sequence identity) to SEQ ID NO: 1. In some embodiments, the Ago protein comprises an amino acid sequence having at least about 80% sequence homology (such as at least about any one of 85%, 90%, 95%, 98% or more sequence homology, or about 100% sequence identity) to SEQ ID NO: 2. In some embodiments, the Ago protein comprises an amino acid sequence having at least about 80% sequence homology (such as at least about any one of 85%, 90%, 95%, 98% or more sequence homology, or about 100% sequence identity) to SEQ ID NO: 11. In some embodiments, the Ago protein comprises an amino acid sequence having at least about 80% sequence homology (such as at least about any one of 85%, 90%, 95%, 98% or more sequence homology, or about 100% sequence identity) to SEQ ID NO: 41.

In some embodiments according to any one of the compositions, delivery systems, or kits described above, the Ago protein comprises a nuclear localization signal (NLS).

In one aspect of the present application, there is provided a cell comprising any one of the compositions described above.

In one aspect of the present application, there is provided a kit comprising any one of the compositions or delivery systems described above, and instructions for modifying a target nucleic acid using the kit, wherein the target nucleic acid comprises the target locus.

These and other aspects and advantages of the present invention will become apparent from the subsequent detailed description and the appended claims. It is to be understood that one, some, or all of the properties of the various embodiments described herein may be combined to form other embodiments of the present invention.

### BRIEF DESCRIPTION OF THE DRAWINGS

FIG. 1 depicts a schematic design of pACYCDuet-eGFP plasmid, target site, and guide DNAs used in in vitro plasmid cleavage assays. The FW and RV guide DNAs are aligned with the target site sequences in the plasmid. A sequence of the 5′-phosphorylated NC (non-specific control) guide DNA is also shown.

FIG. 2A depicts electrophoresis results of target pACYCDuet-eGFP plasmid after incubation with NgAgo-FW gDNA complex purified from 293T cells for 4 h, 8 h or 72 h.

FIG. 2B depicts electrophoresis results of incubation mixture of target pACYCDuet-eGFP plasmid with NgAgo-FW gDNA complex for 8 hours at different temperatures.

FIG. 2C depicts exemplary sequences and a representative sequencing chromatogram of plasmid cleavage products by a NgAgo-gDNA complex.

FIG. 3A depicts the electrophoresis results of a linearized pACYCDuet-eGFP DNA incubated with or without a purified NgAgo-gDNA complex.

FIG. 3B depicts electrophoresis results of an 86 nucleotide ssDNA co-incubated with or without a purified NgAgo-gDNA complex at 37° C. for 8 hours.

FIG. 4A depicts electrophoresis results of incubation mixture of target pACYCDuet-cGFP plasmid with NaAgo-FW gDNA complex for 8 hours at different temperatures.

FIG. 4B depicts electrophoresis results of incubation mixture of target pACYCDuet-eGFP plasmid with NtAgo-FW gDNA complex for 8 hours at different temperatures.

FIG. 4C depicts electrophoresis results of incubation mixture of target pACYCDuet-eGFP plasmid with MaAgo-FW gDNA complex for 8 hours at different temperatures.

FIG. 4D depicts electrophoresis results of incubation mixture of target pACYCDuet-eGFP plasmid with SyAgo-FW gDNA complex for 8 hours at different temperatures.

FIG. 5 depicts a schematic of NgAgo-6p-1 plasmid.

FIG. 6 depicts electrophoresis results of nucleic acids co-purified with GST-NgAgo expressed in E. coli.

FIG. 7A depicts electrophoresis results of an in vitro plasmid cleavage assay using NgAgo re-loaded with various ssDNA guides to target pACYCDuet-eGFP.

FIG. 7B depicts electrophoresis results of an in vitro plasmid cleavage assay using NgAgo re-loaded with a ssDNA guide at 37° C. or 55° C. for 1 hour or 72 hours. The guide reloading process at 55° C. impairs endonuclease activity of NgAgo.

FIG. 7C depicts electrophoresis results of an in vitro plasmid cleavage assay using indicated guide nucleic acids with or without 5′ phosphorylation.

FIG. 8A depicts electrophoresis of nucleic acids bound to NgAgo, in which an NgAgo-encoding plasmid was transfected into 293T cells with or without simultaneous transfection of the indicated 24-nt nucleic acids.

FIG. 8B depicts electrophoresis of nucleic acids bound to NgAgo, in which an NgAgo-encoding plasmid was transfected into 293T cells with subsequent transfection of a 5′ phosphorylated ssDNA guide (FW).

FIG. 8C depicts electrophoresis of nucleic acids bound to NgAgo expressed in 293T cells, extracted after 48 hours of expression, and incubated with a 5′ phosphorylated ssDNA guide at 55° C. for 1 h or at 37° C. for 8 h.

FIG. 8D depicts electrophoresis results of an in vitro plasmid cleavage assay by NgAgo purified from 293T cells. In some conditions, the 293T cells were co-transfected by an NgAgo-encoding plasmid with target-complementary guide (FW), or a random guide (NC). In some conditions, the purified NgAgo or NgAgo-gDNA complex was subsequently co-incubated with target-complementary guide (FW), or a random guide (NC).

FIG. 9 depicts a schematic cartoon of the “one-guide-faithful” rule followed by NgAgo. When an NgAgo expression plasmid is transfected into mammalian cells to express NgAgo protein, the NgAgo is not active for cleavage when co-incubated with FW guide DNA (top). When an NgAgo expression plasmid is co-transfected into mammalian cells with phosphorylated or unphosphorylated ssDNA or ssRNA guides (FW guide), only the 5′ phosphorylated ssDNA guide is loaded onto NgAgo to allow cleavage (middle). When an NgAgo expression plasmid is co-transfected with a nonspecific guide (NC guide), the NgAgo-gDNA complex is unable to later load a second, specific guide (FW guide) to activate cleavage activity (bottom).

FIG. 10 depicts NgAgo directed to the nuclei of HeLa cells by the nuclear localization signal (NLS). Scale bar=100 μm. Images are representative of 20 independent experiments.

FIG. 11A depicts a schematic design of target plasmid pEGFP-N1, with target sites of various gDNAs within the CMV promoter and eGFP gene indicated.

FIG. 11B shows the hybridization sites of ssDNA guides for NgAgo and sgRNAs for Cas9 on the target plasmid pEGFP-N1.

FIG. 11C depicts Western blot analysis of eGFP expression in HeLa cells transfected with target plasmid pEGFP-N1 together with either the NgAgo-expressing plasmid and the indicated ssDNA guides, or Cas9-expressing plasmid and the indicated sgRNA transcription vectors. The results are representative of three independent experiments. Corresponding semi-quantitative results based on densitometry ratios between eGFP and actin bands are shown in the bar graph below on the left. The bar graph on the right shows statistical analysis of fold changes in the GFP expression level caused by the NgAgo-gDNA system vs. the Cas9-sgRNA system (*** denotes p<0.01).

FIG. 11D depicts Western blot analysis of eGFP expression in HeLa cells co-transfected with target plasmid pEGFP-N1, the NgAgo-encoding plasmid and the G3(n) guides of various lengths. Blot is representative of three independent experiments.

FIG. 12 depicts Western blot analysis of eGFP expression in HeLa cells co-transfected with target plasmid pEGFP-N1, various plasmids encoding Ago from different species, and G3 gDNA.

FIG. 13A depicts a schematic design of the target locus in exon 11 of the human DYRK1A gene, and the corresponding guide DNAs.

FIG. 13B depicts T7EI assay results showing NgAgo-gDNA induced double-strand breaks in a DYRK1A locus.

FIG. 13C depicts an example chromatogram showing microdeletion D10 in the human DYRK1A gene (deletion site marked with an apostrophe), and representative sequences of mutated alleles identified from clonal amplicons using the G10 guide. The WT sequence is listed at the top, with the G10 guide DNA target sequence underlined. NgAgo-ssDNA resulted in deletions (D10, D17, D25, and D32), mutations (M1), and insertions (+1, +2). Sequence names are listed to the left and are named according to the type of sequence alterations (D, deletions; M, mutations; +, insertions), followed by the number of nucleotides altered.

FIG. 14A depicts a schematic experimental design showing the NgAgo/gDNA guides/target and Cas9/gRNA guides/target positions, T7EI cleavage positions and predicted PCR product lengths.

FIG. 14B depicts T7EI assay results showing predicted cleavage products by NgAgo-gDNA (G5 and G10) and Cas9-sgRNA(sg-DYRK1A).

FIG. 15A depicts T7EI assay results showing NgAgo-gDNA induced double-strand breaks at the indicated target genes in the human genome.

FIG. 15B shows a representative blot of three independent T7EI experiments showing cleavage of HBA2, GATA4, GRIN2B, HRES1, and APOE by NgAgo-gDNA systems.

FIG. 15C depicts cleavage efficiencies by NgAgo-gDNA (G5-G18) in the DYRK1A loci as determined by the T7EI assay.

FIG. 15D depicts cleavage efficiencies by NgAgo-gDNA (G19-G26) in the ACTIN loci as determined by the T7EI assay.

FIG. 15E depicts cleavage efficiencies by NgAgo-gDNA (G27-G36) in the EMX1 loci as determined by the T7EI assay.

FIG. 16 depicts T7EI assay results showing NgAgo-gDNA induced double-strand breaks in the DYRK1A locus targeted by G10 in various mammalian cell lines.

FIG. 17A depicts T7EI assay results showing NgAgo-gDNA induced double-strand breaks in the DYRK1A locus using modified 24-nt long G10 guides having mismatches.

FIG. 17B depicts T7EI assay results showing NgAgo-gDNA induced double-strand breaks in the DYRK1A locus using modified 21-nt long G10 guides having mismatches.

FIG. 18A depicts the sequence of a GC-rich human HBA2 locus (top) and GA TA44 locus (bottom) aligned with corresponding guide DNAs and sgRNAs.

FIG. 18B depicts T7EI assay results comparing the cleavage efficiencies between NgAgo-gDNA system and Cas9-sgRNA system in GC-rich loci of human HBA2 and GA TA4 genes.

FIG. 19A depicts a schematic design of HDR-mediated donor DNA insertion initiated by NgAgo-gDNA. PCR primer regions are indicated with arrows.

FIG. 19B depicts sequence chromatograms of genomic PCR amplicons indicating successful HDR-mediated donor DNA insertion into the targeted genome locus.

FIG. 19C depicts a schematic design of NHEJ-mediated mutation initiated by NgAgo-gDNA. A promoter (arrow) drives expression of mRFP, which is followed by a target site and a stop codon. eGFP is out of frame and not expressed (top). Following NgAgo-gDNA-induced DSBs and NHEJ-mediated frameshift mutation, eGFP is in-frame and is expressed (bottom).

FIG. 19D depicts flow cytometry analysis of mRFP-TGA-eGFP integrated cells transfected with an empty vector, an NgAgo-encoding plasmid, G52 gDNA, or the NgAgo-encoding plasmid and G52 gDNA. Results are representative of three independent experiments.

FIG. 20A depicts a schematic design of an experiment investigating off-target genome editing effects by NgAgo-gDNA or Cas9-sgRNA.

FIG. 20B depicts a Southern blot showing off-target editing by Cas9 but not by NgAgo.

FIG. 21A depicts a schematic experimental design for inserting an cGFP donor DNA into the DYRK1A locus initiated by NgAgo-gDNA. Primer binding positions and G10 site are indicated with arrows. Expected products include indels, and insertion of the eGFP donor DNA in the inverse direction, which do not express GFP, as well as the correct insertion construct that places the eGFP-encoding sequence in frame with the DYRK1A gene thereby allowing expression of GFP. The correct donor-DNA integrated locus might contain a mutation in the G10 site (G10′ having a box to annotate the mutation) as a result of NgAgo-gDNA cleavage and nucleotide removal of the G10 site.

FIG. 21B depicts a sequencing chromatogram of a PCR amplicon of a modified DYRK1A locus close to the upstream primer dy2.

FIG. 21C depicts a sequencing chromatogram of a PCR amplicon of a modified DYRK1A locus close to the junction between the DYRK1A locus and inserted eGFP. The top figure shows a mutation in the G10 site in the modified DYRK1A locus.

FIG. 21D depicts a sequencing chromatogram of a PCR amplification product of a modified DYRK1A locus close to the downstream primer g2r.

FIG. 21E shows microscopy images of cells after co-transfection with NgAgo-gDNA and eGFP donor DNA. Left panel shows a phase-contrast microscopy image. Right Panel shows a green-channel fluorescence microscopy image.

FIG. 22A shows microscopy images of cells after knock-in of eGFP donor DNA into a beta-ACTIN locus mediated by NgAgo-gDNA. Left panel shows a green-channel fluorescence microscopy image, indicating expression of the engineered protein product. Right panel shows a red-channel fluorescence microscopy image, in which F-Actin in the cells was stained with a TRITC-phalloidine dye.

FIG. 22B shows microscopy images of cells after knock-in of eGFP donor DNA into a beta-ACTIN locus mediated by NgAgo-gDNA. Left panel shows a blue-channel fluorescence microscopy image, in which cell nuclei were stained using DAPI. Middle panel shows an overlay of the microscopy images of the cells in the green channel, red channel, and blue channel. Right panel shows a phase-contrast microscopy image.

FIG. 22C shows a sequencing chromatogram of a PCR amplicon of a modified beta-ACTIN locus close to the junction between the beta-ACTIN locus and inserted eGFP.

FIG. 23A shows microscopy images of cells after knock-in of eGFP donor DNA into the DYRK1A locus mediated by PhAgo-gDNA. Left panel shows a phase-contrast microscopy image. Right Panel shows a green-channel fluorescence microscopy image.

FIG. 23B shows microscopy images of cells after knock-in of eGFP donor DNA into the beta-ACTIN locus mediated by PhAgo-gDNA. Top left panel shows a green-channel fluorescence microscopy image, indicating expression of the engineered protein product. Top right panel shows a red-channel fluorescence microscopy image, in which F-Actin in the cells was stained with a TRITC-phalloidine dye. Bottom top panel shows a blue-channel fluorescence microscopy image, in which cell nuclei were stained using DAPI. Bottom right panel shows a phase-contrast microscopy image.

FIG. 23C shows microscopy images of cells after knock-in of eGFP donor DNA into the DYRK1A locus mediated by MiAgo-gDNA. Left panel shows a phase-contrast microscopy image. Right Panel shows a green-channel fluorescence microscopy image.

FIG. 23D shows microscopy images of cells after knock-in of eGFP donor DNA into the DYRK1A locus mediated by MaAgo-gDNA. Left panel shows a phase-contrast microscopy image. Right Panel shows a green-channel fluorescence microscopy image.

FIG. 24A shows sequence alignment of Ago proteins from different species around the KQK motif in the 5′ phosphate binding site. Conserved amino acid residues KQK are highlighted.

FIG. 24B shows sequence alignment of Ago proteins from different species around the DDE motif in the nuclease active site. Conserved amino acid residues DDE are highlighted.

FIG. 25 depicts a phylogenetic tree of Ago proteins from different species. Percentages shown are pairwise sequence homology between consensus sequences of two branches. Agos marked with an asterisk (*) are unable to cleave target nucleic acids when transfected into mammalian cells together with a gDNA.

FIG. 26A depicts degradation of a target DNA genome by NgAgo-gDNA expressed in bacterial hosts.

FIG. 26B depicts degradation of a linearized PGEX-6P-1 vector by Ago-gDNA in vitro. The Ago-gDNA complexes were purified from E. coli cells transformed with PGEX-6P-1 plasmids containing an NgAgo, MiAgo, or MaAgo expression cassette having a GST tag.

### DETAILED DESCRIPTION OF THE INVENTION

The present invention provides methods, delivery systems, compositions and kits for modifying a target nucleic acid using an Argonaute (Ago) and a guide DNA (gDNA). Some embodiments of the present application provide Ago-based gene or genome editing methods. The present invention is based on the discovery that certain Ago proteins (such as Ago from Natronobacterium gregoryi, also referred herein as “NgAgo”) can be guided by a single-stranded oligonucleotide DNA (i.e., gDNA) to specifically recognize and modify a target locus having a complementary sequence to the gDNA at a physiological temperature. In some embodiments, the Ago-gDNA complex induces a double-strand break (DSB) in the target DNA locus, thereby allowing gene editing at the DSB using cellular DSB repair machinery. In some embodiments, no DNA cleavage is induced. The Ago-gDNA methods described herein are characterized by high specificity, and wide applicability to target loci of various sequences, including sequences with high GC contents. The high specificity of the methods described herein is contributed by several features of the Ago-gDNA systems. In some embodiments, the Ago protein has a low tolerance to mismatch (such as up to 3 mismatches) between the gDNA and the target locus. In some embodiments, a single mismatch between the gDNA and the target locus results in significantly reduced cleavage efficiency by the Ago-gDNA complex. In some embodiments, the guide DNA is 5′ phosphorylated. As 5′ phosphorylated short ssDNAs are rare in mammalian cells, use of such Ago-gDNA systems for modifying target nucleic acids in mammalian cells minimizes nonspecific modifications due to misguiding of the Ago protein by cellular oligonucleotides. In some embodiments, the guide DNA can only be loaded into the Ago-gDNA complex once, e.g., during the expression of the Ago protein, and once loaded, the Ago protein cannot swap its gDNA with another free ssDNA at a physiological temperature (such as 37° C.). This feature, which is also referred herein as “one-guide-faithful” rule, can further reduce off-target effects. Additionally, compared to guide RNAs used in other gene-editing or silencing systems, ssDNA guides of the present methods can be easily designed and prepared, as the Ago proteins do not have any special sequence or secondary structure requirements for gDNAs. Transfection of gDNA into cells is simple, and the dose of gDNA for transfection is adjustable. A variety of modifications to the target nucleic acids can be achieved, including, but not limited to, site-specific cleavage, indels, knock-out, knock-in, substitutions (such as single-nucleotide substitutions), and alteration of gene expression.

Accordingly, one aspect of the present application provides a method of modifying a target nucleic acid, comprising contacting the target nucleic acid with an Argonaute (Ago) protein and a single-stranded guide DNA at a temperature of about 10° C. to about 60° C. (such as about 37° C.), wherein the Ago protein and the guide DNA form a complex that specifically recognizes a target locus in the target nucleic acid, wherein the target locus comprises a sequence that is complementary to the sequence of the guide DNA. In some embodiments, the complex cleaves the target locus. In some embodiments, wherein the target locus is a double-stranded DNA, the Ago protein induces a double-strand break in the target locus.

One aspect of the present application provides a composition comprising a complex comprising an Ago protein and a single-stranded guide DNA, wherein the complex is capable of specifically recognizing a target locus at a temperature of about 10° C. to about 60° C. (such as about 37° C.), and wherein the target locus comprises a sequence that is complementary to the sequence of the guide DNA. In some embodiments, the Ago protein is capable of cleaving the target locus. In some embodiments, wherein the target locus is a double-stranded DNA, the Ago protein is capable of inducing a double-strand break in the target locus.

One aspect of the present application provides a delivery system comprising a complex comprising an Ago protein and a single-stranded guide DNA, and a vehicle suitable for intracellular delivery of the complex, wherein the complex is capable of specifically recognizing a target locus at a temperature of about 10° C. to about 60° C. (such as about 37° C.), and wherein the target locus comprises a sequence that is complementary to the sequence of the guide DNA. In some embodiments, the Ago protein is capable of cleaving the target locus. In some embodiments, wherein the target locus is a double-stranded DNA, the Ago protein is capable of inducing a double-strand break in the target locus.

One aspect of the present application provides a kit comprising a nucleic acid encoding an Ago protein and a single-stranded guide DNA, wherein the complex is capable of specifically recognizing a target locus at a temperature of about 10° C. to about 60° C. (such as about 37° C.), and wherein the target locus comprises a sequence that is complementary to the sequence of the guide DNA. In some embodiments, the Ago protein is capable of cleaving the target locus. In some embodiments, wherein the target locus is a double-stranded DNA, the Ago protein is capable of inducing a double-strand break in the target locus.

Also provided are kits and articles manufacture useful for the methods described herein.

### I. Definitions

Unless otherwise defined, scientific and technical terms used in connection with the present invention shall have the meanings that are commonly understood by those of ordinary skill in the art. Further, unless otherwise required by context or expressly indicated, singular terms shall include pluralities and plural terms shall include the singular.

As used herein, “Argonaute” and “Ago” are used interchangeable and refer to a naturally occurring or engineered protein that can be guided by a single-stranded oligonucleotide DNA (i.e., guide DNA) to specifically recognize a target nucleic acid comprising a complementary sequence to the guide DNA. Some Ago proteins, also referred herein as “Argonaute nucleases,” have DNA-guided endonuclease activity, i.e. cleavage of an internal phosphodiester bond in a target nucleic acid. Some Ago proteins do not cleave the target nucleic acid.

As used herein, “guide DNA”, “gDNA”, or “DNA guide” are used interchangeably to refer to a single-stranded oligonucleotide DNA that can form a complex with an Argonaute protein of the present application and hybridize to a target nucleic acid. The portion of the target nucleic acid that hybridizes to the guide DNA is referred herein interchangeably as the “target locus” or “target site.” The complex of an Ago protein bound to a guide DNA is referred herein as “Ago-gDNA” or “Ago-G.”

As used herein, “donor DNA” refers to a polynucleotide that can be integrated into the site of a double-strand break induced by an Ago-gDNA complex.

The terms “nucleic acid,” “polynucleotide,” and “nucleotide sequence” are used interchangeably to refer to a polymeric form of nucleotides of any length, including deoxyribonucleotides, ribonucleotides, combinations thereof, and analogs thereof. “Oligonucleotide” and “oligo” are used interchangeably to refer to a short polynucleotide, having no more than about 50 nucleotides.

“Complementarity” refers to the ability of a nucleic acid to form hydrogen bond(s) with another nucleic acid by either traditional Watson-Crick or other non-traditional types. A percent complementarity indicates the percentage of residues in a nucleic acid molecule which can form hydrogen bonds (e.g., Watson-Crick base pairing) with a second nucleic acid (e.g., about 5, 6, 7, 8, 9, 10 out of 10, being about 50%, 60%, 70%, 80%, 90%, and 100% complementary respectively). “Perfectly complementary” means that all the contiguous residues of a nucleic acid sequence hydrogen bond with the same number of contiguous residues in a second nucleic acid sequence. “Substantially complementary” as used herein refers to a degree of complementarity that is at least about any one of 60%, 65%, 70%, 75%, 80%, 85%, 90%, 95%, 97%, 98%, 99%, or 100% over a region of about 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

“Mismatch” refers to a nucleotide in a first nucleic acid that does not form a traditional Watson-Crick basepair with a corresponding nucleotide in a second nucleic acid.

As used herein, “target” or “targeting” refers to specific binding of a gDNA or an Ago-gDNA complex to a nucleic acid. “Specific binding” refers to hybridization under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N,Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

“Percentage (%) sequence identity” with respect to a peptide, polypeptide or protein sequence is defined as the percentage of amino acid residues in a candidate sequence that are identical with the amino acid residues in the specific peptide or polypeptide sequence, after aligning the sequences and introducing gaps, if necessary, to achieve the maximum percent sequence identity. “Percentage (%) sequence homology” with respect to a peptide, polypeptide or protein sequence is the percentage of amino acid residues in a candidate sequence that are identical or conservative substitutions to amino acid residues in the specific peptide or polypeptide sequence, after aligning the sequences and introducing gaps, if necessary, to achieve the maximum percent sequence homology. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The terms “polypeptide”, and “peptide” are used interchangeably herein to refer to polymers of amino acids of any length. The polymer may be linear or branched, it may comprise modified amino acids, and it may be interrupted by non-amino acids. A protein may have one or more polypeptides. The terms also encompass an amino acid polymer that has been modified; for example, disulfide bond formation, glycosylation, lipidation, acetylation, phosphorylation, or any other manipulation, such as conjugation with a labeling component.

The terms “transfect,” “transform,” and “deliver” are used interchangeably herein to refer to a process by which exogenous molecules (such as nucleic acids, proteins, or complexes thereof) are transferred or introduced into a cell.

The term “cell” includes the primary subject cell and its progeny. The terms “host cell” refers to cells into which exogenous nucleic acids or protein complexes (such as Ago-gDNA complex) have been introduced, including the progeny of such cells. Cells and host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, the term “isolated” can refer to a nucleic acid or polypeptide that, by the hand of a human, exists apart from its native environment and is therefore not a product of nature. An isolated nucleic acid or polypeptide can exist in a purified form and/or can exist in a non-native environment such as, for example, in a transgenic cell.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter.

The term “about X-Y” used herein has the same meaning as “about X to about Y.”

As used herein, the singular forms “a,” “or,” and “the” include plural referents unless the context clearly dictates otherwise.

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Current Protocols in Molecular Biology or Current Protocols in Immunology, John Wiley & Sons, New York, N.Y. (2009): Ausubel et al, Short Protocols in Molecular Biology, 3^{rd} ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

### II. Methods of Modifying a Target Nucleic Acid

The present application provides methods of modifying a target nucleic acid using an Argonaute and a single-stranded guide DNA. The methods can be used for in vitro or intracellular gene editing, site-specific cleavage, gene silencing, and other nucleic acid modifications.

One aspect of the present application provides a method of modifying a target nucleic acid, comprising contacting the target nucleic acid with an Argonaute (Ago) protein and a single-stranded guide DNA at a temperature of about 10° C. to about 60° C. (such as about 37° C.), wherein the Ago protein and the guide DNA form a complex that specifically recognizes a target locus in the target nucleic acid, and wherein the target locus comprises a sequence that is complementary to the sequence of the guide DNA. In some embodiments, the complex does not cleave the target locus. In some embodiments, the Ago protein and the guide DNA are present in a pre-formed complex. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the guide DNA is phosphorylated at the 5′ terminus. In some embodiments, the guide DNA is about 10 to about 50 (such as about 20 to about 30) nucleotides long. In some embodiments, the contacting is in the presence of a divalent metal ion (such as at least about 0.1 mM).

In some embodiments, there is provided a method of modifying a target nucleic acid, comprising contacting the target nucleic acid with an Argonaute (Ago) protein and a single-stranded guide DNA at a temperature of about 10° C. to about 60° C. (such as about 37° C.), wherein the Ago protein and the guide DNA form a complex that specifically recognizes and cleaves a target locus in the target nucleic acid, and wherein the target locus comprises a sequence that is complementary to the sequence of the guide DNA. In some embodiments, the Ago protein and the guide DNA are present in a pre-formed complex. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the guide DNA is phosphorylated at the 5′ terminus. In some embodiments, the guide DNA is about 10 to about 50 (such as about 20 to about 30) nucleotides long. In some embodiments, the contacting is in the presence of a divalent metal ion (such as at least about 0.1 mM).

In some embodiments, there is provided a method of modifying a target nucleic acid, comprising contacting the target nucleic acid with an Argonaute (Ago) protein and a single-stranded guide DNA at a temperature of about 10° C. to about 60° C. (such as about 37° C.), wherein the Ago protein and the guide DNA form a complex that specifically recognizes a target locus in the target nucleic acid and induces a double-strand break (DSB) in the target locus, and wherein the target locus is a double-stranded DNA comprising a sequence that is complementary to the sequence of the guide DNA. In some embodiments, the Ago protein and the guide DNA are present in a pre-formed complex. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the guide DNA is phosphorylated at the 5′ terminus. In some embodiments, the guide DNA is about 10 to about 50 (such as about 20 to about 30) nucleotides long. In some embodiments, the contacting is in the presence of a divalent metal ion (such as at least about 0.1 mM).

In some embodiments, there is provided a method of modifying a target nucleic acid, comprising contacting the target nucleic acid with an Argonaute (Ago) protein and a 5′ phosphorylated single-stranded guide DNA at a temperature of about 10° C. to about 60° C. (such as about 37° C.), wherein the Ago protein and the guide DNA form a complex that specifically recognizes a target locus in the target nucleic acid, and wherein the target locus comprises a sequence that is complementary to the sequence of the guide DNA. In some embodiments, the complex does not cleave the target locus. In some embodiments, the Ago protein and the guide DNA are present in a pre-formed complex. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the guide DNA is about 10 to about 50 (such as about 20 to about 30) nucleotides long. In some embodiments, the contacting is in the presence of a divalent metal ion (such as at least about 0.1 mM).

In some embodiments, there is provided a method of modifying a target nucleic acid, comprising contacting the target nucleic acid with an Argonaute (Ago) protein and a 5′ phosphorylated single-stranded guide DNA at a temperature of about 10° C. to about 60° C. (such as about 37° C.), wherein the Ago protein and the guide DNA form a complex that specifically recognizes and cleaves a target locus in the target nucleic acid, and wherein the target locus comprises a sequence that is complementary to the sequence of the guide DNA. In some embodiments, the Ago protein and the guide DNA are present in a pre-formed complex. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the guide DNA is about 10 to about 50 (such as about 20 to about 30) nucleotides long. In some embodiments, the contacting is in the presence of a divalent metal ion (such as at least about 0.1 mM).

In some embodiments, there is provided a method of modifying a target nucleic acid, comprising contacting the target nucleic acid with an Argonaute (Ago) protein and a 5′ phosphorylated single-stranded guide DNA at a temperature of about 10° C. to about 60° C. (such as about 37° C.), wherein the Ago protein and the guide DNA form a complex that specifically recognizes a target locus in the target nucleic acid and induces a double-strand break (DSB) in the target locus, and wherein the target locus is a double-stranded DNA comprising a sequence that is complementary to the sequence of the guide DNA. In some embodiments, the Ago protein and the guide DNA are present in a pre-formed complex. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the guide DNA is about 10 to about 50 (such as about 20 to about 30) nucleotides long. In some embodiments, the contacting is in the presence of a divalent metal ion (such as at least about 0.1 mM).

In some embodiments, there is provided a method of modifying a target nucleic acid, comprising contacting the target nucleic acid with an Argonaute (Ago) protein and a 5′ phosphorylated single-stranded guide DNA at a temperature of about 10° C. to about 60° C. (such as about 37° C.), wherein the Ago protein and the guide DNA form a complex that specifically recognizes a target locus in the target nucleic acid, wherein the target locus comprises a sequence that is complementary to the sequence of the guide DNA, and wherein the Ago protein is derived from an organism selected from the group consisting of Natronobacterium gregoryi, Microcyslis aeruginosa, Halogeometricum pallidum, Natrialba asiatica, Natronorubrum tibetense, Natrinema pellirubrum, Halogeometricum borinquense, Thermococcus barophilus, Thermosynechococcus elongatus, Halorubrum lacusprofundi, Microcyslis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolyticus, Aromatoleum aromaticum, Thermococcus onnurineus, Methanopyrus kandleri, Synechococcus elongatus, Anoxybacillus flavithermus, Exiguobacterium sp., Lyngbya sp., Clostridium butyricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the complex cleaves the target locus. In some embodiments, the complex does not cleave the target locus. In some embodiments, the Ago protein and the guide DNA are present in a pre-formed complex. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the contacting is in the presence of a divalent metal ion (such as at least about 0.1 mM).

In some embodiments, there is provided a method of modifying a target nucleic acid, comprising contacting the target nucleic acid with an Argonaute (Ago) protein and a 5′ phosphorylated single-stranded guide DNA at a temperature of about 10° C. to about 60° C. (such as about 37° C.), wherein the Ago protein and the guide DNA form a complex that specifically recognizes a target locus in the target nucleic acid and induces a double-strand break (DSB) in the target locus, wherein the target locus is a double-stranded DNA comprising a sequence that is complementary to the sequence of the guide DNA, and wherein the Ago protein is derived from an organism selected from the group consisting of Natronobacterium gregoryi, Microcyslis aeruginosa, Halogeometricum pallidum, Natrialba asialica, Natronorubrum tibetense, Natrinema pellirubrum, Halogeometricum borinquense, Thermococcus barophilus, Thermosynechococcus elongatus, Halorubrum lacusprofundi, Microcystis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolvticus, Aromatoleum aromaticum, Thermococcus onnurineus, Methanopyrus kandleri, Synechococcus elongatus. Anoxybacillus flavithermus, Exiguobacterium sp., Lyngbya sp., Clostridium butyricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the Ago protein and the guide DNA are present in a pre-formed complex. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the contacting is in the presence of a divalent metal ion (such as at least about 0.1 mM).

In some embodiments, there is provided a method of modifying a target nucleic acid, comprising contacting the target nucleic acid with an Argonaute (Ago) protein and a 5′ phosphorylated single-stranded guide DNA at a temperature of about 10° C. to about 60° C. (such as about 37° C.), wherein the Ago protein and the guide DNA form a complex that specifically recognizes a target locus in the target nucleic acid, wherein the target locus comprises a sequence that is complementary to the sequence of the guide DNA, and wherein the Ago protein comprises an amino acid sequence having at least about 80% sequence homology (such as at least about any of 85%, 90%, 95%, 98%, 99% or more sequence homology, or about 100% sequence identity) to a sequence selected from the group consisting of SEQ ID NOs: 1-42. In some embodiments, the complex cleaves the target locus. In some embodiments, the complex does not cleave the target locus. In some embodiments, the Ago protein and the guide DNA are present in a pre-formed complex. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the contacting is in the presence of a divalent metal ion (such as at least about 0.1 mM).

In some embodiments, there is provided a method of modifying a target nucleic acid, comprising contacting the target nucleic acid with an Argonaute (Ago) protein and a 5′ phosphorylated single-stranded guide DNA at a temperature of about 10° C. to about 60° C. (such as about 37° C.), wherein the Ago protein and the guide DNA form a complex that specifically recognizes a target locus in the target nucleic acid and induces a double-strand break (DSB) in the target locus, wherein the target locus is a double-stranded DNA comprising a sequence that is complementary to the sequence of the guide DNA, and wherein the Ago protein comprises an amino acid sequence having at least about 80% sequence homology (such as at least about any of 85%, 90%, 95%, 98%, 99% or more sequence homology, or about 100% sequence identity) to a sequence selected from the group consisting of SEQ ID NOs: 1-42. In some embodiments, the Ago protein and the guide DNA are present in a pre-formed complex. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the contacting is in the presence of a divalent metal ion (such as at least about 0.1 mM).

In some embodiments, there is provided a method of modifying a target nucleic acid, comprising contacting the target nucleic acid with an Argonaute (Ago) protein and a 5′ phosphorylated single-stranded guide DNA at a temperature of about 10° C. to about 60° C. (such as about 37° C.), wherein the Ago protein and the guide DNA form a complex that specifically recognizes a target locus in the target nucleic acid, wherein the target locus comprises a sequence that is complementary to the sequence of the guide DNA, and wherein the Ago protein is derived from Natronobacterium gregoryi. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 1. In some embodiments, the complex cleaves the target locus. In some embodiments, the complex does not cleave the target locus. In some embodiments, the Ago protein and the guide DNA are present in a pre-formed complex. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the contacting is in the presence of a divalent metal ion (such as at least about 0.1 mM).

In some embodiments, there is provided a method of modifying a target nucleic acid, comprising contacting the target nucleic acid with an Argonaute (Ago) protein and a 5′ phosphorylated single-stranded guide DNA at a temperature of about 10° C. to about 60° C. (such as about 37° C.), wherein the Ago protein and the guide DNA form a complex that specifically recognizes a target locus in the target nucleic acid and induces a double-strand break (DSB) in the target locus, wherein the target locus is a double-stranded DNA comprising a sequence that is complementary to the sequence of the guide DNA, and wherein the Ago protein is derived from Natronobacterium gregoryi. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 1. In some embodiments, the Ago protein and the guide DNA are present in a pre-formed complex. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the contacting is in the presence of a divalent metal ion (such as at least about 0.1 mM).

In some embodiments, there is provided a method of modifying a target nucleic acid, comprising contacting the target nucleic acid with an Argonaute (Ago) protein and a 5′ phosphorylated single-stranded guide DNA at a temperature of about 10° C. to about 60° C. (such as about 37° C.), wherein the Ago protein and the guide DNA form a complex that specifically recognizes a target locus in the target nucleic acid, wherein the target locus comprises a sequence that is complementary to the sequence of the guide DNA, and wherein the Ago protein is derived from Microcystis aeruginosa. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 2. In some embodiments, the complex cleaves the target locus. In some embodiments, the complex does not cleave the target locus. In some embodiments, the Ago protein and the guide DNA are present in a pre-formed complex. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the contacting is in the presence of a divalent metal ion (such as at least about 0.1 mM).

In some embodiments, there is provided a method of modifying a target nucleic acid, comprising contacting the target nucleic acid with an Argonaute (Ago) protein and a 5′ phosphorylated single-stranded guide DNA at a temperature of about 10° C. to about 60° C. (such as about 37° C.), wherein the Ago protein and the guide DNA form a complex that specifically recognizes a target locus in the target nucleic acid and induces a double-strand break (DSB) in the target locus, wherein the target locus is a double-stranded DNA comprising a sequence that is complementary to the sequence of the guide DNA, and wherein the Ago protein is derived from Microcystis aeruginosa. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 2. In some embodiments, the Ago protein and the guide DNA are present in a pre-formed complex. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the contacting is in the presence of a divalent metal ion (such as at least about 0.1 mM).

In some embodiments, there is provided a method of modifying a target nucleic acid, comprising contacting the target nucleic acid with an Argonaute (Ago) protein and a 5′ phosphorylated single-stranded guide DNA at a temperature of about 10° C. to about 60° C. (such as about 37° C.), wherein the Ago protein and the guide DNA form a complex that specifically recognizes a target locus in the target nucleic acid, wherein the target locus comprises a sequence that is complementary to the sequence of the guide DNA, and wherein the Ago protein is derived from Microcystis sp. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 11. In some embodiments, the complex cleaves the target locus. In some embodiments, the complex does not cleave the target locus. In some embodiments, the Ago protein and the guide DNA are present in a pre-formed complex. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the contacting is in the presence of a divalent metal ion (such as at least about 0.1 mM).

In some embodiments, there is provided a method of modifying a target nucleic acid, comprising contacting the target nucleic acid with an Argonaute (Ago) protein and a 5′ phosphorylated single-stranded guide DNA at a temperature of about 10° C. to about 60° C. (such as about 37° C.), wherein the Ago protein and the guide DNA form a complex that specifically recognizes a target locus in the target nucleic acid and induces a double-strand break (DSB) in the target locus, wherein the target locus is a double-stranded DNA comprising a sequence that is complementary to the sequence of the guide DNA, and wherein the Ago protein is derived from Microcyslis sp. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 11. In some embodiments, the Ago protein and the guide DNA are present in a pre-formed complex. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the contacting is in the presence of a divalent metal ion (such as at least about 0.1 mM).

In some embodiments, there is provided a method of modifying a target nucleic acid, comprising contacting the target nucleic acid with an Argonaute (Ago) protein and a 5′ phosphorylated single-stranded guide DNA at a temperature of about 10° C. to about 60° C. (such as about 37° C.), wherein the Ago protein and the guide DNA form a complex that specifically recognizes a target locus in the target nucleic acid, wherein the target locus comprises a sequence that is complementary to the sequence of the guide DNA, and wherein the Ago protein is derived from Pedobacter heparinus. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 41. In some embodiments, the complex cleaves the target locus. In some embodiments, the complex does not cleave the target locus. In some embodiments, the Ago protein and the guide DNA are present in a pre-formed complex. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the contacting is in the presence of a divalent metal ion (such as at least about 0.1 mM).

In some embodiments, there is provided a method of modifying a target nucleic acid, comprising contacting the target nucleic acid with an Argonaute (Ago) protein and a 5′ phosphorylated single-stranded guide DNA at a temperature of about 10° C. to about 60° C. (such as about 37° C.), wherein the Ago protein and the guide DNA form a complex that specifically recognizes a target locus in the target nucleic acid and induces a double-strand break (DSB) in the target locus, wherein the target locus is a double-stranded DNA comprising a sequence that is complementary to the sequence of the guide DNA, and wherein the Ago protein is derived from Pedobacter heparinus. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 41. In some embodiments, the Ago protein and the guide DNA are present in a pre-formed complex. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the contacting is in the presence of a divalent metal ion (such as at least about 0.1 mM).

The methods described herein can be used for modifying target nucleic acids both in vitro and in cells. In some embodiments, the target nucleic acid is an isolated DNA. In some embodiments, the target nucleic acid is a plasmid. In some embodiments, the target nucleic acid is an isolated nucleic acid comprising a double-stranded DNA region. The Argonaute nucleases described herein can be used to introduce a site-specific cleavage in the target nucleic acid, such as double-stranded DNA, in vitro. The cleavage site is within the region of the target nucleic acid that the guide DNA hybridizes to (i.e., “target locus”). Notably, the Argonaute nucleases described herein can use a single guide DNA to induce double-strand breaks in a double-stranded DNA, such as a plasmid. A guide DNA having a sequence complementary to, such as perfectly complementary to or substantially complementary to, the sequence of the target site can be used to induce a double-strand break at the target site in the target nucleic acid.

Restriction enzymes are normally used for site-specific cleavage of nucleic acids in vitro. Unlike restriction enzymes, the Argonaute nucleases do not require the cleavage site to contain a palindromic sequence. As the Argonaute nucleases described herein have no sequence preference, by designing a guide DNA with a suitable sequence, the methods described herein can be used to cleave any site, including GC-rich sites, in a target nucleic acid. Also, because the length of the guide DNA is longer than that of the recognition sequence, the Ago-gDNA based methods for site-specific cleavage has higher specificity and lower off-target effects than restriction enzymes. Notably, the Argonaute nucleases described herein do not have exonuclease activities.

Thus, in some embodiments, there is provided a method of site-specific cleavage of a target nucleic acid (such as a plasmid) in vitro, comprising contacting the target nucleic acid with an Argonaute (Ago) protein and a single-stranded guide DNA (such as a 5′ phosphorylated singled-stranded guide DNA) at a temperature of about 10° C. to about 60° C. (such as about 37° C.), wherein the Ago protein and the guide DNA form a complex that specifically recognizes and cleaves a target locus in the target nucleic acid, and wherein the target locus comprises a sequence that is complementary to the sequence of the guide DNA. In some embodiments, wherein the target locus is a double-stranded DNA, the Ago protein induces a double-strand break in the target locus. In some embodiments, the Ago protein and the guide DNA are present in a pre-formed complex. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the Ago protein is derived from an organism selected from the group consisting of Natronobacterium gregoryi, Microcystis aeruginosa, Halogeometricum pallidum, Natrialba asiatica, Natronorubrum tibetense, Natrinema pellirubrum, Halogeometricum borinquense, Thermococcus barophilus, Thermosynechococcus elongatus, Halorubrum lacusprofundi, Microcystis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolyticus, Aromatoleum aromaticum, Thermococcus onnurineus, Methanopyrus kandleri, Synechococcus elongatus, Anoxybacillus flavithermus, Exiguobacterium sp., Lyngbya sp., Clostridium butyricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the Ago protein comprises an amino acid sequence having at least about 80% sequence homology (such as at least about any of 85%, 90%, 95%, 98%, 99% or more sequence homology, or about 100% sequence identity) to a sequence selected from the group consisting of SEQ ID NOs:1-42. In some embodiments, the guide DNA is about 10 to about 50 (such as about 20 to about 30) nucleotides long. In some embodiments, the contacting is in the presence of a divalent metal ion (such as at least about 0.1 mM).

In some embodiments, there is provided a method of site-specific cleavage of a target nucleic acid (such as plasmid) in vitro, comprising contacting the target nucleic acid with an Argonaute (Ago) protein and a 5′ phosphorylated single-stranded guide DNA at a temperature of about 10° C. to about 60° C. (such as about 37° C.), wherein the Ago protein and the guide DNA form a complex that specifically recognizes a target locus in the target nucleic acid and induces a double-strand break (DSB) in the target locus, wherein the target locus is a double-stranded DNA comprising a sequence that is complementary to the sequence of the guide DNA, and wherein the Ago protein is derived from Natronobacterium gregoryi. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 1. In some embodiments, the Ago protein and the guide DNA are present in a pre-formed complex. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the contacting is in the presence of a divalent metal ion (such as at least about 0.1 mM).

In some embodiments, the target nucleic acid is present in a cell. The target nucleic acid can be an endogenous nucleic acid (such as genomic DNA or RNA) in the cell, or an exogenous nucleic acid, such as a viral nucleic acid, in the cell. The methods described herein are compatible with a variety of cells, including both prokaryotic cells and eukaryotic cells. As the physiological temperatures of non-thermophilic species are in the range of about 10° C. to about 60° C., Argonaute nucleases previously known to have DNA-guided endonuclease activity, such as Argonautes from Thermus thermophilus, cannot cleave target nucleic acids inside live cells from non-thermophilic species. See, for example, WO2014/189628. Here, inventors of the present application identified Argonautes that can be guided by a single-stranded guide DNA to specifically recognize and/or cleave intracellular target nucleic acids at physiological temperatures (such as about 10° C. to about 60° C., or about 37° C.), thereby enabling the methods of modifying intracellular target nucleic acids described herein.

Thus, in some embodiments, there is provided a method of modifying of a target nucleic acid in a cell, comprising contacting the target nucleic acid with an Argonaute (Ago) protein and a single-stranded guide DNA (such as a 5′ phosphorylated singled-stranded guide DNA), wherein the Ago protein and the guide DNA form a complex that specifically recognizes a target locus in the target nucleic acid, and wherein the target locus comprises a sequence that is complementary to the sequence of the guide DNA. In some embodiments, the complex cleaves the target locus. In some embodiments, wherein the target locus is a double-stranded DNA, the Ago protein induces a double-strand break in the target locus. In some embodiments, the complex does not cleave the target locus. In some embodiments, the Ago protein and the guide DNA are present in a pre-formed complex. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the Ago protein is derived from an organism selected from the group consisting of Natronobacterium gregoryi, Microcystis aeruginosa, Halogeometricum pallidum, Natrialba asiatica, Natronorubrum tibetense, Natrinema pellirubrum, Halogeometricum borinquense, Thermococcus barophilus, Thermosynechococcus elongatus, Halorubrum lacusprofundi, Microcystis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolvticus, Aromatoleum aromaticum, Thermococcus onnurineus, Methanopyrus kandleri, Synechococcus elongalus, Anoxybacillus flavithermus, Exiguobacterium sp., Lyngbya sp., Clostridium butyricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the Ago protein comprises an amino acid sequence having at least about 80% sequence homology (such as at least about any of 85%, 90%, 95%, 98%, 99% or more sequence homology, or about 100% sequence identity) to a sequence selected from the group consisting of SEQ ID NOs: 1-42. In some embodiments, the guide DNA is about 10 to about 50 (such as about 20 to about 30) nucleotides long. In some embodiments, the cell is supplemented with a divalent metal ion (such as at least about 0.1 mM). In some embodiments, the nucleic acid encoding the Ago protein is codon-optimized for the organism from which the cell is derived. In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the target nucleic acid is endogenous to the cell. In some embodiments, the target nucleic acid is a genomic DNA. In some embodiments, the target nucleic acid is a RNA, such as an mRNA. In some embodiments, the target nucleic acid is exogenous to the cell. In some embodiments, the target nucleic acid is integrated in the genome of the cell. In some embodiments, the target nucleic acid is not integrated in the genome of the cell. In some embodiments, the target nucleic acid is a viral DNA. In some embodiments, the cell is a prokaryotic cell. In some embodiments, the cell is a eukaryotic cell, such as plant, fungal, yeast, or mammalian cell. In some embodiments, the cell is a human cell. In some embodiments, the cell is derived from a cell line. In some embodiments, the cell is a primary cell. In some embodiments, the cell is an immune cell.

In some embodiments, there is provided a method of modifying of a target nucleic acid in a cell, comprising contacting the target nucleic acid with an Argonaute (Ago) protein and a 5′ phosphorylated single-stranded guide DNA, wherein the Ago protein and the guide DNA form a complex that specifically recognizes a target locus in the target nucleic acid and induces a double-strand break (DSB) in the target locus, wherein the target locus is a double-stranded DNA comprising a sequence that is complementary to the sequence of the guide DNA, and wherein the Ago protein is derived from Natronobacterium gregoryi. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 1. In some embodiments, the Ago protein and the guide DNA are present in a pre-formed complex. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the cell is supplemented with a divalent metal ion (such as at least about 0.1 mM). In some embodiments, the nucleic acid encoding the Ago protein is codon-optimized for the organism from which the cell is derived. In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the target nucleic acid is endogenous to the cell. In some embodiments, the target nucleic acid is a genomic DNA. In some embodiments, the target nucleic acid is a RNA, such as an mRNA. In some embodiments, the target nucleic acid is exogenous to the cell. In some embodiments, the target nucleic acid is integrated in the genome of the cell. In some embodiments, the target nucleic acid is not integrated in the genome of the cell. In some embodiments, the target nucleic acid is a viral DNA. In some embodiments, the cell is a prokaryotic cell. In some embodiments, the cell is a eukaryotic cell, such as plant, fungal, yeast, or mammalian cell. In some embodiments, the cell is a human cell. In some embodiments, the cell is derived from a cell line. In some embodiments, the cell is a primary cell. In some embodiments, the cell is an immune cell.

The Ago protein and the guide DNA can be delivered into the cell by any methods known in the art. As some of the Ago proteins described herein follow the “one-guide-faithful” rule, i.e., once the Ago protein forms a complex with the guide DNA, the Ago protein does not dissociate from the guide DNA or exchange the guide DNA with an unbound guide DNA at a temperature lower than about 50° C. (such as about 37° C.), to ensure efficacy of the methods, the Ago protein and the guide DNA can either be delivered into the cell as a pre-formed complex, or the Ago protein is expressed by the cell in the presence of the guide DNA. In the latter case, a nucleic acid encoding the Ago protein can be transfected into the cell to allow expression of the Ago protein by the cell. The guide DNA can be transfected into the cell prior to or simultaneously as the nucleic acid encoding the Ago protein. In some embodiments, after introduction of the Ago (protein or nucleic acid) and the guide DNA to the cell, the cell is further transfected with the gDNA for one or more (such as about any one of 1, 2, 3, 4, or more) times.

Thus, in some embodiments, there is provided a method of modifying a target nucleic acid in a cell comprising transfecting a single-stranded guide DNA (such as a 5′ phosphorylated singled-stranded guide DNA) and a nucleic acid encoding an Ago protein into the cell, wherein the Ago protein and the guide DNA form a complex that specifically recognizes a target locus in the target nucleic acid, and wherein the target locus comprises a sequence that is complementary to the sequence of the guide DNA. In some embodiments, the complex cleaves the target locus. In some embodiments, wherein the target locus is a double-stranded DNA, the Ago protein induces a double-strand break in the target locus. In some embodiments, the complex does not cleave the target locus. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the Ago protein is derived from an organism selected from the group consisting of Natronobacterium gregoryi, Microcystis aeruginosa, Halogeometricum pallidum, Natrialba asiatica, Natronorubrum tibetense, Natrinema pellirubrum, Halogeometricum borinquense, Thermococcus barophilus, Thermosynechococcus elongatus, Halorubrum lacusprofundi, Microcystis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolyticus, Aromatoleum aromaticum, Thermococcus onnurineus, Methanopyrus kandleri, Synechococcus elongatus, Anoxybacillus flavithermus, Exiguobacterium sp., Lyngbya sp., Clostridium butyricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the Ago protein comprises an amino acid sequence having at least about 80% sequence homology (such as at least about any of 85%, 90%, 95%, 98%, 99% or more sequence homology, or about 100% sequence identity) to a sequence selected from the group consisting of SEQ ID NOs:1-42. In some embodiments, the guide DNA is about 10 to about 50 (such as about 20 to about 30) nucleotides long. In some embodiments, the cell is supplemented with a divalent metal ion (such as at least about 0.1 mM). In some embodiments, the guide DNA and the nucleic acid encoding the Ago protein are transfected into the cell simultaneously. In some embodiments, the guide DNA is transfected into the cell prior to the nucleic acid encoding the Ago protein. In some embodiments, the cell is transfected with the guide DNA for at least two (such as about any one of 2, 3, 4, 5, 6, or more) times. In some embodiments, the molar ratio of the guide DNA to the nucleic acid encoding the Ago protein is at least about 100:1. In some embodiments, the nucleic acid encoding the Ago protein is codon-optimized for the organism from which the cell is derived. In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the nucleic acid encoding the Ago protein is present in a vector, such as a viral vector. In some embodiments, the vector is an ultrapure plasmid (such as at least about 95% supercoiled). In some embodiments, the nucleic acid encoding the Ago protein is operably linked to a promoter. In some embodiments, the nucleic acid encoding the Ago protein is an mRNA.

In some embodiments, there is provided a method of modifying a target nucleic acid in a cell comprising transfecting a 5′ phosphorylated singled-stranded guide DNA and a nucleic acid encoding an Ago protein into the cell, wherein the Ago protein and the guide DNA form a complex that specifically recognizes a target locus in the target nucleic acid and induces a double-strand break (DSB) in the target locus, wherein the target locus is a double-stranded DNA comprising a sequence that is complementary to the sequence of the guide DNA, and wherein the Ago protein is derived from Natronobacterium gregoryi. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 1. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the cell is supplemented with a divalent metal ion (such as at least about 0.1 mM). In some embodiments, the guide DNA and the nucleic acid encoding the Ago protein are transfected into the cell simultaneously. In some embodiments, the guide DNA is transfected into the cell prior to the nucleic acid encoding the Ago protein. In some embodiments, the cell is transfected with the guide DNA for at least two (such as about any one of 2, 3, 4, 5, 6, or more) times. In some embodiments, the molar ratio of the guide DNA to the nucleic acid encoding the Ago protein is at least about 100:1. In some embodiments, the nucleic acid encoding the Ago protein is codon-optimized for the organism from which the cell is derived. In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the nucleic acid encoding the Ago protein is present in a vector, such as a viral vector. In some embodiments, the vector is an ultrapure plasmid (such as at least about 95% supercoiled). In some embodiments, the nucleic acid encoding the Ago protein is operably linked to a promoter. In some embodiments, the nucleic acid encoding the Ago protein is an mRNA.

In some embodiments, there is provided a method of modifying a target nucleic acid in a cell comprising delivering a pre-formed complex comprising an Ago protein and a single-stranded guide DNA (such as a 5′ phosphorylated singled-stranded guide DNA) into the cell, wherein the complex specifically recognizes a target locus in the target nucleic acid, and wherein the target locus comprises a sequence that is complementary to the sequence of the guide DNA. In some embodiments, the complex cleaves the target locus. In some embodiments, wherein the target locus is a double-stranded DNA, the Ago protein induces a double-strand break in the target locus. In some embodiments, the complex does not cleave the target locus. In some embodiments, wherein the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the Ago protein is derived from an organism selected from the group consisting of Natronobacterium gregoryi, Microcystis aeruginosa, Halogeometricum pallidum, Natrialba asiatica, Natronorubrum tibetense, Natrinema pellirubrum, Halogeometricum borinquense, Thermococcus barophilus, Thermosynechococcus elongatus, Halorubrum lacusprofundi, Microcystis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolyticus, Aromatoleum aromaticum, Thermococcus onnurineus, Methanopyrus kandleri, Synechococcus elongatus, Anoxybacillus flavithermus, Exiguobacterium sp., Lyngbya sp., Clostridium butyricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the Ago protein comprises an amino acid sequence having at least about 80% sequence homology (such as at least about any of 85%, 90%, 95%, 98%, 99% or more sequence homology, or about 100% sequence identity) to a sequence selected from the group consisting of SEQ ID NOs:1-42. In some embodiments, the guide DNA is about 10 to about 50 (such as about 20 to about 30) nucleotides long. In some embodiments, the cell is supplemented with a divalent metal ion (such as at least about 0.1 mM). In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the molar ratio between the guide DNA and the Ago protein in the complex is at least about 1:1. In some embodiments, the pre-formed complex is delivered into the cell by electroporation, microinjection, or mechanical deformation via a microfluidic channel. In some embodiments, the pre-formed complex is delivered into the cell via a vehicle selected from the group consisting of a cell-penetrating peptide, a virus-like particle, a nanocarrier, a liposome, a polymer, and a nanoparticle-stabilized nanocapsule. In some embodiments, the Ago protein is fused to the cell-penetrating peptide.

In some embodiments, there is provided a method of modifying a target nucleic acid in a cell comprising delivering a pre-formed complex comprising an Ago protein and a 5′ phosphorylated singled-stranded guide DNA into the cell, wherein the complex specifically recognizes a target locus in the target nucleic acid and induces a double-strand break (DSB) in the target locus, wherein the target locus is a double-stranded DNA comprising a sequence that is complementary to the sequence of the guide DNA, and wherein the Ago protein is derived from Natronobacterium gregoryi. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 1. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the cell is supplemented with a divalent metal ion (such as at least about 0.1 mM). In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the molar ratio between the guide DNA and the Ago protein in the complex is at least about 1:1. In some embodiments, the pre-formed complex is delivered into the cell by electroporation, microinjection, or mechanical deformation via a microfluidic channel. In some embodiments, the pre-formed complex is delivered into the cell via a vehicle selected from the group consisting of a cell-penetrating peptide, a virus-like particle, a nanocarrier, a liposome, a polymer, and a nanoparticle-stabilized nanocapsule. In some embodiments, the Ago protein is fused to the cell-penetrating peptide.

The Ago proteins described herein can modify a target nucleic acid in a cell in a variety of ways. In some embodiments, the method induces a site-specific cleavage in the target nucleic acid. In some embodiments, the method cleaves a genomic DNA in a bacterial cell. In some embodiments, the method cleaves a viral nucleic acid in a cell. In some embodiments, the method alters (such as increase or decrease) the expression level of the target nucleic acid in the cell. In some embodiments, the method reduces or silences the expression level of the target nucleic acid in the cell. In some embodiments, the method uses one or more endogenous DNA repair pathways, such as Non-homologous end joining (NHEJ) or Homology directed recombination (HDR), in the cell to repair the double-strand break induced in the target locus by the Ago protein, thereby introducing mutations or exogenous sequences at the target locus. In some embodiments, the method introduces a mutation at the target locus. Exemplary mutations include, but are not limited to, insertions, deletions, substitutions, and frameshifts. In some embodiments, the method inserts a donor DNA at the target locus. In some embodiments, the insertion of the donor DNA results in introduction of a selection marker or a reporter protein to the cell. In some embodiments, the insertion of the donor DNA results in knock-in of a gene. In some embodiments, the insertion of the donor DNA results in a knockout mutation. In some embodiments, the insertion of the donor DNA results in a substitution mutation, such as a single nucleotide substitution. In some embodiments, the method induces a phenotypic change to the cell.

Thus, in some embodiments, there is provided a method of site-specific cleavage of a target nucleic acid (such as a viral nucleic acid) in a cell comprising transfecting a single-stranded guide DNA (such as a 5′ phosphorylated singled-stranded guide DNA) and a nucleic acid encoding an Ago protein into the cell, wherein the Ago protein and the guide DNA form a complex that specifically cleaves a target locus in the target nucleic acid, and wherein the target locus comprises a sequence that is complementary to the sequence of the guide DNA. In some embodiments, wherein the target locus is a double-stranded DNA, the Ago protein induces a double-strand break in the target locus. In some embodiments, wherein the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the Ago protein is derived from an organism selected from the group consisting of Natronobacterium gregoryi, Microcystis aeruginosa, Halogeometricum pallidum. Natrialba asiatica, Natronorubrum tibetense, Natrinema pellirubrum, Halogeometricum borinquense, Thermococcus barophilus, Thermosynechococcus elongatus, Halorubrum lacusprofundi, Microcystis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolvticus, Aromatoleum aromaticum, Thermococcus onnurineus, Methanopyrus kandleri, Synechococcus elongatus, Anoxybacillus flavithermus, Exiguobacterium sp., Lyngbya sp., Clostridium butyricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the Ago protein comprises an amino acid sequence having at least about 80% sequence homology (such as at least about any of 85%, 90%, 95%, 98%, 99% or more sequence homology, or about 100% sequence identity) to a sequence selected from the group consisting of SEQ ID NOs: 1-42. In some embodiments, the guide DNA is about 10 to about 50 (such as about 20 to about 30) nucleotides long. In some embodiments, the cell is supplemented with a divalent metal ion (such as at least about 0.1 mM). In some embodiments, the guide DNA and the nucleic acid encoding the Ago protein are transfected into the cell simultaneously. In some embodiments, the guide DNA is transfected into the cell prior to the nucleic acid encoding the Ago protein. In some embodiments, the cell is transfected with the guide DNA for at least two (such as about any one of 2, 3, 4, 5, 6, or more) times. In some embodiments, the molar ratio of the guide DNA to the nucleic acid encoding the Ago protein is at least about 100:1. In some embodiments, the nucleic acid encoding the Ago protein is codon-optimized for the organism from which the cell is derived. In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the nucleic acid encoding the Ago protein is present in a vector, such as a viral vector. In some embodiments, the vector is an ultrapure plasmid (such as at least about 95% supercoiled). In some embodiments, the nucleic acid encoding the Ago protein is operably linked to a promoter. In some embodiments, the nucleic acid encoding the Ago protein is an mRNA. In some embodiments, the method induces a phenotypic change in the cell.

In some embodiments, there is provided a method of site-specific cleavage of a target nucleic acid (such as a viral nucleic acid) in a cell comprising transfecting a 5′ phosphorylated singled-stranded guide DNA and a nucleic acid encoding an Ago protein into the cell, wherein the Ago protein and the guide DNA form a complex that specifically recognizes a target locus in the target nucleic acid and induces a double-strand break (DSB) in the target locus, wherein the target locus is a double-stranded DNA comprising a sequence that is complementary to the sequence of the guide DNA, and wherein the Ago protein is derived from Natronobacterium gregoryi. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 1. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the cell is supplemented with a divalent metal ion (such as at least about 0.1 mM). In some embodiments, the guide DNA and the nucleic acid encoding the Ago protein are transfected into the cell simultaneously. In some embodiments, the guide DNA is transfected into the cell prior to the nucleic acid encoding the Ago protein. In some embodiments, the cell is transfected with the guide DNA for at least two (such as about any one of 2, 3, 4, 5, 6, or more) times. In some embodiments, the molar ratio of the guide DNA to the nucleic acid encoding the Ago protein is at least about 100:1. In some embodiments, the nucleic acid encoding the Ago protein is codon-optimized for the organism from which the cell is derived. In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the nucleic acid encoding the Ago protein is present in a vector, such as a viral vector. In some embodiments, the vector is an ultrapure plasmid (such as at least about 95% supercoiled). In some embodiments, the nucleic acid encoding the Ago protein is operably linked to a promoter. In some embodiments, the nucleic acid encoding the Ago protein is an mRNA. In some embodiments, the method induces a phenotypic change in the cell.

In some embodiments, there is provided a method of site-specific cleavage of a target nucleic acid (such as a viral nucleic acid) in a cell comprising delivering a pre-formed complex comprising an Ago protein and a single-stranded guide DNA (such as a 5′ phosphorylated singled-stranded guide DNA) into the cell, wherein the complex specifically cleaves a target locus in the target nucleic acid, and wherein the target locus comprises a sequence that is complementary to the sequence of the guide DNA. In some embodiments, wherein the target locus is a double-stranded DNA, the Ago protein induces a double-strand break in the target locus. In some embodiments, wherein the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the Ago protein is derived from an organism selected from the group consisting of Natronobacterium gregoryi, Microcystis aeruginosa, Halogeometricum pallidum, Natrialba asiatica, Natronorubrum tibetense, Natrinema pellirubrum, Halogeometricum borinquense, Thermococcus barophilus, Thermosynechococcus elongatus, Halorubrum lacusprofundi, Microcystis sp., Synechococcus sp., Clostridium bartlettii, Clostridium pefringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolyticus, Aromatoleum aromaticum, Thermococcus onnurineus, Methanopyrus kandleri, Synechococcus elongatus, Anoxybacillus flavithermus, Exiguobacterium sp., Lyngbya sp., Clostridium butyricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the Ago protein comprises an amino acid sequence having at least about 80% sequence homology (such as at least about any of 85%, 90%, 95%, 98%, 99% or more sequence homology, or about 100% sequence identity) to a sequence selected from the group consisting of SEQ ID NOs:1-42. In some embodiments, the guide DNA is about 10 to about 50 (such as about 20 to about 30) nucleotides long. In some embodiments, the cell is supplemented with a divalent metal ion (such as at least about 0.1 mM). In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the molar ratio between the guide DNA and the Ago protein in the complex is at least about 1:1. In some embodiments, the pre-formed complex is delivered into the cell by electroporation, microinjection, or mechanical deformation via a microfluidic channel. In some embodiments, the pre-formed complex is delivered into the cell via a vehicle selected from the group consisting of a cell-penetrating peptide, a virus-like particle, a nanocarrier, a liposome, a polymer, and a nanoparticle-stabilized nanocapsule. In some embodiments, the Ago protein is fused to the cell-penetrating peptide. In some embodiments, the method induces a phenotypic change in the cell.

In some embodiments, there is provided a method of site-specific cleavage of a target nucleic acid (such as a viral nucleic acid) in a cell comprising delivering a pre-formed complex comprising an Ago protein and a 5′ phosphorylated singled-stranded guide DNA into the cell, wherein the complex specifically recognizes a target locus in the target nucleic acid and induces a double-strand break (DSB) in the target locus, wherein the target locus is a double-stranded DNA comprising a sequence that is complementary to the sequence of the guide DNA, and wherein the Ago protein is derived from Natronobacterium gregoryi. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 1. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the cell is supplemented with a divalent metal ion (such as at least about 0.1 mM). In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the molar ratio between the guide DNA and the Ago protein in the complex is at least about 1:1. In some embodiments, the pre-formed complex is delivered into the cell by electroporation, microinjection, or mechanical deformation via a microfluidic channel. In some embodiments, the pre-formed complex is delivered into the cell via a vehicle selected from the group consisting of a cell-penetrating peptide, a virus-like particle, a nanocarrier, a liposome, a polymer, and a nanoparticle-stabilized nanocapsule. In some embodiments, the Ago protein is fused to the cell-penetrating peptide. In some embodiments, the method induces a phenotypic change in the cell.

In some embodiments, there is provided a method of inhibiting growth of a target cell (such as bacterial cell) comprising transfecting: (a) a nucleic acid encoding an Ago protein into the target cell, and (b) a vector comprising one or more guide DNAs targeting the genomic DNA of the target cell, wherein the Ago protein and the guide DNAs form complexes that specifically recognize and cleaves one or more target loci in the genomic DNA, and wherein the target loci comprise complementary sequences to the one or more guide DNAs. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the Ago protein is derived from an organism selected from the group consisting of Natronobacterium gregoryi, Microcystis aeruginosa, Halogeometricum pallidum, Natrialba asiatica, Natronorubrum tibetense, Natrinema pellirubrum, Halogeometricum borinquense, Thermococcus barophilus, Thermosynechococcus elongalus, Halorubrum lacusprofundi, Microcystis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolyticus, Aromatoleum aromaticum, Thermococcus onnurineus, Methanopyrus kandleri, Synechococcus elongalus, Anoxybacillus flavithermus, Exiguobacterium sp., Lyngbya sp., Clostridium butyricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the Ago protein comprises an amino acid sequence having at least about 80% sequence homology (such as at least about any of 85%, 90%, 95%, 98%, 99% or more sequence homology, or about 100% sequence identity) to a sequence selected from the group consisting of SEQ ID NOs:1-42. In some embodiments, the Ago protein is derived from Natronobacterium gregoryi. In some embodiments, the vector and the nucleic acid encoding the Ago protein are transfected into the cell simultaneously. In some embodiments, the vector is transfected into the cell prior to the nucleic acid encoding the Ago protein. In some embodiments, the vector is linearized prior to the transfection. In some embodiments, the nucleic acid encoding the Ago protein is codon-optimized for the target cell.

In some embodiments, there is provided a method of altering (such as decreasing) expression of a target nucleic acid (such as a gene) in a cell comprising transfecting a single-stranded guide DNA (such as a 5′ phosphorylated singled-stranded guide DNA) and a nucleic acid encoding an Ago protein into the cell, wherein the Ago protein and the guide DNA form a complex that specifically recognizes a target locus in the target nucleic acid, and wherein the target locus comprises a sequence that is complementary to the sequence of the guide DNA. In some embodiments, the complex cleaves the target locus. In some embodiments, wherein the target locus is a double-stranded DNA, the Ago protein induces a double-strand break in the target locus. In some embodiments, the complex does not cleave the target locus. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the Ago protein is derived from an organism selected from the group consisting of Natronobacterium gregoryi, Microcystis aeruginosa, Halogeometricum pallidum, Natrialba asiatica, Natronorubrum tibetense, Natrinema pellirubrum, Halogeometricum borinquense, Thermococcus barophilus, Thermosynechococcus elongatus, Halorubrum lacusprofundi, Microcystis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolyticus, Aromatoleum aromaticum, Thermococcus onnurineus, Methanopyrus kandleri, Synechococcus elongatus, Anoxybacillus flavithermus, Exiguobacterium sp., Lyngbya sp., Clostridium butyricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the Ago protein comprises an amino acid sequence having at least about 80% sequence homology (such as at least about any of 85%, 90%, 95%, 98%, 99% or more sequence homology, or about 100% sequence identity) to a sequence selected from the group consisting of SEQ ID NOs:1-42. In some embodiments, the guide DNA is about 10 to about 50 (such as about 20 to about 30) nucleotides long. In some embodiments, the cell is supplemented with a divalent metal ion (such as at least about 0.1 mM). In some embodiments, the guide DNA and the nucleic acid encoding the Ago protein are transfected into the cell simultaneously. In some embodiments, the guide DNA is transfected into the cell prior to the nucleic acid encoding the Ago protein. In some embodiments, the cell is transfected with the guide DNA for at least two (such as about any one of 2, 3, 4, 5, 6, or more) times. In some embodiments, the molar ratio of the guide DNA to the nucleic acid encoding the Ago protein is at least about 100:1. In some embodiments, the nucleic acid encoding the Ago protein is codon-optimized for the organism from which the cell is derived. In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the nucleic acid encoding the Ago protein is present in a vector, such as a viral vector. In some embodiments, the vector is an ultrapure plasmid (such as at least about 95% supercoiled). In some embodiments, the nucleic acid encoding the Ago protein is operably linked to a promoter. In some embodiments, the nucleic acid encoding the Ago protein is an mRNA. In some embodiments, the target locus is a disease-associated locus. In some embodiments, the method induces a phenotypic change in the cell.

In some embodiments, there is provided a method of altering (such as decreasing) expression of a target nucleic acid (such as a gene) in a cell comprising transfecting a 5′ phosphorylated singled-stranded guide DNA and a nucleic acid encoding an Ago protein into the cell, wherein the Ago protein and the guide DNA form a complex that specifically recognizes a target locus in the target nucleic acid and induces a double-strand break (DSB) in the target locus, wherein the target locus is a double-stranded DNA comprising a sequence that is complementary to the sequence of the guide DNA, and wherein the Ago protein is derived from Natronobacterium gregoryi. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 1. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the cell is supplemented with a divalent metal ion (such as at least about 0.1 mM). In some embodiments, the guide DNA and the nucleic acid encoding the Ago protein are transfected into the cell simultaneously. In some embodiments, the guide DNA is transfected into the cell prior to the nucleic acid encoding the Ago protein. In some embodiments, the cell is transfected with the guide DNA for at least two (such as about any one of 2, 3, 4, 5, 6, or more) times. In some embodiments, the molar ratio of the guide DNA to the nucleic acid encoding the Ago protein is at least about 100:1. In some embodiments, the nucleic acid encoding the Ago protein is codon-optimized for the organism from which the cell is derived. In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the nucleic acid encoding the Ago protein is present in a vector, such as a viral vector. In some embodiments, the vector is an ultrapure plasmid (such as at least about 95% supercoiled). In some embodiments, the nucleic acid encoding the Ago protein is operably linked to a promoter. In some embodiments, the nucleic acid encoding the Ago protein is an mRNA. In some embodiments, the target locus is a disease-associated locus. In some embodiments, the method induces a phenotypic change in the cell.

In some embodiments, there is provided a method of altering (such as decreasing) expression of a target nucleic acid (such as a gene) in a cell comprising delivering a pre-formed complex comprising an Ago protein and a single-stranded guide DNA (such as a 5′ phosphorylated singled-stranded guide DNA) into the cell, wherein the complex specifically recognizes a target locus in the target nucleic acid, and wherein the target locus comprises a sequence that is complementary to the sequence of the guide DNA. In some embodiments, the complex cleaves the target locus. In some embodiments, wherein the target locus is a double-stranded DNA, the Ago protein induces a double-strand break in the target locus. In some embodiments, the complex does not cleave the target locus. In some embodiments, wherein the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the Ago protein is derived from an organism selected from the group consisting of Natronobacterium gregoryi, Microcystis aeruginosa, Halogeometricum pallidum, Natrialba asiatica, Natronorubrum tibetense, Natrinema pellirubrum, Halogeometricum borinquense, Thermococcus barophilus, Thermosynechococcus elongatus, Halorubrum lacusprofundi, Microcystis sp., Synechococcus sp., Clostridium barlettii, Clostridium perfringens, Clostridium sarlagoforme, Clostridium sp., Intestinibacter bartlettii, Ferroglobus placidus, Halobacterium sp., Methanocaldococcus fervens, Pseudomonas luteola, Thermogladius cellulolylicus, Aromatoleum aronaticum, Thermococcus onnurineus, Methanopyrus kandleri, Synechococcus elongatus, Anoxybacillus flavithermus, Exiguobacterium sp., Lyngbya sp., Clostridium butyricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the Ago protein comprises an amino acid sequence having at least about 80% sequence homology (such as at least about any of 85%, 90%, 95%, 98%, 99% or more sequence homology, or about 100% sequence identity) to a sequence selected from the group consisting of SEQ ID NOs:1-42. In some embodiments, the guide DNA is about 10 to about 50 (such as about 20 to about 30) nucleotides long. In some embodiments, the cell is supplemented with a divalent metal ion (such as at least about 0.1 mM). In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the molar ratio between the guide DNA and the Ago protein in the complex is at least about 1:1. In some embodiments, the pre-formed complex is delivered into the cell by electroporation, microinjection, or mechanical deformation via a microfluidic channel. In some embodiments, the pre-formed complex is delivered into the cell via a vehicle selected from the group consisting of a cell-penetrating peptide, a virus-like particle, a nanocarrier, a liposome, a polymer, and a nanoparticle-stabilized nanocapsule. In some embodiments, the Ago protein is fused to the cell-penetrating peptide. In some embodiments, the target locus is a disease-associated locus. In some embodiments, the method induces a phenotypic change in the cell.

In some embodiments, there is provided a method of altering (such as decreasing) expression of a target nucleic acid (such as a gene) in a cell comprising delivering a pre-formed complex comprising an Ago protein and a 5′ phosphorylated singled-stranded guide DNA into the cell, wherein the complex specifically recognizes a target locus in the target nucleic acid and induces a double-strand break (DSB) in the target locus, wherein the target locus is a double-stranded DNA comprising a sequence that is complementary to the sequence of the guide DNA, and wherein the Ago protein is derived from Natronobacterium gregoryi. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 1. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the cell is supplemented with a divalent metal ion (such as at least about 0.1 mM). In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the molar ratio between the guide DNA and the Ago protein in the complex is at least about 1:1. In some embodiments, the pre-formed complex is delivered into the cell by electroporation, microinjection, or mechanical deformation via a microfluidic channel. In some embodiments, the pre-formed complex is delivered into the cell via a vehicle selected from the group consisting of a cell-penetrating peptide, a virus-like particle, a nanocarrier, a liposome, a polymer, and a nanoparticle-stabilized nanocapsule. In some embodiments, the Ago protein is fused to the cell-penetrating peptide. In some embodiments, the target locus is a disease-associated locus. In some embodiments, the method induces a phenotypic change in the cell.

In some embodiments, there is provided a method of introducing a mutation (such as indel or frameshift mutation) in a target nucleic acid (such as genomic DNA) in a cell comprising transfecting a single-stranded guide DNA (such as a 5′ phosphorylated singled-stranded guide DNA) and a nucleic acid encoding an Ago protein into the cell, wherein the Ago protein and the guide DNA form a complex that specifically recognizes a target locus in the target nucleic acid, and induces a double-strand break in the target locus, wherein the target locus is a double-stranded DNA comprising a sequence that is complementary to the sequence of the guide DNA. In some embodiments, wherein the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the Ago protein is derived from an organism selected from the group consisting of Natronobacterium gregoryi, Microcystis aeruginosa, Halogeometricum pallidum, Natrialba asiatica, Natronorubrum tibetense. Natrinema pellirubrum, Halogeometricum borinquense, Thermococcus barophilus, Thermosynechococcus elongalus, Halorubrum lacusprofundi, Microcystis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolyticus, Aromatoleum aromaticum, Thermococcus onnurineus, Methanopyrus kandleri, Synechococcus elongalus, Anoxybacillus flavithermus, Exiguobacterium sp., Lyngbya sp., Clostridium butryricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the Ago protein comprises an amino acid sequence having at least about 80% sequence homology (such as at least about any of 85%, 90%, 95%, 98%, 99% or more sequence homology, or about 100% sequence identity) to a sequence selected from the group consisting of SEQ ID NOs:1-42. In some embodiments, the guide DNA is about 10 to about 50 (such as about 20 to about 30) nucleotides long. In some embodiments, the cell is supplemented with a divalent metal ion (such as at least about 0.1 mM). In some embodiments, the guide DNA and the nucleic acid encoding the Ago protein are transfected into the cell simultaneously. In some embodiments, the guide DNA is transfected into the cell prior to the nucleic acid encoding the Ago protein. In some embodiments, the cell is transfected with the guide DNA for at least two (such as about any one of 2, 3, 4, 5, 6, or more) times. In some embodiments, the molar ratio of the guide DNA to the nucleic acid encoding the Ago protein is at least about 100:1. In some embodiments, the nucleic acid encoding the Ago protein is codon-optimized for the organism from which the cell is derived. In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the nucleic acid encoding the Ago protein is present in a vector, such as a viral vector. In some embodiments, the vector is an ultrapure plasmid (such as at least about 95% supercoiled). In some embodiments, the nucleic acid encoding the Ago protein is operably linked to a promoter. In some embodiments, the nucleic acid encoding the Ago protein is an mRNA. In some embodiments, the target locus is a disease-associated locus. In some embodiments, the method induces a phenotypic change in the cell.

In some embodiments, there is provided a method of introducing a mutation (such as indel or frameshift mutation) in a target nucleic acid (such as genomic DNA) in a cell comprising transfecting a 5′ phosphorylated singled-stranded guide DNA and a nucleic acid encoding an Ago protein into the cell, wherein the Ago protein and the guide DNA form a complex that specifically recognizes a target locus in the target nucleic acid and induces a double-strand break (DSB) in the target locus, wherein the target locus is a double-stranded DNA comprising a sequence that is complementary to the sequence of the guide DNA, and wherein the Ago protein is derived from Natronobacterium gregoryi. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 1. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the cell is supplemented with a divalent metal ion (such as at least about 0.1 mM). In some embodiments, the guide DNA and the nucleic acid encoding the Ago protein are transfected into the cell simultaneously. In some embodiments, the guide DNA is transfected into the cell prior to the nucleic acid encoding the Ago protein. In some embodiments, the cell is transfected with the guide DNA for at least two (such as about any one of 2, 3, 4, 5, 6, or more) times. In some embodiments, the molar ratio of the guide DNA to the nucleic acid encoding the Ago protein is at least about 100:1. In some embodiments, the nucleic acid encoding the Ago protein is codon-optimized for the organism from which the cell is derived. In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the nucleic acid encoding the Ago protein is present in a vector, such as a viral vector. In some embodiments, the vector is an ultrapure plasmid (such as at least about 95% supercoiled). In some embodiments, the nucleic acid encoding the Ago protein is operably linked to a promoter. In some embodiments, the nucleic acid encoding the Ago protein is an mRNA. In some embodiments, the target locus is a disease-associated locus. In some embodiments, the method induces a phenotypic change in the cell.

In some embodiments, there is provided a method of introducing a mutation (such as indel or frameshift mutation) in a target nucleic acid (such as genomic DNA) in a cell comprising delivering a pre-formed complex comprising an Ago protein and a single-stranded guide DNA (such as a 5′ phosphorylated singled-stranded guide DNA) into the cell, wherein the complex specifically recognizes a target locus in the target nucleic acid and induces a double-strand break in the target locus, and wherein the target locus is a double-stranded DNA comprising a sequence that is complementary to the sequence of the guide DNA. In some embodiments, wherein the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the Ago protein is derived from an organism selected from the group consisting of Natronobacterium gregoryi, Microcystis aeruginosa, Halogeometricum pallidum, Natrialba asiatica, Natronorubrum tibetense, Natrinema pellirubrum, Halogeometricum borinquense, Thermococcus barophilus, Thermosynechococcus elongatus, Halorubrum lacusprofundi, Microcystis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolyticus, Aromatoleum aromaticum, Thermococcus onnurineus, Methanopyrus kandleri, Synechococcus elongatus, Anoxybacillus flavithermus, Exiguobacterium sp., Lyngbya sp., Clostridium butyricum, Halorubrum kocurii. Burkholderia ambifaria, Burkholderia graminis, Haloarcula marismortui, Mesorhizobium loti, Rhodobacterales bacterium and Pedobacter heparinus. In some embodiments, the Ago protein comprises an amino acid sequence having at least about 80% sequence homology (such as at least about any of 85%, 90%, 95%, 98%, 99% or more sequence homology, or about 100% sequence identity) to a sequence selected from the group consisting of SEQ ID NOs: 1-42. In some embodiments, the guide DNA is about 10 to about 50 (such as about 20 to about 30) nucleotides long. In some embodiments, the cell is supplemented with a divalent metal ion (such as at least about 0.1 mM). In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the molar ratio between the guide DNA and the Ago protein in the complex is at least about 1:1. In some embodiments, the pre-formed complex is delivered into the cell by electroporation, microinjection, or mechanical deformation via a microfluidic channel. In some embodiments, the pre-formed complex is delivered into the cell via a vehicle selected from the group consisting of a cell-penetrating peptide, a virus-like particle, a nanocarrier, a liposome, a polymer, and a nanoparticle-stabilized nanocapsule. In some embodiments, the Ago protein is fused to the cell-penetrating peptide. In some embodiments, the target locus is a disease-associated locus. In some embodiments, the method induces a phenotypic change in the cell.

In some embodiments, there is provided a method of introducing a mutation (such as indel or frameshift mutation) in a target nucleic acid (such as genomic DNA) in a cell comprising delivering a pre-formed complex comprising an Ago protein and a 5′ phosphorylated singled-stranded guide DNA into the cell, wherein the complex specifically recognizes a target locus in the target nucleic acid and induces a double-strand break (DSB) in the target locus, wherein the target locus is a double-stranded DNA comprising a sequence that is complementary to the sequence of the guide DNA, and wherein the Ago protein is derived from Natronobacterium gregoryi. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 1. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the cell is supplemented with a divalent metal ion (such as at least about 0.1 mM). In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the molar ratio between the guide DNA and the Ago protein in the complex is at least about 1:1. In some embodiments, the pre-formed complex is delivered into the cell by electroporation, microinjection, or mechanical deformation via a microfluidic channel. In some embodiments, the pre-formed complex is delivered into the cell via a vehicle selected from the group consisting of a cell-penetrating peptide, a virus-like particle, a nanocarrier, a liposome, a polymer, and a nanoparticle-stabilized nanocapsule. In some embodiments, the Ago protein is fused to the cell-penetrating peptide. In some embodiments, the target locus is a disease-associated locus. In some embodiments, the method induces a phenotypic change in the cell.

In some embodiments, the method further comprises contacting the target nucleic acid with a donor DNA comprising a sequence homologous to the sequence of the target locus under a condition that allows integration of the donor DNA at the target locus. The donor DNA can be delivered into the cell using any known methods in the art. In some embodiments, the donor DNA is delivered into the cell simultaneously as the nucleic acid encoding the Ago protein and/or the guide DNA, or sequentially (e.g., after) the nucleic acid encoding the Ago protein and/or the guide DNA. In some embodiments, the donor DNA is delivered to the cell simultaneously as the pre-formed complex comprising the Ago protein and the guide DNA, or sequentially (e.g., before or after) the pre-formed complex.

In some embodiments, there is provided a method of inserting a donor DNA in a target nucleic acid (such as genomic DNA) in a cell comprising transfecting a single-stranded guide DNA (such as a 5′ phosphorylated singled-stranded guide DNA), a nucleic acid encoding an Ago protein, and the donor DNA into the cell, wherein the Ago protein and the guide DNA form a complex that specifically recognizes a target locus in the target nucleic acid, and induces a double-strand break in the target locus, wherein the donor DNA is integrated at the DSB in the target locus, wherein the target locus is a double-stranded DNA comprising a sequence that is complementary to the sequence of the guide DNA, and wherein the donor DNA comprises a sequence homologous to the sequence of the target locus. In some embodiments, wherein the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the Ago protein is derived from an organism selected from the group consisting of Natronobacterium gregoryi, Microcystis aeruginosa, Halogeometricum pallidum, Natrialba asiatica, Natronorubrum tibetense, Natrinema pellirubrum, Halogeometricum borinquense, Thermococcus barophilus, Thermosynechococcus elongatus, Halorubrum lacusprofundi, Microcystis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolvticus, Aromatoleum aromaticum, Thermococcus onnurineus, Methanopyrus kandleri, Synechococcus elongatus. Anoxybacillus flavithermus, Exiguobacterium sp., Lyngbya sp., Clostridium butyricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the Ago protein comprises an amino acid sequence having at least about 80% sequence homology (such as at least about any of 85%, 90%, 95%, 98%, 99% or more sequence homology, or about 100% sequence identity) to a sequence selected from the group consisting of SEQ ID NOs: 1-42. In some embodiments, the guide DNA is about 10 to about 50 (such as about 20 to about 30) nucleotides long. In some embodiments, the cell is supplemented with a divalent metal ion (such as at least about 0.1 mM). In some embodiments, the guide DNA and the nucleic acid encoding the Ago protein are transfected into the cell simultaneously. In some embodiments, the guide DNA is transfected into the cell prior to the nucleic acid encoding the Ago protein. In some embodiments, the cell is transfected with the guide DNA for at least two (such as about any one of 2, 3, 4, 5, 6, or more) times. In some embodiments, the molar ratio of the guide DNA to the nucleic acid encoding the Ago protein is at least about 100:1. In some embodiments, the nucleic acid encoding the Ago protein is codon-optimized for the organism from which the cell is derived. In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the nucleic acid encoding the Ago protein is present in a vector, such as a viral vector. In some embodiments, the vector is an ultrapure plasmid (such as at least about 95% supercoiled). In some embodiments, the nucleic acid encoding the Ago protein is operably linked to a promoter. In some embodiments, the nucleic acid encoding the Ago protein is an mRNA. In some embodiments, the target locus is a disease-associated locus. In some embodiments, the method induces a phenotypic change in the cell. In some embodiments, wherein the donor DNA comprises an exogenous sequence (such as an exogenous gene), the method introduces a knock-in of the exogenous sequence at the target locus. In some embodiments, wherein the donor DNA comprises a substitution mutation (such as a single nucleotide substitution), the method introduces the substitution mutation at the target locus. In some embodiments, the method introduces a knockout mutation at the target locus.

In some embodiments, there is provided a method of inserting a donor DNA in a target nucleic acid (such as genomic DNA) in a cell comprising transfecting a 5′ phosphorylated singled-stranded guide DNA, a nucleic acid encoding an Ago protein, and the donor DNA into the cell, wherein the Ago protein and the guide DNA form a complex that specifically recognizes a target locus in the target nucleic acid and induces a double-strand break (DSB) in the target locus, wherein the donor DNA is integrated at the DSB in the target locus, wherein the target locus is a double-stranded DNA comprising a sequence that is complementary to the sequence of the guide DNA, wherein the donor DNA comprises a sequence homologous to the sequence of the target locus, and wherein the Ago protein is derived from Natronobacterium gregoryi. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 1. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the cell is supplemented with a divalent metal ion (such as at least about 0.1 mM). In some embodiments, the guide DNA and the nucleic acid encoding the Ago protein are transfected into the cell simultaneously. In some embodiments, the guide DNA is transfected into the cell prior to the nucleic acid encoding the Ago protein. In some embodiments, the cell is transfected with the guide DNA for at least two (such as about any one of 2, 3, 4, 5, 6, or more) times. In some embodiments, the molar ratio of the guide DNA to the nucleic acid encoding the Ago protein is at least about 100:1. In some embodiments, the nucleic acid encoding the Ago protein is codon-optimized for the organism from which the cell is derived. In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the nucleic acid encoding the Ago protein is present in a vector, such as a viral vector. In some embodiments, the vector is an ultrapure plasmid (such as at least about 95% supercoiled). In some embodiments, the nucleic acid encoding the Ago protein is operably linked to a promoter. In some embodiments, the nucleic acid encoding the Ago protein is an mRNA. In some embodiments, the target locus is a disease-associated locus. In some embodiments, the method induces a phenotypic change in the cell. In some embodiments, wherein the donor DNA comprises an exogenous sequence (such as an exogenous gene), the method introduces a knock-in of the exogenous sequence at the target locus. In some embodiments, wherein the donor DNA comprises a substitution mutation (such as a single nucleotide substitution), the method introduces the substitution mutation at the target locus. In some embodiments, the method introduces a knockout mutation at the target locus.

In some embodiments, there is provided a method of inserting a donor DNA in a target nucleic acid (such as genomic DNA) in a cell comprising delivering the donor DNA and a pre-formed complex comprising an Ago protein and a single-stranded guide DNA (such as a 5′ phosphorylated singled-stranded guide DNA) into the cell, wherein the complex specifically recognizes a target locus in the target nucleic acid and induces a double-strand break in the target locus, wherein the donor DNA is integrated at the DSB in the target locus, wherein the target locus is a double-stranded DNA comprising a sequence that is complementary to the sequence of the guide DNA, and wherein the donor DNA comprises a sequence homologous to the sequence of the target locus. In some embodiments, wherein the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the Ago protein is derived from an organism selected from the group consisting of Natronobacterium gregoryi, Microcystis aeruginosa, Halogeometricum pallidum, Natrialba asiatica, Natronorubrum tibetense, Natrinema pellirubrum, Halogeometricum borinquense, Thermococcus barophilus. Thermosynechococcus elongatus, Halorubrum lacusprofundi, Microcystis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolyticus, Aromatoleum aromaticum, Thermococcus onnurineus, Methanopyrus kandleri, Synechococcus elongatus, Anoxybacillus flavithermus, Exiguobacterium sp., Lyngbya sp., Clostridium butyricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the Ago protein comprises an amino acid sequence having at least about 80% sequence homology (such as at least about any of 85%, 90%, 95%, 98%, 99% or more sequence homology, or about 100% sequence identity) to a sequence selected from the group consisting of SEQ ID NOs: 1-42. In some embodiments, the guide DNA is about 10 to about 50 (such as about 20 to about 30) nucleotides long. In some embodiments, the cell is supplemented with a divalent metal ion (such as at least about 0.1 mM). In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the molar ratio between the guide DNA and the Ago protein in the complex is at least about 1:1. In some embodiments, the pre-formed complex is delivered into the cell by electroporation, microinjection, or mechanical deformation via a microfluidic channel. In some embodiments, the pre-formed complex is delivered into the cell via a vehicle selected from the group consisting of a cell-penetrating peptide, a virus-like particle, a nanocarrier, a liposome, a polymer, and a nanoparticle-stabilized nanocapsule. In some embodiments, the Ago protein is fused to the cell-penetrating peptide. In some embodiments, the target locus is a disease-associated locus. In some embodiments, the method induces a phenotypic change in the cell. In some embodiments, wherein the donor DNA comprises an exogenous sequence (such as an exogenous gene), the method introduces a knock-in of the exogenous sequence at the target locus. In some embodiments, wherein the donor DNA comprises a substitution mutation (such as a single nucleotide substitution), the method introduces the substitution mutation at the target locus. In some embodiments, the method introduces a knockout mutation at the target locus.

In some embodiments, there is provided a method of inserting a donor DNA in a target nucleic acid (such as genomic DNA) in a cell comprising delivering the donor DNA and a pre-formed complex comprising an Ago protein and a 5′ phosphorylated singled-stranded guide DNA into the cell, wherein the complex specifically recognizes a target locus in the target nucleic acid and induces a double-strand break (DSB) in the target locus, wherein the donor DNA is integrated at the DSB in the target locus, wherein the target locus is a double-stranded DNA comprising a sequence that is complementary to the sequence of the guide DNA, wherein the donor DNA comprises a sequence homologous to the sequence of the target locus, and wherein the Ago protein is derived from Natronobacterium gregoryi. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 1. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the cell is supplemented with a divalent metal ion (such as at least about 0.1 mM). In some embodiments, the Ago protein comprises a nuclear localization signal (NLS). In some embodiments, the cell is treated with one or more antibiotics. In some embodiments, the cell is free from contamination by (other) non-viral microorganisms. In some embodiments, the molar ratio between the guide DNA and the Ago protein in the complex is at least about 1:1. In some embodiments, the pre-formed complex is delivered into the cell by electroporation, microinjection, or mechanical deformation via a microfluidic channel. In some embodiments, the pre-formed complex is delivered into the cell via a vehicle selected from the group consisting of a cell-penetrating peptide, a virus-like particle, a nanocarrier, a liposome, a polymer, and a nanoparticle-stabilized nanocapsule. In some embodiments, the Ago protein is fused to the cell-penetrating peptide. In some embodiments, the target locus is a disease-associated locus. In some embodiments, the method induces a phenotypic change in the cell. In some embodiments, wherein the donor DNA comprises an exogenous sequence (such as an exogenous gene), the method introduces a knock-in of the exogenous sequence at the target locus. In some embodiments, wherein the donor DNA comprises a substitution mutation (such as a single nucleotide substitution), the method introduces the substitution mutation at the target locus. In some embodiments, the method introduces a knockout mutation at the target locus.

In some embodiments, the method comprises one or more steps assessing the cell after the modification. In some embodiments, the method comprises assessing the cell for a phenotypic change. In some embodiments, wherein the donor DNA comprises a selection marker, such as a reporter protein, the method comprises assessing the cell for expression of the selection marker. In some embodiments, the method comprises sequencing the target nucleic acid. In some embodiments, the method comprises modifying the target nucleic acid in a plurality of cells, and selecting a cell having a modified target nucleic acid based on one or more of the following: (1) a phenotypic change to the cell; (2) expression of a selection marker, such as a reporter protein, wherein the donor DNA comprises the selection marker; and/or (3) sequence of the modified target nucleic acid in the cell.

Thus, in some embodiments, there is provided a method comprising modifying a target nucleic acid in a plurality of cells, comprising: (a) transfecting a single-stranded guide DNA (such as a 5′ phosphorylated singled-stranded guide DNA) and a nucleic acid encoding an Ago protein into the plurality of cells, wherein the Ago protein and the guide DNA form a complex that specifically recognizes a target locus in the target nucleic acid, and wherein the target locus comprises a sequence that is complementary to the sequence of the guide DNA; and (b) selecting a cell having a modified target nucleic acid based on one or more of the following: (1) a phenotypic change to the cell; (2) expression of a selection marker (such as a reporter protein), wherein the method further comprises contacting the target nucleic acid with a donor DNA comprising a sequence homologous to the sequence of the target locus under a condition that allows integration of the donor DNA at the target locus, and wherein the donor DNA comprises the selection marker; and/or (3) sequence of the modified target nucleic acid in the cell. In some embodiments, the complex cleaves the target locus. In some embodiments, wherein the target locus is a double-stranded DNA, the Ago protein induces a double-strand break in the target locus. In some embodiments, the complex does not cleave the target locus. In some embodiments, the guide DNA does not dissociate from the Ago protein at a temperature lower than about 50° C. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the Ago protein is derived from an organism selected from the group consisting of Natronobacterium gregoryi, Microcystis aeruginosa, Halogeometricum pallidum, Natrialba asiatica, Natronorubrum tibetense, Natrinema pellirubrum, Halogeometricum borinquense, Thermococcus barophilus, Thermosynechococcus elongalus, Halorubrum lacusprofundi, Microcystis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolyticus, Aromatoleum aromaticum, Thermococcus onnurineus, Methanopyrus kandleri, Synechococcus elongalus, Anoxybacillus flavithermus, Exiguobacterium sp., Lyngbya sp., Clostridium butryricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the Ago protein comprises an amino acid sequence having at least about 80% sequence homology (such as at least about any of 85%, 90%, 95%, 98%, 99% or more sequence homology, or about 100% sequence identity) to a sequence selected from the group consisting of SEQ ID NOs:1-42. In some embodiments, the guide DNA is about 10 to about 50 (such as about 20 to about 30) nucleotides long. In some embodiments, the plurality of cells is supplemented with a divalent metal ion (such as at least about 0.1 mM). In some embodiments, the guide DNA and the nucleic acid encoding the Ago protein are transfected into the plurality of cells simultaneously. In some embodiments, the guide DNA is transfected into the plurality of cells prior to the nucleic acid encoding the Ago protein. In some embodiments, the plurality of cells is transfected with the guide DNA for at least two (such as about any one of 2, 3, 4, 5, 6, or more) times. In some embodiments, the molar ratio of the guide DNA to the nucleic acid encoding the Ago protein is at least about 100:1. In some embodiments, the nucleic acid encoding the Ago protein is codon-optimized for the species from which the plurality of cells is derived. In some embodiments, the Ago protein comprises a nuclear localization signal (NLS). In some embodiments, the plurality of cells is treated with one or more antibiotics. In some embodiments, the plurality of cells is free from contamination by (other) non-viral microorganisms. In some embodiments, the nucleic acid encoding the Ago protein is present in a vector, such as a viral vector. In some embodiments, the vector is an ultrapure plasmid (such as at least about 95% supercoiled). In some embodiments, the nucleic acid encoding the Ago protein is operably linked to a promoter. In some embodiments, the nucleic acid encoding the Ago protein is an mRNA. In some embodiments, the modifying comprises introducing a mutation (such as an indel or frameshift mutation) at the target locus. In some embodiments, the modifying comprises altering expression of the target nucleic acid. In some embodiments, the modifying comprises introducing a knockout mutation at the target locus. In some embodiments, the modifying comprises inducing a phenotypic change to the cell. In some embodiments, the modifying comprises introducing a knock-out mutation, a knock-in of an exogenous sequence, or a substitution (such as single-nucleotide substitution) mutation at the target locus.

In some embodiments, there is provided a method comprising modifying a target nucleic acid in a plurality of cells, comprising: (a) transfecting a 5′ phosphorylated singled-stranded guide DNA and a nucleic acid encoding an Ago protein into the plurality of cells, wherein the Ago protein and the guide DNA form a complex that specifically recognizes a target locus in the target nucleic acid and induces a double-strand break (DSB) in the target locus, wherein the target locus is a double-stranded DNA comprising a sequence that is complementary to the sequence of the guide DNA, and wherein the Ago protein is derived from Natronobacterium gregoryi; and (b) selecting a cell having a modified target nucleic acid based on one or more of the following: (1) a phenotypic change to the cell; (2) expression of a selection marker (such as a reporter protein), wherein the method further comprises contacting the target nucleic acid with a donor DNA comprising a sequence homologous to the sequence of the target locus under a condition that allows integration of the donor DNA at the target locus, and wherein the donor DNA comprises the selection marker, and/or (3) sequence of the modified target nucleic acid in the cell. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 1. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the plurality of cells is supplemented with a divalent metal ion (such as at least about 0.1 mM). In some embodiments, the guide DNA and the nucleic acid encoding the Ago protein are transfected into the plurality of cells simultaneously. In some embodiments, the guide DNA is transfected into the plurality of cells prior to the nucleic acid encoding the Ago protein. In some embodiments, the plurality of cells is transfected with the guide DNA for at least two (such as about any one of 2, 3, 4, 5, 6, or more) times. In some embodiments, the molar ratio of the guide DNA to the nucleic acid encoding the Ago protein is at least about 100:1. In some embodiments, the nucleic acid encoding the Ago protein is codon-optimized for the species from which the plurality of cells is derived. In some embodiments, the Ago protein comprises a nuclear localization signal (NLS). In some embodiments, the plurality of cells is treated with one or more antibiotics. In some embodiments, the plurality of cells is free from contamination by (other) non-viral microorganisms. In some embodiments, the nucleic acid encoding the Ago protein is present in a vector, such as a viral vector. In some embodiments, the vector is an ultrapure plasmid (such as at least about 95% supercoiled). In some embodiments, the nucleic acid encoding the Ago protein is operably linked to a promoter. In some embodiments, the nucleic acid encoding the Ago protein is an mRNA. In some embodiments, the modifying comprises introducing a mutation (such as an indel or frameshift mutation) at the target locus. In some embodiments, the modifying comprises altering expression of the target nucleic acid. In some embodiments, the modifying comprises introducing a knockout mutation at the target locus. In some embodiments, the modifying comprises inducing a phenotypic change to the cell. In some embodiments, the modifying comprises introducing a knock-out mutation, a knock-in of an exogenous sequence, or a substitution (such as single-nucleotide substitution) mutation at the target locus.

In some embodiments, there is provided a method of modifying a target nucleic acid in a plurality of cells comprising: (a) delivering a pre-formed complex comprising an Ago protein and a single-stranded guide DNA (such as a 5′ phosphorylated singled-stranded guide DNA) into the plurality of cells, wherein the complex specifically recognizes a target locus in the target nucleic acid, and wherein the target locus comprises a sequence that is complementary to the sequence of the guide DNA: and (b) selecting a cell having a modified target nucleic acid based on one or more of the following: (1) a phenotypic change to the cell; (2) expression of a selection marker (such as a reporter protein), wherein the method further comprises contacting the target nucleic acid with a donor DNA comprising a sequence homologous to the sequence of the target locus under a condition that allows integration of the donor DNA at the target locus, and wherein the donor DNA comprises the selection marker, and/or (3) sequence of the modified target nucleic acid in the cell. In some embodiments, the complex cleaves the target locus. In some embodiments, the complex does not cleave the target locus. In some embodiments, wherein the target locus is a double-stranded DNA, the Ago protein induces a double-strand break in the target locus. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the Ago protein is derived from an organism selected from the group consisting of Natronobacterium gregoryi, Microcystis aeruginosa, Halogeometricum pallidum, Natrialba asiatica, Natronorubrum tibetense, Natrinema pellirubrum, Halogeometricum borinquense, Thermococcus barophilus, Thermosynechococcus elongatus, Halorubrum lacusprofundi, Microcystis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolyticus, Aromatoleum aromaticum, Thermococcus onnurineus, Methanopyrus kandleri, Synechococcus elongatus, Anoxybacillus flavithermus, Exiguobacterium sp., Lyngbya sp., Clostridium butyricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the Ago protein comprises an amino acid sequence having at least about 80% sequence homology (such as at least about any of 85%, 90%, 95%, 98%, 99% or more sequence homology, or about 100% sequence identity) to a sequence selected from the group consisting of SEQ ID NOs:1-42. In some embodiments, the guide DNA is about 10 to about 50 (such as about 20 to about 30) nucleotides long. In some embodiments, the plurality of cells is supplemented with a divalent metal ion (such as at least about 0.1 mM). In some embodiments, the Ago protein comprises a nuclear localization signal (NLS). In some embodiments, the plurality of cells is treated with one or more antibiotics. In some embodiments, the plurality of cells is free from contamination by (other) non-viral microorganisms. In some embodiments, the molar ratio between the guide DNA and the Ago protein in the complex is at least about 1:1. In some embodiments, the pre-formed complex is delivered into the plurality of cells by electroporation, microinjection, or mechanical deformation via a microfluidic channel. In some embodiments, the pre-formed complex is delivered into the plurality of cells via a vehicle selected from the group consisting of a cell-penetrating peptide, a virus-like particle, a nanocarrier, a liposome, a polymer, and a nanoparticle-stabilized nanocapsule. In some embodiments, the Ago protein is fused to the cell-penetrating peptide. In some embodiments, the modifying comprises introducing a mutation (such as an indel or frameshift mutation) at the target locus. In some embodiments, the modifying comprises altering expression of the target nucleic acid. In some embodiments, the modifying comprises introducing a knockout mutation at the target locus. In some embodiments, the modifying comprises inducing a phenotypic change to the cell. In some embodiments, the modifying comprises introducing a knock-out mutation, a knock-in of an exogenous sequence, or a substitution (such as single-nucleotide substitution) mutation at the target locus.

In some embodiments, there is provided a method of modifying a target nucleic acid in a plurality of cells comprising: (a) delivering a pre-formed complex comprising an Ago protein and a 5′ phosphorylated singled-stranded guide DNA into the plurality of cells, wherein the complex specifically recognizes a target locus in the target nucleic acid and induces a double-strand break (DSB) in the target locus, wherein the target locus is a double-stranded DNA comprising a sequence that is complementary to the sequence of the guide DNA, and wherein the Ago protein is derived from Natronobacterium gregoryi; and (b) selecting a cell having a modified target nucleic acid based on one or more of the following: (1) a phenotypic change to the cell; (2) expression of a selection marker (such as a reporter protein), wherein the method further comprises contacting the target nucleic acid with a donor DNA comprising a sequence homologous to the sequence of the target locus under a condition that allows integration of the donor DNA at the target locus, and wherein the donor DNA comprises the selection marker; and/or (3) sequence of the modified target nucleic acid in the cell. In some embodiments, the Ago protein comprises an amino acid sequence having at least about 80% sequence homology (such as at least about any of 85%, 90%, 95%, 98%, 99% or more sequence homology, or about 100% sequence identity) to SEQ ID NO: 1. In some embodiments, the sequence of the target locus comprises no more than about 3 mismatches (such as no mismatch) to the sequence of the guide DNA. In some embodiments, the target locus has a GC content of at least about 60%. In some embodiments, the guide DNA is about 10 to about 50 (such as about 20 to about 30) nucleotides long. In some embodiments, the plurality of cells is supplemented with a divalent metal ion (such as at least about 0.1 mM). In some embodiments, the Ago protein comprises a nuclear localization signal (NLS). In some embodiments, the plurality of cells is treated with one or more antibiotics. In some embodiments, the plurality of cells is free from contamination by (other) non-viral microorganisms. In some embodiments, the molar ratio between the guide DNA and the Ago protein in the complex is at least about 1:1. In some embodiments, the pre-formed complex is delivered into the plurality of cells by electroporation, microinjection, or mechanical deformation via a microfluidic channel. In some embodiments, the pre-formed complex is delivered into the plurality of cells via a vehicle selected from the group consisting of a cell-penetrating peptide, a virus-like particle, a nanocarrier, a liposome, a polymer, and a nanoparticle-stabilized nanocapsule. In some embodiments, the Ago protein is fused to the cell-penetrating peptide. In some embodiments, the modifying comprises introducing a mutation (such as an indel or frameshift mutation) at the target locus. In some embodiments, the modifying comprises altering expression of the target nucleic acid. In some embodiments, the modifying comprises introducing a knockout mutation at the target locus. In some embodiments, the modifying comprises inducing a phenotypic change to the cell. In some embodiments, the modifying comprises introducing a knock-out mutation, a knock-in of an exogenous sequence, or a substitution (such as single-nucleotide substitution) mutation at the target locus.

In some embodiments, there is provided use of an Argonaute nuclease in gene editing, wherein the Argonaute nuclease is capable of using a 5′ phosphorylated single-stranded oligonucleotide DNA as a guide to cleave a double-stranded target DNA and induce a double-stranded break (DSB) in the target DNA at about 10-60° C. In some embodiments, there is provided use of an Argonaute nuclease in gene editing, wherein the Argonaute nuclease uses a 5′ phosphorylated single-stranded oligonucleotide DNA as a guide and forms a complex with the guide DNA, wherein the complex cleaves both strands of a double-stranded target DNA comprising a complementary sequence to the guide DNA, thereby providing a double-strand break in the target DNA. In some embodiments, wherein the target DNA is a target gene in a cell, the use comprises repairing the double-strand break in the target gene using the endogenous Non-homologous End Joining (NHEJ) repair pathway in the cell, thereby introducing a mutation to knockout or modify the target gene or a functional region thereof. Alternatively, in some embodiments, wherein the target DNA is a target gene in a cell, the use comprises repairing the double-stranded break in the target gene using the endogenous Homology directed recombination (HDR) pathway in the cell in the presence of an exogenous DNA fragment comprising homologous sequences to the target gene, thereby inserting the exogenous DNA fragment into the DSB to achieve customized modification of the target gene or a functional region thereof.

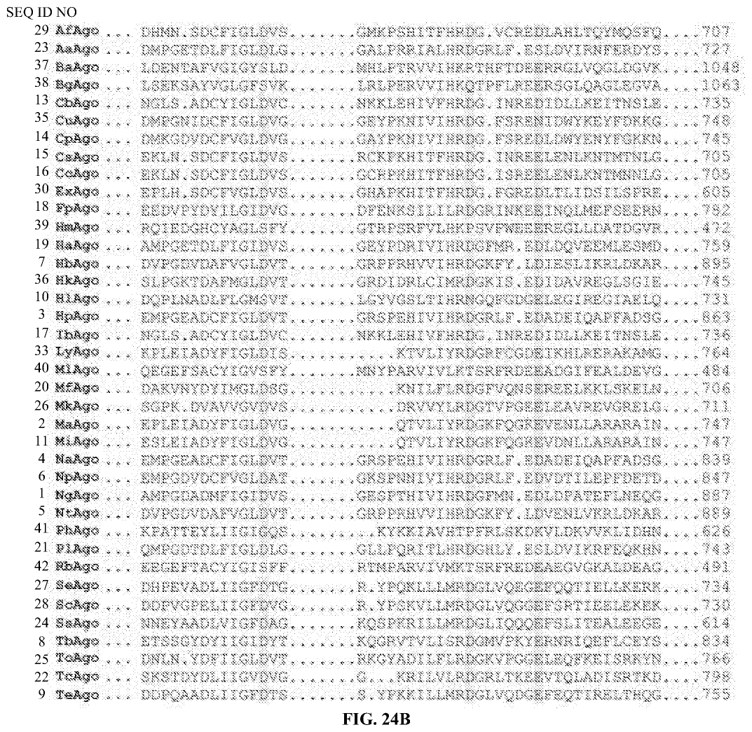

In some embodiments of any one of the use described above, the Argonaute nuclease comprises at least 5 of the 6 key conservative amino acid residues in FIGS. 24A-24B, wherein the 6 key conservative residues include 3 conservative residues in the 5′ phosphate binding site in the MID domain, and 3 conservative residues in the nuclease active site of the PIWI domain. In some embodiments, the Argonaute nuclease is an Argonaute nuclease selected from the group consisting of: (1) Argonaute nuclease having an NCBI Accession Number of AFZ73749.1 (SEQ ID NO: 1); (2) Argonaute nuclease having a GenBank Accession Number of ELZ29017.1 (SEQ ID NO: 3); (3) Argonaute nuclease having an NCBI Accession Number of WP_006111085.1 (SEQ ID NO: 4); (4) Argonaute nuclease having an NCBI Accession Number of WP_006090832.1 (SEQ ID NO: 5); (5) Argonaute nuclease having an NCBI Accession Number of WP_012265209.1 (SEQ ID NO: 2); (6) Argonaute nuclease having an NCBI Accession Number of WP_006183335.1 (SEQ ID NO: 6); (7) Argonaute nuclease having an NCBI Accession Number of WP_006054116.1 (SEQ ID NO: 7); (8) Argonaute nuclease having an NCBI Accession Number of WP_048159825.1 (SEQ ID NO: 8); (9) Argonaute nuclease having an NCBI Accession Number of WP_011056792.1 (SEQ ID NO: 9); (10) Argonaute nuclease having an NCBI Accession Number of WP_012659190.1 (SEQ ID NO: 10); (11) Argonaute nuclease having an NCBI Accession Number of WP_002747795.1 (SEQ ID NO: 11); (12) Argonaute nuclease having an NCBI Accession Number of WP_011378069.1 (SEQ ID NO: 12); (13) Argonaute nuclease having a GenBank Accession Number of CDA 11056.1 (SEQ ID NO: 13); (14) Argonaute nuclease having an NCBI Accession Number of WP_003477422.1 (SEQ ID NO: 14); (15) Argonaute nuclease having an NCBI Accession Number of WP_016205751.1 (SEQ ID NO: 15); (16) Argonaute nuclease having a GenBank Accession Number of CDB74854.1 (SEQ ID NO: 16); (17) Argonaute nuclease having an NCBI Accession Number of WP_007287731.1 (SEQ ID NO: 17); (18) Argonaute nuclease having an NCBI Accession Number of WP_012966655.1 (SEQ ID NO: 18); (19) Argonaute nuclease having a GenBank Accession Number of AHG02841.1 (SEQ ID NO: 19); (20) Argonaute nuclease having an NCBI Accession Number of WP_015791216.1 (SEQ ID NO: 20); (21) Argonaute nuclease having an NCBI Accession Number of WP_019364073.1 (SEQ ID NO: 21); (22) Argonaute nuclease having a GenBank Accession Number of AFK51052.1 (SEQ ID NO: 22); (23) Argonaute nuclease having an NCBI Accession Number of WP_011238781.1 (SEQ ID NO: 23); (24) Argonaute nuclease having an NCBI Accession Number of WP_012306644.1 (SEQ ID NO: 24); (25) Argonaute nuclease having an NCBI Accession Number of WP_012572468.1 (SEQ ID NO: 25): (26) Argonaute nuclease having a GenBank Accession Number of AAM02524.1 (SEQ ID NO: 26); (27) Argonaute nuclease having an NCBI Accession Number of WP_011244830.1 (SEQ ID NO: 27); (28) Argonaute nuclease having a GenBank Accession Number of ABD00306.1 (SEQ ID NO: 28); (29) Argonaute nuclease having an NCBI Accession Number of WP_012575214.1 (SEQ ID NO: 29); (30) Argonaute nuclease having a GenBank Accession Number of ACQ71053.1 (SEQ ID NO: 30); (31) Argonaute nuclease having a GenBank Accession Number of BAD80710.1 (SEQ ID NO: 31); (32) Argonaute nuclease having a GenBank Accession Number of ABB57564.1 (SEQ ID NO: 32). (33) Argonaute nuclease having a GenBank Accession Number of EAW33836.1 (SEQ ID NO: 33); (34) Argonaute nuclease having a GenBank Accession Number of ABD03669.1 (SEQ ID NO: 34); (35) Argonaute nuclease having a GenBank Accession Number of ALS17562.1 (SEQ ID NO: 35); and (36) Argonaute nuclease having an NCBI Accession Number of WP_049912037.1 (SEQ ID NO: 36).

In some embodiments of any one of the use described above, the Argonaute nuclease has at least about 80% sequence homology to any one of the 36 Argonaute nucleases disclosed above, and the Argonaute nuclease is capable of using a 5′ phosphorylated single-stranded oligonucleotide DNA as a guide to cleavage both strands of a double-stranded target DNA and induce a double-strand break at about 10-60° C., preferably at about 37° C. In some preferred embodiments, the Argonaute nuclease has at least about 90% sequence homology, such as at least 95% sequence homology to any one of the 36 Argonaute nucleases disclosed above.

In some embodiments, there is provided use of a gene sequence encoding any one of the 36 Argonaute nucleases or an Argonaute nuclease having at least about 80% sequence homology thereof, a plasmid or viral vector comprising the gene sequence, or a protein complex comprising an Argonaute nuclease and a 5′ phosphorylated single-stranded oligonucleotide DNA in gene editing, including targeted editing of intracellular viral DNA, in vitro targeted editing of DNA, and targeted editing of chromosomal DNA. Also provided is use of a gene sequence encoding the Argonaute nuclease in gene editing, construction of an expression vector, or preparation of a gene editing kit.

The present application provides use of an Argonaute nuclease, a gene sequence encoding the Argonaute nuclease, a plasmid thereof, or a protein complex in targeted genome editing, wherein upon induction of a double-strand break in the genome by the Argonaute nuclease, and in the presence of an exogenous DNA fragment having homologous sequences, the exogenous DNA fragment can be inserted into the double-strand break via cellular endogenous Homology directed recombination pathway, thereby achieving customized modification of the target gene or a functional region thereof.

### A. Argonautes

The methods described herein are based on Argonautes that are capable of using a single-stranded guide DNA to specifically recognize a target nucleic acid comprising a perfectly or substantially complementary sequence to the guide DNA at a temperature of about 10° C. to about 60° C., such as any one of about 10° C. to about 20° C., about 20° C. to about 30° C., about 30° C. to about 40° C., about 40° C. to about 50° C., about 50° C. to about 60° C., about 20° C. to about 40° C., about 10° C. to about 50° C., or about 15° C. to about 45° C. In some embodiments, the Argonaute is active at about any one of 10° C., 15° C., 20° C., 25° C., 30° C., 32° C., 34° C., 36° C., 37° C., 38° C., or 40° C. In some embodiments, the activity of the Argonaute is reduced by at least about any one of 10%, 20%, 30%, 40%, 50%, 60%, 70% 80%, 90% or more at least about 50° C.

The activity of the Argonaute includes specific binding and cleavage of a target locus. In some embodiments, the Argonaute is an endonuclease that cleaves the target nucleic acid at the target locus that hybridizes to the guide DNA. In some embodiments, the Ago-gDNA complex cleaves one strand of a double-stranded target DNA. In some embodiments, the Ago-gDNA complex cleaves both strands of a double-stranded target DNA. In some embodiments, the Ago-gDNA complex cleaves RNA, such as an mRNA. In some embodiments, the Argonaute does not have nuclease activity, i.e., the Argonaute forms a complex with the gDNA to specifically recognize (i.e., specifically bind) to the target locus without cleaving the target locus. Nuclease activity of the Ago-gDNA complex can be determined using known methods in the art, such as in vitro plasmid cleavage assay, T7E 1 assay, and sequencing. See, for example, experimental protocols in the Example section. Target nucleic acid binding activity of the Ago-gDNA complex can be determined using known methods in the art, such as Electrophoretic Mobility Shift Assay (EMSA).

The Argonautes suitable for the methods in the present application may further have one or more of the following characteristics: (1) the guide DNA is phosphorylated at the 5′ end; (2) once forming a complex, the guide DNA does not dissociate from the Ago protein at a temperature lower than about 50° C.; (3) the Ago nuclease requires only a single gDNA to induce a double-strand break in a double-stranded target DNA; (4) the Ago-gDNA complex can specifically recognize and/or cleave a target locus having no more than about 3 mismatches to the sequence of the gDNA; (5) the Ago-gDNA complex can specifically recognize and/or cleavage a target locus having a GC content of at least about 60%; (6) the Ago protein comprises at least 2 of the 3 conservative amino acids in the KQK motif of the 5′-phosphate binding site; and (7) the Ago protein comprises at least 2 of the 3 conservative amino acids in the DDE motif of the nuclease active site.

The Argonaute proteins having the properties described above may be derived from naturally-occurring Argonautes from a variety of organisms. In some embodiments, the Argonaute is derived from a prokaryotic species, such as an archaea or a bacterium. In some embodiments, the Ago is derived from a non-thermophilic bacterium. In some embodiments, the Ago is derived from a mesophilic bacterium. In some embodiments, the Ago is not derived from a thermophilic bacterium. In some embodiments, the Ago is derived from an algal species. In some embodiments, the Ago is derived from Natronobacterium gregoryi, Microcystis aeruginosa, Halogeometricum pallidum. Natrialba asiatica, Natronorubrum tibetense, Natrinema pellirubrum, Halogeometricum borinquense, Thermococcus barophilus, Thermosynechococcus elongatus, Halorubrum lacusprofundi, Microcystis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolvticus, Aromatoleum aromaticum, Thermococcus onnurineus, Methanopyrus kandleri, Synechococcus elongatus, Anoxybacillus flavithermus, Exiguobacterium sp., Lyngbya sp., Clostridium butyricum, Halorubrum kocurii, Burkholderia ambifaria, Burkholderia graminis, Haloarcula marismortui, Mesorhizobium loti, Rhodobacterales bacterium, or Pedobacter heparinus. In some embodiments, the Ago is derived from Synechococcus sp., Lyngbya sp., Microcystis sp., Halogeometricum borinquense, Natrinema pellirubrum, Natronobacterium gregoryi. Natronorubrum tibetense, Thermosynechococcus elongatus, Halogeometricum pallidum, Pedobacter heparinus, Rhodobacterales bacterium, Mesorhizobium loti, Haloarcula marismortui, Burkholderia graminis, Burkholderia ambifaria, Natrialba asiatica, or Microcystis aeruginosa. In some embodiments, the Ago is derived from Natronobacterium gregoryi, such as N. gregoryi SP2. In some embodiments, the Ago is derived from Microcystis aeruginosa, such as Microcyslis aeruginosa NIES 843. In some embodiments, the Ago is derived from Microcystis sp., such as Microcystis sp. 7806. In some embodiments, the Ago is derived from Pedobacter heparinus, such as Pedobacter heparinus DSM 2366. In some embodiments, the Ago is not derived from Thermus thermophilus or Rhodobacter sphaeroides. In some embodiments, the Ago is not derived from Synechococcus elongatus.

In some embodiments, the Argonaute is a type I prokaryotic Argonaute. In some embodiments, the type I prokaryotic Argonaute carries a DNA-targeting guide DNA. In some embodiments, the DNA nucleic acid-targeting nucleic acid targets one strand of a double stranded DNA (dsDNA) to produce a nick or a break of the dsDNA. In some embodiments, the nick or break triggers host DNA repair. In some embodiments, the host DNA repair is non-homologous end joining (NHEJ) or homology directed recombination (HDR). In some embodiments, the type I prokaryotic Argonaute is a long type I prokaryotic Argonaute. In some embodiments, the long type I prokaryotic Argonaute possesses an N-PAZ-MID-PIWI domain architecture. In some embodiments the long type I prokaryotic Argonaute possesses a catalytically active PIWI domain. In some embodiments, the long type I prokaryotic Argonaute possesses a catalytic tetrad comprising an aspartate-glutamate-aspartate-aspartate/histidine (DEDX) motif. In some embodiments, the catalytic tetrad binds one or more divalent metal ions, such as Mg²⁺, or Mn²⁺. In some embodiments, the type I prokaryotic Argonaute anchors the 5′ phosphate end of a guide DNA.

The Argonaute protein may comprise one or more domains. The Argonaute protein may comprise a domain selected from a PAZ domain, a MID domain, and a PIWI domain or any combination thereof. The Argonaute protein may comprise a domain architecture of N-PAZ-MID-PIWI-C. The PAZ domain may comprise an oligonucleotide-binding fold to secure a 3′ end of a guide DNA. Release of the 3′-end of the guide DNA from the PAZ domain may facilitate the transitioning of the Argonaute ternary complex into a cleavage active conformation. The MID domain may bind a 5′ phosphate and a first nucleotide of the gDNA. In some embodiments, the MID domain comprises a 5′ phosphate binding site having at least 2 or all 3 of the 3 conserved residues in the KQK motif as shown in FIG. 24A. In some embodiments, a lysine (K) residue in the KQK motif is replaced by an arginine (R). In some embodiments, a glutamine (Q) residue in the KQK motif is replaced by an asparagine (N), or a positively charged residue, such as lysine (K) or Arginine®. The target nucleic acid can remain bound to the Argonaute through many rounds of cleavage by means of anchorage of the 5′ phosphate in the MID domain.

The Argonaute protein can comprise a nucleic acid-binding domain. The nucleic acid-binding domain can comprise a region that contacts a nucleic acid. The nucleic acid-binding domain can bind DNA or RNA, or both DNA and RNA. In some embodiments, the Argonaute protein binds a DNA and cleaves the DNA. In some embodiments, the Argonaute protein binds a single-stranded gDNA and cleaves a double-stranded DNA. In some embodiments, the Argonaute protein binds two single-stranded gDNAs and cleaves a double-stranded DNA. In some embodiments, the nucleic acid-binding domain comprises a PAZ domain, which can use its oligonucleotide-binding fold to secure the 3′ end of the designed nucleic acid-targeting nucleic acid.

The Argonaute can comprise a nucleic acid-cleaving domain, such as a PIWI domain. In some embodiments, the Ago comprises a PIWI domain comprising a nuclease active site. In some embodiments, the nuclease active site binds a divalent cation. In some embodiments, the nuclease active site binds two divalent cations. For example, a first divalent cation may initiate a nucleophilic attack and activate a water molecule, and a second divalent cation may stabilize the transition state and leaving group. In some embodiments, the Argonaute comprises a nuclease active site having at least 2 or all 3 of the 3 conserved residues in the DDE motif as shown in FIG. 24B. In some embodiments, an aspartic acid (D) residue in the DDE motif is replaced by a glutamic acid (E). In some embodiments, a glutamic acid (E) residue in the DDE motif is replaced by an aspartic acid (D). In some embodiments, the nuclease active site further comprises one or more basic residues, such as histidine, arginine, lysine or combinations thereof. The histidine, arginine and/or lysine may play a role in catalysis and/or cleavage. In some embodiments, the nuclease active site comprises four negatively charged, evolutionary conserved amino acids, such as aspartate-glutamate-aspartate-aspartate/histidine (DEDX, SEQ ID NO: 157), which form a catalytic tetrad that binds two divalent metal ions (such as Mg²⁺ ions) and cleave a target nucleic acid into products bearing a 3′ hydroxyl and 5′ phosphate group. In some embodiments, depending on the type of Ago, the method is carried out in the presence of a divalent metal ion, such as Mg²⁺, at a concentration of at least about any one of 0.1 mM, 0.2 mM, 0.5 mM, 1 mM, 2 mM, 3 mM, 4 mM, 5 mM, 10 mM, 15 mM, 20 mM or more. Cleavage of the target nucleic acid by Argonaute can occur at a single phosphodiester bond in one strand, or at two phosphodiester bonds in both strands of the target nucleic acid. In some embodiments, the Ago protein comprises a functional nuclease active site that does not contain the DDE or DEDX (SEQ ID NO: 157) motif. In some embodiments, the Ago protein does not comprise a functional nuclease active site.

Exemplary Argonautes and their protein sequences are shown in Table 1 below. In some embodiments, the Argonaute is AaAgo, AfAgo, BaAgo, BgAgo, CbAgo, CcAgo, CpAgo, CsAgo, CuAgo, ExAgo, FpAgo, HaAgo, HbAgo, HkAgo, HIAgo, HmAgo, HpAgo, IbAgo, LyAgo, MaAgo, MfAgo, Migo, MkAgo, MIAgo, Ago, NgAgo, NpAgo, NtAgo, PhAgo, PIAgo, RbAgo, ScAgo, SeAgo, SsAgo, SyAgo, TbAgo, TcAgo. TeAgo, ToAgo, or a functional derivative thereof. In some embodiments, the Argonaute is NgAgo, or a functional derivative thereof. In some embodiments, the Argonaute is PhAgo, or a functional derivative thereof. In some embodiments, the Argonaute is MiAgo, or a functional derivative thereof. In some embodiments, the Argonaute is MaAgo, or a functional derivative thereof. In some embodiments, the Argonaute is not TtAgo or RsAgo. In some embodiments, the Argonaute is not SeAgo.

TABLE 1
Exemplary Argonaute protein sequences.

| | | | | |
|---|---|---|---|---|
| SEQ | | | | |
| ID | | | | |
| NO | Ago | Species | Strain | Accession No. |

Agos that cleave target nucleic acids at physiological temperatures (10° C.-60° C., e.g., 37° C.).

| | | | | |
|---|---|---|---|---|
| 1 | NgAgo | Natronobacterium gregoryi | SP2 | NCBI: AFZ73749.1 |
| 2 | MaAgo | Microcystis aeruginosa | NIES 843 | NCBI: WP_012265209.1 |
| 3 | HpAgo | Halogeometricum pallidum | JCM 14848 | GenBank: ELZ29017.1 |
| 4 | NaAgo | Natrialba asiatica | DSM 12278 | NCBI: WP_006111085.1 |
| 5 | NtAgo | Natronorubrum tibetense | GA33 | NCBI: WP_006090832.1 |
| 6 | NpAgo | Natrinema pellirubrum | DSM 15624 | NCBI: WP_006183335.1 |
| 7 | HbAgo | Halogeometricum | DSM 11551 | NCBI: WP_006054116.1 |
| | | borinquense | | |
| 8 | TbAgo | Thermococcus barophilus | MP | NCBI: WP_048159825.1 |
| 9 | TeAgo | Thermosynechococcus | Bp-1 | NCBI: WP_011056792.1 |
| | | elongatus | | |
| 10 | HlAgo | Halorubrum lacusprofundi | ATCC 49239 | NCBI: WP_012659190.1 |
| 11 | MiAgo | Microcystis sp. | 7806 | NCBI: WP_002747795.1 |
| 12 | SyAgo | Synechococcus sp. | PCC7942 | NCBI: NCBI: |
| | | | | WP_011378069.1 |
| 13 | CbAgo | Clostridium bartlettii | CAG1329 | GenBank: CDA11056.1 |
| 14 | CpAgo | Clostridium perfringens | WAL-14572 | NCBI: WP_003477422.1 |
| 15 | CsAgo | Clostridium sartagoforme | AAU1 | NCBI: WP_016205751.1 |
| 16 | CcAgo | Clostridium sp. | CAG265 | GenBank: CDB74854.1 |
| 17 | IbAgo | Intestinibacter bartlettii | DSM 16795 | NCBI: WP_007287731.1 |
| 18 | FpAgo | Ferroglobus placidus | DSM 10642 | NCBI: WP_012966655.1 |
| 19 | HaAgo | Halobacterium sp. | DL1 | GenBank: AHG02841.1 |
| 20 | MfAgo | Methanocaldococcus fervens | AG86 | NCBI: WP_015791216.1 |
| 21 | PlAgo | Pseudomonas luteola | | NCBI: WP_019364073.1 |
| 22 | TcAgo | Thermogladius cellulolyticus | 1633 | GenBank: AFK51052.1 |
| 23 | AaAgo | Aromatoleum aromaticum | EbN1 | NCBI: WP_011238781.1 |
| 24 | SsAgo | Synechococcus sp. | PCC 7002 | NCBI: WP_012306644.1 |
| 25 | ToAgo | Thermococcus onnurineus | NA1 | NCBI: WP_012572468.1 |
| 26 | MkAgo | Methanopyrus kandleri | AV19 | GenBank: AAM02524.1 |
| 27 | SeAgo | Synechococcus elongatus | PCC 6301 | NCBI: WP_011244830.1 |
| 28 | ScAgo | Synechococcus sp. | JA-3-3Ab | GenBank: ABD00306.1 |
| 29 | AfAgo | Anoxybacillus flavithermus | WK1 | NCBI: WP_012575214.1 |
| 30 | ExAgo | Exiguobacterium sp. | AT1b | GenBank: ACQ71053.1 |
| 31 | | Synechococcus elongatus | PCC 6301 | GenBank: BAD80710.1 |
| 32 | | Synechococcus elongatus | PCC 7942 | GenBank: ABB57564.1 |
| 33 | LyAgo | Lyngbya sp. | PCC 8106 | GenBank: EAW33836.1 |
| 34 | | Synechococcus sp. | JA-2-3B′a(2-13) | GenBank: ABD03669.1 |
| 35 | CuAgo | Clostridium butyricum | | GenBank: ALS17562.1 |
| 36 | HkAgo | Halorubrum kocurii | | NCBI: WP_049912037.1 |
| 37 | BaAgo | Burkholderia ambifaria | MEX-5 | |
| 38 | BgAgo | Burkholderia graminis | | |
| 39 | HmAgo | Haloarcula marismortui | ATCC 43049 | |
| 40 | MlAgo | Mesorhizobium loti | M4FF303099 | |
| 41 | PhAgo | Pedobacter heparinus | DSM 2366 | |
| 42 | RbAgo | Rhodobacterales bacterium | HTCC2654 | |

Thermophilic Agos that cleave target nucleic acids at elevated temperatures (>60° C.) only.

| | | |
|---|---|---|
| 43 | TtAgo | Thermus thermophilus |
| 44 | RsAgo | Rhodobacter sphaeroides |

In some embodiments, the Argonaute protein comprises a sequence having at least about any one of 70%, 75%, 80%, 85%, 90%, 95%, 97%, 99%, or more, or 100% sequence homology to wild-type Argonautes of Table 1 (e.g., NgAgo) in the MID domain, PAZ domain, and/or PIWI domain. In some embodiments, the Argonaute protein comprises a sequence having at least about any one of 30%, 40%, 50%, 60%, 70%, 75%, 80%, 85%, 90%, 95%, 97%, 99%, or more, or about 100% sequence identity to wild-type Argonautes of Table 1 (e.g., NgAgo, MiAgo, MaAgo, or PhAgo) in the MID domain, PAZ domain, and/or PIWI domain. In some embodiments, the Argonaute protein comprises a sequence having at least about any one of 70%, 75%, 80%, 85%, 90%, 95%, 97%, 99%, or more, or 100% sequence homology to a sequence selected from SEQ ID NOs: 1-42. In some embodiments, the Argonaute protein comprises a sequence having at least about any one of 30%, 40%, 50%, 60%, 70%, 75%, 80%, 85%, 90%, 95%, 97%, 99%, or more, or about 100% sequence identity to a sequence selected from SEQ ID NOs: 1-42. In some embodiments, the Ago protein has a sequence selected from SEQ ID NOs: 1-42. In some embodiments, the Argonaute protein comprises a sequence having at least about any one of 70%, 75%, 80%, 85%, 90%, 95%, 97%, 99%, or more, or 100% sequence homology to SEQ ID NO: 1. In some embodiments, the Argonaute protein comprises a sequence having at least about any one of 30%, 40%, 50%, 60%, 70%, 75%, 80%, 85%, 90%, 95%, 97%, 99%, or more, or about 100% sequence identity to SEQ ID NO: 1. In some embodiments, the Ago protein has SEQ ID NO: 1. In some embodiments, the Argonaute protein comprises a sequence having at least about any one of 70%, 75%, 80%, 85%, 90%, 95%, 97%, 99%, or more, or 100% sequence homology to SEQ ID NO: 2. In some embodiments, the Argonaute protein comprises a sequence having at least about any one of 30%, 40%, 50%, 60%, 70%, 75%, 80%, 85%, 90%, 95%, 97%, 99%, or more, or about 100% sequence identity to SEQ ID NO: 2. In some embodiments, the Ago protein has SEQ ID NO: 2. In some embodiments, the Argonaute protein comprises a sequence having at least about any one of 70%, 75%, 80%, 85%, 90%, 95%, 97%, 99%, or more, or 100% sequence homology to SEQ ID NO: 11. In some embodiments, the Argonaute protein comprises a sequence having at least about any one of 30%, 40%, 50%, 60%, 70%, 75%, 80%, 85%, 90%, 95%, 97%, 99%, or more, or about 100% sequence identity to SEQ ID NO: 11. In some embodiments, the Ago protein has SEQ ID NO: 11. In some embodiments, the Argonaute protein comprises a sequence having at least about any one of 70%, 75%, 80%, 85%, 90%, 95%, 97%, 99%, or more, or 100% sequence homology to SEQ ID NO: 41. In some embodiments, the Argonaute protein comprises a sequence having at least about any one of 30%, 40%, 50%, 60%, 70%, 75%, 80%, 85%, 90%, 95%, 97%, 99%, or more, or about 100% sequence identity to SEQ ID NO: 41. In some embodiments, the Ago protein has SEQ ID NO:41.

In some embodiments, the Argonaute is a modified form of a wildtype Ago protein in Table 1. The modified form of the wild type Argonaute can comprise an amino acid change (e.g., deletion, insertion, or substitution) that reduces the nuclease activity of the Argonaute. For example, the modified Argonaute can have less than about any one of 90%, 80%, 70%, 60%, 50%, 40%, 30%, 20%, 10%, 5%, 1% or less of the nuclease activity of the wild-type Argonaute (e.g., NgAgo, MiAgo, MaAgo, or PhAgo). The modified form of the Argonaute can have no substantial nuclease activity. For example, one or more of the conserved residues in the DDE motif in the nuclease active site of the wildtype Ago can be mutated, e.g., to alanine, to provide an Argonaute that has no nuclease activity. One skilled in the art will recognize that mutations other than alanine substitutions are suitable. In some embodiments, sequences can be inserted to an Argonaute protein to reduce its activity. In some embodiments, the modified form of the wild type Argonaute can have more than about any one of 10%, 20%, 30%, 40%, 50%, 60%, 70%, 80%, 90%, or more nuclease activity of the wild-type Argonaute (e.g., NgAgo, MiAgo, MaAgo, or PhAgo).

Unless specified otherwise, “Argonaute” or “Ago” may refer to the polypeptide(s) corresponding to a wildtype Argonaute protein or a functional derivative thereof, or the polynucleotide(s) encoding the polypeptide. “Functional derivative” refers to a modified form of a protein having substantially the same function or activity as the wildtype protein, but comprising one or more changes to the amino acid sequence or chemical composition, including, but not limited to, mutations (e.g., deletion, insertion, substitution, etc.), non-natural amino acid variants, fusions to other polypeptides (e.g., affinity tags, signal peptides, etc.), conjugation to non-amino acid moieties (e.g., dyes), and a chimeric protein having other functional modalities.

In some embodiments, the Ago protein is a fusion protein. A fusion protein can comprise one or more of the same non-native sequences not naturally found in the wildtype Ago. In some embodiments, the Ago protein is fused to one or more affinity tags, such as HA and FLAG tags, which can facilitate purification of the Ago protein. In some embodiments, the Ago protein is fused to a fluorescent protein, such as GFP or RFP, for visualization or tracking of the Ago protein inside cells. In some embodiments, the Ago protein is fused to a self-penetrating peptide to facilitate intracellular delivery of the Ago-gDNA complex. In some embodiments, the Ago protein is fused to a subcellular localization signal of the Ago, e.g., a nuclear localization signal (NLS) for targeting to the nucleus, a mitochondrial localization signal for targeting to the mitochondria, a chloroplast localization signal for targeting to a chloroplast, an endoplasmic reticulum (ER) retention signal, and the like. The fusion moiety (such as affinity tag, fluorescent protein, self-penetrating peptide, and/or localization signal) may be fused to either the N-terminus, or the C-terminus, or both termini of the Ago protein using standard recombinant methods known in the art.

Further provided herein are isolated Argonaute proteins and use of any one of the Argonaute proteins described herein for gene editing, including, but not limited to, modification of target nucleic acids, targeted editing of intracellular viral DNA, in vitro targeted editing of DNA, and targeted editing of chromosomal DNA. Also provided is use of any one of the Argonaute proteins in preparation of a gene editing kit or an analytic or interference agent.

### Nucleic Acids Encoding Argonaute Proteins

The Argonaute proteins described herein can be obtained using standard recombinant techniques, or expressed recombinantly in the host cell, i.e., the cell containing the target nucleic acid to be modified by the Ago protein.

The nucleic acid encoding the Argonaute protein can be isolated and sequenced from any of the species listed in Table 1. Alternatively, an Argonaute coding sequence can be designed based on a naturally occurring Ago sequence, such as any one of SEQ ID No: 1-42, and a nucleic acid having the designed sequence can be synthesized using nucleotide synthesizer or PCR techniques. In some embodiments, the Ago coding sequence is further engineered, such as by site-directed mutagenesis, for expression of a functional derivative or mutant variant of the wildtype Argonaute protein. In some embodiments, the nucleic acid comprising an Ago coding sequence fused to one or more additional component, such as a signal sequence. In some embodiments, for nuclear expression of the Ago protein in the host cell, a nuclear localization sequence is fused to the N-terminus of the Ago sequence.

In some embodiments, the nucleic acid encoding the Ago protein is codon optimized for expression in host cells, such as eukaryotic cells. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (trans) molecules. The predominance of selected trans in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 20Q0” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, codon optimization is not required. In some embodiments, codon optimization is preferable.

In some embodiments, the nucleic acid encoding the Ago protein is subcloned into a recombinant vector capable of replicating and expressing heterologous polynucleotides in a host cell. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. In some embodiments, a vector for expression of the Ago protein in prokaryotic cells is provided. In some embodiments, a vector for expression of the Ago protein in eukaryotic cells, such as mammalian cells, is provided.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses.

In some embodiments, the vector is a plasmid. Examples of plasmids include, but are not limited to, pGEX6P-1, and pcDNA3.1. In some embodiments, the plasmid is supercoiled. In some embodiments, the plasmid is ultrapure. As used herein, “ultrapure” plasmid refers to plasmid preparations that is substantially free from contamination by endotoxins or non-viral microorganisms, and the plasmid is at least about any one of 90%, 95%, 97%, 99%, or more supercoiled. Ultrapure plasmids can be prepared using commercial plasmid purification kits. Supercoiling of a plasmid can be determined by gel electrophoresis.

In some embodiments, the vector is a viral vector. Examples of viral vectors include, but are not limited to, adenoviral vectors, adeno-associated virus vectors, lentiviral vector, retroviral vectors, vaccinia vector, herpes simplex viral vector, and derivatives thereof. Viral vector technology is well known in the art and is described, for example, in Sambrook et al. (2001, Molecular Cloning: A Laboratory Manual, Cold Spring Harbor Laboratory, New York), and in other virology and molecular biology manuals.

A number of viral based systems have been developed for gene transfer into mammalian cells. For example, retroviruses provide a convenient platform for gene delivery systems. The heterologous nucleic acid can be inserted into a vector and packaged in retroviral particles using techniques known in the art. The recombinant virus can then be isolated and delivered to the host cell in vitro. In some embodiments, adenovirus vectors are used. In some embodiments, lentivirus vectors are used. In some embodiments, self-inactivating lentiviral vectors are used.

In general, a suitable vector contains an origin of replication functional in at least one organism, a promoter sequence, convenient restriction endonuclease sites, and one or more selectable markers. Additional components of the vector may include, but are not limited to, a ribosome binding site (RBS), a signal sequence, and a transcription termination sequence. In general, vectors containing replicon and control sequences which are derived from species compatible with the host cell are used in connection with these hosts.

In some embodiments, the nucleic acid is operably linked to a promoter. A large number of promoters recognized by a variety of potential host cells are well known. In some embodiments, the promoter is an inducible promoter. In some embodiments, the promoter is a constitutive promoter. Inducible promoter is a promoter that initiates increased levels of transcription of the coding sequence under its control in response to changes in the culture condition, e.g. the presence or absence of a nutrient or a change in temperature.

Promoters suitable for use with prokaryotic host cells include the phoA promoter, -lactamase and lactose promoter systems, alkaline phosphatase promoter, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the Ago protein.

Polypeptide transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

In some embodiments, the vector for expression in higher eukaryotes comprises an enhancer sequence. Many enhancer sequences are now known from mammalian genes (globin, elastase, albumin, α-fetoprotein, and insulin), or from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the polypeptide encoding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. For example, at the 3′ end of most eukaryotic is an AATAAA sequence that may be the signal for addition of the poly A tail to the 3′ end of the coding sequence. These regions contain nucleotide segments transcribed as polyadenylated fragments in the untranslated portion of the polypeptide-encoding mRNA. All of these sequences may be inserted into eukaryotic expression vectors.

In some embodiments, the nucleic acid encoding the Ago protein is a RNA. In some embodiments, the nucleic acid is a RNA vector. In some embodiments, the nucleic acid is an mRNA encoding the Ago protein. In some embodiments, the mRNA encoding the Ago protein comprises one or more modifications to enhance its stability, enhance its expression, and/or reduce its immunogenicity. In some embodiments, the mRNA comprises a modified backbone and/or modified internucleoside linkages. In some embodiments, the mRNA comprises one or more phosphorothioate linkages. In some embodiments, the mRNA comprises one or more modified nucleobases, such as 5-methyl cytosine and/or pseudouridine. In some embodiments, the mRNA comprises a 5′ cap.

Further provided herein are nucleic acids and vectors (such as plasmids or viral vectors) encoding any one of the Argonaute proteins described herein, and use of any one of the nucleic acids and vectors for expression of the Ago proteins, or for gene editing, including, but not limited to, modification of target nucleic acids, targeted editing of intracellular viral DNA, in vitro targeted editing of DNA, and targeted editing of chromosomal DNA. Also provided is use of any one of the nucleic acids and vectors in preparation of a gene editing kit or an analytic or interference agent.

### B. Guide DNA

The Argonautes described herein uses a guide DNA that is a single-stranded oligonucleotide DNA having a sequence designed to be perfectly complementary or substantially complementary to the target nucleic acid to be modified. In some embodiments, a single guide DNA is required for the Ago protein to induce a double-strand break in a double-stranded target nucleic acid. In some embodiments, two guide DNAs each targeting an opposite strand of a double-stranded target nucleic acid are required for the Ago protein to induce a double-strand break in the target nucleic acid. In some embodiments, the Ago protein uses a plurality of guide DNAs targeting different sequences in the target nucleic acid.

In some embodiments, the Ago protein and the guide DNA(s) do not naturally occur together. In some embodiments, the guide DNA comprises a sequence that does not substantially hybridize to any part of the genomic sequence of the host cell. In some embodiments, the guide DNA comprises a modification, such as 5′ phosphorylation, or modification to the base or backbone portion of a nucleotide, that does not naturally occur in the host cell.

In some embodiments, the Ago protein does not have any preference for specific sequence or nucleotides in the guide DNA. In some embodiments, the 5′ terminus of the guide DNA is phosphorylated. 5′ phosphorylated guide DNA can be prepared by chemical synthesis, or by phosphorylating an oligonucleotide DNA using a kinase, such as T4 PNK. In some embodiments, the 5′ phosphorylated guide DNA is produced endogenously by a bacterial cell. For example, a plasmid can be transfected into the bacterial cell to produce 5′ phosphorylated guide DNAs targeting sequences derived from the plasmid. In some embodiments, the plasmid is a linearized plasmid. In some embodiments, the gDNA is substantially purified, such as at least about any one of 80%, 85%, 90%, 95%, 99%, or more pure. Guide DNA can be purified by known methods in the art, such as HPLC, gel electrophoresis, or using DNA purification kits.

The length of the guide DNA may influence the activity (such as specific binding and/or nuclease activity) of the Ago-gDNA complex. In some embodiments, the guide DNA has about 10 to about 50 nucleotides (“nt”), such as any one of about 10 nt to about 20 nt, about 20 nt to about 30 nut, about 30 nt to about 40 nt, about 40 nt to about 50 nt, about 15 nt to about 30 nt, about 20 nt to about 40 nt, about 15 nt to about 25 nt, or about 20 nt to about 35 nt. In some embodiments, the guide DNA has about any one of 18, 19, 20, 21, 22, 23, 24, 25, 26, 27, 28, 29 or 30 nucleotides. In some embodiments, the guide DNA has about 20 nucleotides to about 27 nucleotides, or about 23 nucleotides to 25 nucleotides. In some embodiments, wherein the Ago protein is derived from NgAgo, the guide DNA is 24 nucleotides long. The optimal length of the gDNA may vary depending on the species from which the Ago protein is derived, and can be determined by a skilled person in the art using gDNAs of different length in an in vitro or in cell activity assay, such as binding, plasmid cleavage, or reporter gene (e.g., GFP) silencing.

In some embodiments, the activity (such as specific binding and/or nuclease activity) of the Ago-gDNA complex is sensitive to nucleotide mismatches between the gDNA and the target locus. In some embodiments, the efficiency of modification or cleavage of the target locus is reduced by at least about any of 10%, 20%, 30%, 40%, 50%, 60%, 70%, 80%, 90%, 95% or more when the gDNA comprises one mismatch to the target locus. In some embodiments, the efficiency of modification or cleavage of the target locus is reduced by at least about any of 10%, 20%, 30%, 40%, 50%, 60%, 70%, 80%, 90%, 95% or more when the gDNA comprises two mismatches, such as two consecutive mismatches, to the target locus. In some embodiments, the efficiency of modification or cleavage of the target locus is reduced by at least about any of 10%, 20%, 30%, 40%, 50%, 60%, 70%, 80%, 90%, 95% or more when the gDNA comprises three mismatches, such as three consecutive mismatches, to the target locus. In some embodiments, the gDNA is perfectly complementary to the target locus. In some embodiments, the gDNA has one mismatch to the target locus. In some embodiments, the gDNA has two mismatches to the target locus. In some embodiments, the gDNA has three mismatches to the target locus. In some embodiments, the gDNA does not have a mismatch in any one of positions 8, 9, 10, or 11 from the 5′ terminus, compared to the sequence of the target locus. In some embodiments, the gDNA does not have any three consecutive nucleotides that are mismatches to the target locus.

In some embodiments, the guide DNA comprises a modified backbone and/or modified internucleoside linkages. In some embodiments, the guide DNA comprises one or more phosphorothioate linkages. Various salts (e.g., potassium chloride or sodium chloride), mixed salts, and free acid forms can also be included. In some embodiments, the guide DNA comprises one or more modified nucleobases.

### C. Ago-gDNA Complex

In some embodiments, the Ago protein and the gDNA form a complex in the cell. In some embodiments, the Ago protein and the gDNA are provided to an in vitro target nucleic acid, or a cell in a pre-formed complex. Generally, the Ago protein and the gDNA bind to each other in a molar ratio of about 1:1. As used herein, binding of the Ago protein to gDNA to form an Ago-gDNA complex is referred herein as “loading” of gDNA to the Ago protein. A pre-formed complex is referred herein as an Ago “pre-loaded” with a gDNA. In some embodiments, the Ago-gDNA complex follows a “one-guide faithful” rule, i.e., the Ago protein does not dissociate from the bound gDNA in the complex, or switch a pre-loaded gDNA with a free, unbound gDNA. In some embodiments, the Ago protein does not dissociate from a pre-loaded gDNA at a temperature lower than about any one 40° C., 45° C., 50° C., or 55° C. In some embodiments, the Ago protein does not dissociate from a pre-loaded gDNA or bind to a free gDNA at about 37° C. over an incubation period of at least about any one of 4, 8, 12, 16, 24, or more hours. In some embodiments, a pre-formed Ago-gDNA complex does exchange the gDNA with a different gDNA in the cell.

The Ago-gDNA complex may be prepared using any suitable method. In some embodiments, the Ago-gDNA complex is prepared by mixing the Ago protein with the gDNA. In some embodiments, the Ago protein and the gDNA are mixed at a temperature of at least about any one of 40° C., 45° C., 50° C., 55° C., or more to provide the Ago-gDNA complex.

In some embodiments, the Ago-gDNA complex is prepared by expressing the Ago protein in the presence of the gDNA. In some embodiments, a nucleic acid (such as a vector) encoding the Ago protein and a gDNA are co-transfected into a host cell to provide the Ago-gDNA complex. In some embodiments, the host cell is a cell that does not have endogenous 5′ phosphorylated gDNA. In some embodiments, the host cell is a mammalian cell, such as 293T cell. In some embodiments, the host cell is a bacterial cell, such as E. coli. In some embodiments, a nucleic acid (such as a plasmid) encoding the Ago protein and a linearized vector comprising the gDNA are co-transfected into a bacterial cell to provide the Ago-gDNA complex. In some embodiments, a nucleic acid (such as a vector) encoding the Ago protein is expressed in an in vitro protein translation system in the presence of a gDNA to provide the Ago-gDNA complex. In some embodiments, the Ago-gDNA complex is purified from the host cell or the in vitro protein translation system using known protein purification methods in the art, such as affinity chromatography, and/or size exclusion chromatography. In some embodiments, the Ago protein comprises a polypeptide affinity tag, which can be used for purifying the Ago-gDNA complex from the host cell or the in vitro protein translation system. Suitable polypeptide affinity tags include, but are not limited to, a His tag (e.g., a 6× His tag), a hemagglutinin (HA) tag, a FLAG tag, a Myc tag, a GST tag, a MBP tag, and chitin binding protein tag, a calmodulin tag, a V5 tag, and a streptavidin binding tag. In some embodiments, the Ago-gDNA is substantially pure. For example, the composition comprising the pre-formed Ago-gDNA complex comprises at least about any one of 80%, 85%, 90%, 95%, 99%, or more Ago-gDNA complex.

### D. Target Nucleic Acid

The methods disclosed herein are applicable for a variety of target nucleic acids. In some embodiments, the target nucleic acid is a DNA. In some embodiments, the target nucleic acid is a RNA, such as mRNA. In some embodiments, the target nucleic acid is single-stranded. In some embodiments, the target nucleic acid is double-stranded. In some embodiments, the target nucleic acid comprises both single-stranded and double-stranded regions. In some embodiments, the target nucleic acid is linear. In some embodiments, the target nucleic acid is circular. In some embodiments, the target nucleic acid comprises one or more modified nucleotides, such as methylated nucleotides, damaged nucleotides, or nucleotides analogs. In some embodiments, the target nucleic acid is not modified.

The target nucleic acid may be of any length, such as about at least any one of 100 bp, 200 bp, 500 bp, 1000 bp, 2000 bp, 5000 bp, 10 kb, 20 kb, 50 kb, 100 kb, 200 kb, 500 kb, 1 Mb, or longer. The target nucleic acid may also comprise any sequence. In some embodiments, the target nucleic acid is GC-rich, such as having at least about any one of 40%, 45%, 50%, 55%, 60%, 65%, or higher GC content. In some embodiments, the target nucleic acid is not GC-rich. In some embodiments, the target nucleic acid has one or more secondary structures or higher-order structures. In some embodiments, the target nucleic acid is not in a condensed state, such as in a chromatin, to render the target locus inaccessible by the Ago-gDNA complex.

In some embodiments, the target nucleic acid is present in a cell. In some embodiments, the target nucleic acid is present in the nucleus of the cell. In some embodiments, the target nucleic acid is endogenous to the cell. In some embodiments, the target nucleic acid is a genomic DNA. In some embodiments, the target nucleic acid is a chromosomal DNA. In some embodiments, the target nucleic acid is a protein-coding gene or a functional region thereof, such as a coding region, or a regulatory element, such as a promoter, enhancer, a 5′ or 3′ untranslated region, etc. In some embodiments, the target nucleic acid is a non-coding gene, such as transposon, miRNA, tRNA, ribosomal RNA, ribozyme, or lincRNA. In some embodiments, the target nucleic acid is a plasmid.

In some embodiments, the target nucleic acid is exogenous to a cell. In some embodiments, the target nucleic acid is a viral nucleic acid, such as viral DNA or viral RNA. In some embodiments, the target nucleic acid is a horizontally transferred plasmid. In some embodiments, the target nucleic acid is integrated in the genome of the cell. In some embodiments, the target nucleic acid is not integrated in the genome of the cell. In some embodiments, the target nucleic acid is a plasmid in the cell. In some embodiments, the target nucleic acid is present in an extrachromosomal array.

In some embodiments, the target nucleic acid is an isolated nucleic acid, such as an isolated DNA or an isolated RNA. In some embodiments, the target nucleic acid is present in a cell-free environment. In some embodiments, the target nucleic acid is an isolated vector, such as a plasmid. In some embodiments, the target nucleic acid is an ultrapure plasmid.

The target locus is a segment of the target nucleic acid that hybridizes to the gDNA. In some embodiments, the target locus is cleaved by the Ago-gDNA complex. In some embodiments, the target nucleic acid has only one copy of the target locus. In some embodiments, the target nucleic acid has more than one copy, such as at least about any one of 2, 3, 4, 5, 10, 100, or more copies of the target locus. For example, a target locus comprising a repeated sequence in a genome of a viral nucleic acid or a bacterium may be targeted by an Ago-gDNA to inhibit or kill the virus or the bacterium.

In some embodiments, the target locus is a DNA locus. In some embodiments, the target locus is a RNA locus. In some embodiments, the target locus is double stranded. In some embodiments, the target locus is single-stranded. In some embodiments, the target locus is a double-stranded DNA.

The target locus may comprise any sequence, as the Ago protein has no preferences to bind a particular sequence or sequence motif. In some embodiments, the target locus is GC rich. In some embodiments, the target locus has a GC content of at least about any one of 40%, 50%, 60%, 70%, 80%, or more. In some embodiments, the target locus is a GC-rich fragment in a non-GC-rich target nucleic acid. In some embodiments, the target locus is present in a readily accessible region of the target nucleic acid. In some embodiments, the target locus is in an exon of a target gene. In some embodiments, the target locus is across an exon-intron junction of a target gene. In some embodiments, the target locus is present in a non-coding region, such as a regulatory region of a gene. In some embodiments, wherein the target nucleic acid is exogenous to a cell, the target locus comprises a sequence that is not found in the genome of the cell.

In some embodiments, the target nucleic acid, and/or the target locus includes a sequence associated with a signaling biochemical pathway, e.g., a signaling biochemical pathway-associated gene or polynucleotide. Examples of target nucleic acids include disease-associated genes or polynucleotides. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation(s) that is directly responsible or is in linkage disequilibrium with a gene(s) that is responsible for the etiology of a disease. The transcribed or translated products may be known or unknown, and may be at a normal or abnormal level. Mutations in these genes and pathways can result in production of improper proteins or proteins in improper amounts which affect function. In some embodiments, the target locus is a disease-associated locus. In some embodiments, the target locus comprises a mutation or genetic variation in a disease-associated gene. Examples of disease-associated genes and polynucleotides, and disease-associated loci are available from MeKusick-Nathans Institute of Genetic Medicine, Johns Hopkins University (Baltimore, Md.) and National Center for Biotechnology Information, National Library of Medicine (Bethesda, Md.), available on the World Wide Web.

The present application contemplates methods of generating isogenic lines of cells of mammalian cells for the study of genetic variations in a disease. In some embodiments, the method provides a single-nucleotide substitution at the target locus, which can be used to study the effect of single nucleotide polymorphisms. The present application also contemplates genome modification of microbes, cells, plants, animals or synthetic organisms for the generation of biomedically, agriculturally, and industrially useful products. The methods may be used as a biological research tool, for understanding the genome, e.g. gene knockout or knock-in studies. The methods may also be used as a therapeutic for targeting specific strains of bacterial infections, or viral infection.

### E. Donor DNA

In some embodiments, the method comprises contacting the target nucleic acid with a donor DNA comprising one or more homologous sequences to the target nucleic acid. Without being bound by any theory of hypothesis, double-strand breaks in a target nucleic acid induced by the Ago-gDNA complex can initiate or stimulate the endogenous Homology Directed Recombination (HDR) repair pathway in the cell, which integrates the donor DNA into the cleavage target locus. In some embodiments, the donor DNA comprises a 5′ homology arm, a 3′ homology arm, and an exogenous sequence to the cell that is disposed in between the 5′ homology arm and the 3′ homology arm. In some embodiments, the homology sequence, such as homology arm(s), comprise a sequence that is at least about any one of 80%, 85%, 90%, 95%, 99%, or more, or 100% identical to the sequence flanking the cleavage site in the target nucleic acid. In some embodiments, the homology sequence, such as homology arm(s), is at least about any one of 10, 20, 30, 40, 50, 100, or more nucleotides long. In some embodiments, the donor DNA comprises a substitution sequence of the target nucleic acid encompassing the target locus. In some embodiments, the substitution sequence differs from the sequence of the target nucleic acid by no more than about any of 10, 5, 4, 3, 2, or 1 nucleotide(s).

In some embodiments, the donor DNA comprises a sequence encoding a selection marker. The selection marker can be used to select cells having the donor DNA integrated in the target locus. Exemplary selection markers include, but are not limited to, proteins that: (a) confer resistance to antibiotics or other toxins, e.g., ampicillin, neomycin, methotrexate, tetracycline, hygromycin, and G418, (b) complement auxotrophic deficiencies, and (c) supply critical nutrients not available from complex media. In some embodiments, a drug, such as G418, is used in a selection regimen to arrest the growth, or kill cells that do not have the donor DNA integrated in the target locus. Those cells that are successfully modified with the donor DNA produce a protein conferring drug resistance and thus survive the selection regimen. Examples of selectable markers suitable for mammalian cells also include DHFR, thymidine kinase, metallothionein-I and -II, preferably primate metallothionein genes, adenosine deaminase, ornithine decarboxylase, etc.

In some embodiments, the selection marker is a reporter protein that allows selection by confirming expression of the reporter protein. Examples of reporter proteins include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP, e.g., eGFP), HcRed, DsRed, cyan fluorescent protein (CFP), yellow fluorescent protein (YFP), and autofluorescent proteins including blue fluorescent protein (BFP). In some embodiments, the donor DNA does not comprise a promoter for the reporter protein. Thus, the reporter protein is only expressed when the donor DNA is integrated in frame with an endogenous gene of the cell.

The donor DNA may be of any suitable length, such as at least about any one of 100 bp, 200 bp, 300 bp, 500 bp, 1 kb, 2 kb, 5 kb, 10 kb, or longer. The donor DNA may be prepared by chemical synthesis or PCR amplification from a template. In some embodiments, the donor DNA is substantially purified, such as at least about any one of 80%, 85%, 90%, 95%, 99%, or more pure. In some embodiments, the donor DNA is present in a vector. In some embodiments, the donor DNA is present in a plasmid, such as an ultrapure plasmid, or a linearized plasmid. The donor DNA can be purified by known methods in the art, such as HPLC, gel electrophoresis, or using DNA purification kits.

### F. Cell

The methods described herein can be used to modify target nucleic acids in a variety of cells. In some embodiments, the cell is an isolated cell. In some embodiments the cell is in cell culture. In some embodiments, the cell is ex vivo. In some embodiments, the cell is obtained from a living organism, and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the bacterial cell is not related to the bacterial species from which the Ago protein is derived. In some embodiments, the cell is an archaeal cell or derived from an archaeal cell. In some embodiments, the cell is an eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, 293T, MF7, K562, HeLa, and transgenic varieties thereof. Cell lines are available from a variety of sources known to those with skill in the art (see, e.g., the American Type Culture Collection (ATCC) (Manassas, Va.)). In some embodiments, a cell transfected with one or more nucleic acids (such as Ago-coding vector and gDNA) or Ago-gDNA complex described herein is used to establish a new cell line comprising one or more vector-derived sequences to establish a new cell line comprising modification to the target nucleic acid. In some embodiments, cells transiently or non-transiently transfected with one or more nucleic acids (such as Ago-coding vector and gDNA) or Ago-gDNA complex described herein, or cell lines derived from such cells are used in assessing one or more test compounds.

In some embodiments, the cell is a primary cell. For example, cultures of primary cells can be passaged 0 times, 1 time, 2 times, 4 times, 5 times, 10 times, 15 times or more. In some embodiments, the primary cells are harvest from an individual by any known method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution, (e.g. normal saline, phosphate-buffered saline (PBS), Hank's balanced salt solution, etc.), conveniently supplemented with fetal calf serum or other naturally occurring factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

In some embodiments, the cell is an immune cell, such as T cells, Natural killer cells, and macrophages. In some embodiments, the cell is a human T cell obtained from a patient or a donor. The methods provided herein can be used to modify a target nucleic acid in a primary T cell for use in immunotherapy.

In some embodiments, the cell is a stem cell or progenitor cell. Cells can include stem cells (e.g., adult stem cells, embryonic stem cells, iPS cells) and progenitor cells (e.g., cardiac progenitor cells, neural progenitor cells, etc.). Cells can include mammalian stem cells and progenitor cells, including rodent stem cells, rodent progenitor cells, human stem cells, human progenitor cells, etc.

In some embodiments, the cell is a diseased cell. A diseased cell can have altered metabolic, gene expression, and/or morphologic features. A diseased cell can be a cancer cell, a diabetic cell, and a apoptotic cell. A diseased cell can be a cell from a diseased subject. Exemplary diseases can include blood disorders, cancers, metabolic disorders, eye disorders, organ disorders, musculoskeletal disorders, cardiac disease, and the like.

In some embodiments, the cell is free or substantially free from contamination. Contaminations include endotoxins, chelating agents (such as EDTA), and micro-organisms, such as mycoplasma, chlamydia, archaea, protozoa, and fungi. In some embodiments, the Ago-gDNA is sensitive to contamination of intracellular bacteria such as mycoplasma. Intracellular bacteria can be widespread and leave no visible signs of presence in cell lines. Intracellular bacteria should be carefully excluded from the cells before carrying out the methods described herein. In some embodiments, the cell, such as a cell line obtained from a commercial source, is treated with one or more antibiotics, such as penicillin and streptomycin, to remove contamination by micro-organisms. In some embodiments, the cell culture medium is heat inactivated to remove contamination by micro-organisms. In some embodiments, chelators, such as EDTA, is avoided in buffers for detaching and seeding cells into plates. In some embodiments, depending on the cell type, a divalent ion, such as Mg²⁺, is supplemented to the cell at a concentration of at least about any one of 0.1 mM, 0.2 mM, 0.5 mM, 1 mM, 2 mM, 3 mM, 4 mM, 5 mM, or more.

In some embodiments, the Argonaute induces double-stranded breaks or single-stranded breaks in a nucleic acid, (e.g. genomic DNA). The double-stranded break can stimulate cellular endogenous DNA-repair pathways, including Homology Directed Recombination (HDR), Non-Homologous End Joining (NHEJ), or Alternative Non-Homologues End-Joining (A-NHEJ). NHEJ can repair cleaved target nucleic acid without the need for a homologous template. This can result in deletion or insertion of one or more nucleotides at the target locus. HDR can occur with a homologous template, such as the donor DNA. The homologous template can comprise sequences that are homologous to sequences flanking the target nucleic acid cleavage site. In some cases, HDR can insert an exogenous polynucleotide sequence into the cleave target locus. The modifications of the target DNA due to NHEJ and/or HDR can lead to, for example, mutations, deletions, alterations, integrations, gene correction, gene replacement, gene tagging, transgene knock-in, gene disruption, and/or gene knock-outs.

In some embodiments, the cell culture is synchronized to enhance the efficiency of the methods. In some embodiments, cells in S and G2 phases are used for HDR-mediated gene editing. In some embodiments, the cell can be subjected to the method at any cell cycle. In some embodiments, cell over-plating significantly reduces the efficacy of the method. In some embodiments, the method is applied to a cell culture at no more than about any one of 40%, 45%, 50%, 55%, 60%, 65%, or 70% confluency.

In some embodiments, binding of the Ago-gDNA complex to the target locus in the cell recruits one or more endogenous cellular molecules or pathways other than DNA repair pathways to modify the target nucleic acid. For example, in some embodiments, the Ago-gDNA complex recruits the RISC complex to silence an mRNA. In some embodiments, catalytically inactive Ago can be used to silence a target mRNA. In some embodiments, binding of the Ago-gDNA complex blocks access of one or more endogenous cellular molecules or pathways to the target nucleic acid, thereby modifying the target nucleic acid. For example, binding of the Ago-gDNA complex may block endogenous transcription or translation machinery to decrease the expression of the target nucleic acid.

### G. Intracellular Delivery

In some embodiments, the method comprises delivering one or more nucleic acids (e.g., nucleic acids encoding the Ago protein, gDNA, donor DNA, etc.), one or more transcripts thereof, and/or a pre-formed Ago-gDNA complex to a cell. Exemplary intracellular delivery methods, include, but are not limited to: viruses or virus-like agents; chemical-based transfection methods, such as those using calcium phosphate, dendrimers, liposomes, or cationic polymers (e.g., DEAE-dextran or polyethylenimine); non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnectofection or magnet assisted transfection, particle bombardment; and hybrid methods, such as nucleofection. In some embodiments, the present application further provides cells produced by such methods, and organisms (such as animals, plants, or fungi) comprising or produced from such cells.

### Co-Transfection of Nucleic Acids

In some embodiments, the method comprises transfecting the nucleic acid (such as vector) encoding the Ago protein, the guide DNA, and optionally the donor DNA into the cell. In some embodiments, the nucleic acid encoding the Ago protein is transfected simultaneously with the guide DNA. In some embodiments, the nucleic acid encoding the Ago protein is transfected after the guide DNA, such as at least about any one of 1, 2, 3, 4, 5, 6, 7, 8, or more hours after transfection of the guide DNA. In some embodiments, the guide DNA is transfected into a host cell carrying a vector or stably integrated with a nucleic acid encoding the Ago protein. In some embodiments, the guide DNA is transfected into the cell for two or more times (e.g., 2, 3, 4, 5, 6, or more times). In some embodiments, a first batch of the gDNA is transfected simultaneously as the nucleic acid encoding the Ago protein into the cell, and subsequently one or more additional batches of the gDNA were transfected into the cell after at least about any one of 2, 4, 6, 8, 10, 12, 16, 20, 24, or more hours after the first transfection. In some embodiments, the donor DNA is transfected simultaneously with the nucleic acid encoding the Ago protein and the guide DNA. In some embodiments, the nucleic acid encoding the Ago protein and the guide DNA are first transfected into the cell (such as simultaneously or sequentially), followed by transfection of the guide DNA into the cell after at least about any one of 2, 4, 6, 8, 10, 12, 16, 20, 24, or more hours after the first transfection.

The nucleic acid encoding the Ago protein, the guide DNA, and the donor DNA can be transfected into the cell using the same method or different methods. Suitable methods can be chosen by a skilled person in the art among a variety of known methods. Suitable doses of the nucleic acid encoding the Ago protein, the guide DNA, and the donor DNA can be chosen and adjusted depending on the method(s) of transfection, order of transfection, ad nature of the nucleic acids transfected. In some embodiments, at least about any one of 10 ng, 50 ng, 100 ng, 200 ng, 300 ng, 500 ng, 750 ng, 1 mg or more of each nucleic acid encoding the Ago protein, the guide DNA and optionally the donor DNA is transfected into the cell. In some embodiments, about 100 ng to 500 ng of each nucleic acid encoding the Ago protein, the guide DNA and optionally the donor DNA is transfected into the cell. In some embodiments, wherein a plasmid encoding the Ago protein and the guide DNA are transfected simultaneously into the cell, the weight ratio between the guide DNA and the plasmid is at least about 1:1, 1:2, 1:3, 1:4, or 1:5. In some embodiments, the molar ratio between the guide DNA and the nucleic acid (such as vector) encoding the Ago protein transfected into the cell is at least about any one of 10:1, 20:1, 50:1, 100:1, 150:1, 200:1, 300:1, 500:1, 750:1, 1000:1 or more. In some embodiments, the molar ratio between the guide DNA and the donor DNA transfected into the cell is at least about any one of 1:5, 1:4, 1:3, 1:2, 1:1, 2:1, 3:1, 4:1, 5:1 or more. In some embodiments, the molar ratio between the donor DNA and the nucleic acid (such as vector) encoding the Ago protein transfected into the cell is at least about any one of 10:1, 20:1, 50:1, 100:1, 150:1, 200:1, 300:1, 500:1, 750:1, 1000:1 or more.

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787, and 4,897,355, and lipofection reagents are sold commercially (e.g., TRANSFECTAMINE™ and LIPOFECTAMIN®). In some embodiments, LIPOFECTAMINE® 2000 is used to transfect the nucleic acid encoding Ago (such as vector), the gDNA, and/or the donor DNA.

Conventional viral based systems for nucleic acid delivery include retroviral, lenti virus, adenoviral, adeno-associated and herpes simplex virus vectors. Integration in the host genome is possible with the retrovirus, lentivirus, and adeno-associated virus methods, often resulting in long term expression of the inserted transgene. Additionally, high transduction efficiencies have been observed in many different cell types. The tropism of a retrovirus can be altered by incorporating foreign envelope proteins, expanding the potential target population of target cells. Lentiviral vectors are retroviral vectors that are able to transduce or infect non-dividing cells and typically produce high viral titers. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the nucleic acids into the target cell to provide permanent transgene expression. Widely used retroviral vectors include those based upon murine leukemia vims (MuLV), gibbon ape leukemia virus (GaLV), Simian Immuno deficiency vims (SIV), human immuno deficiency vims (HIV), and combinations thereof. In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system.

Packaging cells are typically used to form virus particles that are capable of infecting a host cell. Such cells include 293T cells, which package adenovirus, and ψ2 cells or PA317 cells, which package retrovirus. Viral vectors are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

### Delivery of Ago-gDNA Complex

In some embodiments, the method comprises delivering a pre-formed Ago-gDNA complex into the cell. Any methods known in the art for protein or peptide delivery can be used to deliver the Ago-gDNA complex. In some embodiments, the molar ratio between the gDNA and the Ago protein in the complex is at least about 1:1. In some embodiments, the method further comprises transfecting the donor DNA to the cell at least about any one of 2, 4, 6, 8, 10, 12, 16, 20, 24, or more hours after the delivery of the Ago-gDNA complex to the cell. In some embodiments, the method comprises delivery of the pre-formed Ago-gDNA complex simultaneously, such as in the same composition, into the cell.

Methods for intracellular delivery of protein or protein complexes, such as pre-formed Ago-gDNA complex, include, but are not limited to, mechanical methods, such as microinjection, electroporation and mechanical deformation of cells using a microfluidic device; carrier-based methods, such as cell-penetrating peptides (CPPs), virus-like particles, supercharged proteins, nanocarriers, supramolecular carrier-based delivery systems, and nanoparticle-stabilized nanocapsules. See, for example, Fu et. al. Bioconjugate Chem. 2014, 25, 1602-1608. Some mechanical methods, such as microinjection and electroporation, can be invasive, and low-throughput. In some embodiments, the Ago-gDNA complex is delivered into the cell by inserting proteins through the cell membrane while passing cells through a microfluidic system, such as CELL SQUEEZE® (see, for example, U.S. Patent Application Publication No. 20140287509).

For carrier-based delivery, in some embodiments, the Ago protein is conjugated to the carrier, for example, by covalent linkage or recombinant fusion to the carrier. In some embodiments, the Ago-gDNA complex is delivered to the cell via one or more cell-penetrating peptides (CPP). Exemplary CPPs include, but are not limited to, TAT peptide, polyarginine peptide (such as R9 peptide), Pep-1, penetratin, NrTPs, and derivatives thereof. In some embodiments, the CPP is fused to the N terminus or the C terminus of the Ago protein by recombinant methods, or by post-translational modification of the Ago protein. In some embodiments, one or more pH- and temperature-induced modulators, synthetic endosomal lysis agents, and/or photoinduced physical disruption are applied to the cell to facilitate endosomal escape of the Ago-gDNA complex delivered by a cell-penetrating peptide.

In some embodiments, the Ago-gDNA complex is delivered to the cell via a supercharged protein. Supercharged proteins are a class of engineered or naturally occurring proteins with unusually high positive or negative net theoretical charge (typically >1 net charge unit per kilo-Dalton of molecular weight). For example, supercharged GFP can be used to deliver macromolecules into cells. See, for example, Cronican et al. Chem. Biol. 2011, 18: 833-838. In some embodiments, the supercharged protein is fused to either the N-terminus or the C-terminus of the Ago protein. In some embodiments, the Ago-gDNA complex is delivered to the cell via a covalently attached nanocarrier, such as a magnetic nanoparticle, or mesoporous silica nanoparticle. See, for example, Donae and Burda. Adv. Drug Delivery Rev., 2013, 65: 607-621.

In some embodiments, the Ago-gDNA is associated with the carrier to provide a supramolecular delivery system. In some embodiments, the Ago-gDNA complex is delivered to the cell via a virus-like particle (VLP). VLPs are formed by self-assembly of virus capsid proteins, which are similar in size and conformation to intact infectious virions, but possess nonviral properties, including being nonreplicating, nonpathogenic, and genomeless. See, for example, Muratori et al. Methods Mol. Biol. 2010, 614:111-124; and Kaczmarczyk et al. PNAS 2011, 108: 16998-17003. In some embodiments, the Ago-gDNA complex is delivered to the cell via a liposomal carrier, such as lysine-based cationic liposomes. Liposomes encapsulated Ago-gDNA complex can be prepared using known methods in the art, including, for example, free-thawing cycling processes. In some embodiments, the Ago-gDNA complex is delivered to the cell via a lipoplex. Lipoplexes may comprise surfactants, proteins, lipids, polymers, or a combination of these materials, and include solid lipid particles, oily suspensions, submicron lipid emulsions, lipid implants, lipid microbubbles, inverse lipid micelles, lipid microtubules, lipospheres, and lipid microcylinders. Commercially available cationic lipid reagents, such as BIOPORTER®, can be used to deliver proteins into the cytoplasm of living cells. Solid lipid particles composed of four different types of lipids and triglycerides with different chain-lengths of fatty acyl groups can also be used to deliver proteins into cells. In some embodiments, the Ago-gDNA complex is delivered to the cell via polymers, such as polyethylcnimine (PEI), and other dendrimers, such as carboxymethyl chitosan-poly(amidoamine). In some embodiments, the Ago-gDNA complex is delivered to the cell via a nanoplex. Nanoplexes comprise chemically modified nanoparticle, proteins, polymer, or other components. In some embodiments, the Ago-gDNA complex is delivered to the cell via a nanoparticle-stabilized nanocapsule. See, for example, Yang et al. Angew. Chem. Int. Ed. 2011, 50, 477-481.

### H. Cell Assessment and Selection

In some embodiments, the cell is cultured for at least about any one of 8 hours, 12 hours, 24 hours, 48 hours, 60 hours, 3 days, 4 days, 6 days, or more, such as about 48 hours to 60 hours, after delivery of the nucleic acid encoding the Ago protein and the gDNA, or the Ago-gDNA complex, and optionally the donor DNA into the cell. In some embodiments, the cell is allowed to grow to no more than about any one of 95%, 90%, 85%, or 80% confluency before assessment and/or selection. In some embodiments, the cell is selected based on one or more features, including, but not limited to expression of selection markers (e.g., antibiotic resistance protein, fluorescent tag, etc.), expression level of target nucleic acid, phenotypic change to the cell, or sequence of the target nucleic acid (e.g., a PCR amplicon of the target locus). In some embodiments, a single clone having successful modification of the target nucleic acid is isolated.

In some embodiments, a phenotypic change to the cell is assessed. In some embodiments, wherein an exogenous gene is knocked-in to the cell, a phenotypic change associated with the exogenous gene is assessed to select cells having successful integration of the exogenous gene. In some embodiments, wherein a mutation is introduced to a gene, a phenotypic change associated with the mutation or the gene is assessed. For example, if the gene is involved in development, a developmental defect in the cell or the organism derived from the cell that is associated with the mutation may be assessed to identify cells having successful introduction of the mutation. Other exemplary phenotypic changes suitable for screening include growth rate of the cell, cell cycle progression, and metabolic phenotypes. Phenotypic changes can be determined using known assays chosen for the target nucleic acid and its associated phenotype, including, for example, microscopy.

In some embodiments, the expression level of the target nucleic acid is assessed. Expression levels can be determined at either the RNA level or the protein level for a gene. Many methods are known in the art to assess expression levels, including, but not limited to, Western blots and immunostaining for protein levels, and quantitative RT-PCR, RNAseq, and in situ hybridization for RNA levels. In some embodiments, the expression level is decreased by at least about any one of 10%, 20%, 30%, 40%, 50%, 60%, 70%, 80%, 90%, or more.

In some embodiments, wherein the donor DNA comprises a selection marker, the expression level of the selection marker is assessed. In some embodiments, wherein the selection marker is an antibiotic resistance gene, the appropriate antibiotic is supplemented to the cell, e.g., at about 48 to 60 hours after the delivery of the Ago-gDNA complex or the Ago-encoding gene with the gDNA into the cell, to select for cells having successful integration of the donor DNA into the target nucleic acid. In some embodiments, wherein the selection marker is a fluorescent protein (e.g., mRFP, eGFP, etc.), expression of the selection marker is assessed by fluorescence microscopy, or by Fluorescence-assisted cell sorting (FACS).

In some embodiments, the target locus is amplified and assessed to select a cell having the desired mutation, or knock-in of exogenous sequence. PCR Primers can be designed to amplify regions of modification in the target nucleic acid, for example, at junctions between the target locus and the inserted sequence from the donor DNA, to provide amplicons for analysis. In some embodiments, the PCR amplicons can be analyzed by gel electrophoresis, restriction digestion, or by sequencing (such as Sanger sequencing), to confirm that the target locus is modified as designed.

### III. Compositions, Delivery Systems, and Kits

The present application also provides compositions, delivery systems, an kits for carrying out any one of the methods described herein.

In some embodiments, there is provided a composition comprising a complex comprising an Ago protein and a single-stranded guide DNA (such as a 5′ phosphorylated single-stranded guide DNA), wherein the complex is capable of specifically recognizing a target locus at a temperature of about 10° C. to about 60° C. (such as about 37° C.), and wherein the target locus comprises a sequence that is complementary to the sequence of the guide DNA. In some embodiments, the Ago protein is capable of cleaving the target locus. In some embodiments, wherein the target locus is a double-stranded DNA, the Ago protein is capable of inducing a double-strand break in the target locus. In some embodiments, the Ago protein is not capable of cleaving the target locus. In some embodiments, the guide DNA does not dissociate from the Ago protein at a temperature lower than about 50° C. In some embodiments, the guide DNA is about 10 to about 50 nucleotides (such as about 20 to about 30 nucleotides) long. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the molar ratio between the guide DNA and the Ago protein is at least about 1:1. In some embodiments, the guide DNA and the Ago protein do not naturally occur in the same organism. In some embodiments, the Ago protein comprises a nuclear localization signal (NLS).

In some embodiments, there is provided a composition comprising a complex comprising an Ago protein and a single-stranded guide DNA (such as a 5′ phosphorylated single-stranded guide DNA), wherein the Ago protein is derived from an organism selected from the group consisting of Natronobacterium gregoryi, Microcystis aeruginosa, Halogeometricum pallidum, Natrialba asialica, Natronorubrum tibetense, Natrinema pellirubrum, Halogeometricum borinquense, Thermococcus barophilus, Thermosynechococcus elongatus, Halorubrum lacusprofundi, Microcystis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolyticus, Aromatoleum aromaticum, Thermococcus onnurineus, Methanopyrus kandleri, Synechococcus elongatus, Anoxybacillus flavithermus, Exiguobacterium sp., Lyngbya sp., Clostridium butyricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the Ago protein comprises an amino acid sequence having at least about 80% (such as at least about any of 85%, 90%, 95%, 98%, 99% or more sequence homology, or about 100% sequence identity) sequence homology to a sequence selected from the group consisting of SEQ ID NOs:1-42. In some embodiments, the guide DNA and the Ago protein do not naturally occur in the same organism. In some embodiments, the complex is capable of specifically recognizing a target locus at a temperature of about 10° C. to about 60° C. (such as about 37° C.), and wherein the target locus comprises a sequence that is complementary to the sequence of the guide DNA. In some embodiments, the Ago protein is capable of cleaving the target locus. In some embodiments, wherein the target locus is a double-stranded DNA, the Ago protein is capable of inducing a double-strand break in the target locus. In some embodiments, the Ago protein is not capable of cleaving the target locus. In some embodiments, the guide DNA does not dissociate from the Ago protein at a temperature lower than about 50° C. In some embodiments, the guide DNA is about 10 to about 50 nucleotides (such as about 20 to about 30 nucleotides) long. In some embodiments, the sequence of the target locus comprises no more than about 3 mismatches (such as no mismatch) to the sequence of the guide DNA. In some embodiments, the target locus has a GC content of at least about 60%. In some embodiments, the molar ratio between the guide DNA and the Ago protein is at least about 1:1. In some embodiments, the Ago protein comprises a nuclear localization signal (NLS).

In some embodiments, there is provided a composition comprising a complex comprising an Ago protein and a single-stranded guide DNA (such as a 5′ phosphorylated single-stranded guide DNA), wherein the Ago protein is derived from Natronobacterium gregoryi. In some embodiments, the Ago protein comprises an amino acid sequence having at least about 80% (such as at least about any of 85%, 90%, 95%, 98%, 99% or more sequence homology, or about 100% sequence identity) sequence homology to SEQ ID NO: 1. In some embodiments, the guide DNA and the Ago protein do not naturally occur in the same organism. In some embodiments, the complex is capable of specifically recognizing a target locus at a temperature of about 10° C. to about 60° C. (such as about 37° C.), and wherein the target locus comprises a sequence that is complementary to the sequence of the guide DNA. In some embodiments, the Ago protein is capable of cleaving the target locus. In some embodiments, wherein the target locus is a double-stranded DNA, the Ago protein is capable of inducing a double-strand break in the target locus. In some embodiments, the Ago protein is not capable of cleaving the target locus. In some embodiments, the guide DNA does not dissociate from the Ago protein at a temperature lower than about 50° C. In some embodiments, the guide DNA is about 10 to about 50 nucleotides (such as about 20 to about 30 nucleotides) long. In some embodiments, the sequence of the target locus comprises no more than about 3 mismatches (such as no mismatch) to the sequence of the guide DNA. In some embodiments, the target locus has a GC content of at least about 60%. In some embodiments, the molar ratio between the guide DNA and the Ago protein is at least about 1:1. In some embodiments, the Ago protein comprises a nuclear localization signal (NLS).

In some embodiments, there is provided a delivery system comprising a complex comprising an Ago protein and a single-stranded guide DNA (such as a 5′ phosphorylated single-stranded guide DNA), and a vehicle suitable for intracellular delivery of the complex, wherein the complex is capable of specifically recognizing a target locus at a temperature of about 10° C. to about 60° C. (such as about 37° C.), and wherein the target locus comprises a sequence that is complementary to the sequence of the guide DNA. In some embodiments, the vehicle is selected from the group consisting of a cell-penetrating peptide, a virus-like particle, a nanocarrier, a liposome, a polymer, and a nanoparticle-stabilized nanocapsule. In some embodiments, the Ago protein is fused to the cell-penetrating peptide. In some embodiments, the Ago protein is capable of cleaving the target locus. In some embodiments, wherein the target locus is a double-stranded DNA, the Ago protein is capable of inducing a double-strand break in the target locus. In some embodiments, the Ago protein is not capable of cleaving the target locus. In some embodiments, the guide DNA does not dissociate from the Ago protein at a temperature lower than about 50° C. In some embodiments, the guide DNA is about 10 to about 50 nucleotides (such as about 20 to about 30 nucleotides) long. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the molar ratio between the guide DNA and the Ago protein is at least about 1:1. In some embodiments, the guide DNA and the Ago protein do not naturally occur in the same organism. In some embodiments, the Ago protein comprises a nuclear localization signal (NLS).

In some embodiments, there is provided a delivery system comprising a complex comprising an Ago protein and a single-stranded guide DNA (such as a 5′ phosphorylated single-stranded guide DNA), and a vehicle suitable for intracellular delivery of the complex, wherein the Ago protein is derived from an organism selected from the group consisting of Natronobacterium gregoryi, Microcystis aeruginosa, Halogeometricum pallidum, Natrialba asiatica, Natronorubrum tibetense, Natrinema pellirubrum, Halogeometricum borinquense, Thermococcus barophilus, Thermosynechococcus elongatus, Halorubrum lacusprofundi, Microcyslis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolyticus, Aromatoleum aromaticum, Thermococcus onnurineus, Methanopyrus kandleri, Synechococcus elongatus, Anoxybacillus flavithermus, Exiguobacterium sp., Lyngbya sp., Clostridium butyricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the Ago protein comprises an amino acid sequence having at least about 80% (such as at least about any of 85%, 90%, 95%, 98%, 99% or more sequence homology, or about 100% sequence identity) sequence homology to a sequence selected from the group consisting of SEQ ID NOs:1-42. In some embodiments, the vehicle is selected from the group consisting of a cell-penetrating peptide, a virus-like particle, a nanocarrier, a liposome, a polymer, and a nanoparticle-stabilized nanocapsule. In some embodiments, the Ago protein is fused to the cell-penetrating peptide. In some embodiments, the guide DNA and the Ago protein do not naturally occur in the same organism. In some embodiments, the complex is capable of specifically recognizing a target locus at a temperature of about 10° C. to about 60° C. (such as about 37° C.), and wherein the target locus comprises a sequence that is complementary to the sequence of the guide DNA. In some embodiments, the Ago protein is capable of cleaving the target locus. In some embodiments, wherein the target locus is a double-stranded DNA, the Ago protein is capable of inducing a double-strand break in the target locus. In some embodiments, the Ago protein is not capable of cleaving the target locus. In some embodiments, the guide DNA does not dissociate from the Ago protein at a temperature lower than about 50° C. In some embodiments, the guide DNA is about 10 to about 50 nucleotides (such as about 20 to about 30 nucleotides) long. In some embodiments, the sequence of the target locus comprises no more than about 3 mismatches (such as no mismatch) to the sequence of the guide DNA. In some embodiments, the target locus has a GC content of at least about 60%. In some embodiments, the molar ratio between the guide DNA and the Ago protein is at least about 1:1. In some embodiments, the Ago protein comprises a nuclear localization signal (NLS).

In some embodiments, there is provided a delivery system comprising a complex comprising an Ago protein and a single-stranded guide DNA (such as a 5′ phosphorylated single-stranded guide DNA), and a vehicle suitable for intracellular delivery of the complex, wherein the Ago protein is derived from Natronobacterium gregoryi. In some embodiments, the Ago protein comprises an amino acid sequence having at least about 80% (such as at least about any of 85%, 90%, 95%, 98%, 99% or more sequence homology, or about 100% sequence identity) sequence homology to SEQ ID NO: 1. In some embodiments, the vehicle is selected from the group consisting of a cell-penetrating peptide, a virus-like particle, a nanocarrier, a liposome, a polymer, and a nanoparticle-stabilized nanocapsule. In some embodiments, the Ago protein is fused to the cell-penetrating peptide. In some embodiments, the guide DNA and the Ago protein do not naturally occur in the same organism. In some embodiments, the complex is capable of specifically recognizing a target locus at a temperature of about 10° C. to about 60° C. (such as about 37° C.), and wherein the target locus comprises a sequence that is complementary to the sequence of the guide DNA. In some embodiments, the Ago protein is capable of cleaving the target locus. In some embodiments, wherein the target locus is a double-stranded DNA, the Ago protein is capable of inducing a double-strand break in the target locus. In some embodiments, the Ago protein is not capable of cleaving the target locus. In some embodiments, the guide DNA does not dissociate from the Ago protein at a temperature lower than about 50° C. In some embodiments, the guide DNA is about 10 to about 50 nucleotides (such as about 20 to about 30 nucleotides) long. In some embodiments, the sequence of the target locus comprises no more than about 3 mismatches (such as no mismatch) to the sequence of the guide DNA. In some embodiments, the target locus has a GC content of at least about 60%. In some embodiments, the molar ratio between the guide DNA and the Ago protein is at least about 1:1. In some embodiments, the Ago protein comprises a nuclear localization signal (NLS).

In some embodiments, there is provided a kit comprising a nucleic acid encoding an Ago protein and a single-stranded guide DNA (such as a 5′ phosphorylated single-stranded guide DNA), wherein the Ago protein and the guide DNA forms a complex that is capable of specifically recognizing a target locus at a temperature of about 10° C. to about 60° C., and wherein the target locus comprises a sequence that is complementary to the sequence of the guide DNA. In some embodiments, the nucleic acid encoding the Ago protein is operably linked to a promoter. In some embodiments, the nucleic acid encoding the Ago protein is present in a vector, such as a viral vector. In some embodiments, the vector is an ultrapure plasmid. In some embodiments, the nucleic acid encoding the Ago protein is an mRNA. In some embodiments, the nucleic acid encoding the Ago protein is codon-optimized for a species of interest. In some embodiments, the Ago protein is capable of cleaving the target locus. In some embodiments, wherein the target locus is a double-stranded DNA, the Ago protein is capable of inducing a double-strand break in the target locus. In some embodiments, the Ago protein is not capable of cleaving the target locus. In some embodiments, the guide DNA does not dissociate from the Ago protein at a temperature lower than about 50° C. In some embodiments, the guide DNA is about 10 to about 50 nucleotides (such as about 20 to about 30 nucleotides) long. In some embodiments, the sequence of the target locus comprises no more than about 3 mismatches (such as no mismatch) to the sequence of the guide DNA. In some embodiments, the target locus has a GC content of at least about 60%. In some embodiments, the Ago protein comprises at least 2 of the 3 conservative amino acids in the KQK motif of the 5′-phosphate binding site. In some embodiments, the Ago protein comprises at least 2 of the 3 conservative amino acids in the DDE motif of the nuclease active site. In some embodiments, the molar ratio between the guide DNA and the Ago protein is at least about 1:1. In some embodiments, the Ago protein comprises a nuclear localization signal (NLS). In some embodiments, the kit further comprises a donor DNA.

In some embodiments, there is provided a kit comprising a nucleic acid encoding an Ago protein and a single-stranded guide DNA (such as a 5′ phosphorylated single-stranded guide DNA), wherein the Ago protein is derived from an organism selected from the group consisting of Natronobacterium gregoryi, Microcystis aeruginosa, Halogeometricum pallidum, Natrialba asiatica, Natronorubrum tibetense. Natrinema pellirubrum, Halogeometricum borinquense, Thermococcus barophilus, Thermosynechococcus elongalus, Halorubrum lacusprofundi, Microcystis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolyticus, Aromatoleum aromaticum, Thermococcus onnurineus, Methanopyrus kandleri, Synechococcus elongalus, Anoxybacillus flavithermus, Exiguobacterium sp., Lyngbya sp., Clostridium butryricum, Halorubrum kocurii, Burkholderia ambifaria, Burkholderia graminis, Haloarcula marismortui, Mesorhizobium loti, Rhodobacterales bacterium and Pedobacter heparinus. In some embodiments, the Ago protein comprises an amino acid sequence having at least about 80% (such as at least about any of 85%, 90%, 95%, 98%, 99% or more sequence homology, or about 100% sequence identity) sequence homology to a sequence selected from the group consisting of SEQ ID NOs: 1-42. In some embodiments, the nucleic acid encoding the Ago protein is operably linked to a promoter. In some embodiments, the nucleic acid encoding the Ago protein is present in a vector, such as a viral vector. In some embodiments, the vector is an ultrapure plasmid. In some embodiments, the nucleic acid encoding the Ago protein is an mRNA. In some embodiments, the nucleic acid encoding the Ago protein is codon-optimized for a species of interest. In some embodiments, the Ago protein and the guide DNA forms a complex that is capable of specifically recognizing a target locus at a temperature of about 10° C. to about 60° C. (such as about 37° C.), and wherein the target locus comprises a sequence that is complementary to the sequence of the guide DNA. In some embodiments, the Ago protein is capable of cleaving the target locus. In some embodiments, wherein the target locus is a double-stranded DNA, the Ago protein is capable of inducing a double-strand break in the target locus. In some embodiments, the Ago protein is not capable of cleaving the target locus. In some embodiments, the guide DNA does not dissociate from the Ago protein at a temperature lower than about 50° C. In some embodiments, the guide DNA is about 10 to about 50 nucleotides (such as about 20 to about 30 nucleotides) long. In some embodiments, the sequence of the target locus comprises no more than about 3 mismatches (such as no mismatch) to the sequence of the guide DNA. In some embodiments, the target locus has a GC content of at least about 60%. In some embodiments, the molar ratio between the guide DNA and the Ago protein is at least about 1:1. In some embodiments, the Ago protein comprises a nuclear localization signal (NLS). In some embodiments, the kit further comprises a donor DNA.

In some embodiments, there is provided a kit comprising a nucleic acid encoding an Ago protein and a single-stranded guide DNA (such as a 5′ phosphorylated single-stranded guide DNA), wherein the Ago protein is derived from Natronobacterium gregoryi. In some embodiments, the Ago protein comprises an amino acid sequence having at least about 80% (such as at least about any of 85%, 90%, 95%, 98%, 99% or more sequence homology, or about 100% sequence identity) sequence homology to SEQ ID NO: 1. In some embodiments, the nucleic acid encoding the Ago protein is operably linked to a promoter. In some embodiments, the nucleic acid encoding the Ago protein is present in a vector, such as a viral vector. In some embodiments, the vector is an ultrapure plasmid. In some embodiments, the nucleic acid encoding the Ago protein is an mRNA. In some embodiments, the nucleic acid encoding the Ago protein is codon-optimized for a species of interest. In some embodiments, the Ago protein and the guide DNA forms a complex that is capable of specifically recognizing a target locus at a temperature of about 10° C. to about 60° C. (such as about 37° C.), and wherein the target locus comprises a sequence that is complementary to the sequence of the guide DNA. In some embodiments, the Ago protein is capable of cleaving the target locus. In some embodiments, wherein the target locus is a double-stranded DNA, the Ago protein is capable of inducing a double-strand break in the target locus. In some embodiments, the Ago protein is not capable of cleaving the target locus. In some embodiments, the guide DNA does not dissociate from the Ago protein at a temperature lower than about 50° C. In some embodiments, the guide DNA is about 10 to about 50 nucleotides (such as about 20 to about 30 nucleotides) long. In some embodiments, the sequence of the target locus comprises no more than about 3 mismatches (such as no mismatch) to the sequence of the guide DNA. In some embodiments, the target locus has a GC content of at least about 60%. In some embodiments, the molar ratio between the guide DNA and the Ago protein is at least about 1:1. In some embodiments, the Ago protein comprises a nuclear localization signal (NLS). In some embodiments, the kit further comprises a donor DNA.

Further provided are cells, kits, and articles of manufacture comprising any of the compositions or delivery systems described herein.

In some embodiments, the kit comprises one or more reagents for use in any one of the methods described herein. Reagents may be provided in any suitable container. For example, the kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises a donor DNA.

In some embodiments, the kit further comprises instructions for carrying out any one of the methods described herein. The kits described herein can be used for modification of a target nucleic acid, genome editing, introducing mutations (e.g., indels, frameshift, knock-out or knock-in, and substitution) at a target locus, altering the phenotype of a cell, as analytic or interference agents.

### EXEMPLARY EMBODIMENTS

The exemplary embodiments below are intended to be purely exemplary of the invention and should therefore not be considered to limit the invention in any way.

### Embodiment 1

In some embodiments, there is provided a method of modifying a target nucleic acid, comprising contacting the target nucleic acid with an Argonaute (Ago) protein and a single-stranded guide DNA at a temperature of about 10° C. to about 60° C., wherein the Ago protein and the guide DNA form a complex that specifically recognizes a target locus in the target nucleic acid, and wherein the target locus comprises a sequence that is complementary to the sequence of the guide DNA.

### Embodiment 2

In some further embodiments of embodiment 1, the Ago protein cleaves the target locus.

### Embodiment 3

In some further embodiments of embodiment 2, wherein the target locus is a double-stranded DNA, the Ago protein induces a double-strand break in the target locus.

### Embodiment 4

In some further embodiments of any one of embodiments 1-3, the temperature is about 37° C.

### Embodiment 5

In some further embodiments of any one of embodiments 1-4, the Ago protein and the guide DNA are present in a pre-formed complex.

### Embodiment 6

In some further embodiments of embodiment 5, the guide DNA does not dissociate from the Ago protein at a temperature lower than about 50° C.

### Embodiment 7

In some further embodiments of any one of embodiments 1-6, the sequence of the target locus comprises no more than about 3 mismatches to the sequence of the guide DNA.

### Embodiment 8

In some further embodiments of any one of embodiments 1-7, the target locus has a GC content of at least about 60%.

### Embodiment 9

In some further embodiments of any one of embodiments 1-8, the Ago protein comprises at least 2 of the 3 conservative amino acids in the KQK motif of the 5′-phosphate binding site.

### Embodiment 10

In some further embodiments of any one of embodiments 1-9, the Ago protein comprises at least 2 of the 3 conservative amino acids in the DDE motif of the nuclease active site.

### Embodiment 11

In some further embodiments of any one of embodiments 1-10, the Ago protein is derived from an organism selected from the group consisting of Natronobacterium gregoryi, Microcystis aeruginosa, Halogeometricum pallidum, Natrialba asiatica, Natronorubrum tibetense, Natrinema pellirubrum, Halogeometricum borinquense, Thermococcus barophilus, Thermosynechococcus elongatus, Halorubrum lacusprofundi, Microcyslis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolyticus, Aromatoleum aromaticum, Thermococcus onnurineus, Methanopyrus kandleri, Synechococcus elongatus, Anoxybacillus flavithermus, Exiguobacterium sp., Lyngbya sp., Clostridium butyricum, Halorubrum kocurii, Burkholderia ambifaria, Burkholderia graminis, Haloarcula marismortui, Mesorhizobium loti, Rhodobacterales bacterium and Pedobacter heparinus.

### Embodiment 12

In some further embodiments of embodiment 11, the Ago protein is derived from Natronobacterium gregoryi.

### Embodiment 13

In some further embodiments of embodiment 11, the Ago protein is derived from Pedobacter heparinus.

### Embodiment 14

In some further embodiments of embodiment 11, the Ago protein is derived from Microcystis sp.

### Embodiment 15

In some further embodiments of embodiment 11, the Ago protein is derived from Microcystis aeruginosa.

### Embodiment 16

In some further embodiments of any one of embodiments 1-11, the Ago protein comprises an amino acid sequence having at least about 80% sequence homology to a sequence selected from the group consisting of SEQ ID NOs:1-42.

### Embodiment 17

In some further embodiments of embodiment 16, the Ago protein comprises an amino acid sequence having at least about 80% sequence homology to SEQ ID NO: 1.

### Embodiment 18

In some further embodiments of embodiment 16, the Ago protein comprises an amino acid sequence having at least about 80% sequence homology to SEQ ID NO: 2.

### Embodiment 19

In some further embodiments of embodiment 16, the Ago protein comprises an amino acid sequence having at least about 80% sequence homology to SEQ ID NO: 11.

### Embodiment 20

In some further embodiments of embodiment 16, the Ago protein comprises an amino acid sequence having at least about 80% sequence homology to SEQ ID NO: 41.

### Embodiment 21

In some further embodiments of any one of embodiments 16-20, the at least about 80% sequence homology is 100% sequence identity.

### Embodiment 22

In some further embodiments of any one of embodiments 1-21, the guide DNA is phosphorylated at the 5′ terminus.

### Embodiment 23

In some further embodiments of any one of embodiments 1-22, the guide DNA is about 10 to about 50 nucleotides long.

### Embodiment 24

In some further embodiments of any one of embodiments 1-23, the contacting is in the presence of a divalent metal ion.

### Embodiment 25

In some further embodiments of embodiment 24, the concentration of the divalent metal ion is at least about 0.1 mM.

### Embodiment 26

In some further embodiments of any one of embodiments 1-25, the target nucleic acid is an isolated DNA.

### Embodiment 27

In some further embodiments of any one of embodiments 1-25, the target nucleic acid is present in a cell.

### Embodiment 28

In some further embodiments of embodiment 27, the method comprises transfecting the cell with the guide DNA and a nucleic acid encoding the Ago protein.

### Embodiment 29

In some further embodiments of embodiment 28, the guide DNA and the nucleic acid encoding the Ago protein are transfected into the cell simultaneously.

### Embodiment 30

In some further embodiments of embodiment 28, the guide DNA is transfected into the cell prior to the nucleic acid encoding the Ago protein.

### Embodiment 31

In some further embodiments of any one of embodiments 28-30, the cell is transfected with the guide DNA for at least two times.

### Embodiment 32

In some further embodiments of any one of embodiments 28-31, the molar ratio of the guide DNA to the nucleic acid encoding the Ago protein is at least about 100:1.

### Embodiment 33

In some further embodiments of any one of embodiments 28-32, the nucleic acid encoding the Ago protein is operably linked to a promoter.

### Embodiment 34

In some further embodiments of any one of embodiments 28-33, the nucleic acid encoding the Ago protein is present in a vector.

### Embodiment 35

In some further embodiments of embodiment 34, the vector is a viral vector.

### Embodiment 36

In some further embodiments of embodiment 34 or 35, the vector is an ultrapure plasmid.

### Embodiment 37

In some further embodiments of any one of embodiments 28-32, the nucleic acid encoding the Ago protein is an mRNA.

### Embodiment 38

In some further embodiments of any one of embodiments 28-37, the nucleic acid encoding the Ago protein is codon-optimized for the organism from which the cell is derived.

### Embodiment 39

In some further embodiments of embodiment 27, the method comprises delivering a pre-formed complex comprising the Ago protein and the guide DNA into the cell.

### Embodiment 40

In some further embodiments of embodiment 39, the pre-formed complex is delivered into the cell by electroporation, microinjection, or mechanical deformation via a microfluidic channel.

### Embodiment 41

In some further embodiments of embodiment 39, the pre-formed complex is delivered into the cell via a vehicle selected from the group consisting of a cell-penetrating peptide, a virus-like particle, a nanocarrier, a liposome, a polymer, and a nanoparticle-stabilized nanocapsule.

### Embodiment 42

In some further embodiments of embodiment 41, the Ago protein is fused to the cell-penetrating peptide.

### Embodiment 43

In some further embodiments of any one of embodiments 27-42, the method comprises treating the cell with one or more antibiotics.

### Embodiment 44

In some further embodiments of any one of embodiments 27-43, the Ago protein comprises a nuclear localization signal (NLS).

### Embodiment 45

In some further embodiments of any one of embodiments 27-44, the target nucleic acid is endogenous to the cell.

### Embodiment 46

In some further embodiments of embodiment 45, the target nucleic acid is a genomic DNA.

### Embodiment 47

In some further embodiments of any one of embodiments 27-44, the target nucleic acid is exogenous to the cell.

### Embodiment 48

In some further embodiments of embodiment 47, the target nucleic acid is a viral DNA.

### Embodiment 49

In some further embodiments of embodiment 47 or 48, the target nucleic acid is integrated in the genome of the cell.

### Embodiment 50

In some further embodiments of embodiment 47 or 48, the target nucleic acid is not integrated in the genome of the cell.

### Embodiment 51

In some further embodiments of any one of embodiments 27-50, the cell is a prokaryotic cell.

### Embodiment 52

In some further embodiments of any one of embodiments 27-50, the cell is a eukaryotic cell.

### Embodiment 53

In some further embodiments of embodiment 52, the cell is a mammalian cell.

### Embodiment 54

In some further embodiments of embodiment 53, the cell is a human cell.

### Embodiment 55

In some further embodiments of embodiment 52, the cell is a yeast cell, a fungus cell, or a plant cell.

### Embodiment 56

In some further embodiments of any one of embodiments 52-55, the cell is derived from a cell line.

### Embodiment 57

In some further embodiments of any one of embodiments 52-55, the cell is a primary cell.

### Embodiment 58

In some further embodiments of embodiment 57, the cell is an immune cell.

### Embodiment 59

In some further embodiments of any one of embodiments 27-58, the cell is free from contamination by non-viral microorganisms.

### Embodiment 60

In some further embodiments of any one of embodiments 1-59, the modifying comprises site-specific cleavage of the target nucleic acid.

### Embodiment 61

In some further embodiments of any one of embodiments 27-59, the modifying comprises introducing a mutation at the target locus selected from an insertion, a deletion, and a frameshift mutation.

### Embodiment 62

In some further embodiments of any one of embodiments 27-59, the method further comprises contacting the target nucleic acid with a donor DNA comprising a sequence homologous to the sequence of the target locus under a condition that allows integration of the donor DNA at the target locus.

### Embodiment 63

In some further embodiments of embodiment 62, the donor DNA encodes a selection marker.

### Embodiment 64

In some further embodiments of embodiment 63, the selection marker is a reporter protein.

### Embodiment 65

In some further embodiments of embodiment 63 or 64, the method further comprises assessing the cell for expression of the selection marker.

### Embodiment 66

In some further embodiments of any one of embodiments 27-65, the modifying comprises inducing a phenotypic change to the cell.

### Embodiment 67

In some further embodiments of embodiment 66, the method further comprises assessing the phenotypic change to the cell.

### Embodiment 68

In some further embodiments of any one of embodiments 27-67, the method further comprises sequencing the target nucleic acid after the modifying.

### Embodiment 69

In some further embodiments of any one of embodiments 27-68, the modifying comprises altering expression of the target nucleic acid.

### Embodiment 70

In some further embodiments of any one of embodiments 27-69, the modifying comprises introducing a knockout mutation at the target locus.

### Embodiment 71

In some further embodiments of any one of embodiments 62-70, the modifying comprises knocking in an exogenous sequence at the target locus, wherein the donor DNA comprises the exogenous sequence.

### Embodiment 72

In some further embodiments of any one of embodiments 62-70, the modifying comprises introducing a substitution mutation at the target locus, wherein the donor DNA comprises the substitution mutation.

### Embodiment 73

In some further embodiments of embodiment 72, the substitution mutation is a single nucleotide substitution.

### Embodiment 74

In some further embodiments of any one of embodiments 27-73, the target locus is a disease-associated locus.

### Embodiment 75

In some embodiments, there is provided a composition comprising a complex comprising an Ago protein and a single-stranded guide DNA, wherein the complex is capable of specifically recognizing a target locus at a temperature of about 10° C. to about 60° C., and wherein the target locus comprises a sequence that is complementary to the sequence of the guide DNA.

### Embodiment 76

In some embodiments, there is provided a delivery system comprising a complex comprising an Ago protein and a single-stranded guide DNA, and a vehicle suitable for intracellular delivery of the complex, wherein the complex is capable of specifically recognizing a target locus at a temperature of about 10° C. to about 60° C., and wherein the target locus comprises a sequence that is complementary to the sequence of the guide DNA.

### Embodiment 77

In some further embodiments of embodiment 76, the vehicle is selected from the group consisting of a cell-penetrating peptide, a virus-like particle, a nanocarrier, a liposome, a polymer, and a nanoparticle-stabilized nanocapsule.

### Embodiment 78

In some further embodiments of embodiment 76 or 77, the Ago protein is fused to the cell-penetrating peptide.

### Embodiment 79

In some further embodiments of any one of embodiments 75-78, the molar ratio between the guide DNA and the Ago protein is at least about 1:1.

### Embodiment 80

In some embodiments, there is provided a kit comprising a nucleic acid encoding an Ago protein and a single-stranded guide DNA, wherein the Ago protein and the guide DNA forms a complex that is capable of specifically recognizing a target locus at a temperature of about 10° C. to about 60° C., and wherein the target locus comprises a sequence that is complementary to the sequence of the guide DNA.

### Embodiment 81

In some further embodiments of embodiment 80, the nucleic acid encoding the Ago protein is operably linked to a promoter.

### Embodiment 82

In some further embodiments of embodiment 80 or 81, the nucleic acid encoding the Ago protein is present in a vector.

### Embodiment 83

In some further embodiments of embodiment 82, the vector is a viral vector.

### Embodiment 84

In some further embodiments of embodiment 82 or 83, the vector is an ultrapure plasmid.

### Embodiment 85

In some further embodiments of embodiment 84, the nucleic acid encoding the Ago protein is an mRNA.

### Embodiment 86

In some further embodiments of any one of embodiments 80-85, the nucleic acid encoding the Ago protein is codon-optimized for an organism of interest.

### Embodiment 87

In some further embodiments of any one of embodiments 75-86, the Ago protein is capable of cleaving the target locus.

### Embodiment 88

In some further embodiments of any one of embodiments 75-87, wherein the target locus is a double-stranded DNA, the Ago protein is capable of inducing a double-strand break in the target locus.

### Embodiment 89

In some further embodiments of any one of embodiments 75-88, the temperature is about 37° C.

### Embodiment 90

In some further embodiments of any one of embodiments 75-89, the guide DNA does not dissociate from the Ago protein at a temperature lower than about 50° C.

### Embodiment 91

In some further embodiments of any one of embodiments 75-90, the guide DNA and the Ago protein do not naturally occur in the same organism.

### Embodiment 92

In some further embodiments of any one of embodiments 75-91, the guide DNA is phosphorylated at the 5′ terminus.

### Embodiment 93

In some further embodiments of any one of embodiments 75-92, the guide DNA is about 10 to about 50 nucleotides long.

### Embodiment 94

In some further embodiments of any one of embodiments 75-93, the sequence of the target locus comprises no more than about 3 mismatches to the sequence of the guide DNA.

### Embodiment 95

In some further embodiments of any one of embodiments 75-94, the target locus has a GC content of at least about 60%.

### Embodiment 96

In some further embodiments of any one of embodiments 75-95, the Ago protein comprises at least 2 of the 3 conservative amino acids in the KQK motif of the 5′-phosphate binding site.

### Embodiment 97

In some further embodiments of any one of embodiments 75-96, the Ago protein comprises at least 2 of the 3 conservative amino acids in the DDE motif of the nuclease active site.

### Embodiment 98

In some further embodiments of any one of embodiments 75-97, the Ago protein is derived from an organism selected from the group consisting of Natronobacterium gregoryi, Microcystis aeruginosa, Halogeometricum pallidum, Natrialba asiatica, Natronorubrum tibetense, Natrinema pellirubrum, Halogeometricum borinquense, Thermococcus barophilus, Thermosynechococcus elongalus, Halorubrum lacusprofundi, Microcystis sp., Synechococcus sp., Clostridium bartlettii, Clostridium perfringens, Clostridium sartagoforme, Clostridium sp., Intestinibacter bartlettii, Ferroglobus placidus, Halobacterium sp., Methanocaldococcus fervens, Pseudomonas luteola, Thermogladius cellulolyticus, Aromatoleum aromaticum, Thermococcus onnurineus, Methanopyrus kandleri, Synechococcus elongalus, Anoxybacillus flavithermus, Exiguobacterium sp., Lyngbya sp., Clostridium butryricum, Halorubrum kocurii, Burkholderia ambifaria, Burkholderia graminis, Haloarcula marismortui, Mesorhizobium loti, Rhodobacterales bacterium and Pedobacter heparinus.

### Embodiment 99

In some further embodiments of embodiment 98, the Ago protein is derived from Natronobacterium gregoryi.

### Embodiment 100

In some further embodiments of embodiment 98, the Ago protein is derived from Pedobacter heparinus.

### Embodiment 101

In some further embodiments of embodiment 98, the Ago protein is derived from Microcystis sp.

### Embodiment 102

In some further embodiments of embodiment 98, the Ago protein is derived from Microcystis aeruginosa.

### Embodiment 103

In some further embodiments of any one of embodiments 75-98, the Ago protein comprises an amino acid sequence having at least about 80% sequence homology to a sequence selected from the group consisting of SEQ ID NOs:1-42.

### Embodiment 104

In some further embodiments of embodiment 103, the Ago protein comprises an amino acid sequence having at least about 80% sequence homology to SEQ ID NO: 1.

### Embodiment 105

In some further embodiments of embodiment 103, the Ago protein comprises an amino acid sequence having at least about 80% sequence homology to SEQ ID NO: 2.

### Embodiment 106

In some further embodiments of embodiment 103, the Ago protein comprises an amino acid sequence having at least about 80% sequence homology to SEQ ID NO: 11.

### Embodiment 107

In some further embodiments of embodiment 103, the Ago protein comprises an amino acid sequence having at least about 80% sequence homology to SEQ ID NO: 41.

### Embodiment 108

In some further embodiments of any one of embodiments 103-107, the at least about 80% sequence homology is 100% sequence identity.

### Embodiment 109

In some further embodiments of any one of embodiments 75-108, the Ago protein comprises a nuclear localization signal (NLS).

### Embodiment 110

In some embodiments, there is provided a cell comprising the composition of any one of embodiments 75, 79, and 87-109.

### Embodiment 111

In some embodiments, there is provided a kit comprising the composition of any one of embodiments 75, 79, and 87-109 and instructions for modifying a target nucleic acid using the kit, wherein the target nucleic acid comprises the target locus.

### EXAMPLES

The examples below are intended to be purely exemplary of the invention and should therefore not be considered to limit the invention in any way.

### Example 1: In Vitro Cleavage of a Target Plasmid by Ago-gDNA Complexes

In this example, Argonaute proteins from various species were isolated and subcloned into an expression plasmid, which was co-transfected into 293T cells together with a guide DNA to provide gDNA-pre-loaded Ago complexes (“Ago-gDNA”). The purified Ago-gDNA complexes were each incubated with a target plasmid in vitro to assess the DNA-guided DNA endonuclease activities of the Agos at different temperatures.

### 1. NpAgo

The complete coding sequence of the NgAgo was isolated from the genomic DNA of halo-alkaliphilic archaebacterium Natronobacterium gregoryi SP2 by PCR using primers FLAG-HindII-F and HA-BamHI-R, cleaved with restriction enzymes HindIII and BamHI, and subcloned into the subcloned into a pcDNA3.1/Hygro(+) plasmid (Invitrogen) to provide plasmid FLAG-NgAgo-HA-pcDNA3.1. FLAG and HA tag sequences were fused to the 5′ and 3′ of the coding sequence of NgAgo, respectively. The amino acid sequence of NgAgo (SEQ ID NO: 1) is shown below:

(NgAgo)
SEQ ID NO: 1
MTVIDLDSTTTADELTSGHTYDISVTLTGVYDNTDEQHPRMSLAFEQDNG
ERRYITLWKNTTPKDVFTYDYATGSTYIFTNIDYEVKDGYENLTATYQTT
VENATAQEVGTTDEDETFAGGEPLDHHLDDALNETPDDAETESDSGHVMT
SFASRDQLPEWTLHTYTLTATDGAKTDTEYARRTLAYTVRQELYTDHDAA
PVATDGLMLLTPEPLGETPLDLDCGVRVEADETRTLDYTTAKDRLLAREL
VEEGLKRSLWDDYLVRGIDEVLSKEPVLTCDEFDLHERYDLSVEVGHSGR
AYLHINFRHRFVPKLTLADIDDDNIYPGLRVKTTYRPRRGHIVWGLRDEC
ATDSLNTLGNQSVVAYHRNNQTPINTDLLDAIEAADRRVVETRRQGHGDD
AVSFPQELLAVEPNTHQIKQFASDGFHQQARSKTRLSASRCSEKAQAFAE
RLDPVRLNGSTVEFSSEFFTGNNEQQLRLLYENGESVLTFRDGARGAHPD
ETFSKGIVNPPESFEVAVVLPEQQADTCKAQWDTMADLLNQAGAPPTRSE
TVQYDAFSSPESISLNVAGAIDPSEVDAAFVVLPPDQEGFADLASPTETY
DELKKALANMGIYSQMAYFDRFRDAKIFYTRNVALGLLAAAGGVAFTTEH
AMPGDADMFIGIDVSRSYPEDGASGQINIAATATAVYKDGTILGHSSTRP
QLGEKLQSTDVRDIMKNAILGYQQVTGESPTHIVIHRDGFMNEDLDPATE
FLNEQGVEYDIVEIRKQPQTRLLAVSDVQYDTPVKSIAAINQNEPRATVA
TFGAPEYLATRDGGGLPRPIQIERVAGETDIETLTRQVYLLSQSHIQVHN
STARLPITTAYADQASTHATKGYLVQTGAFESNVGFL

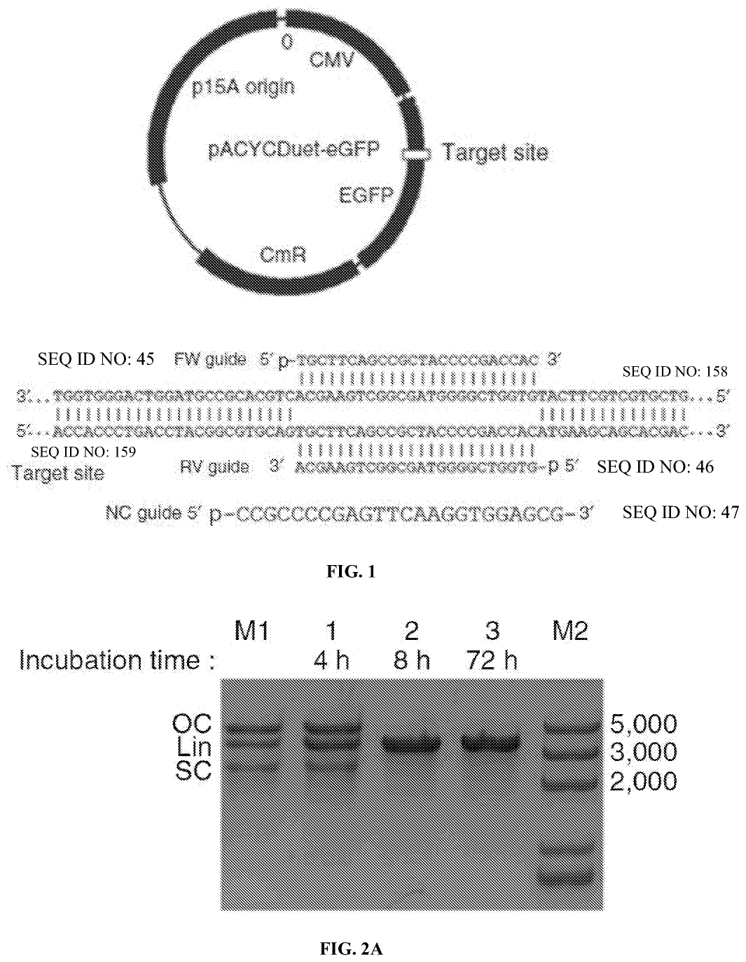

A 5′ phosphorylated single-stranded guide DNA (“FW guide”, Table 2) was designed to be complementary to a target site of the GFP gene in the pACYCDuet-eGFP plasmid (Novagen) (FIG. 1).

The FLAG-NgAgo-HA-pcDNA3.1 plasmid and the FW guide was co-transfected into human 293T cells. A complex (“NgAgo-gDNA”) comprising the FLAG-NgAgo-HA protein bound to the guide DNA was purified from the co-transfected cells using a FLAG, HA Tandem Affinity Purification Kit (Sigma-Aldrich). Briefly, 48 h after transfection of FLAG-NgAgo-HA-pcDNA3.1 plasmid and the guide DNA, 5×10⁷ 293T cells were lysed with 5 ml RIPA buffer and sonicated on ice. After centrifugation, the supernatant was collected and mixed with 100 μl anti-FLAG M2 resin, followed by gentle rocking overnight at 4° C. Then, the resin was washed with 1 ml RIPA once and 1 ml reaction buffer without BSA (in vitro cleavage assay) 3 times. Eluted FLAG-NgAgo-HA from the resin with 300 μl 0.2 mg/ml 3×FLAG peptide (resolved in reaction buffer without BSA). The eluates were examined by PAGE to evaluate the quality and quantity. If the FLAG-NgAgo-HA protein was above 80% purity, ultrafiltration was done to concentrate the FLAG-NgAgo-HA protein to about 1 mg/ml for subsequent enzymatic analysis; if the purity of the sample was not ideal, 100 μl anti-HA resin was used to repeat the purification steps as described above.

5 μg of purified NgAgo-gDNA complex was incubated with 400 ng pACYCDuet-eGFP plasmid in a 50 μL reaction mixture (10 mm Tris pH 8.0, 20 mM NaCl, 0.5 mM MgCl₂, 0.4% glycerol, 2 mM DTT and 20 mg/μL BSA) at 4° C., 20° C., 30° C., 40° C., 50° C., or 60° C. for 8 hours. The reaction mixture was then treated with proteinase K at 52° C. for 2 hours to degrade proteins in the reaction mixture, and the resulting product was analyzed by electrophoresis in 1% agarose gel.

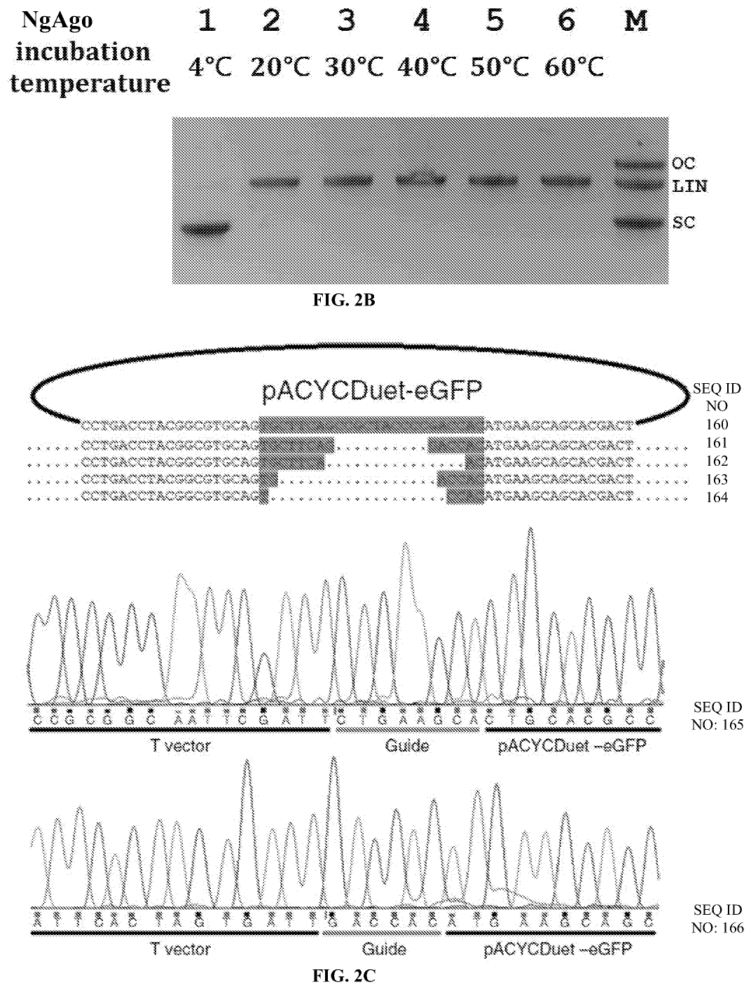

Results of the in vitro plasmid cleavage assay by NgAgo-gDNA complex are shown in FIGS. 2A and 2B. The relative sizes of the plasmid in its supercoiled (SC) state (i.e., uncleaved), linearized (Lin) state (i.e., both strands cleaved), and open circular (OC) state (i.e., one strand cleaved) are shown in the lane labeled “M1,” and molecular weight markers are shown in lane “M2”. As shown in FIG. 2A, after 4 hours of incubation with the NgAgo-gDNA complex, there remained to be uncleaved supercoiled and single-strand cleaved open circular species, while some plasmids were cleaved in both strands to result in the linearized form. After 8 hours and 72 hours, the plasmid appeared only as linearized DNA, indicating that the NgAgo was able to completely cleave both strands of the plasmid using a single pre-loaded guide DNA. Similarly, the NgAgo-gDNA complex was able to induce double-strand breaks in the target plasmid to yield a linearized product at all temperatures tested except for at 4° C. (FIG. 2B).

To determine the position of the cleavage site on the plasmid following in vitro cleavage by the FW gDNA-preloaded NgAgo, linearized plasmids were excised from the agarose gel after 72 hours of incubation and purified by gel extraction. After PCR polyadenylation of the purified DNA, the resultant product was cloned into pGEM-T-Vector (Promega). Chloromycetin and ampicillin double-resistant clones were selected for plasmid extraction followed by sequencing (Sangon Biotech).

FIG. 2C shows the sequence of the wild type target site of the pACYCDuet-eGFP plasmid on top, aligned below with four representative sequencing results of plasmid cleavage products of NgAgo-gDNA after 72 hour incubation, with the FW guide sequence shaded. Also shown are two representative sequencing chromatograms, with sequences corresponding to pGEM-T vector, FW Guide, and pACYCDuet-eGFP DNA indicated below. Sequences are representative results of 20 independent experiments. The results showed that the target site was successfully cleaved by the NgAgo. Rather than simply breaking a single phosphodiester bond, NgAgo randomly removed between 1 and 20 nucleotides within the target site. Nucleotide removal at the target site is not due to an exonuclease activity of NgAgo. As shown in FIG. 2B, prolonging enzymatic reaction to 72 h did not result in further degradation of the linearized target, even though complete linearization was achieved after 8 h under the same conditions. This finding is consistent with the TtAgo reaction, which also removes nucleotides in its targets (Wang, Y. et al. Nature 461, 754-761 (2009)).

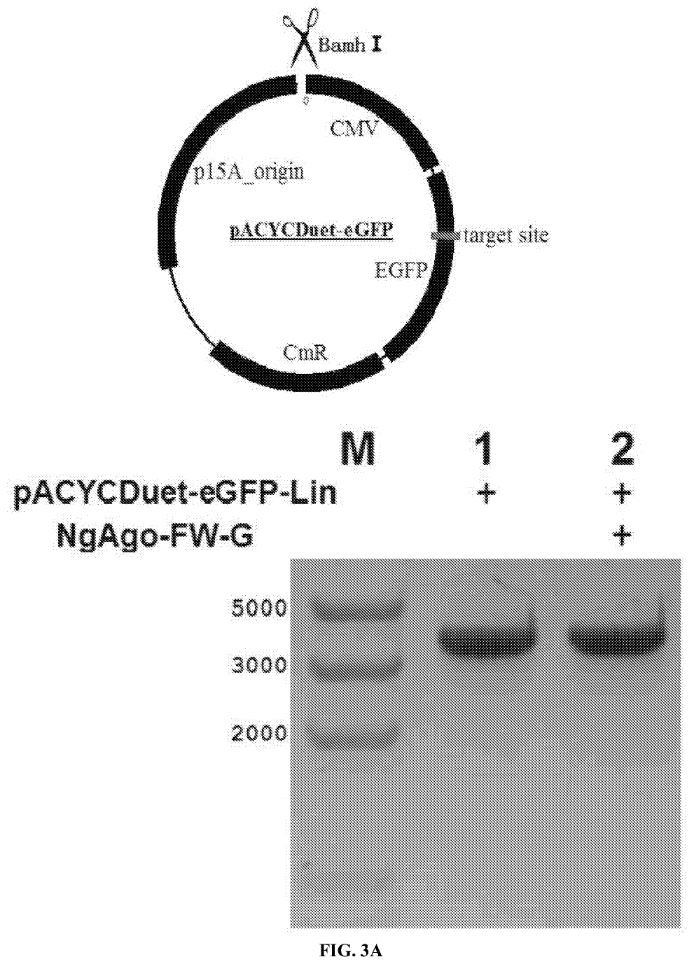

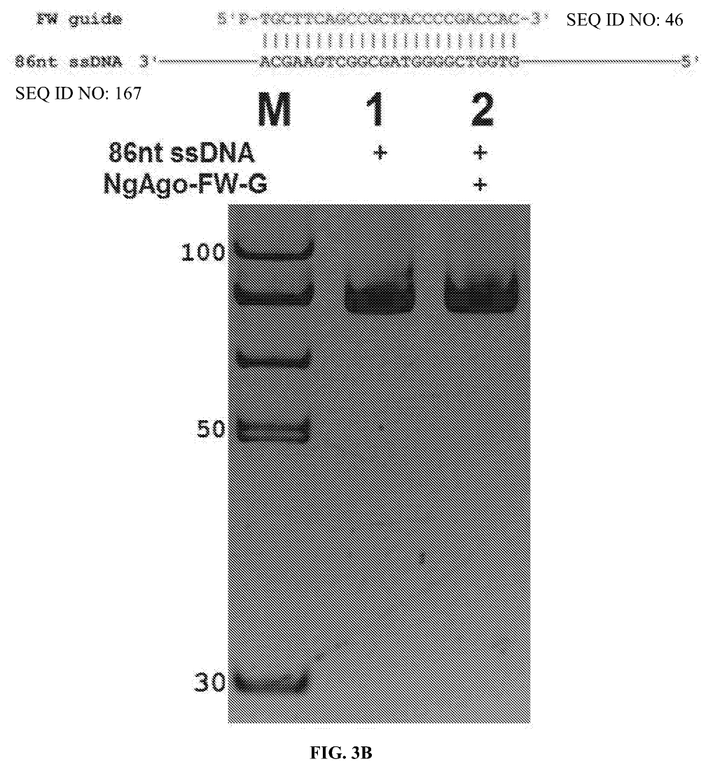

To further investigate whether NgAgo has the ability to cleave linearized DNA, the pACYCDuet-eGFP plasmid was linearized by cleavage with the BamHI restriction enzyme at a restriction site located on the plasmid (FIG. 3A, top). The linearized plasmid was co-incubated for 8 hours at 37° C. with or without NgAgo purified from 293T cells and preloaded with FW (“NgAgo-FW-G”). There was no discernable cleavage when linearized plasmid was incubated with or without NgAgo-FW (FIG. 3A, lanes 2 and 1, respectively).

The ability of NgAgo-FW-G to cleave an 86 nucleotide ssDNA fragment (SEQ ID NO: 167) was also tested. The 86 nt ssDNA was co-incubated with or without NgAgo-FW-G for 8 hours at 37° C. The results indicated that there was no cleavage of the 86 nt target sequence (FIG. 3B, lanes 2 and 1, respectively). Lane M shows a molecular weight marker with sizes indicted in nt on the left. These data indicate that NgAgo is not an exonuclease.

### 2. NaAgo

Natrialba asiatica Argonaute (“NaAgo”) gene sequence was isolated from the genomic DNA of bacterium Natrialba asiatica DSM 12278 (ATCC Accession Number 700177; CGMCC (Chinese General Microbiological Culture Collection Center) Accession Number 1.2199), and subcloned to yield a FLAG-NaAgo-HA-pcDNA3.1 plasmid as described above. The amino acid sequence of NaAgo (SEQ ID NO: 4; NCBI Accession No. WP_006111085.1) is shown below.

(NaAgo)
SEQ ID NO: 4
MKTQDDIAHKQPITIEVQILKELDKPSPKMATRFLVADRDGNRFSLAIWK
NNALSDYDWTIGQWYRLENARGNVFNGKQSLNGSSKMRATPLEASEEDET
STDDVGRVDTILGNMSPDQAYLSLFPISRSFDTLSVYEYSIEAAEAFEDA
PDTVTYRCAGRLRRITGAGVAYAGSMRIVSTRKLPDKLADPFSLSEPTER
ELNATDARDRHRIERLLKSLVKAAIDDSTYDPYQINRIRARTPSITAGDG
LFEACYEFAARVDVMPSGDAFVGIEVRYHTRSQVTADVYEDKTAELVGTI
VEHDPERYNISGTGRVVGFTDHHFTDALDELGGLSLADWYAQKDRVPEGV
LEALREKNPRLVDIQYQEDEPARIHVPDLLRVAPRKEVVKELDPAFHRRW
DREAKMLPDKRFRHAIEFVDHLGSLPDIDATVAPEPLGPSLSYMSTAVDR
EKNLRFKDGRTATTPSSGIRSGVYQQPTSFDIAYVYPTESEQESKQFISN
FENKLSQCQCEPTAARHVPYELGGELSYLAVINELESVDAVLAVVPPRDD
DRITAGDITDPYPEFKKGLGKQKIPSQMIVTENLGTRWVMNNTAMGLIAG
AGGVPWRVDEMPGEADCFIGLDVTRDPETGQHLGASANVVYADGTVFASK
TQTLQSGETFDEQSIIDVIKDVFQEFVRREGRSPEHIVIHRDGRLFEDAD
EIQAPFADSGVSIDILDIRKSGAPRIAQYEDNSFKIDEKGRLFISQDDTH
GFIATTGKPEFDDSDNLGTPKTLRVVRRAGDTPMLTLLKQVYWLSEAHVG
SVSRSVRLPITTYYADRCAEHAREGYLLHGELIEGVPYL

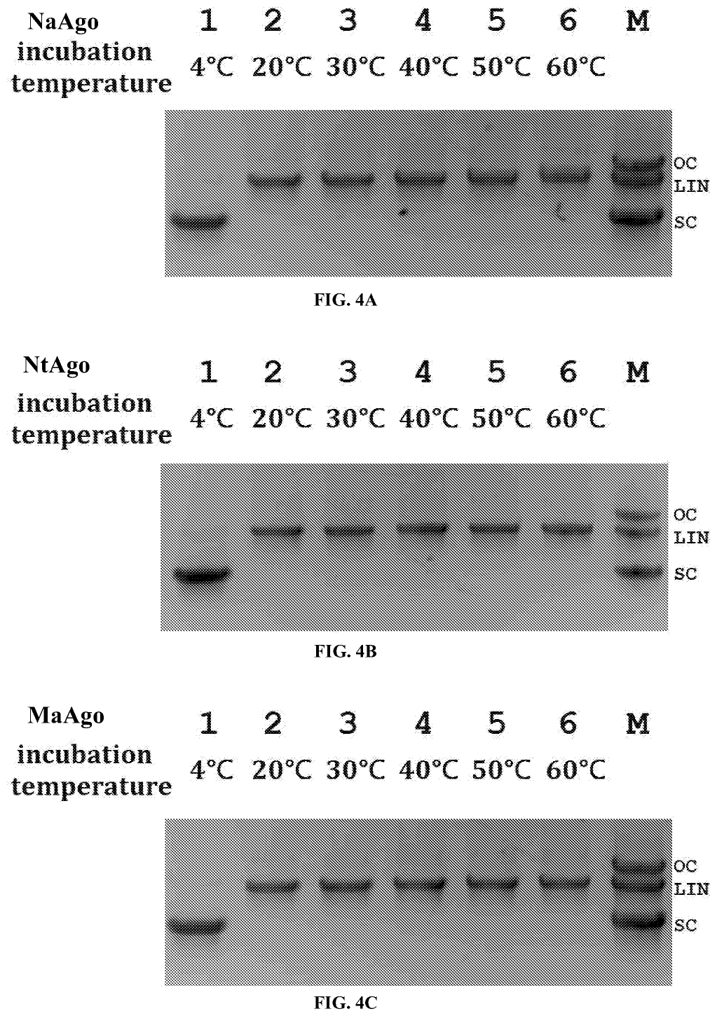

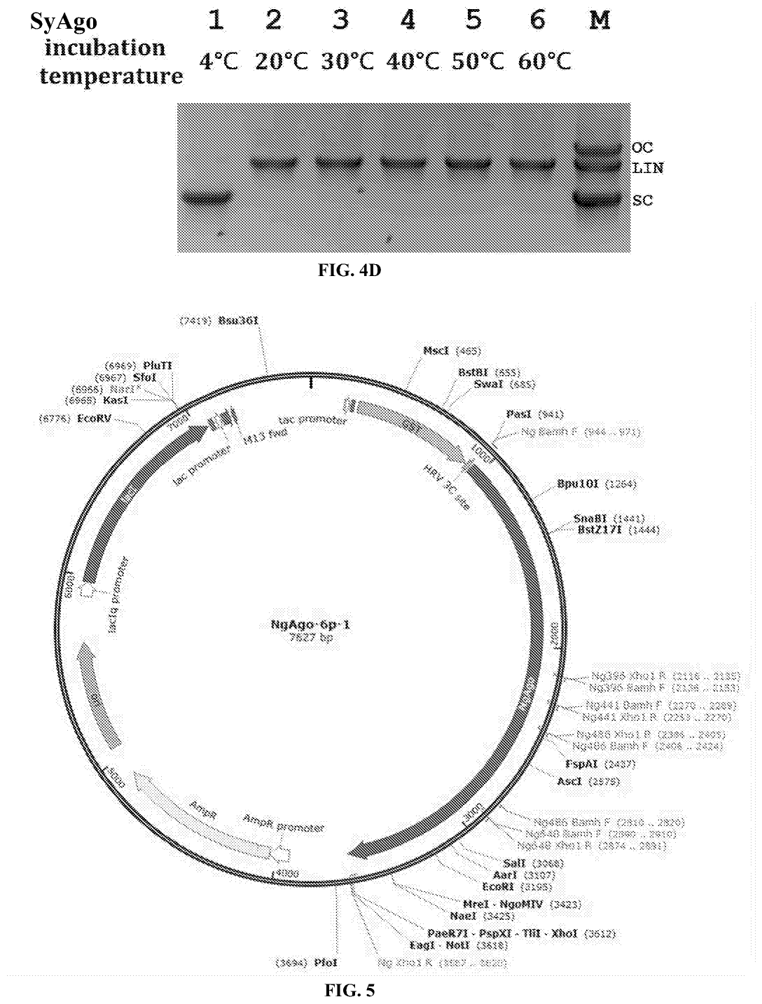

FLAG-NaAgo-HA-pcDNA3.1 and FW guide DNA were co-transfected into 293T cells, and the NaAgo-gDNA complex was purified and incubated with the target pACYCDuet-eGFP plasmid as described above. Electrophoresis of the in vitro cleavage reaction showed that NaAgo-gDNA complex was able to induce double-strand breaks in the target plasmid to yield a linearized product at all temperatures tested except for at 4° C. (FIG. 4A).

### 3. NtAgo

Natronorubrum tibetense Argonaute (“NtAgo”) gene sequence was isolated from the genomic DNA of bacterium Natronorubrum tibetense GA33 (CGMCC Accession Number 1.2123), and subcloned to yield a FLAG-NtAgo-HA-pcDNA3.1 plasmid as described above. The amino acid sequence of NtAgo (SEQ ID NO: 5; NCBI Accession No. WP_006090832.1) is shown below.

(NtAgo)
SEQ ID NO: 5
MAVKTDIEDGKQIDISLRVTGTDEWDHDAIARKVQLEDVEGTPVELTVFH
NNEIADFEWDDERWYVLENVVGNEYRGEMQLNPGYDLIVTPLDEPPAAAE
NGGAENTSATQSSESGDSGSSTEADQSAESESARESEVTSEPRPTADGGG
ELLHQQPLSEGNYLLQFELGDLPELTVHEYELRATGSGGINPDDFTNGIE
GFTAKAANYYQSRINSPVTTADASRRRIYATEKLHGKISIHGYTVKPVHQ
GETTLEARSYTDDGPLQEFVKQDVKRAVAGRFEVSGIDSIIEPTPQRTAN
SGLFEAYRKYKCRIRVDADGTVICGVNVAYHLESTFSAADWVQRGHDIAE
VTVEHDTDLYDSARTATVKEVIDMDYDDVLDGPGVPMSEYHEQHVEQDVI
NSMQAGDPIIADLQYGSDEDSIFPQLLEYCKVIPTFDQLGSVDDTFLDVI
HNESRMEPEERFSVVTSFVDLLGPTPYFSFDPVPQPTNAGYREHKTPNTP
NLRFGDGKTGFYGAGGLERKGYGIYKAPESFDIIALYPEDEEDDARPYVL
SLLNKLADYDAGPTVFDQETYELGSEFHYSQHAQKASDYDAALIVVPDAD
KAAAADYDDPYPEFKRRLGQLGVPSQMISVDNLGNDNYRGNICSSLIGKA
GGVPWRIDDVPGDVDAFVGLDVTYDHATKQHLGAAANVIMADGTILASEA
VTKQAGETFDEDDVANVIKHVLEIFAEEEGRPPRHVVIHRDGKFYLDVEN
LVKRLDKARDLIQRFDLVEIRKSGNPRIAAYDESESRFDIADKGIAFHVH
NGDHSYLTTTGGREGSPGTPRPLQIVKRHGSTDLDTLAEQTYWLSEAHVG
SLSRSTRLPITTYYADKCADFAMKGYLTKGSVIRGVPYI

FLAG-NtAgo-HA-pcDNA3.1 and FW guide DNA were co-transfected into 293T cells, and the NtAgo-gDNA complex was purified and incubated with the target pACYCDuet-eGFP plasmid as described above. Electrophoresis of the in vitro cleavage reaction showed that NtAgo-gDNA complex was able to induce double-strand breaks in the target plasmid to yield a linearized product at all temperatures tested except for at 4° C. (FIG. 4B).

### 4. MaAgo

Microcystis aeruginosa Argonaute (“MaAgo”) gene sequence was isolated from the genomic DNA of bacterium Microcystis aeruginosa NIES 843 (FACHB Accession Number 315), and subcloned to yield a FLAG-MaAgo-HA-pcDNA3.1 plasmid as described above. The amino acid sequence of MaAgo (SEQ ID NO: 2; NCBI Accession No. WP_012265209.1) is shown below.

(MaAgo)
SEQ ID NO: 2
MNYTAANTANSPIFLSEISSLTLKNSCLNCFQLNHQVTRKIGNRFSWQFS
RKFPDVVVIFEDNCFWVLAKDEKSIPSLQQWKEALSDIQEVLREDIGDHY
YSIHWLKDFQITALVTAQLAVRILKIFGKFSDPIVFPKDSQISENQVQVR
REVNFWAEIINDTDPAICLTVDSSIVYSGDLEQFYENHPYRQDAVKLLVG
LKVKDRETNGTAKIIRIAGRIGERREDLLTKATGSISRRKLEEAHLGQPV
VAVQFGKNPQEYIYPLAALKPWVTDEDESLFQVNYGNLLKATKIFYAERQ
ELLKLYKQEAQKALNNFGFQLREKSINSQEYPELFWTPSISIEQTPILFG
QGERGEKREIIKGLSKGGVYKRHREYVDPARKIRLAILKPANLKVGDFRE
QLEKRLKLYKFETILPPENQINFSVEGLGFEKRARLEEAVDRLIGVEIPV
DIALVFLPQEDRNADNTEEGSLYSWIKRKFLGRGVITQMIYEKTLNDKSN
YKNILNQVVPGILAKLGNLPYVLAEPLEIADYFIGLDVGRMPKKNLPGSL
NVCASVRLYGKQGEFVRCRVEDSLTEGEEIPQRILENCLPQAELKNQTVL
IYRDGKFQGKEVENLLARARAINAKFILVECYKTGIPRLYNLQQKQINAP
SKGLALALSNREVILITSQVSEQIGVPRPLRLKVHELGEQRNLKQLVDTT
LKLTLLHYGSLKDPRLPIPLYGADIIAYRRLQGIYPSLLEDDCQFWL

FLAG-MaAgo-HA-pcDNA3.1 and FW guide DNA were co-transfected into 293T cells, and the MaAgo-gDNA complex was purified and incubated with the target pACYCDuet-eGFP plasmid as described above. Electrophoresis of the in vitro cleavage reaction showed that NtAgo-gDNA complex was able to induce double-strand breaks in the target plasmid to yield a linearized product at all temperatures tested except for at 4° C. (FIG. 4C).

### 5. SyAgo

Synechococcus Argonaute (“SyAgo”) gene sequence was isolated from the genomic DNA of bacterium Synechococcus 7942 (FACHB Accession Number 805), and subcloned to yield a FLAG-SyAgo-HA-pcDNA3.1 plasmid as described above. The amino acid sequence of SyAgo (SEQ ID NO: 12; NCBI Accession No. WP_011378069.1) is shown below.

(SyAgo)
SEQ ID NO: 12
MDLLSNLRRSSIVLNRFYVKSLSQSDLTAYEYRCIFKKTPELGDEKRLLA
SICYKLGAIAVRIGSNIITKEAVRPEKLQGHDWQLVQMGTKQLDCRNDAH
RCALETFERKFLERDLSASSQTEVRKAAEGGLIWWVVGAKGIEKSGNGWE
VHRGRRIDVSLDAEGNLYLEIDIHHRFYTPWTVHQWLEQYPEIPLSYVRN
NYLDERHGFINWQYGRFTQERPQDILLDCLGMSLAEYHLNKGATEEEVQQ
SYVVYVKPISWRKGKLTAHLSRRLSPSLTMEMLAKVAEDSTVCDREKREI
RAVFKSIKQSINQRLQEAQKTASWILTKTYGISSPAIALSCDGYLLPAAK
LLAANKQPVSKTADIRNKGCAKIGETSFGYLNLYNNQLQYPLEVHKCLLE
IANKNNLQLSLDQRRVLSDYPQDDLDQQMFWQTWSSQGIKTVLVVMPWDS
HHDKQKIRIQAIQAGIATQFMVPLPKADKYKALNVTLGLLCKAGWQPIQL
ESVDHPEVADLIIGFDTGTNRELYYGTSAFAVLADGQSLGWELPAVQRGE
TFSGQAIWQTVSKLIIKFYQICQRYPQKLLLMRDGLVQEGEFQQTIELLK
ERKIAVDVISVRKSGAGRMGQEIYENGQLVYRDAAIGSVILQPAERSFIM
VTSQPVSKTIGSIRPLRIVHEYGSTDLELLALQTYHLTQLHPASGFRSCR
LPWVLHLADRSSKEFQRIGQISVLQNISRDKLIAV

FLAG-SyAgo-HA-pcDNA3.1 and FW guide DNA were co-transfected into 293T cells, and the SyAgo-gDNA complex was purified and incubated with the target pACYCDuet-eGFP plasmid as described above. Electrophoresis of the in vitro cleavage reaction showed that NtAgo-gDNA complex was able to induce double-strand breaks in the target plasmid to yield a linearized product at all temperatures tested except for at 4° C. (FIG. 4D).

### Example 2: In Vitro Cleavage of a Target Plasmid by NgAgo Expressed in E. coli

In this example, NgAgo was expressed and purified from E. coli, and incubated with guide nucleic acids to assess its in vitro endonuclease activities and requirements.

Expression and Purification of GST-NgAgo in E. coli

To confirm that NgAgo uses ssDNA guides, NgAgo was expressed in Escherichia coli, as these bacteria produce both 5′ phosphorylated ssRNAs and ssDNAs. A GST-tagged recombinant NgAgo protein was generated by isolating NgAgo from the genomic DNA of N. gregoryi SP2 with PCR using the primers NgAgo-6P-1 F and NgAgo-6P-1 R, cleaving with restriction enzymes BamHI, Bgl II, and Xho I, and cloning into the pGEX6P-1 plasmid (GE Healthcare). NgAgo-6P-1 plasmid (FIG. 5) was transformed into the E. coli strain BL21 DE3 (Clontech) to express GST-NgAgo protein. Sephrose-4B-purified GST-NgAgo was eluted with reduced glutathione buffer.

### Electrophoresis of Nucleic Acids Bound to Bacterially-Derived GST-NgAgo

The purified protein was digested by proteinase K at 52° C. for 2 h. Nucleic acids were separated from protein by Roti phenol, chloroform, isoamyl alcohol (pH 7.5-8.0, Carl Roth GmbH) and further purified with ethanol precipitation. The air-dried precipitants from 5 ml protein solution were resuspended in 100 μl distilled water and then subjected to DNase I or RNase A digestion. The nucleic acids were then purified by Roti phenol, chloroform, isoamyl alcohol and precipitated by isopropanol. After being resuspended in 50 μl TE buffer (pH 8.0), nucleic acids were analyzed by denatured PAGE and visualized by silver staining.

Silver staining of nucleic acids bound to GST-NgAgo shows bands of approximately 20-30 nucleotides in size present in the sample not subjected to digestion (FIG. 6, second lane from left). Nuclease digestion with RNAse A did not result in the disappearance of these bands (FIG. 6, third lane from left). When subjected to DNase I, the nucleic acids were digested (FIG. 6, right two lanes). Left lane is a single-stranded molecular weight marker (M), showing size in nucleotides (nt). This confirms that NgAgo binds to ssDNA.

In Vitro Cleavage Assay with Various Guide DNAs

To test whether the NgAgo expressed by E. coli can cleave the target site of the pACYCDuet-eGFP plasmid (FIG. 1) in the presence of ssDNA guides, an in vitro plasmid cleavage assay was performed. Before the cleavage assay, the native nucleic acids bound to the purified GST-NgAgo were replaced with the designed ssDNA guides (FW, RV, or NC, FIG. 1). The pACYCDuet-cGFP plasmid (Novagen) has no homologous sequences to the GST-NgAgo-encoding plasmid pGEX6P-1. Each ssDNA guide was 5′ phosphorylated and 24-nucleotide (nt) long. Two guides, FW and RV, are complimentary to each other and to the target site sequence. The non-complimentary guide (NC) contains a random sequence with no overlap with pACYCDuet-eGFP sequence.

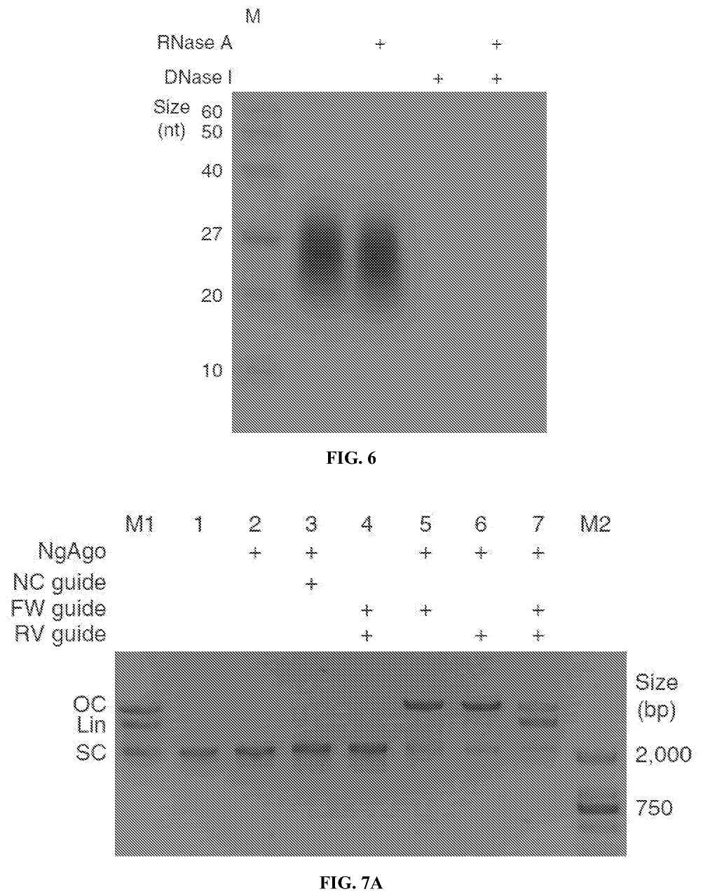

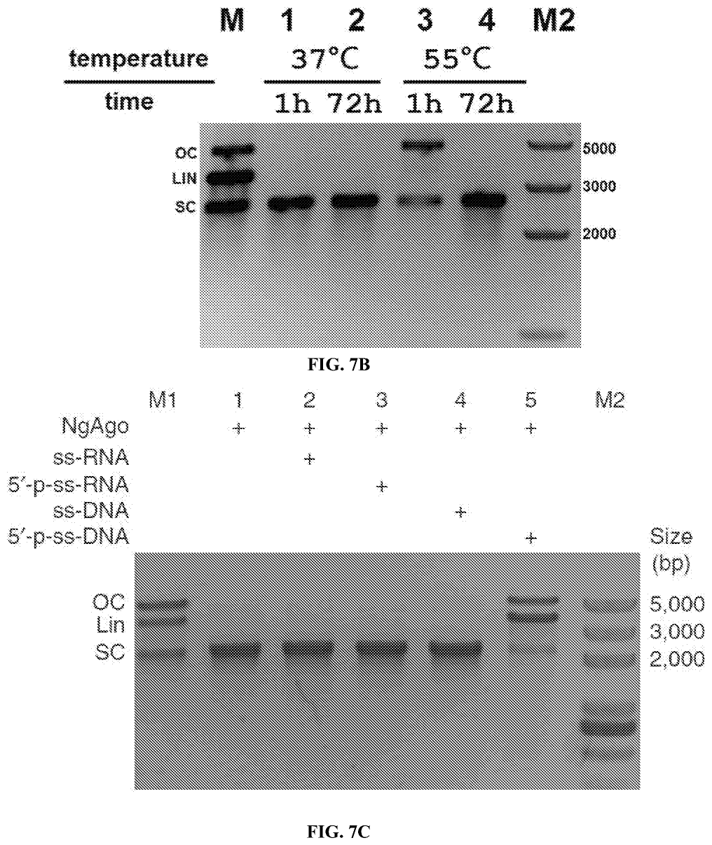

To reload guides, 5 μg GST-NgAgo was incubated with 300 ng nucleic acid guides at 55° C. in 50 μl reaction buffer (10 mM Tris PH 8.0, 20 mM NaCl, 0.5 mM MgCl2, 0.4% glycerol, 2 mM DTT and 20 μg/ml BSA) for 1 h. 400 ng pACYCDuet-eGFP plasmid was dissolved in 20 μl reaction buffer and added into the NgAgo solution for incubation at 37° C. for 8 h. Subsequently, the mixture was treated with proteinase K at 52° C. for 2 h to remove protein, and then subjected to electrophoresis in 1% agarose gel. The relative sizes of plasmid that is supercoiled (SC), linearized (Lin), and open circular (OC) are shown (FIG. 7A, left lane, “M”).

NgAgo did not cleave the negatively supercoiled plasmid DNA when alone or in the presence of non-complementary NC guide ssDNA (FIG. 7A, lanes 2 and 3, respectively). When supplied with either complementary FW or RV guides, NgAgo nicks one strand of the negatively supercoiled plasmid DNA, permitting rotation around the phosphodiester backbone and resulting in an open circular conformation (FIG. 7A, lanes 5 and 6, respectively). When supplied with both FW and RV guides, NgAgo cleaved both strands of plasmid DNA, linearizing the plasmid (FIG. 7A, lane 7). Unlike the guide-reloaded NgAgo, the NgAgo loaded with one guide DNA in vivo could efficiently linearize a target plasmid, indicating that NgAgo makes a double-strand break when loaded with single guide at 37° C. but not when reloaded with a guide at 55° C. Without being bound by theory or hypothesis, NgAgo becomes partially denatured and loses some activity when treated at 55° C. Indeed, a prolonged incubation of E. coli with guide DNA at 55° C. for 72 hours led to a complete loss of its nuclease activity (FIG. 7B).

The in vitro cleavage activity of NgAgo was further tested in the presence of ssRNA guides and ssDNA guides without 5′ phosphorylation. NgAgo was unable to cleave the plasmid when supplied with ssRNA with or without 5′ phosphorylation (FIG. 7C, lanes 3 and 2, respectively). It was also unable to cleave the plasmid when supplied with ssDNA without 5′ phosphorylation (FIG. 7C, lane 4). Only when NgAgo was supplied with a 5′ phosphorylated ssDNA guide was the supercoiled plasmid linearized. Thus. NgAgo can cleave double-stranded DNA targets at 37° C. only when supplied with a 5′ phosphorylated ssDNA guide.

### Example 3: In Vivo Guide Loading of NgAgo in Human 293T Cells

This example assesses guide nucleic acid loading of NgAgo in vitro and in 293T cells.

### Simultaneous Transfection of NgAgo and Guide Nucleic Acids

FLAG-NgAgo-HA-pcDNA3.1 plasmid was transected simultaneously with or without various guide nucleic acids to human 293T cells. FLAG-NgAgo-HA protein was then purified from the 293T cell as described in Example 1. Associated nucleic acids were isolated and analyzed by electrophoresis.

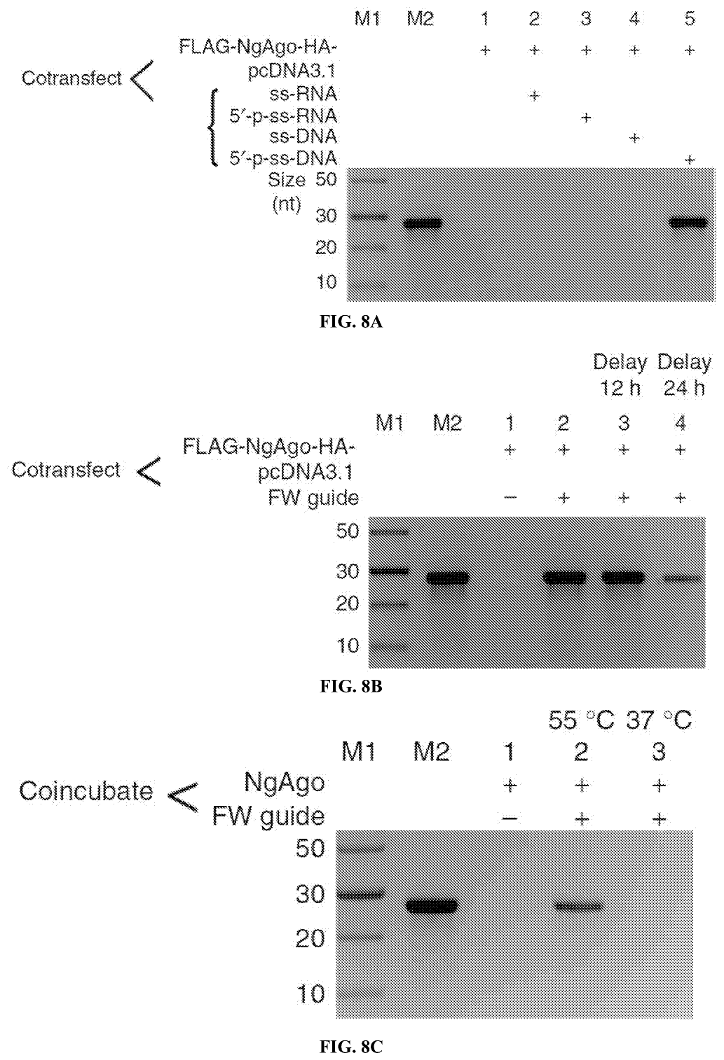

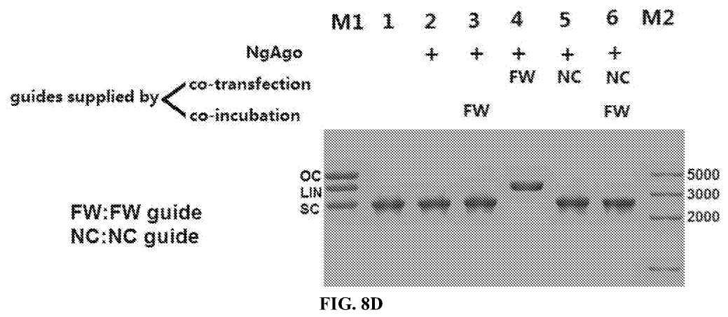

As shown in FIG. 8A, lane 1, without co-transfection of exogenous nucleic acids, there were no detectable endogenous nucleic acids associated with NgAgo purified from 293T cells. This implies that there is very limited 5′ phosphorylated ssDNA present in human cells, suggesting that endogenous ssDNAs will not mislead NgAgo to off-target sites.

To determine if NgAgo could associate with exogenous nucleic acids in human cells, 293T cells were co-transfected with NgAgo-encoding plasmid and the synthetic 24-nt ssDNA or ssRNA oligonucleotides with or without 5′ phosphorylation. NgAgo could only associate with the 5′-phosphorylated ssDNA (FIG. 8A, lane 5), but not ssRNAs (lanes 2 and 3) or ssDNA without 5′-phosphorylation (lane 4). Lane M1 shows the ssDNA ladder and M2 is a positive control of the ssDNA guide.

### Transfection of NgAgo Followed by Guide DNA

To further examine the timing of the NgAgo association with 5′-phosphorylated ssDNA in human cells, FLAG-NgAgo-HA-pcDNA3.1 was first transfected into 293T, and the delivery of 5′-phosphorylated ssDNA (FW guide) into the cells was postponed by Oh, 12 h or 24 h after transfection of NgAgo-encoding plasmid (FIG. 8B, lanes 2, 3 and 4, respectively). When FW guide was transfected into 293T cells 12 h after FLAG-NgAgo-HA-pcDNA3.1 transfection, there was not a significant decrease in the ability of FW guide to bind to the NgAgo (FIG. 8B, lane 3). However, when the FW guide was transfected after a delay of 24 h. NgAgo-ssDNA binding decreased substantially, which suggests that ssDNA loading occurs in a short time window when NgAgo is expressed (FIG. 8B, lane 4). M1 shows ssDNA ladder, M2 shows positive control FW ssDNA, and lane 1 shows NgAgo incubated with no FW guide DNA.

Co-Incubation of Purified NgAgo with Guide DNA

In contrast, NgAgo only efficiently loads guide DNA in vitro at elevated temperature. FLAG-NgAgo-HA-pcDNA3.1 plasmid was transected into 293T cells without any exogenous nucleic acids. FLAG-NgAgo-HA was then purified from 293T cells and was incubated with FW ssDNAs at 55° C. for 1 h or 37° C. for 8 h. As shown in FIG. 8C (lane 3), ssDNA could not be loaded onto 293T-purified NgAgo in vitro at the physiological temperature of 37° C., even after an 8 hour incubation. At the nonphysiological temperature of 55° C., FW guide was loaded onto NgAgo after 1 hour (FIG. 8C, lane 2). FIG. 8C lane M1 shows ssDNA ladder, with sizes in nucleotides (nt) depicted at left, lane M2 shows positive control FW ssDNA, and lane 1 shows NgAgo incubated with no FW guide DNA.

### In Vitro Cleavage Activity of NPAgo-gDNA Complex

The various NgAgo-gDNA complexes obtained by in vivo or in vitro guide loading were tested in an in vitro cleavage assay as described in Example 1. The NgAgo derived from the cells co-transfected with target-complementary FW guide (FIG. 8D, Lane 4) but not that derived from the cells co-transfected with a random NC guide (FIG. 8D, Lane 5) could cause double-strand breaks and linearize the plasmid. The NgAgo derived from the cells without guide co-transfection could not cleave the target even if the purified NgAgo was later co-incubated with the FW guide (FIG. 8D, Lane 3). Furthermore, NgAgo derived from cells with a non-specific ssDNA guide (such as NC) cannot cleave plasmid DNA, even when later incubated in vitro with the FW guide at 37° C. for 8 h (FIG. 8D, lane 6). These data indicate that NgAgo is faithful to its original guide and does not allow gDNA swapping at 37° C. This feature of NgAgo minimizes the possibility that it will be loaded with unexpected “guides.” Similarly, mammalian Ago2 protein cannot exchange its bound oligonucleotides with free oligos at 37° C. (Elkayam, E. et al. Cell 150, 100-110 (2012); Schirle, N. T., Sheu-Gruttadauria, J. & MacRae, I. J. Science 346, 608-613 (2014)).

### “One-Guide-Faithful” Rule Followed by NgAgo

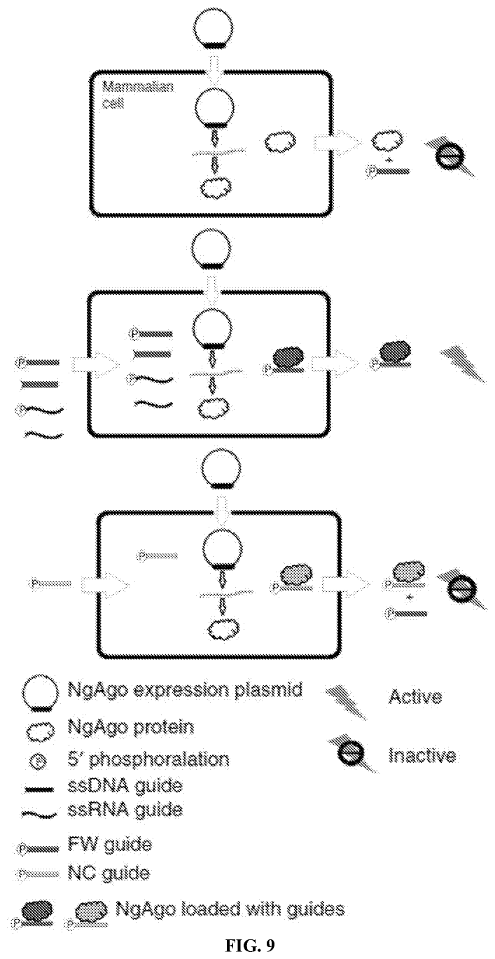

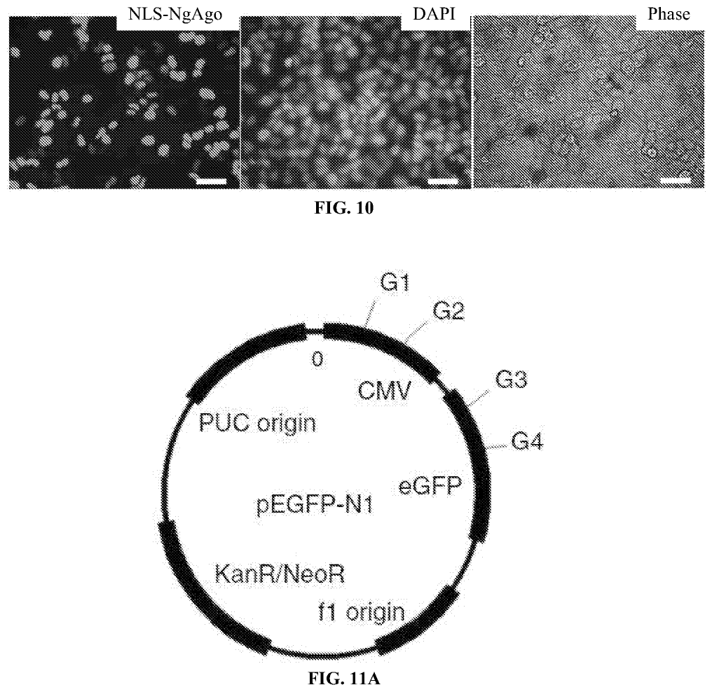

FIG. 9 summarizes the “one-guide-faithful” property of NgAgo based on the guide-loading experiments described above. The top panel of FIG. 9 shows transfection of the NgAgo expression plasmid into mammalian cells, where it is expressed, generating a NgAgo protein. After purification of NgAgo protein from the cell, it is co-incubated with FW guide ssDNA, but this does not result in active plasmid cleaving capability. This illustrates that the NgAgo cannot be loaded with ssDNA guide at 37° C. (FIG. 8C lane 3) and that co-incubation of purified NgAgo with FW guide does not result in plasmid cleavage activity (FIG. 8D, lane 3).

The middle panel of FIG. 9 shows co-transfection of NgAgo expression plasmid into mammalian cells with 5′ phosphorylated and unphosphorylated FW ssDNA and ssRNA guides. The NgAgo protein expressed in these cells can only bind to 5′ phosphorylated FW ssDNA guide, and when NgAgo-gDNA complex is purified from the cells, it is active in plasmid cleavage. This illustrates the association of 5′ phosphorylated FW ssDNA guide with purified NgAgo (FIG. 8A, lane 5), but not with unphosphorylated FW (FIG. 8A, lane 4) or with ssRNA (FIG. 8A, lanes 2 and 3). This schematic further illustrates the ability of in vivo pre-loaded NgAgo-FW gDNA complex to cleave plasmid DNA in vitro (FIG. 8D, lane 4).

The bottom panel of FIG. 9 shows co-transfection of NgAgo expression plasmid into mammalian cells with non-complementary NC ssDNA guide. The NgAgo protein associates with the NC guide, but the purified NgAgo-NC gDNA is subsequently unable to cleave plasmid DNA, even after in vitro incubation with the FW guide. This illustrates results shown in FIG. 8D, lanes 5 and 6.

### Example 4: Intracellular Modification of a Target Plasmid by Ago-gDNA

This example describes intracellular activity of Ago-gDNA on a target plasmid in HeLa cells.

Construct of NgAgo with Nuclear Localization

To test the ability of NgAgo to modify target nucleic acids in human cells, a modified NgAgo was designed by attaching a nuclear localization signal (NLS) to its N-terminus to ensure nuclear compartmentalization. The NLS-NgAgo construct was cloned into a pcDNA3.1 plasmid to provide the NLS-NgAgo-pcDNA3.1 plasmid.

To visualize localization of the NLS-NgAgo protein, an NLS-NgAgo-red plasmid was generated, in which fluorescent protein DsRed was fused to the NLS-NgAgo construct. NgAgo was isolated from the genomic DNA of N. gregoryi SP2 with PCR using the primers NLS-NgAgo-HindIII-F2 and NLS-NgAgo-BamHI-R, cleaved with restriction enzymes HindIII and BamHI, and subcloned into the pDSRed-Monomer-N1 plasmid (Clontech) (Table 4). The engineered NLS-NgAgo was transfected into Hela cells. Cells were visualized using bright-field microscopy image (FIG. 10, right), nuclei are visualized with DAPI staining (middle), and NLS-NgAgo-red was detected by fluorescence microscopy. The results showed that NLS-NgAgo was localized in the nuclei of HeLa cells.

### Comparison of NPAgo-eDNA and Cas9-ssRNA Activities on a Target Plasmid

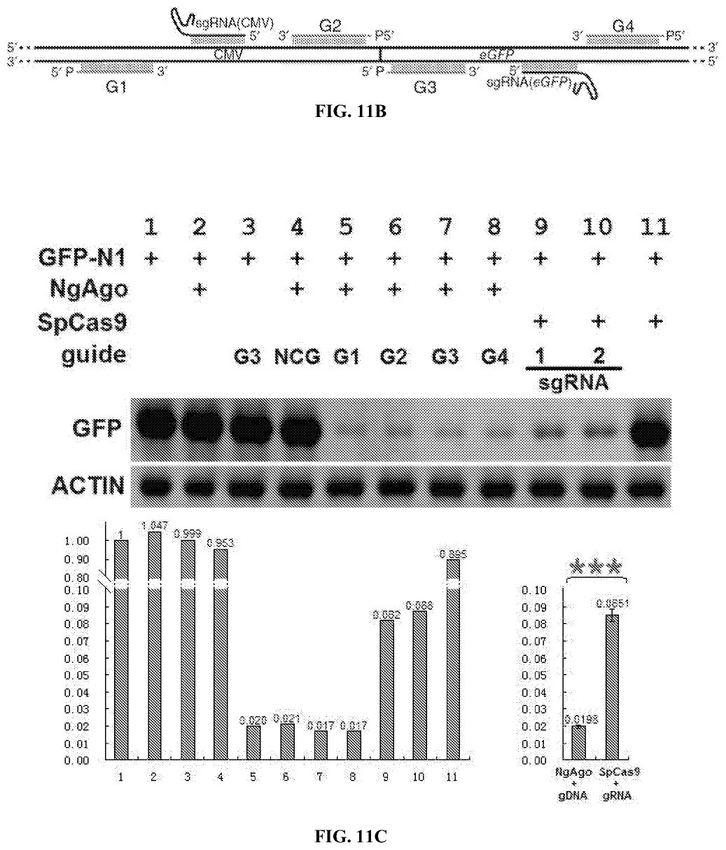

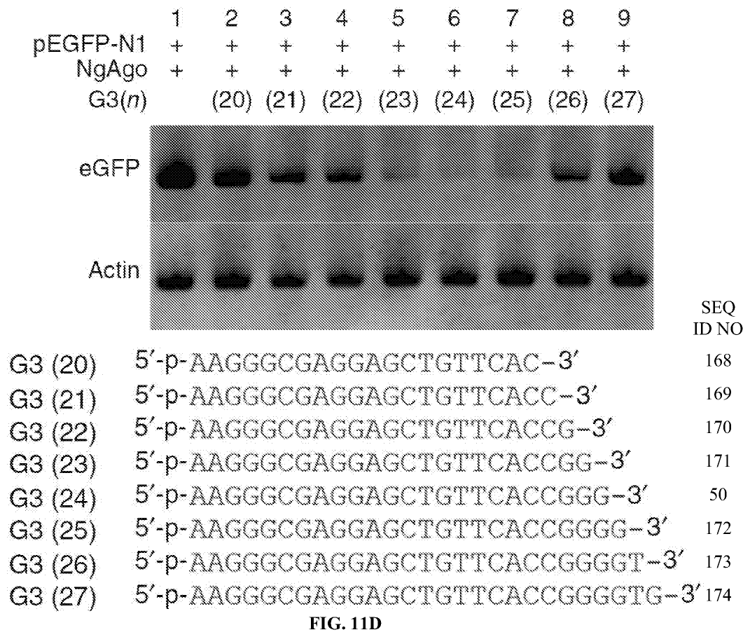

To investigate whether the NgAgo-gDNA system is an efficient tool for targeted DNA modification in mammalian cells, an intracellular plasmid cleavage assay was performed. ssDNA guides were designed to target two regions on the pEGFP-N1 plasmid. FIG. 11A shows a schematic for the circularized pEGFP-N1 plasmid, with the location of ssDNA guides G1, G2 within the coding region of the CMV promoter and ssDNA guides G3, and G4 within the eGFP gene. To compare the NgAgo-gDNA system with the Cas9-sgRNA system, single guide sgRNAs for Cas9 targeting of the pEGFP-N1 plasmid were also designed. Table 2 shows the sequences of 5′ phosphorylated NgAgo ssDNA guides G1, G2, G3, and G4, the negative control non-complimentary guide NCG ssDNA, as well as the DNA sequences encoding Cas9 single guide sgRNA sequences sgRNA1 and sgRNA2. FIG. 11B depicts a schematic of the linear pEGFP-N1 plasmid, with the positions of the NgAgo guides and Cas9 guides shown relative to the target CMV promoter and eGFP gene.

To compare the efficiency of target plasmid modification by the NgAgo-gDNA system with that of the Cas9-sgRNA system in mammalian cells, both systems were transfected into the HeLa human epithelial cell line. Briefly, EGFP-N1 plasmid, 200 ng NLS-NgAgo-pcDNA3.1 plasmid and 100 ng of each of the four ssDNA guides were co-transfected into 2×10⁵ HeLa cells using LIPOFECTAMINE® 2000 in 24-well plates. Similarly, EGFP-N1 plasmid, Cas9-expressing plasmid SpCas9-pCDNA3.1, and either sgRNA1 (targeting CMV) or sgRNA2 (targeting eGFP) vector were co-transfected into HeLa cells. 36 hour after transfection, cells were collected, and lysates were analyzed by Western blots to determine eGFP expression levels. Quantification was performed by gray scale scan (Image-pro Plus), and the eGFP expression levels were normalized to β-actin expression levels. Anti-eGFP (sc-9996) and anti-Actin antibodies (sc-47778) were purchased from Santa Cruz Biotechnology, Inc., and each was used in 1:1000 dilution.

The amount of eGFP protein expressed from the target pEGFP-N1 plasmid in HeLa cells was not reduced when cells were co-transfected with only NgAgo, only the G3 guide, or both NgAgo and the non-complementary NCG guide (FIG. 11C, lanes 2-4). However, when cells were co-transfected with pEGFP-N1, NgAgo, and any of the ssDNA guides (G1, G2, G3, and G4), the expression level of eGFP was significantly reduced (FIG. 11C, lanes 5-8). In comparison, the expression levels of eGFP in cells transfected with spCas9-encoding plasmid and sgRNA transcription vectors encoding sgRNA1 (CMV) or sgRNA2(eGFP) (FIG. 11C, lanes 9-10) were only moderately reduced when compared to cells transfected with target pEGFP-N1 and spCas9 alone (FIG. 11C, lane 11). Thus, when providing cells with the same amount of NgAgo and Cas9 plasmids, the NgAgo-gDNA system was as efficient at suppressing eGFP expression as the Cas9-sgRNA system.

Optimal gDNA Length for Intracellular Activity

To determine the optimal length of ssDNA guides in guiding NgAgo for plasmid modification, guide DNAs (G3(n) guides) were designed based on the G3 guide, but varied in length from 20 nucleotides to 27 nucleotides. HeLa cells were transfected with pEGFP-N1 plasmid, the NLS-NgAgo-pcDNA3.1 plasmid, and each of the G3(n) guides. 36 hours after the transfection, cells were harvested and cell lysates were analyzed by Western blots to determine the expression levels of eGFP. Despite difference in efficiency, gDNAs having 20-27 nucleotides can all bind to NgAgo and allow cleavage of the target DNA (FIG. 11D). The cells transfected with the 24-nt guide DNA showed the greatest suppression of eGFP expression, suggesting that 24-nucleotides is the optimal length for guide DNA of NgAgo (FIG. 11D, lane 6).

### Modification of Target Plasmid by Other Ago-gDNA Systems

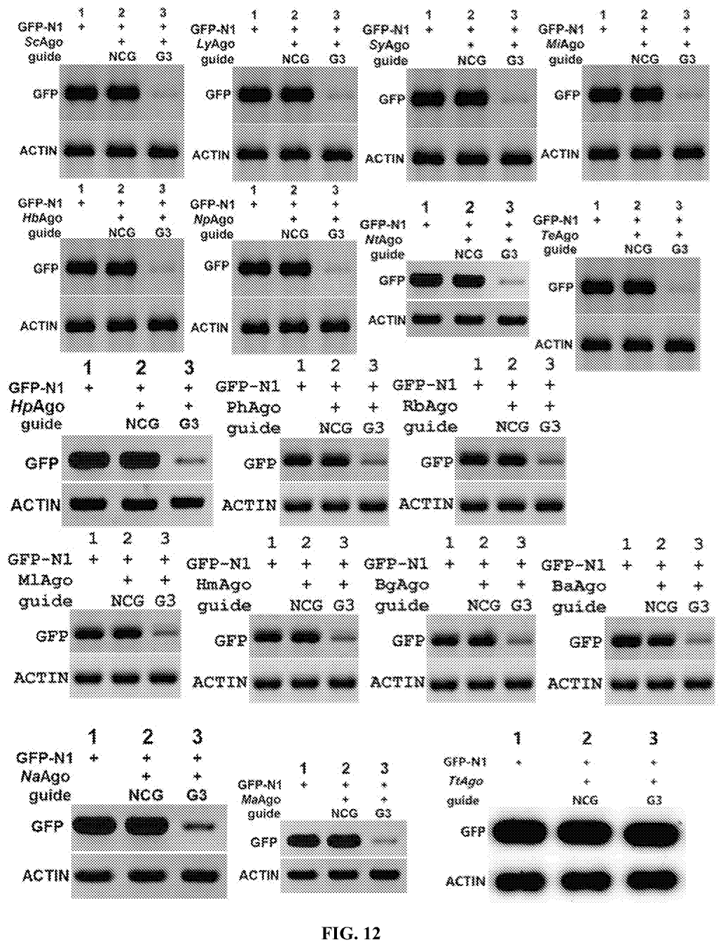

Using similar methods as described above for NgAgo, MaAgo, HpAgo, NaAgo, NtAgo, NpAgo, HbAgo. MiAgo, SyAgo, LyAgo, ScAgo, TeAgo, PhAgo, MIAgo, HmAgo. BgAgo, BaAgo, and RbAgo were found to have similar modification efficiency on the target plasmid pEGFP-N1 in HeLa cells (FIG. 12). G3 gDNA was used in these experiments. In comparison, TtAgo (last panel) was not able to suppress GFP expression in HeLa cells because the enzyme is obtained from a thermophilic bacterium species and is only active at elevated temperatures. The amino acid sequences of HpAgo, NpAgo, HbAgo, MiAgo, LyAgo, ScAgo, TeAgo, PhAgo, MIAgo, HmAgo, BgAgo, BaAgo and RbAgo are shown below.

(HpAgo)
SEQ ID NO: 3
MVKRYISFHLFPRIKLCGVYLCLRMNTKDDIAHKQPITIEVQVLKELDKP
SPKMATRLLVADRAGNRFPLAIWKNNALSDYDWTIGQWYRLENARGNVFN
GKQSLNGSSNMRATPLEASEEDETRADDVGRVDTILGNLSPNQAYLSLFP
ISRSFDTLSVYEYSIEAAEAFEDDPDTVTYQCAGRLRRITGAGVAYAGPM
QIVSTRKLPDKLADPFSLSEPTERELKAADARDRHRIERLLKSLVKAAID
DSTYDPYQINRIRARTPAITAGDGLFEACYEFAARVDVMPSGDAFVGIEV
RYHARSQVTADVYEDKTGELVGTIVEHDPERYNVSGTGRVVGFTDHYFTD
ALDELGGLSLADWYAQKDRVPEGVLEALREKNPRLVDIQYQEDEPAQIHV
PELLRVAPRKEVVKELDPTFHRRWDREAKMLPDKRFRHAIEFVDHLGSLP
DIDATVAPEPLGPSLSYMSTAVDREENLRFKDGRTATTPSSGIRSGVYQQ
PTSFDIAYVYPTESEQESKQFISNFENKLSRCHCEPTATRHVPYELGGEL
SYLAVINELESVDAVLAVVPPRNDDRIAAGDITDPYPEFKKGLGKQKVPS
QMVVTENLDTRWVMNNTAMGLIAGAGGVPWRVDEMPGEADCFIGLDVTRD
PETGQHLGASANVVYADGTVFASKTQTLQSGETFDEQSIIDVIKDVFQEF
VRREGRSPEHIVIHRDGRLFEDADEIQAPFADSGVSIDILDIRKSGAPRI
ARYEDNSFKIDEKGRLFISQDDTHGFIATTGKPEFDDSDNLGTPKTLRVV
RRAGDTPMLTLLKQVYWLSEAHIGSVSRSVRLPITTYYADRCAEHAREGY
LLHGELIEGVPYL
(NpAgo)
SEQ ID NO: 6
MPTQSDIEDGERIDIQVKVLSELDRPSEKMAKRLRVRDTDGNEFPLTIWK
NNALCDFAWERGRWYELENARGNEFRGEKSLNGSSRLHADPVDNPIDSDR
SQQSTTAESTDKQFDSLEDGLPYLSLFPIDREFETVDVYEYRIEADGPFD
DDPMDATYTLAAYLRSCSDAAVTHAGIFSVIATNRLTNALPDPFELTDES
RVTLRADDETDNECLVRLLQQVFKTAVDDETYETGRVDRIRTQDPVITGQ
DGLFEACLAYTARLEILPSGKAFVGIDISYHARSQVTVDKYVDRINASVD
ELIDTPVEHDPERYEKSGSGRLKGFADVTFTDPVDDFGNQSLADWYEQKG
RISDDMLERLRSEDPQLVEIQYNPNSDETNLHVPQLLRVAPRKEIVKKLA
PTFHRKWDRAAKMLPDDRFRKATRFVARLDSLSEVDAQIEPNPVGPNISF
MSTEVDRSDNLRFGDDQTTTLPNNGLKRYGIYRRPSSLHLHYLVPERYTD
EFASFREQLERQLATIGCSPDDISYDEYGLGNAINYNTTAAAVDDVDIVL
AVVPAPDNDFIRNGTIDDPYPEFKKSLGKQTIPSQMVREDNLDDRWILRN
TALGVIAGAGGVPWRVDEMPGDVDCFVGLDATRDPETGQFLGASANVVLS
DGTVFVSKTQSLQSGETFDENAIVDVLKDVHREFVREEGKSPNNIVIHRD
GRLFEDVDTILEPFDETDIDIDILDVRKSGAPRAAVYQDDQFQVDHKGRL
FVAQSGDYGFLTTTGRPEFDEDDGLGTPRSLRIVRRAGETPMRTLLEQVY
WLSESHVGSAQRSTRLPITTYYADRCAEHAREGYLVNGELIRGVPYL
(HbAgo)
SEQ ID NO: 7
MAVKADIEDGEEIDIALHVTGIDEWEHDAIARKIQLEDVDGAAIDLTVFH
NNDVSDFEWEIGEWYLLENVVGNEFRGEMQLNPGYDLTVTLLNDPPAAAG
NDKSPGSVPPEEPVDQSGESGSSGAAASTSGEPGDAEFVRGSEVDDGSRP
TADGGGKLLHQQPLSDGNYLLQFELGSLPELPVHEYELRATGSGGIDPDD
FTNGIEGFTAKAANYYQSRIGSPVTTADASRRRIYATEKLHGTISMHGYT
VKPVHQGETTLEARSYTNDGPLQEFVKQDVKRAVAGRFEVSGIDSIIEPT
PQRTANSGLFEAYRKYKCRIRVDADGTVICGVNVAYHLESTFSAAEWVQR
GHDIADVTVEHDTDLYDSARTATVKEIIDMDYDDMLDGPGVPMSEYHEKH
VEQDVIDSMRAGNPVIADLQYGSGEDSIFPQLLEYCKVIPTFDQLGRVDE
TFLNVIHNESRMKPEERFNVVTSFVDLLGPTPYFDFGPVPQPTNAGYREQ
KTPNIPNLRFGDGRTGYYGAGGLERKGYGVYKAPESFDIIALYPDSEQAA
ARPYVLSVLGKLAEYDGKPTKFDQETYELGSEFHYSQHAQKTSDYDAALI
VVPDKDKAAAADYDDPYPEFKRRLGQLGVPSQMITIDNLGNDSYLGNISS
SLIGKAGGVPWRIDDVPGDVDAFVGLDVTYDHATKQHLGAAANVIMADGT
ILASEAVTKQAGETFDEDDVANVIKHVLEIFAEEEGRPPRHVVIHRDGKF
YLDIESLIKRLDKARDLIQRFDLVEIRKSGNPRIAEYDESKSRFDIADKG
VAFHVHNGDHSYLTTTGGKEGSPGTPRPLQIVKRHGSTDLDTLAEQTYWL
SEAHVGSLSRSTRLPITTYYADKCADFAMKGYLTKGSVIRGVPYI
(MiAgo)
SEQ ID NO: 11
MNYTETKTANSPIFLSEISSLTLNNNCLNCFKLNHQVTRKIGNRFSWQFS
RKFPAVVVIFEDNCFWVLAKDEKLLPSPQQWKEALSDIQEVLREDIGDHY
YSIHWLKDFQITALVTAQLAVRILKIFGKFSYPIVFPKDSQISENQVQVR
REVNFWAEIINDTDPAICLTLESSIVYSGDLEQFYENHPYRQDAAKLLVG
LKVKTIETNGTAKIIRIAGTIGERREELLTKATGSISRRKLEEAHLGQPV
VAVQFGKNSQEYIYPLAALKPCMTDKDESLFQVNYGELLKETKIFYAERQ
ERLKLYKQEAQNTLNNFGFRLGEKSINSREYPELFWNPSISLEQTPILFG
KGERGEKIKTLKGLSKGGVYKRHREYLDPARKIRLAILKPANLKVGDFRE
QLEKRLELYKFETILPAENQINFSVEGVGFEKRARLEEAVDQLIRGEIPV
DIALVFLPQEDRNADNTEEGSLYSWIKKKFLERRVMTQMIYEKTLNDKSN
YKNILNQVVPGILAKLGNLPYVLAESLEIADYFIGLDVGRMPKKNLPGSL
NVCASVRLYGKQGEFVRCRVEDSLTEGEEIPQRILENCLPQAELKNQTVL
IYRDGKFQGKEVDNLLARARAINAKFILVECYKTGIPRLYNFEQKQINAP
SKGLAFALSKREVILITSQVSEQIGVPRPLRLKVHELGDQVNLKQLVDTT
LKLTLLHYGSLKEPRLPIPLYGADAIAYRRLQGICPSLLEDDCQFWL
(LyAgo)
SEQ ID NO: 33
MNTTSQAPQNSTSSSIYLSEIFPLTILKPNLICFRLTPEVDREVGNRLSW
RFSQKFAEIVVIWENKYFWVLAKPTQKMPSPEQWRQALGEILEQLKEDIG
DHYYSIQWVRDPQVTASTLAQLAVRVLKIRKPFSPDIIFSENQVQVQSEV
DFWPETIELANTLTPAITLTLKSRFLYRGTLAEFYANHPYRNKPKELLVG
LKVRDRETNSSATIVEIAAIDEDRRKELIEKATGAVSRQALEEAPDDQPL
VSVRFGKNQKLFDYPMEALCPSITKETASKFDVNYGDLIKQTKPPYQERQ
NHLTQSKQKAEESLAVYGFQIDKSINNLDYPSLFQTLQFNLEDTELLFGK
DSSGKHFVSKRGSVLKGLSQGGVYRRHQDYENYSTPIRISLLNLCNSKVG
KFVSQVEERLKQYKFETIRFEKDSLDRRKEIKVDNLDSAEARVAVEKALD
ELMVIPTNIVLTFLPESDRHTDDTEDGSFYSFVSSRLLRRGISSQVIYED
TLKNPNNYSYILNQVIPGILAKLGNLPFILAKPLEIADYFIGLDISRTPK
KRKSGSLNVCASVRLYGKYGEFIRYRLEDALTQGEEIDKRTLERFLPAAD
LSGKTVLIYRDGRFCGDEIKHLRERAKAMGSKFILVECIKSGIPRLYEVQ
ELTVKDKKKPILKAPPKGLALRLSSHEVMLVTTEVKSEKMGLPNPLRLKV
IPEEGQQVSLESLVEATLKLTLLHHGSLKEPRLPIPLYGSDIIAYRRLQG
ISPGELDGDRQFWL
(ScAgo)
SEQ ID NO: 28
MPMRMLQQEVPVILNRFLVKELTQEDLTFYEYDCRPNPPPELGEEQRAIA
RVCNRLDVIAARLGSRIITQERVSPSQLKTPEWELEERGLRVLTCANAQE
RSALESFERKRIGLRLKSQYKKTEVEWIGGGLLWWVTAQKGVELSGEGWE
IHRGRMIDVAVEPDGKLYLEVDIHYRFYTPWTLHEWLESYPDVPIEIVRN
TYFDANGKRLTWKYLGILSNQSPREIRLPEQNISLAEYHLQKYNALVEEV
ESSWVVEVASKERRYPHLSRRLSPALTLEMLASLEDNRSPGGKVSAAVIE
CIRKSLKERFEESEETARTIIKEVYKLSPEEIKPLKTQGYILPKPKLLGS
GRRPVENPARVRYRGCAKVGETKFACLNLYDDKREYPSEVLNCLLEVAQK
SGAEIEVDFYATQQDLPKGDLARKRFWQTWAEQGIKTVLVVMPWSPNERK
QRIRMEALEAGIATQFMIPGADPYKALNVVLGLLCKAAWQPVLLEPLDDP
VGPELIIGFDVGTNRRLYYGASAFAVLADGQSLGWELPAVQRGETFSGDA
IYQTVSKLVDRFYDKLSRYPSKVLLMRDGLVQGGEFSRTIEELEKEKIAV
DIVGIRKSGTGRMGIEEGKGKYKDAKIGTVVFDYSRKSFTLITSQPIRKG
GNSLGSARPLRAIHEHGKTPLEVLALQTYHLSQLHPASGFQACRLPWVLH
FADKSGKEFQRLGDNIFSILQNIDRQKLIAV
(TeAgo)
SEQ ID NO: 9
MPRETCYDKRTTPSQYGWLPIDSLSVMPTQFQEVEVILNRFFVKKLSRPD
LTFHEYQCQFTQVPEQGSEQKAISSVCYKLGVTAVRLGSCIITREFIDPE
RMRTKDWQLQLIGCRELSCQNYRERQALETFERKILEEKLKETFKKTIIE
KDYELGLIWWISGEEGLEKTGHGWEVHRGRQIDLKIETDEKLYLEIDIHH
RFYTPFKLEWWLSEYPNIQIKYVRNTYKDKKKWILENFADKSPNEIQIEA
LGISLAEYHRQEGATQQEIDESRVVIVKKISDYKAKPVYHLSQRLSPILT
METLAQIAEQGREKKEIQGVFDYIRKNIGTRLQESQKIAQVIFKNVYNLS
SQPEIMKVNGFVMPRAKLLARNNKEVNQTARIKSFGCAKIGETKFGCLNL
FDNKPEYPEEVHKCLLAIARSSGVQIKIDSYFTGSDYPKDDLAQQRFWQQ
WAAQGIKTVLVVMPWSPHEEKTRLRIQALKAGIATQFMIPTPQDNPYKAL
NVALGLLCKAKWQPVYLKPLDDPQAADLIIGFDTSTNRRLYYGTSAFAIL
ANGQSLGWELPDIQRGETFSGQSIWQVVSKLVLKFQDNYDSYPKKILLMR
DGLVQDGEFEQTIRELTHQGIDVDILSVRKSGSGRMGRELTSGNTAITYD
DAEVGTVIFYSATDSFILQTTEVIKTKTGPLGSARPLRVVRHYGNTPLEL
LALQTYHLTQLHPASGFRSCRLPWVLHLADRSSKEFQRIGQISLLQNVDR
EKLIAV
(PhAgo)
SEQ ID NO: 41
MRNILLNFLKFENQDFGTTVFRKEAEAGVFKDGFSYYDFEVDGRNVKYEI
SDTALEGYTSFTLPSYLNVGLVSKKLYEKIIDASNQVNGRFILHPEKEYN
RRIHFEIEPHPKGRKCVWVEPYFLKSKQVWGLLIGFQFIVSQNVLSGGYK
VDRDIQIASGSLNARGLSNLDFYLFKYNHITTFIKNILPGINLNLNNAIN
SSLFPVESYLLDAKQYMFKDNRIDNSSYFGLSKYAPLQPVSKETTFYFIY
RKSDRGIAVNLLKGLRGESHPNTFSGIQKFFKIPFTNDHIKGFALDDYNE
PNISKVVEDIKAEVNNVLPVIITNSKKEESDDKLYFSLKHRFTNEGIPCQ
VVTKDLIINDNALKFSLGNIALQMFAKAGGIPWKMKPATTEYLIIGIGQS
YNIEITEDGNKVEKNITYSVLTDSSGIFKDLQVLSEGVATDDSYYTQLVN
NIAGIINNGKYKKIAVHTPFRLSKDKVLDKVVKLIDHNIELSVLVINDKT
DFFGFDASNNGLVPFESTFLKLSSQEYLVWFEGIQPSNPKITKRFGNPLS
IKFWYTNNPQFFQDIDYKESLLQDCINLSGANWRGFKAKQLPVSVFYCQR
ISEFIGKFRQYELSHIDINNLKPWFL
(MlAgo)
SEQ ID NO: 40
MKFETKIFDEPLLEFGNQHYHPDPRLGLFEAGPLQTPLGDVINIAVVGSA
KTVKDSRDFLQAAAVGFAGKSEKHPNLHPPFPGLGNQNPFRCRFEIPDGA
VTAIPQARIERIRKEPHHGKAVEMAVDEIIEQLQTIDEGSSRPDVAIIAL
PVELIERVWNAKVDSDATLEEEDSSGSDAPNFRGMLKAKAMHFRFPIQIV
WEDVLDERAVIPLKIKESSARQIQDQASRTWNLLTTLYYKGSGRVPWRRA
PQEGEFSACYIGVSFYREAGGQQLFTSAAQMFDERGRGFILKGKGAQTES
RGRHPYLTQDDAKTLIADALAAYKKHHMNYPARVIVLKTSRFRDEEADGI
FEALDEVGTELRDLVWVQESSFVKVFRDGNYPVMRGTFVKLDGKGLLYTN
GSIPYYGTYPGMYDPKPLLICPYKTSDSTVAQIANEIFGLTKINWNSTQM
NQKLPIPIRAARKVGEVLKYITDEKVSSDYRRYI
(HmAgo)
SEQ ID NO: 39
MTPQDTPFTLRHLEEPEIQFEGGTETSPKRGLIRYGPRLYEEGHHTIKLG
IIGDRDSIRRLTELLHDMEVGIHPGTSDNPWQVPYPGLGKSSPLNLSINA
KKGWRRQIRRRDIQSVTSKSTPRDRMERFLKLVQKDIEIIERDSVQPNAI
IVCIPQEVMDACTPENQDHARIQSEGSDLRNRIKLIGMEARIPTQLIKPS
TLAIRTDRQRASRAWNLTVGLLYKSQRGHPWKTRQIEDGHCYAGLSFYRE
RDEGDDVIRAALAHVFHGRDHIILQSDPLPDITEDENGSPHLSYEAARQV
GEQILEYYEAQKGTRPSRFVLHKPSVFWEEEREGLLDATDGVRDLDLVWV
RRRPKVRLFPPTDYPAMRGTLLSVPDDDVHYLYTSGYVPEETTYQGSGVP
SPIEIRPDEICETPSLEICKEILFFTKLDWNTSDYAIRMPATVSVAKRVG
TILSEVDTESISEVRPQYFYYM
(BgAgo)
SEQ ID NO: 38
MHNRAALQTGSSVRRMGVDALVRSLAVSQDRPLMLFLGAGASMTSGMPSA
NQCIWEWKRDIFLSNNPGIEEQFSELSLPSVRDRIQTWLDRQRCYPVAGH
PDEYGAYIEACFSRSDDRRRYFERWVKQSTPHTGYRLLAELAASGLIQTV
WTTNFDGLIARAAAATNLTPIEIGVDSQQRLYRAPGKGELACVSMHGDYR
YDRLKNSSGELAQVEVQLRDSLIEALRTHTVVVAGYSGRDESVMQAFHQY
AISGPVRTDLPLFWTQYGEAPPLDTVNALLSTNHGEPSRFLVPGVSFDDL
MRRLALYLSKGPARDRVNKILDEHATTPVNQLTAFGLPPLPPTGLIKSNA
IPLTPPQELLEFDLLQWPASGTVWATLRELGDRHNFVAAPFRSKIYAIAM
AESLRVAFGENLKGEIKRVPLNDDDLRYEDGVINQLVRRATVLALSAKAN
CPSDGESLIWTSEKVEDLRLDRVDWKVHQAVLVQIRPLGSELALVLKPTL
YVTDRSGAIAPKDTERLVKQRVLGYQHNKEFNGATEAWRRRLVPQRDFRV
RFPDHENGIDLTFSGRPLFARITDERERTVSLSAAQESAARQAGLQLAEP
RLKFARKSAAGLAFDTHPVRGLINNRPFDSSLTTTGIASSIRVGIIAPAR
DATRVHQYLSQLHVAAQPGKDADYLPPFPGFASAYQCPIEIPSVGEQSFV
QLDEPDSMTPSSARALAGAITRSIASLSASQRPDVTIIYVPDRWAPLRNY
MIDDEEFDLHDFVKAAAIPKGCATQFVEEDTLRNTQQQCRVRWWLSLALY
VKSMRTPWTLEGLSEKSAYVGLGFSVKRKTTQNAGAHVVLGCSHLYSPNG
IGLQFRLSKIEDPIMRNKNPFMSFDDARRLGEGIRELFFAAHLRLPERVV
IHKQTPFLREERSGLQAGLEGVACVELLQIFVDDTLRYVASHPTSDGKFE
IDNYPIRRGTTVVIDDHTALLWVHGASTALNPGRHYFQGKRRIPAPLVIR
RHAGTTDLMTIADEVLGLSKMNFNSFDLYGQLPATIETSRRVARIGALLD
RFSDHSYDYRLFM
(BaAgo)
SEQ ID NO: 37
MSDVVEKVQWAAIPQMSIDAFVRSIAVNQNRPVCLLLGAGASITSGMPSA
QRCIWEWKRDIFITKNPTLRDSLDELSLPGTRRIIQSWLDLQARYPVEGS
PDEYSFYAEECYPTSLDRRTFFHRFISEAKPHIGYKLTALLAEAGCVRTI
WTTNFDGLVARACTAADVVCVEVGIDTAHRASRAQNDNELRLVSLHGDFR
YDALKNTADELREQDAALRKEFLHELKDYDLIVIGYSGRDESLMRVLSAA
YADRSSCRLYWCGYGAEPGTEVQRLISSIDPSRESAFYINTKGFDDVISR
LAVRRLSGKQLAFAHELIETMAPTVGQRMAFAVPPLSPSALVKGNAYRLS
YPGNALKLDIELPELGSWREWLAERMPPTLGQSVVFENGALCLADTAVAS
RVFDEALRRPPRRIEISDENIVTDGRITSLFRRAIVKAAAKTLNVRTDFR
RRIWEPIHYQTRELDNVRYLIHRALSMNIVGIDGIPHVVLTPEIVATMED
GGVAPFEPQKALRVAIYGFQHNDKFDGDLSYWTRQLVEKALDADGGGAFT
ISKIPLYAGLAQKGKPPLPPTLAKHAKQSGIIVPDAPLVFSAKVGTSEVR
NPNPLHGLVLNRPWDHSLTATGLCPSTETAVICPADASTRFERFLRGLQE
VARPEQSERDYLHDFPGFPAAFGLPLKIPVRGDSTWMTIDDSVSTDALTG
AKQLAHRICEGLDHLRRARPSDTVVIFVPKRWEPFKVVDTQHERFNLHDY
IKAYAARHSQSTQFVREETVLNSYTCRVRWWLSLALYVKAMRTPWRLDAL
DENTAFVGIGYSLDSEAERGNHVLLGCSHIYSARGEGLQFRLGRIENPVM
RGRNPFMSEDDARRTGDTIRQLFYDSKMHLPTRVVIHKRTHFTDEERRGL
VQGLDGVKNIELIEINVEDSLRYLSSKFKDGKLDIDTFPVYRGTTIVESD
DTALLWVHGATPSAQNKYWRYYQGKRRIPAPLRIRRFLGQSDVVQCATEI
LGLSKMNWNTLDYYSRMPATLDSASSIAKFGTYLDGFSSAPYDYRLLI
(RbAgo)
SEQ ID NO: 42
MSDFKTKIFPEPELEFGDQHHHPDPRLGLLQAGPLQTNLGDTIKVGVVGS
ALTVEKSGEFLNAIEDGFEGKTEKHPNLHPDFPGLRNQNPYRCRFEMVAA
EDGVLTKGQIEKIAKEPSDARAVEIAVDAVMAQLEKLEAHHERPDVVMVS
LPVKLIERVWRNERARDDEGIEGEAADAKAGRETSPNFRGLLKARAMDLR
FSIQIVWEDVINPDAKIPRKIKENSDRQTQDRADLAWNLMTTLYYKGSGK
VPWRRLPEEGEFTACYIGISFFKDAETDEIWTSAAQMFDERGRGFILRGG
PAQSESRGRHPFLTIDEAHKLTESALAAYKSVHRTMPARVIVMKTSRFRE
DEAEGVGKALDEAGVELRDLVWIHESYSVKVLRDGDFPVLRGTFVELDGN
GLLYTNGSIPYYGTYPGLYVPNPLLLCPHPQSESTIEQIAKEVFSLTKVN
WNSTQMNQRLPIPIRAARKVGDVLKYVPSGQKVSSDYRKYI

### Example 5: Endogenous Loci Cleavage by NgAgo-gDNA in Mammalian Cells

This example describes the ability of NgAgo-gDNA to cleave endogenous sites in the human genome. The cleavage activity of NgAgo-gDNA was quantified based on the mutation rates resulting from the imperfect repair (i.e., indel mutation) of double-stranded breaks by Non-homologous End Joining (NHEJ) pathway using the T7EI assay as described in Example 7. Genome cleavage efficiency by NgAgo-gDNA was compared with that by Cas9-sgRNA.

### Cleavage of DYRK1A Loci in 293T Cells

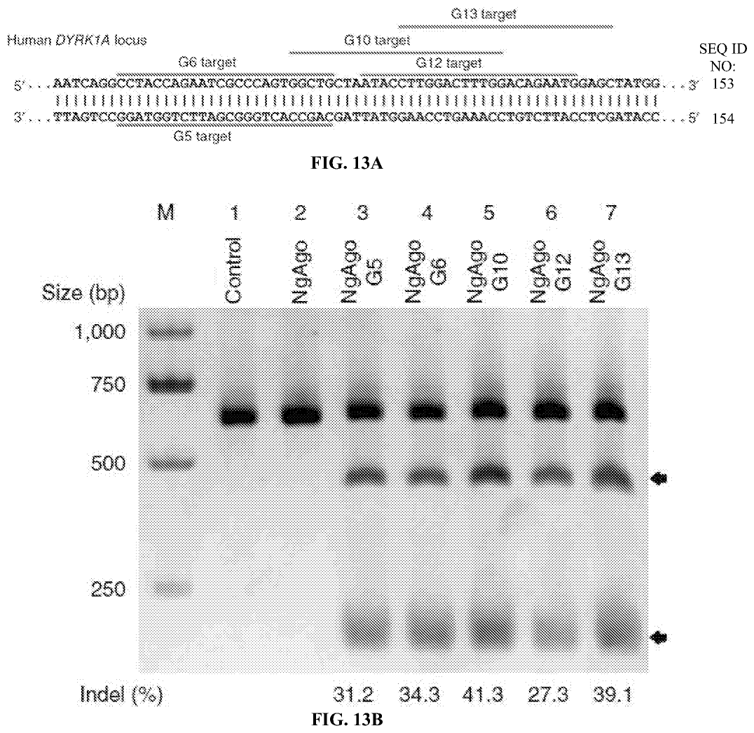

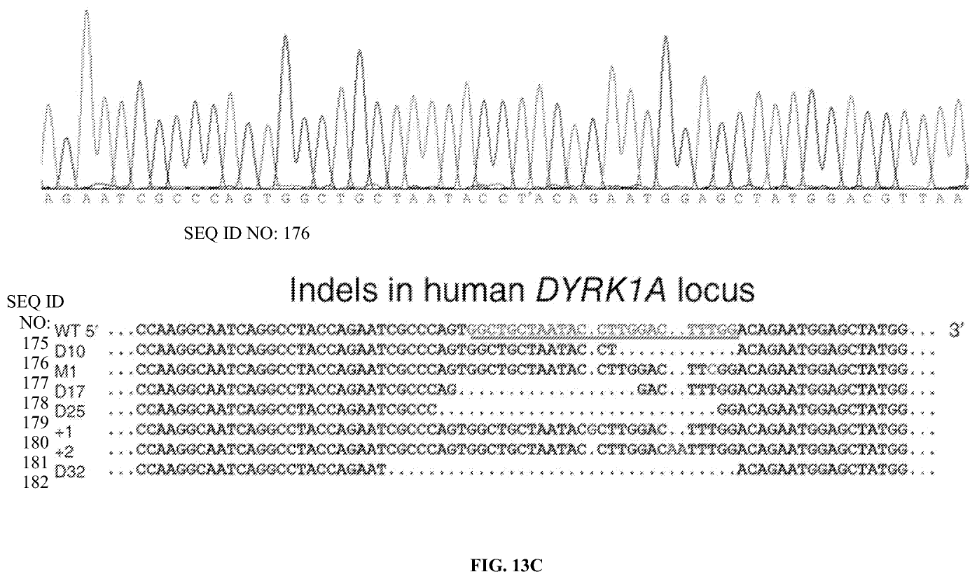

Five gDNAs were designed to target exon 11 of the human DYRK1A gene. FIG. 13A shows sequences for ssDNA guides G5, G6, G10, G12, and G13 (Table 2) and their targeting positions along the sequence of the human DYRK1A locus. 293T cells were co-transfected with NLS-NgAgo-pcDNA3.1 plasmid and the indicated gDNAs. Genomic DNA was extracted, and amplified by PCR using primers DYRK1A test F and DYRK1A test R (Table 3) to result in a 584 bp product. For each T7EI reaction, 500 ng of PCR product was denatured, reannealed and digested with T7 endonuclease I, which cleaved mismatched heteroduplex DNA as a result of NEJH repair of DSBs induced by the NgAgo-gDNA. The reaction was analyzed by denatured PAGE and visualized by silver staining.

FIG. 13B depicts the results of the T7EI assay showing NgAgo-gDNA cleavage of the human DYRK1A gene. The control (lane 1) shows the unmodified 584 bp PCR product from the 293T cell genomic DNA, and molecular weight marker is shown in lane M. All gDNAs were able to guide highly efficient target cleavage by NgAgo (lanes 3-7). Arrows show the positions of T7EI cleavage products. The cleavage efficiency at the DYRK1A locus by NgAgo-gDNA was between 27.3% and 39.1%.

Sequencing of mutated alleles from clonal amplicons using the G10 guide confirmed that indels were introduced to the target loci following cleavage by NgAgo-gDNAs. FIG. 13C shows representative sequences of the mutated alleles (bottom panel) together with an example chromatogram (top panel) having a 10 bp microdeletion (location marked by an apostrophe), which corresponds to sequence D10 aligned with the WT DYRK1A sequence.

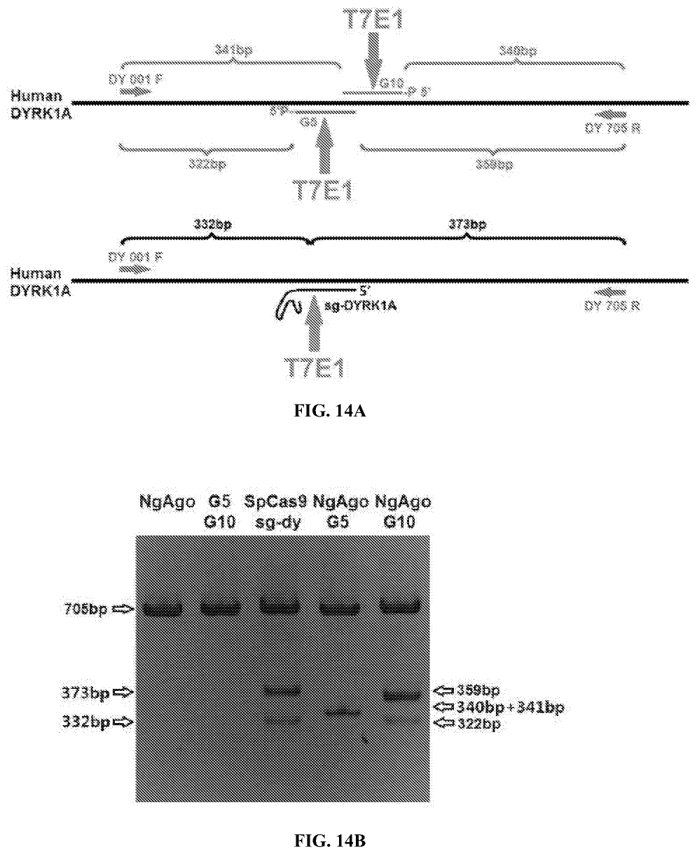

To further validate the T7EI assay, a different pair of primers were used for the T7EI assay on the DYK1A locus. FIG. 14A shows a schematic of the DYRK1A locus annotated with the primer annealing sites, G5 and G10 guide DNAs, and sg-DRK1A sgRNA, as well as the corresponding T7EI cleavage sites. FIG. 14B shows results of the T7EI assay demonstrating cleavage by the NgAgo-gDNA or Cas9-sgRNA systems. The T7EI cleavage products had the predicted lengths.

### Cleavage of Other Loci in 293T Cells

The NgAgo-gDNA system was also tested using 47 guide DNAs targeting 8 mammalian genomic loci (ACTIN, EMX1, HBA2, GATA4, GRIN2B, HRES1, and APOE). Cleavage efficiency for each locus was determined using the T7EI assay. Sequences of the guide DNAs are listed in Table 2. Primers for amplifying target regions and length of amplification products are listed in Table 3.

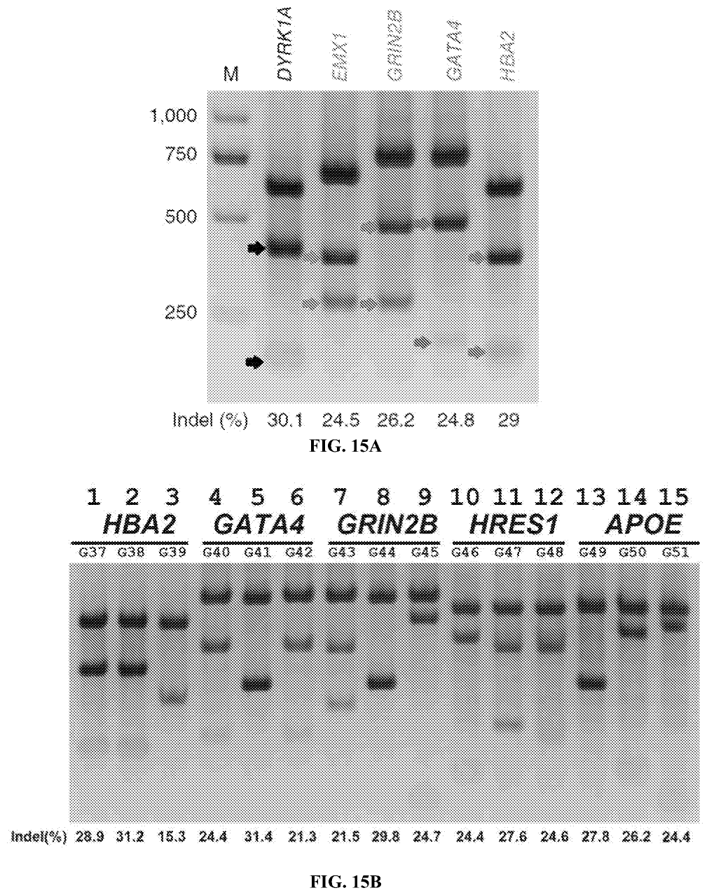

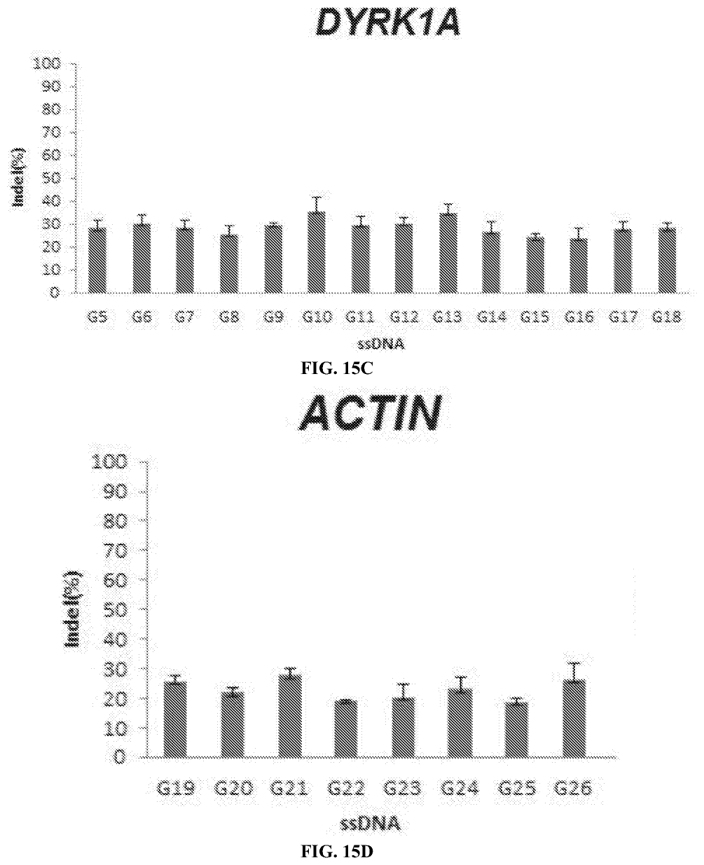

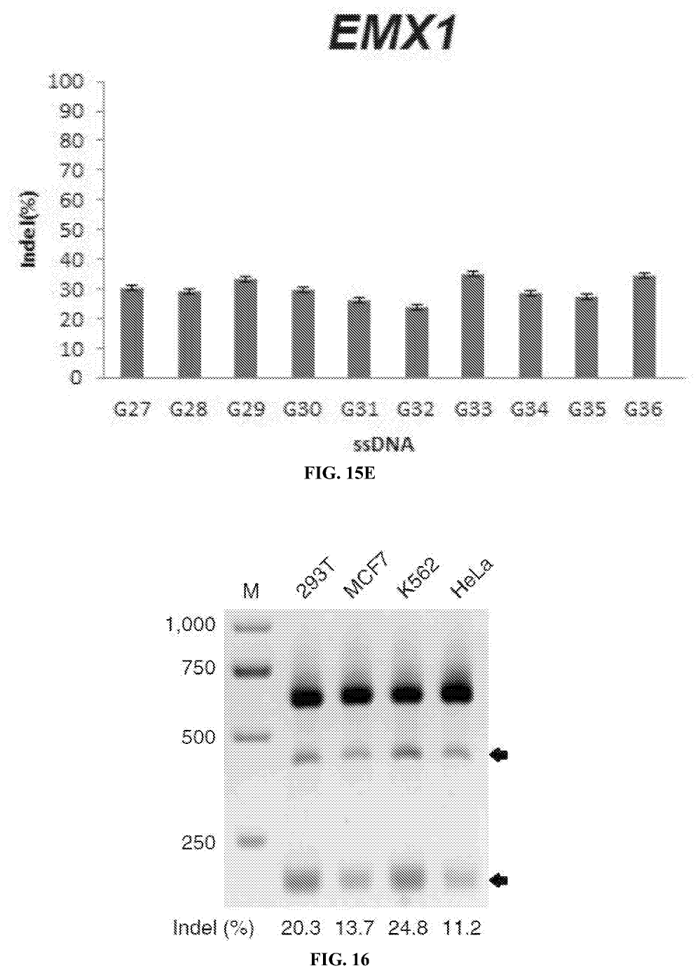

The results of the T7EI assay in FIG. 15A demonstrate cleavage of DYRK1A, EMX1, GRIN2B, GATA4, and HBA2 by the NgAgo-gDNAs. The top band represents uncleaved DNA, and arrows indicate T7EI cleavage products. The cleavage efficiencies were similar for all loci tested (24.5%-30.1%). Guide DNAs used in this experiment were G10 (DYRK1A), G31 (EMX1), G43 (GRIN2B), G40 (GA T44), and G37 (HBA2) respectively.

The results of the T7EI assay in FIG. 15B demonstrate cleavage of HBA2, GATA4, GRIN2B, HRES1, and APOE by the NgAgo-gDNAs. The cleavage efficiencies were comparable for all guide DNAs and at all loci tested (21.3-41.3%).

The percentages of indels measured by the T7EI assay were further compared for NgAgo-gDNA systems using a variety of ssDNA guides targeting DYRK1A (FIG. 15C), ACTIN (FIG. 15D), and EMX (FIG. 15E). The cleavage efficiencies were comparable for all guide DNAs, and there were no observed preferences of NgAgo for sequences with specific properties.

### Cleavage of DYRK1A Locus in Different Cell Lines

The cleavage efficiency of the DYRK1A locus by NgAgo-gDNA was tested in different human cell lines. Each cell line was transfected with NLS-NgAgo-pcDNA3.1 plasmid and ssDNA guide G10. The T7EI endonuclease assay was performed to determine cleavage efficiency. As shown in FIG. 16, the NgAgo-gDNA system could efficiently cleave the DYRK1A locus in 293T (human embryonic kidney), MCF7 (human breast cancer), K562 (human myeloid), and HeLa (human epithelial) cells.

Cleavage of DYRK1A Locus Using Mismatched gDNAs

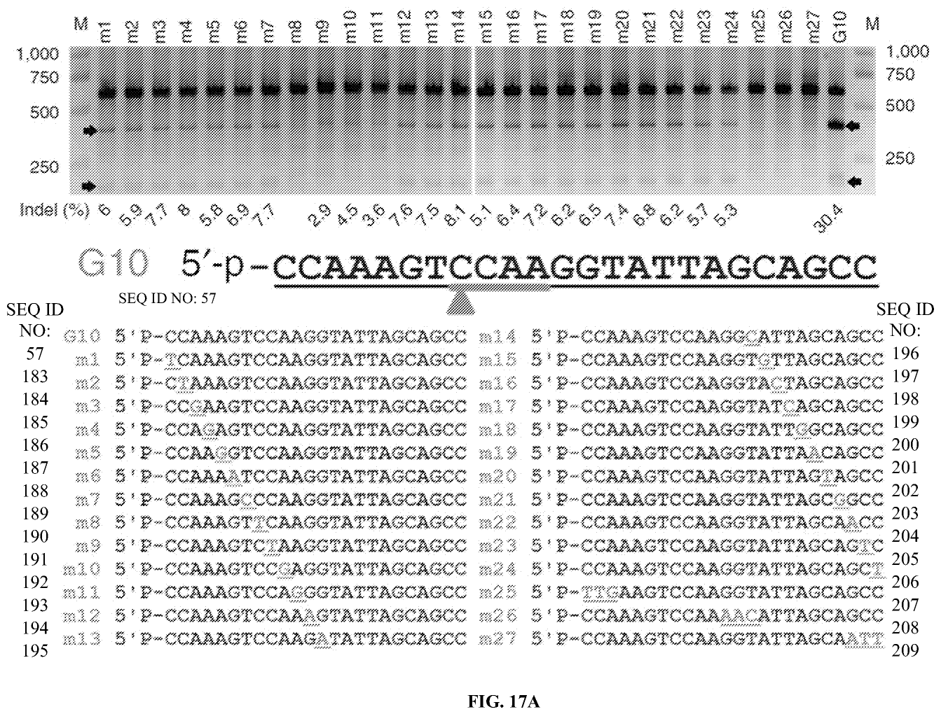

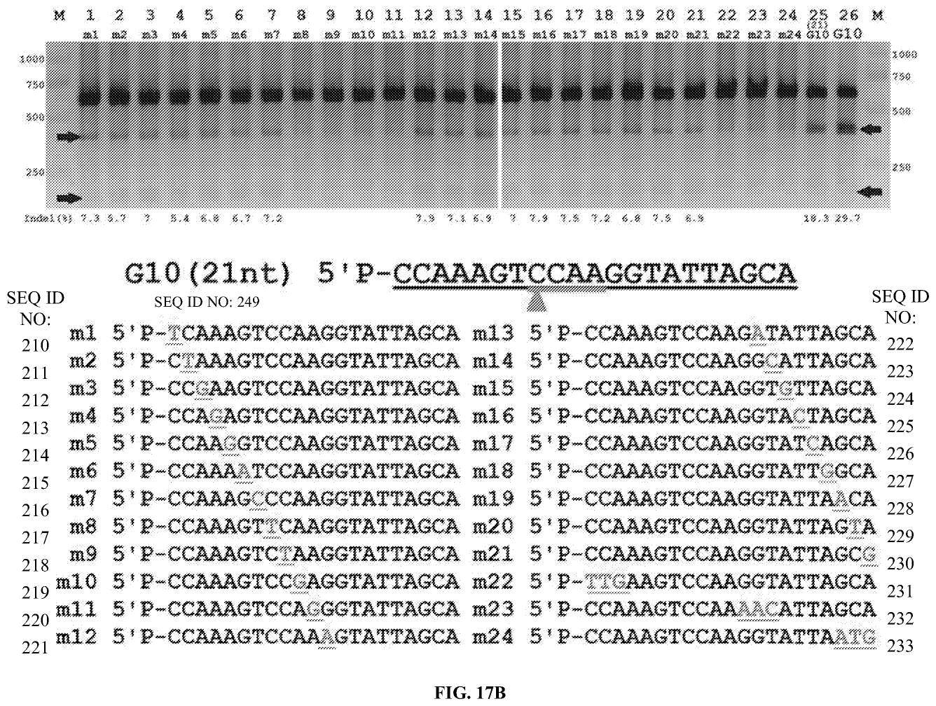

To investigate the effects of nucleotide mismatch between gDNA and target sequences on NgAgo activity, single-nucleotide and triple-nucleotide mismatches were introduced to 24-nt gDNA G10 targeting the DYRK1A locus. FIG. 17A shows the 24-nt G10 ssDNA guide sequence (top) aligned with 24 different guides, each designed to contain a single mismatched nucleotide (m1-m24, with mismatched nucleotide underlined), as well as 3 guide DNAs (m25-m27) each having a consecutive triple-nucleotide mismatch. T7EI assay results showed that NgAgo-mediated target cleavage was sensitive to a single-nucleotide mismatch at every position of the G10 guide. While NgAgo-G10 gDNA resulted in 30.4% indels, (G10 lane, far right), the cleavage efficiency was reduced to between ˜0 and 8.1% using mismatched guides. This reflects a reduction in cleavage efficiency of 73-100%, with the largest reduction observed using guides m8-m11. Moreover, mismatches at three consecutive nucleotides at any position tested completely abolished cleavage.

Additionally, various mismatched gDNAs (m1-m24) were designed based on a shortened 21-nt long ssDNA guide G10 targeting the same DYRK1A locus to test the effects of nucleotide mismatches on the efficiency of NgAgo-mediated target cleavage. Results of the T7EI assay are shown in FIG. 17B. Similar to the results with the 24 nt long gDNAs, the cleavage efficiency was sensitive to single nucleotide mismatches between the gDNA and the target locus, with greatest reduction of efficiency observed using gDNAs m8-m11. Little cleavage was observed using gDNAs having consecutive triple-nucleotide mismatches (m22-m24).

### Comparison of the Cleavage Efficiency Between NPAgo-gDNA and Cas9-sgRNA

NgAgo-gDNA has advantages over Cas9-sgRNA in targeting GC-rich loci, due to its reliance on DNA guides rather than RNA guides. RNAs (but not DNAs) are prone to form secondary structures, which may alter conformation and interrupt binding to target loci. To compare the efficiencies of NgAgo-gDNA and Cas9-sgRNA in cleaving GC-rich regions, these systems were tested on HBA2 and GA TA4 loci.

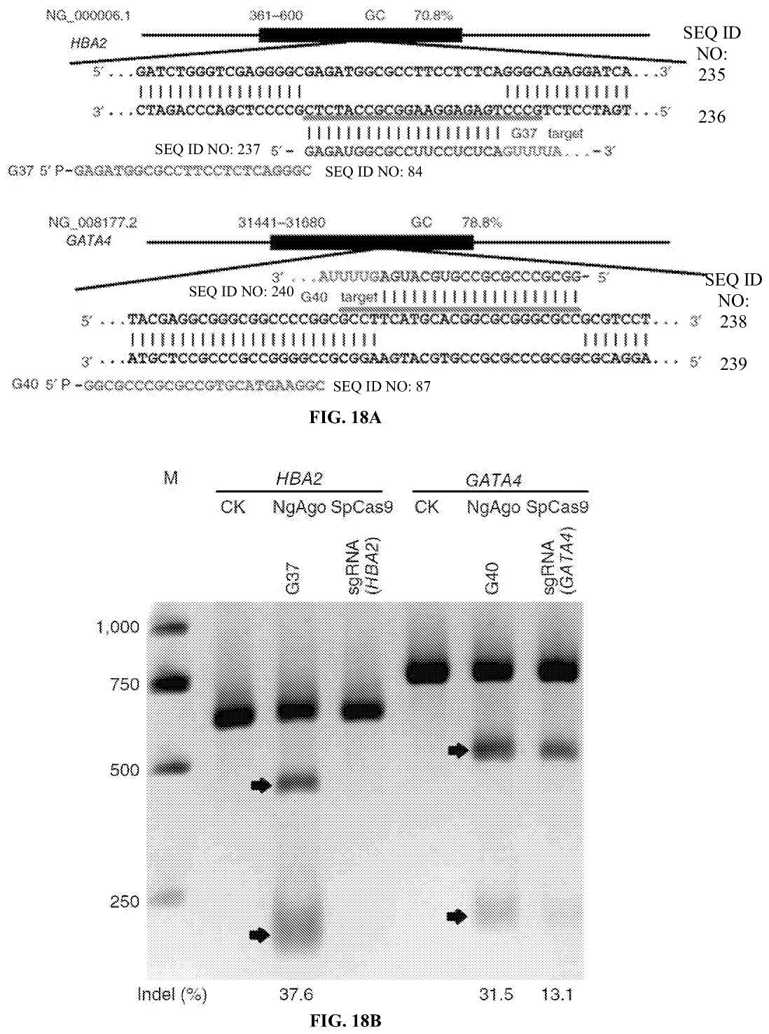

The HBA2 gene contains a locus spanning nucleotides 361-600 that is 70.8% GC-rich. FIG. 18A top panel shows the sequence of the HBA2 locus with the target site underlined, the corresponding sgRNA (HBA2) sequence aligned below, and the G37 gDNA sequence. The GA TA4 gene contains a locus spanning nucleotides 31441-31680 that is 78.8% GC. FIG. 18A bottom panel shows the sequence of the GA TA4 locus with the target site underlined, the corresponding sgRNA (GATA4) sequence aligned above, and the G40 gDNA sequence. The T7EI assay was performed to determine cleavage efficiency at each locus.

Results of the T7EI assay are shown in FIG. 18B. Uncleaved 293T DNA was used as a control (“CK” lanes) and cleavage products are indicated with arrows, with indel percentages indicated beneath the lanes. SpCas9-sgRNA resulted in no detectable cleavage of the HBA2 locus (lane 4 from left). NgAgo-gDNA cleaved the same GC-rich locus with an efficiency of 37.5% (lane 3 from left). Cas9-sgRNA was able to cleave the GATA4 locus with an efficiency of 13.1%, while NgAgo-gDNA cleaved the same locus with a higher efficiency of 31.5% (far left two lanes). Thus, the NgAgo-gDNA system was more effective at cleaving GC-rich loci than the Cas9-sgRNA system, which had limited cleavage activities at GC-rich loci. Additionally, whereas Cas9 requires target loci to be present immediately upstream of a PAM sequence, Argonautes have no target sequence restrictions based on our observation and others' reports (Swarts, D. C. et al. Nature 507, 258-261 (2014); Swarts, D. C. et al. Nucleic Acids Res. 43, 5120-5129 (2015)). Thus, NgAgo-gDNA can be applied to a broader range of genomic loci than the Cas9-sgRNA system.

### Example 6: Genome Editing in Mammalian Cells Mediated by NgAgo-gDNA

In eukaryotic cells, double-strand breaks (DSBs) are repaired primarily by two pathways: Non-Homologous End-Joining (NHEJ) and Homology directed recombination (HDR). As NgAgo-gDNA can induce double-strand breaks in genomic DNA of mammalian cells, NgAgo-gDNA can be used as a genome editing tool by taking advantage of the endogenous NHEJ or HDR repair pathways.

### HDR-Mediated Genome Editing

HDR is a widely-used strategy to knock-in, or capture, donor DNA to generate specific modifications to the genome. To test whether the NgAgo-gDNA system can be used to initiate HDR-mediated genome editing, a donor DNA fragment was designed to target exon 11 of the human DYRK1A gene.

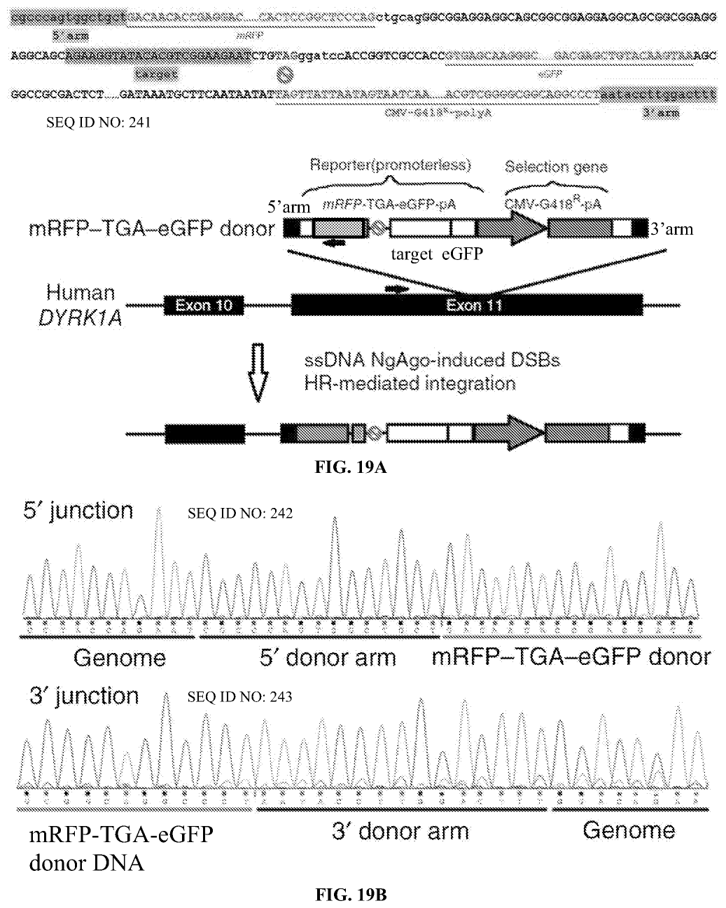

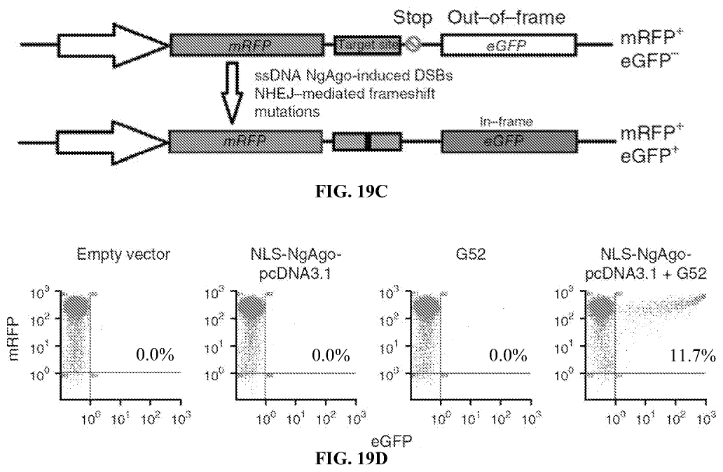

FIG. 19A shows a schematic of HDR-mediated insertion of donor DNA by NgAgo-gDNA. The mRFP-TGA-eGFP donor DNA was designed to contain a reporter region (mRFP-TGA-eGFP), which had two reading frames, an mRFP gene, and an out-of-frame eGFP gene separated by a TGA terminator. The reporter region was promoterless. The donor DNA also contained a G418 resistance gene under the control of the CMV promoter (CMV-G418^{R}-pA) to facilitate selection of edited cells. The reporter was flanked by a 5′ arm and a 3′ arm homologous to exon 11 of the DYRK1A gene. An annotated sequence of the donor DNA is shown at the top of FIG. 19A. Without being bound by any theory of hypothesis, NgAgo-gDNA can induce a DSB in DYRK1A, initiating homology-directed repair (HDR), which captures the donor DNA at the DSB via the homologous arms. This results in insertion of the donor sequence into exon 11 of the DYRK1A gene. Upon in-frame insertion of the donor DNA into the target locus, the reporter region is driven by the DYRK1A promoter, and thus mRFP and G418 are expressed. However, because of the TGA terminator, eGFP is not expressed.

For integration of mRFP-TGA-eGFP construct, 293T cells in each well of a 24-well plate were transfected using LIPOFECTAMINE® 2000 with 200 ng NLS-NgAgo-pcDNA3.1 plasmid, 100 ng G10 ssDNA guide and 500 ng mRFP-TGA-eGFP donor DNA. Three days after transfection, cell expressing mRFP could be observed by fluorescence microscopy, indicating successful insertion of the exogenous donor DNA by HDR. Genomic DNA was extracted from the cells using the Quick Extract DNA kit (Epicentre). The modified DYRK1A genomic loci were PCR-amplified with primers: 5′ junction amplicon: DYRK1A-test-F and Rm-test-R; 3′ Junction amplicon: PolyA-test-F and DYRK1A-test-R (Table 3). The PCR products were then cloned into T vector and sequenced.

The sequencing results confirmed accurate insertion of donor DNA into the DYRK1A locus following NgAgo-gDNA cleavage. An example sequencing chromatogram of the 5′ junction shows a sequence containing the 5′ donor arm and the start of the mRFP-TGA-eGFP donor (FIG. 19B top). An example sequencing chromatogram of the 3′ junction shows a sequence containing the end of the donor DNA and the 3′ donor arm (FIG. 19B, bottom).

Cells were subsequently treated with G418 for positive clone selection. After two weeks of G418 treatment, most cells expressed mRFP. Cell colonies with mRFP expression were isolated.

### NHEJ-Mediated Genome Editing

Positive clones having a stably integrated mRFP-TGA-eGFP reporter were next subjected to a second NgAgo-gDNA complex targeting the TGA terminator site to demonstrate NHEJ-mediated genome editing. Here, the integrated reporter region (FIG. 19C) contained a promoter driving expression of the mRFP gene, followed by the G52 target sequence, the TGA stop codon, and an out-of-frame eGFP gene so that only mRFP was expressed by the cell clones. The NgAgo-G52 gDNA complex could induce a double-strand break at the G52 target site upstream of the TGA stop codon. Without being bound by any theory or hypothesis, the DSB can be imprecisely repaired in the cell by NHEJ, and in some cases, a frameshift mutation can be introduced at the target site, thereby shifting the frame of the stop codon and shifting eGFP into frame. As a result, cells expressing both mRFP and eGFP can be generated.

The NLS-NgAgo-pcDNA3.1 plasmid and G52 ssDNA guide were co-transfected into the stable mRFP-TGA-eGFP integrated cells. Two days later, cells were harvested and flow cytometry analysis was performed to evaluate the expression of the mRFP-eGFP fusion protein. Cells transfected with empty vector, NLS-NgAgo-pcDNA3.1 alone, or G52 gDNA alone showed only mRFP expression (FIG. 19D). However, when mRFP-TGA-eGFP cells were transfected with both NLS-NgAgo-pcDNA3.1 plasmid and G52 gDNA, 11.7% of cells expressed both RFP and GFP (FIG. 19D, right), indicating successful out-of-frame mutation introduced to the G52 target site.

### Off-Target Genome Editing by NPAgo-gDNA and Cas9-sgRNA

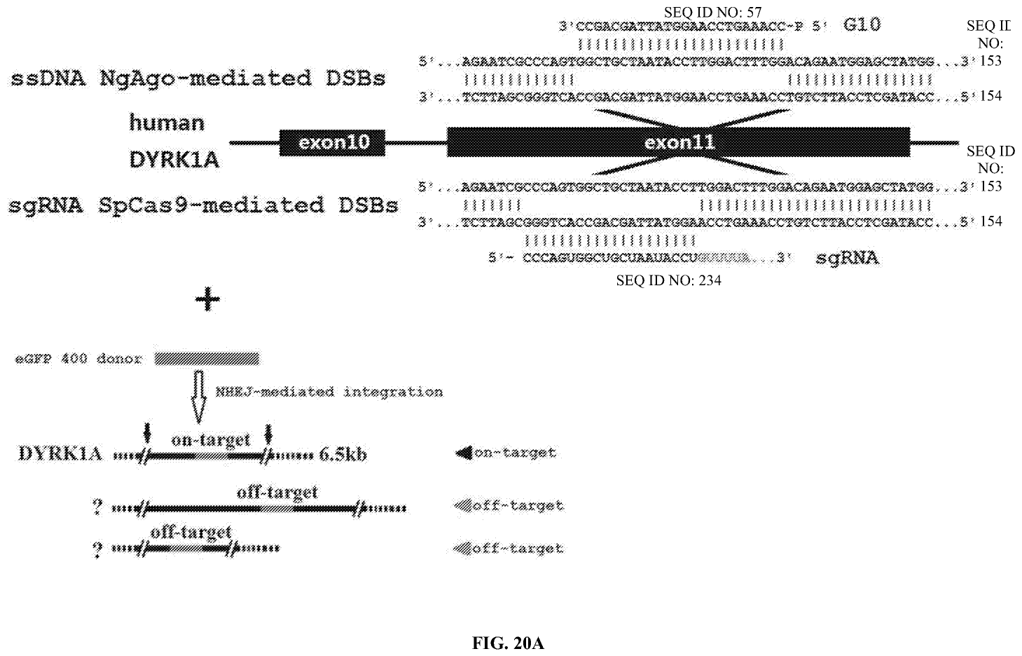

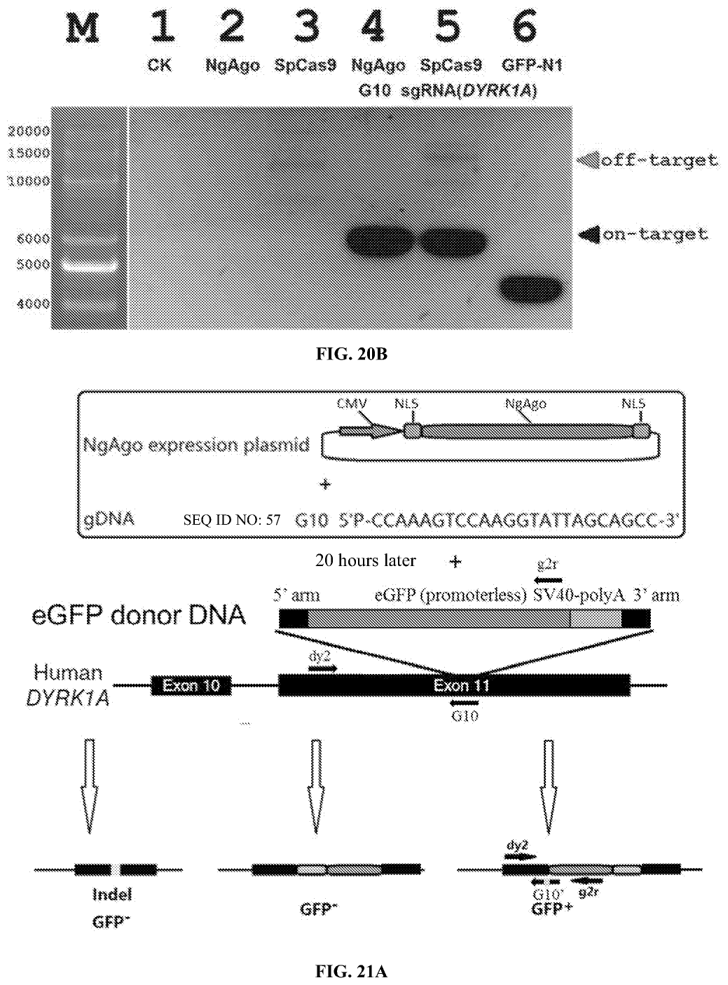

To investigate off-target effects by NgAgo-gDNA and Cas9-sgRNA, a 400 bp fragment of DNA was amplified from the eGFP gene to generate an eGFP400 donor DNA that lacked any homology to the DYRK1A target. The eGFP400 donor DNA was PCR-amplified from pEGFP-N1 plasmid using primers eGFP79 and eGFP483 (Table 3). The 293T cells were co-transfected with either the NgAgo-gDNA system (NLS-NgAgo-pcDNA3.1 plasmid and G10 guide) or the Cas9-sgRNA system (SpCas9-pCDNA3.1 and sgRNA (DYRK1A) vector) together with the eGFP400 donor DNA. FIG. 20A (top panel) shows the sequence of the locus in exon 11 of human DYRK1A gene aligned with the G10 gDNA and sgRNA. The NgAgo-gDNA and Cas9-sgRNA systems could induce DSBs at the target locus, and the eGFP400 donor DNA could be integrated into the genome, either within the DYRK1A target site (on-target), or elsewhere (off-target) (FIG. 20A, bottom).

Total genomic DNA was extracted from the transfected cells, digested using restriction enzymes, and analyzed by Southern blot analysis. Briefly, probes were generated by PCR using primers eGFP79 and eGFP483. The PCR products were then labeled with ³²P-dNTP using Klenow Fragment (New England BioLabs). 48 hours after transfection, engineered 293T cells were collected and total cellular DNA was isolated using a DNeasy kit (Qiagen) according to the manufacturer's instructions. Approximately 2.5 μg DNA was digested with DNA endonucleases BglII, SalI, Sac I, Xho I, Afl II and Eco47 III (New England BioLabs). The digested fragments were separated by 0.6% agarose gel. The DNA was then transferred to a nylon membrane and UV-cross-linked. After pre-hybridization with salmon sperm DNA (about 100 base pairs), The DNA was incubated with a ³²P-labeled probe in hybridization buffer (7% SDS, 0.5 M sodium phosphate buffer, pH 7.4) at 55° C. for 8 h. Membrane was then washed three times in 1×SSC with 0.1% SDS and exposed to X-ray film.

FIG. 20B shows the Southern blot. The ³²P-labeled probes did not hybridize to the control sample (lane 1), or to the DNA from cells transfected with only NgAgo-encoding plasmid (lane 2). Lane 6 shows probes hybridizing to the GFP-N1 positive control. The Bgl II reaction generated a 6.5 kb fragment containing the on-target fragment, which was detected in DNA from cells transfected with either NgAgo and G10 gDNA, or Cas9 and sgRNA (lanes 4 and 5). Higher molecular weight bands corresponding to off-target integration were detected in Cas9-expressing cells (lanes 3, 5), but not in NgAgo-expressing cells.

Knock-in of eGFP Donor DNA in Endogenous Loci

A second knock-in experiment was carried out by inserting an eGFP donor DNA into the target locus (SEQ ID NOs: 153, 154) of exon 11 of the human DYRK1A gene using NgAgo-G 10 gDNA, PhAgo-G10 gDNA, MiAgo-G10 gDNA, or MaAgo-G10 gDNA. Additionally, the eGFP donor DNA was knocked into a different target locus in the human beta-ACTIN gene using NgAgo-G21 gDNA, or PhAgo-G21 gDNA.

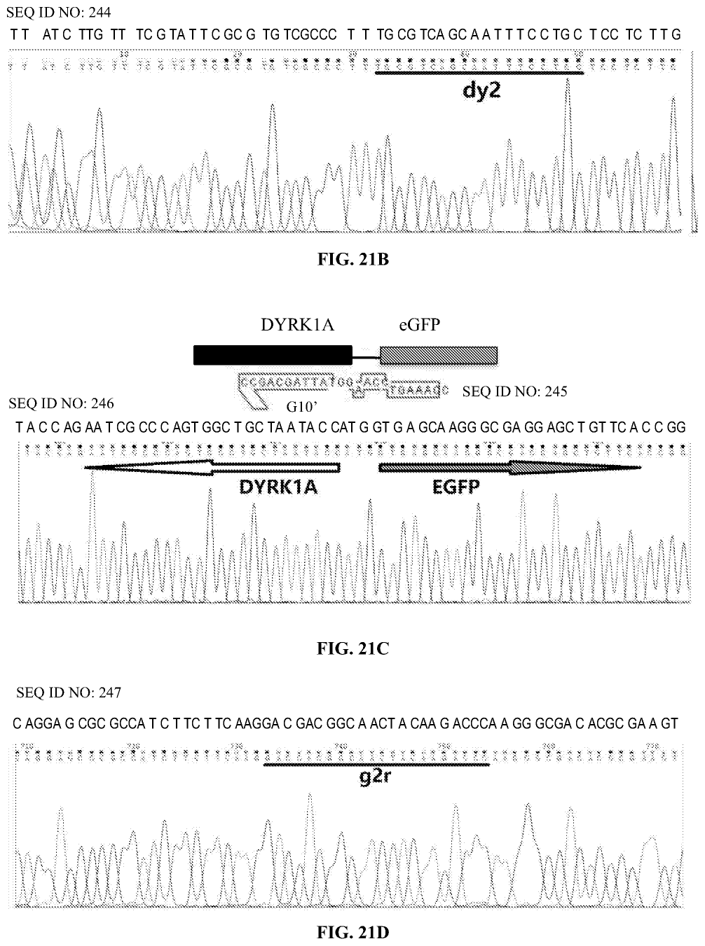

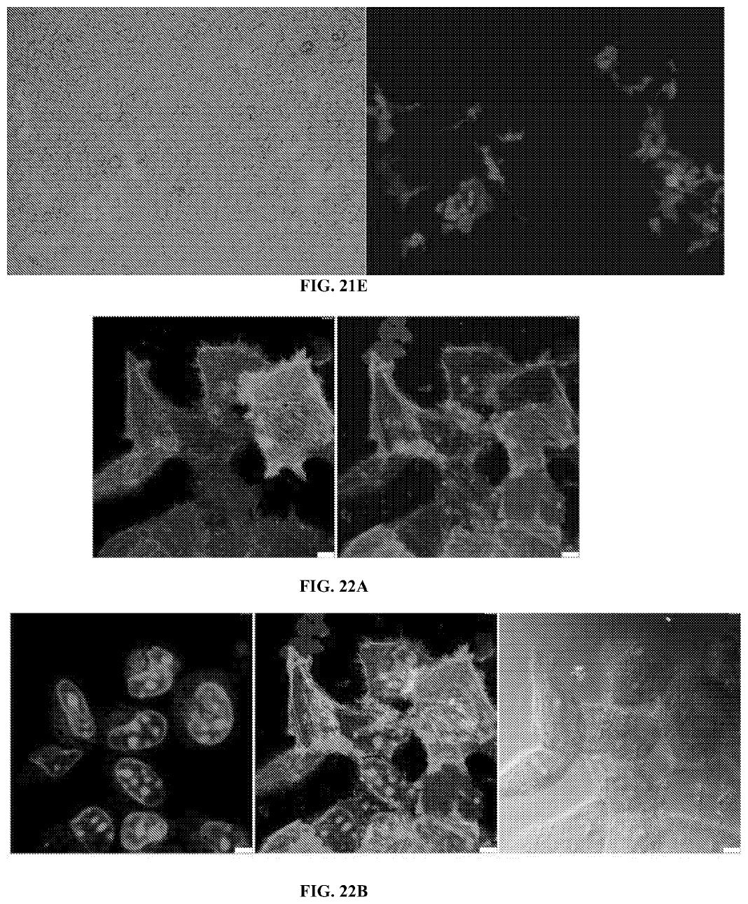

Using the PhAgo-G10 gDNA as an example, FIG. 21A shows a schematic of knock-in of the eGFP donor DNA at the DYRK1A locus mediate by NgAgo-gDNA. The plasmid encoding the NgAgo protein was codon-optimized for expression in human cells, and the N-terminus and the C-terminus of the NgAgo was each fused to a copy of NLS. The eGFP donor DNA was designed to contain a promoterless eGFP-coding gene, and an SV40 polyA signal, which was flanked by a 5′ arm and a 3′ arm homologous to exon 11 of the DYRK1A gene. The eGFP donor DNA was amplified using donor F and donor R primers and an eGFP template of SEQ ID NO: 155. Without being bound by any theory or hypothesis, NgAgo-gDNA induced DSBs in DYRK1A, initiating homology-directed repair (HDR), and inserting the donor DNA via its homologous regions. For knock-in to the beta-ACTIN locus, the eGFP donor DNA was amplified using 3-ACTIN test F primer (SEQ ID NO: 114) and -ACTIN test R primer (SEQ ID NO: 115) and an eGFP template of SEQ ID NO: 155.

For integration of the eGFP donor DNA, 293T cells in each well of a 24-well plate were transfected using Lipofectamine with 300 ng NLS-Ago plasmid, 300 ng guide DNA in a first transfection, and 300 ng eGFP donor DNA in a second transfection 20 hours after the first transfection. 48-60 hours after the second transfection, genomic DNA was extracted from the cells. The modified DYRK1A genomic loci were PCR-amplified with primers dy1 and g1r, and the modified beta-ACTIN genomic loci were PCR-amplified with primers ACTIN test F and g1r (Table 3). For each 50 μl PCR reaction, 1 μl of genomic DNA (200 ng/μl), 1.5 μl of forward primer (10 μmol/μl), 1.5 μl reverse primer (10 μmol/μl), and 25 μl of 2× Taq PCR starmix (GenStar), and 21 μl H2O were combined. The PCR product was further subjected to a second round of PCR amplification using primers dy2 and g2r for the modified DYRK1A locus, or ACTIN test F and g2r for the modified beta-ACTIN locus (Table 3). For each second PCR reaction, 0.1-1 μl of the PCR product from the first PCR reaction, 0.5 μl of forward primer (10 μmol/μl), 0.5 μl reverse primer (10 μmol/μl), and 25 μl of 2× Taq PCR starmix (GenStar), and 21-21.9 μl H2O were combined. The PCR program was as follows: 96° C. for 3 min; 30 cycles of 94° C. for 30 sec, 57° C. for 30 sec, and 72° C. for 20 sec; 72° C. 5 min. PCR products were purified with a PCR purification kit. The PCR products were then cloned into T vector and sequenced.

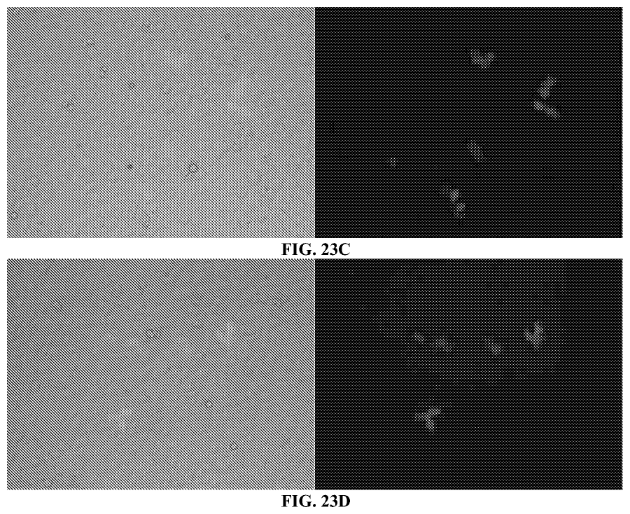

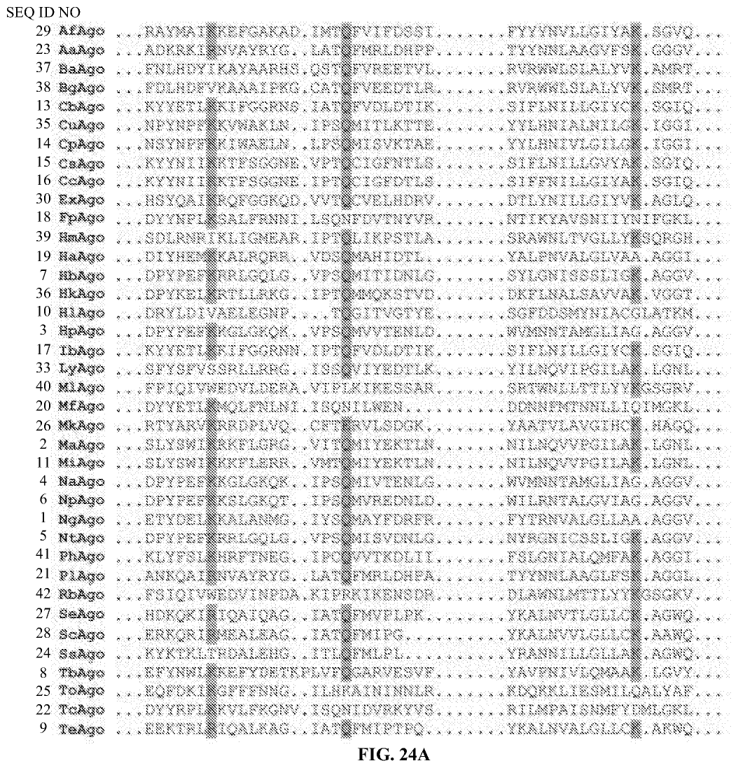

Sequencing analysis confirmed correct insertion of donor eGFP DNA into the DYRK1A locus following NgAgo-gDNA cleavage. SEQ ID NO: 156 was an exemplary sequence of a clone having the modified DYRK1A locus. Sections of the sequencing chromatogram of this clone are shown in FIGS. 21B-21D, including an upstream sequence close to the dy2 primer binding region (FIG. 21B), the 5′ junction between DYRK1A and the start of the eGFP sequence (FIG. 21C), and a downstream sequence surrounding the g2r primer binding region (FIG. 21D). At the DYRK1A-eGFP junction is a mutated G10 sequence due to cleavage and nucleotide removal by the NgAgo-gDNA (FIG. 21C, top). Additionally, fluorescence microscopy of the cells one month after the co-transfection (FIG. 21E) showed colonies of cells expressing eGFP. PhAgo-gDNA, MiAgo-gDNA, and MaAgo-gDNA were also effective in knocking the donor eGFP DNA into the DYRK1A locus as indicated by expression of eGFP in the cells (FIGS. 23A, 23C and 23D).

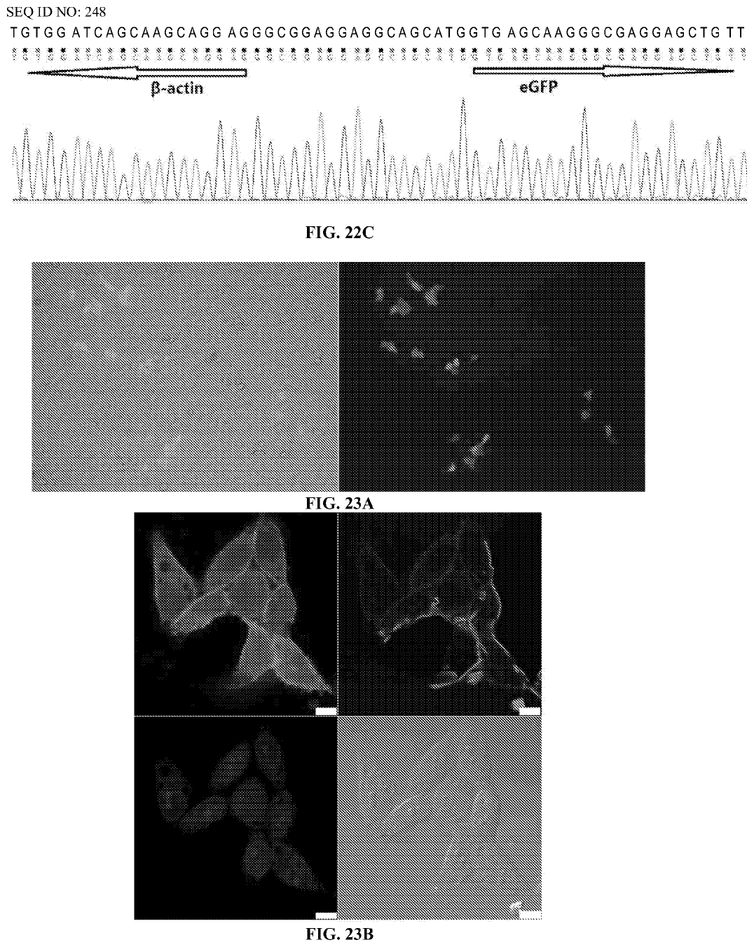

Furthermore, microscopy (FIGS. 22A-22B) and sequencing analysis (FIG. 22C) demonstrated that NgAgo-G21 gDNA was effective in knocking the eGFP donor DNA into the beta-ACTIN locus. PhAgo-gDNA was also effective in knocking the donor eGFP DNA into the beta-ACTIN locus as indicated by co-expression of eGFP and F-actin in the cells (FIG. 23B).

### Example 7. Experimental Methods and Nucleic Acid Sequences

Part of the experimental results in Examples 1-6 were reported in Gao F. et al. “DNA-guided genome editing using the Natronobacterium gregoryi Argonaute,” Nat. Biotechnol. 34: 768-773 (2016), the content of which is incorporated herein by reference in its entirety. The following exemplary protocols are related to NgAgo-gDNA in 293T cells. The protocols can be modified to assess other Ago-gDNA systems in any cell lines of interest.

### Ago-gDNA Mediated Target Modification

Cell Culture:

293T (ATCC CRL-3216), HeLa (ATCC CCL-2), MCF7 (ATCC HTB-22) cells were maintained in high-glucose Dulbecco's modified Eagle's medium (DMEM, HyClone), and K562 cells (ATCC CCL-243) were maintained in PRMI-1640 medium. Media were supplemented with 10% FBS (HyClone), 100 U/ml penicillin and 100 μg/ml streptomycin at 37° C. with 5% CO₂. Cells were seeded to 24-well plate (Costar) with 60% confluency 8 hours before transfection. Thirty minutes prior to transfection, cells were washed twice with phosphate buffered saline (PBS) and medium was changed to high-glucose DMEM medium containing 2% FBS. The medium was heat inactivated to remove contamination by microorganisms, such as mycoplasma, chlamydia, archaea, protozoa, and fungi.

Preparation of gDNA:

Single-stranded DNA oligonucleotides were obtained commercially. To prepare a 5′ phosphorylated guide DNAs, the ssDNA oligos were 5′ phosphorylated using T4 PNK (BioLab) as follows. A 120 μl reaction mixture was prepared by mixing 2 μl T4 PNK, 12 μl T4 ligase buffer (containing ATP), 100 μl ssDNA (about 33 μg) in water, and 6 μl water. If T4 ligase buffer without ATP was used, 2 μl 25 mM ATP was added to the reaction mixture, and 4 μl water was added instead to a final volume of 120 μl. The reaction mixture was incubated at 37° C. overnight. After 5′ phosphorylation, the resulting gDNA could be used directly without purification. The gDNA was diluted by water (pH 8) to 300 μl to achieve a final concentration of 10 nM or 100 ng/μl. Alternatively, 5′ phosphorylated single-stranded oligonucleotide guide DNA were purchased commercially.

Transfection:

NLS-NgAgo expressing plasmid was extracted using WIZARD® Plus SV Minipreps DNA Purification System (Promega), and was adjusted to a concentration of 100 ng/μl in 0.5×TE buffer (5 mM Tris-HCl, 0.5 mM EDTA, pH 8.0). 5′-phosphorylated ssDNA guides were dissolved to a concentration of 100 ng/μl in 0.5×TE buffer (pH 8.0). For each well of a 24-well plate, 200-250 ng NLS-NgAgo-pcDNA3.1 plasmid and 100-300 ng guide DNA were diluted in 50 μl Opti-MEM (Gibco), and 1.25 μl LIPOFECTAMINE® 2000 was diluted in 50 μl Opti-MEM. The DNA mix and lipofectamine mix were incubated for 5 min. The DNA mix and the lipofectamine mix were combined by gentle pipetting and incubated for 20 min. The DNA/lipofectamine mixture was then added into each well of cells.

For integration of donor DNA, two transfections were carried out. In the first transfection, in each well of a 24-well plate, 300 ng NLS-NgAgo plasmid and 300 ng gDNA were diluted in 50 μl Opti-MEM (Gibco), and 1.25 μl lipofectamine 2000 was diluted in 50 μl Opti-MEM. The DNA mix and the lipofectamine mix were for 5 min. The DNA mix and the lipofectamine mix were then combined with gentle pipetting, and incubated for 20 minutes. The DNA/lipofectamine mixture was then added into each well of cells. The second transfection was carried out 20 hours after the first transfection. Thirty minutes before the second transfection, cells were washed twice with PBS and medium was changed to high-glucose DMEM medium containing 2% FBS. For each well of a 24-well plate, 1 μg donor DNA was diluted in 50 μl Opti-MEM (Gibco), and 1.25 μl LIPOFECTAMINE® 2000 was diluted in 50 μl Opti-MEM. The DNA mix and the lipofectamine mix were incubated for 5 min. The DNA mix and the lipofectamine mix were then combined with gentle pipetting, and incubated for 20 minutes. The DNA/lipofectamine mixture was then added into each well of cells.

Since NgAgo follows “one-guide faithful” rule. (i.e. guide can only be loaded when NgAgo protein is in the process of expression), to improve the efficiency of gDNA loading to NgAgo, multiple transfections of gDNA can be conducted (e.g., 8, 12, or 24 hours after the primary transfection). Cells were incubated for 48-60 hours prior to harvesting. 90% confluency of the cells upon harvesting was ideal. Cell over-plating significantly weakens the efficacy of genome editing. For example, HDR-mediated editing was found to occur only during S and G2 phases. Lipofectamine 3000 should not be used here as P3000 interferes with ssDNA transfection.

### Genomic DNA Extraction and PCR Amplification of Modified Loci

Genomic DNA Extraction:

48-60 hours after transfection, with cells at an ideal confluency of 90%, cells were harvested by trypsin digestion. Four wells of cells were combined into a 1.5 ml Eppendorf tube. For genomic DNA extraction, 500 μl of cell lysate buffer (50 mM Tris, 100 mM EDTA, 0.5% SDS, pH 8) and 10 μl proteinase K (10 mg/ml) were added into each tube and mixed gently. Tubes were incubated in a water-bath at 55° C. for 2 hours. For phenol-chloroform extraction, 200 μl Tris-Phenol and 200 μl trichloromethane were added into each tube and mixed gently and sufficiently. After incubation for 5 min, samples were spun at 12,000 rpm for 15 minutes to separate aqueous phase from phenol phase. The aqueous phase was carefully collected into a clean Eppendorf tube. Phenol-chloroform extraction was repeated once and the aqueous phase was pooled. 500 μl trichloromethane was added into the collected aqueous phase, mixed gently and sufficiently. After a 5 minute incubation, the sample was spun at 12,000 rpm for 15 minutes to separate aqueous phase from the phenol phase. The aqueous phase was carefully removed into a clean Eppendorf tube. The entire phenol-chloroform extraction was repeated once. To precipitate the DNA, 900 μl ethanol was added to the collected aqueous phase and incubated at −20° C. for 30 minutes. The sample was centrifuged at 12,000 rpm for 10 minutes, and the DNA pellet was washed three times with 500 μl 75% ethanol. The DNA pellet was air dried, and 50 μl Tris-EDTA buffer was added. The genomic DNA concentration was adjusted to 100 ng/μl.

PCR Amplification:

For each 20 μl PCR reaction, 1 μl of genomic DNA, 0.5 μl of forward primer (10 μmol/μl), 0.5 μl reverse primer (10 μmol/μl), and 10 μl of 2× Taq PCR starmix (GenStar), and 8 μl H2O were combined. The PCR program was as follows: 96° C. for 3 min; 30 cycles of 94° C. for 30 sec, 57° C. for 30 sec, and 72° C. for 20 sec; 72° C. 5 min. PCR products were purified with a PCR purification kit.

### T7EI Assay

Double-strand breaks in the genome of mammalian cells are primarily repaired by the endogenous Non-homologous End Joining (NHEJ) pathway, which results in indels. The T7 Endonuclease I (T7EI) enzyme recognizes and cleaves non-perfectly matched DNA, such as indels. Thus, the T7EI assay described below can be used to determine rate of indels introduced by NEJH, which correlates with the rates of double-strand cleavage by the Ago-gDNA systems or the Cas9-sgRNA system.

T7EI Reaction:

Purified PCR products were denatured, reannealed, and digested with T7 endonuclease I. 150 ng of PCR products were combined with 1 μl 10×NEBuffer 2 (New England Biolabs), and ddH2O to a final volume of 9.6 μl. The mixture was then incubated at 95° C. for 3 min, followed by a temperature gradient of 95˜85° C.˜2° C./second, and then 85˜25° C.-0.1° C./second. The sample was then incubated at 4° C. for 1 hour. 9.6 μl of the annealing product was mixed with 0.4 μl T7EI (New England Biolabs), and incubated at 37° C. for 30 minutes. 10 μl of T7EI reaction product was mixed with 2 μl 6× loading buffer (Biolab), which was subjected to denatured PAGE electrophoresis at 140V for 30 minutes, and visualized by silver staining.

Band intensities on the PAGE gel were analyzed using Image Lab (Bio-Rad). Cleavage efficiency (i.e. percentage of indels) can be determined by the formula (1−(1−(b+c/a+b+c))1/2)×100, in which a is the band intensity of DNA substrate and b and c are the band intensities of cleavage products.

### Denatured PAGE and Silver Staining of Nucleic Acids

Denatured polyacrylamide gel electrophoresis (PAGE) gels (1 mm plate and comb) were prepared with 1 ml 30% (w/v) Acrylamide-Bis (19:1) solution with 4M urea, 1.5 ml of 5× tris-borate EDTA buffer (TBE), 85 μl of 10% Ammonium Persulfate (APS), 3.8 μl of Tetramethylethylenediamine (TEMED), and 4 ml of ddH2O. Electrophoresis was run at constant voltage of 140V for 30 min using the Mini-PROTEAN® system (Bio-Rad).

Silver staining was used to visualize nucleic acids following polyacrylamide gel electrophoresis. Following electrophoresis, the PAGE gel was directly placed into Fix Solution (10% acetic acid in MilliQ water), with gentle shaking for 30 min. The gel was then washed three times with ultra-pure MilliQ water for 4 min and then stained in ice-chilled staining solution (0.1 g silver nitrate and 150 μl formaldehyde in 100 ml MilliQ water) with shaking for 25 min. Following staining, the gel was washed three times (10 seconds each) with MilliQ water and placed in ice-chilled developing solution (6 g sodium carbonate, 300 μl formaldehyde and 13 μl 30% sodium thiosulfate in 200 ml MilliQ water) with gentle shaking until bands appeared clearly. The developing solution was then decanted and Fix solution was added to terminate the reaction.

### Nucleic Acid Sequences

Sequences of guide DNAs used in Examples 1-6 are listed in Table 2. PCR primers, including those used in the T7EI assays are listed in Table 3. Other oligos, plasmids and constructs are shown in Table 4.

TABLE 2
Guide DNAs.

| SEQ | | | |
|---|---|---|---|
| ID | | | |
| NO | Guide | Sequence | Gene |
| 45 | FWG | 5′P-TGCTTCAGCCGCTACCCCGACCAC | GFP |
| 46 | RVG | 5′P-GTGGTCGGGGTAGCGGCTGAAGCA | GFP |
| 47 | NCG | 5′P-CCGCCCCGAGTTCAAGGTGGAGCG | random |
| 48 | G1 | 5′P-CGGTAAACTGCCCACTTGGCAGTA | CMV |
| 49 | G2 | 5′P-CCAAGTAGGAAAGTCCCATAAGGT | CMV |
| 50 | G3 | 5′P-AAGGGCGAGGAGCTGTTCACCGGG | GFP |
| 51 | G4 | 5′P-GTGGTCGGGGTAGCGGCTGAAGCA | GFP |
| 52 | G5 | 5′P-CCTACCAGAATCGCCCAGTGGCTG | DYRK1A |
| 53 | G6 | 5′P-CAGCCACTGGGCGATTCTGGTAGG | DYRK1A |
| 54 | G7 | 5′P-ATCGCCCAGTGGCTGCTAATACCT | DYRK1A |
| 55 | G8 | 5′P-AGGTATTAGCAGCCACTGGGCGAT | DYRK1A |
| 56 | G9 | 5′P-GGCTGCTAATACCTTGGACTTTGG | DYRK1A |
| 57 | G10 | 5′P-CCAAAGTCCAAGGTATTAGCAGCC | DYRK1A |
| 58 | G1l | 5′P-ATACCTTGGACTTTGGACAGAATG | DYRK1A |
| 59 | G12 | 5′P-CATTCTGTCCAAAGTCCAAGGTAT | DYRK1A |
| 60 | G13 | 5′P-GCTCCATTCTGTCCAAAGTCCAAG | DYRK1A |
| 61 | G14 | 5′P-AGACGGTCAAATTAACGTCCATAG | DYRK1A |
| 62 | G15 | 5′P-CTGCTCCTCTTGGTTGGTCAGGCA | DYRK1A |
| 63 | G16 | 5′P-TGCCTGACCAACCAAGAGGAGCAG | DYRK1A |
| 64 | G17 | 5′P-CTCCTCTTGGTTGGTCAGGCACTG | DYRK1A |
| 65 | G18 | 5′P-CAGTGCCTGACCAACCAAGAGGAG | DYRK1A |
| 66 | G19 | 5′P-CGCTGTCCACCTTCCAGCAGATGT | ACTIN |
| 67 | G20 | 5′P-ACATCTGCTGGAAGGTGGACAGCG | ACTIN |
| 68 | G21 | 5′P-CAGCAAGCAGGAGTATGACGAGTC | ACTIN |
| 69 | G22 | 5′P-GACTCGTCATACTCCTGCTTGCTG | ACTIN |
| 70 | G23 | 5′P-GTCCGGCCCCTCCATCGTCCACCG | ACTIN |
| 71 | G24 | 5′P-CGGTGGACGATGGAGGGGCCGGAC | ACTIN |
| 72 | G25 | 5′P-CCCTCCATCGTCCACCGCAAATGC | ACTIN |
| 73 | G26 | 5′P-AGCATTTGCGGTGGACGATGGAGG | ACTIN |
| 74 | G27 | 5′P-CCCACGAGGGCAGAGTGCTGCTTG | EMX1 |
| 75 | G28 | 5′P-CAAGCAGCACTCTGCCCTCGTGGG | EMX1 |
| 76 | G29 | 5′P-GCCAATGGGGAGGACATCGATGTC | EMX1 |
| 77 | G30 | 5′P-GACATCGATGTCCTCCCCATTGGC | EMX1 |
| 78 | G31 | 5′P-TGTCACCTCCAATGACTAGGGTGG | EMX1 |
| 79 | G32 | 5′P-CCACCCTAGTCATTGGAGGTGACA | EMX1 |
| 80 | G33 | 5′P-GCAACCACAAACCCACGAGGGCAG | EMX1 |
| 81 | G34 | 5′P-CTGCCCTCGTGGGTTTGTGGTTGC | EMX1 |
| 82 | G35 | 5′P-TGCTGGCCAGGCCCCTGCGTGGGC | EMX1 |
| 83 | G36 | 5′P-GCCCACGCAGGGGCCTGGCCAGCA | EMX1 |
| 84 | G37 | 5′P-GAGATGGCGCCTTCCTCTCAGGGC | HBA2 |
| 85 | G38 | 5′P-GCGCCTTCCTCTCAGGGCAGAGGA | HBA2 |
| 86 | G39 | 5′P-CTCTTCTCTGCACAGCTCCTAAGC | HBA2 |
| 87 | G40 | 5′P-GGCGCCCGCGCCGTGCATGAAGGC | GATA4 |
| 88 | G41 | 5′P-AGCTCCGGTGGGGCCGCGTCTGGT | GATA4 |
| 89 | G42 | 5′P-GGTCCCTGGCGGCCGCCGCCGCCG | GATA4 |
| 90 | G43 | 5′P-GATAAGGTCCTTGAATTGCAGTAT | GRIN2B |
| 91 | G44 | 5′P-TTGCAGGGAGTCGACGAGTTGAAG | GRIN2B |
| 92 | G45 | 5′P-ATGAATGAGACCGACCCAAAGAGC | GRIN2B |
| 93 | G46 | 5′P-GCGGGGCCGGGCCTGGGCTGCGGG | HRES1 |
| 94 | G47 | 5′P-ACCGTAGGTTTCGGACATGGCCGT | HRES1 |
| 95 | G48 | 5′P-CTCCACCCTCCGTCCGGCCGCGAC | HRES1 |
| 96 | G49 | 5′P-CGCGTGCGGGCCGCCACTGTGGGC | APOE |
| 97 | G50 | 5′P-CATGGCCTGCACCTCGCCGCGGTA | APOE |
| 98 | G51 | 5′P-GCCTCAAGAGCTGGTTCGAGCCCC | APOE |
| 99 | G52 | 5′P-AGAAGGTATACACGTCGGAAGAAT | Knock-in |
| | | | construct |

TABLE 3
PCR Primers

| SEQ | | | |
|---|---|---|---|
| ID | | | |
| NO | Primer | Sequence | Length |
| 100 | DYRK1A | GTTCTTTCAGGTGCGTCA | 584BP |
| | test F | | |
| 101 | DYRK1A | GGGACTCTTCTCTATCAGCC | |
| | test R | | |
| 102 | HBA2 | ACGGCTCTGCCCAGGTTA | 577BP |
| | test F | | |
| 103 | HBA2 | CATTGTTGGCACATTCCG | |
| | test R | | |
| 104 | GATA4 | CCCCTTTGATTTTTGATCTTCG | 705BP |
| | test F | | |
| 105 | GATA4 | TGTGCAGGACCGGGCTGT | |
| | test R | | |
| 106 | GRIN2B | CAGGAGGGCCAGGAGATTTG | 696BP |
| | test F | | |
| 107 | GRIN2B | TGAAATCGAGGATCTGGGCG | |
| | test R | | |
| 108 | EMX1 | CCATCCCCTTCTGTGAATGT | 639BP |
| | test F | | |
| 109 | EMX1 | GGAGATTGGAGACACGGAGA | |
| | test R | | |
| 110 | APOE | GGAACTGGAGGAACAACTGAC | 680BP |
| | test F | | |
| 111 | APOE | TCGGCGTTCAGTGATTGT | |
| | test R | | |
| 112 | HRES1 | ATGCGCTGTGCACAGCGC | 676BP |
| | test F | | |
| 113 | HRES1 | TCAGGGAAATCGGGACTCAGC | |
| | test R | | |
| 114 | β-ACTIN | CACGAAACTACCTTCAACTCC | 700BP |
| | test F | | |
| 115 | β-ACTIN | GACTTCCTGTAACAACGCATC | |
| | test R | | |
| 116 | DYRK1A- | GGTCACTGTTGAAACTCATCC | |
| | test-F | | |
| 117 | Rm- | CTTGTAGATGAAGGTGCCG | |
| | test-R | | |
| 118 | PolyA- | CTAACTGAAACACGGAAGGAG | |
| | test-F | | |
| 119 | DYRK1A- | CTTGTAGCGGTTCAGTGTGT | |
| | test-R | | |
| 120 | eGFP79 | AAGTTCAGCGTGTCCG | |
| 121 | eGFP483 | GCCGTTTCTTCTGCTTG | |
| 122 | dy1 | GCCCTTGAATGTATTTGGGA | |
| 123 | dy2 | GTGCGTCAGCAATTTCCTGC | |
| 124 | g1r | ATGCGGTTCACCAGGGTGTC | |
| 125 | g2r | GGGTCTTGTAGTTGCCGTCGTC | |
| 126 | donor F | GGAATTCGTGAGCAAGGGCGAGGAG | |
| 127 | donor R | CCGCTTAAGGCCGATTTCGGCCTATTGG | |

TABLE 4
Other oligos, plasmids and constructs.

| | | SEQ | | | | |
|---|---|---|---|---|---|---|
| | | ID | | | | restriction |
| Plasmid | oligos | NO | sequence | template | Backbone | sites |
| NgAgo- | NgAgo- | 128 | GAAGATCTACAGTGATT | genomic | pGEX | BamhI, |
| 6P-1 | 6P-1-F | | GACCTCGATTCG | DNA of N. | 6P-1 | Bgl II, |
| | NgAgo- | 129 | CCGCTCGAGCTAGAGGA | gregoryi | | and |
| | 6P-1-R | | ATCCGACATTAGACTCG | SP2 | | XhoI |
| FLAG- | Flag- | 130 | CCCAAGCTTGCCACCAT | genomic | pcDN | HindIII |
| NgAgo- | HindIII-F | | GGATTACAAGGATGACG | DNA of N. | A3.1/ | and |
| HA- | | | ACGATAAGACAGTGATT | gregoryi | Hygro | BamHI |
| pcDNA | | | GACCTCGATTCG | SP2 | (+) | |
| 3.1 | HA | 131 | CGGGATCCTTAAGCGTA | | | |
| | BamHI-R | | ATCTGGAACATCGTATG | | | |
| | | | GGTAGAGGAATCCGACA | | | |
| | | | TTAGACTCG | | | |
| NLS- | NLS- | 132 | CCAAAAAAGAAGAGAAA | genomic | pDsRed- | HindIII |
| NgAgo- | NgAgo-F1 | | GGTAGCCACAGTGATTG | DNA of N. | Monomer- | and |
| red | | | ACCTCGATTCG | gregoryi | N1 | BamHI |
| | NgAgo- | 133 | CCCAAGCTTGCCACCAT | SP2 | | |
| | NLS- | | GGTGCCAAAAAAGAAGA | | | |
| | HindIII- | | GAAAGGTAGCC | | | |
| | F2 | | | | | |
| | NgAgo- | 134 | CGGGATCCCGGAGGAAT | | | |
| | Bam-R | | CCGACATTAGACTCG | | | |
| NLS- | NgAgo- | 135 | CCCAAGCTTGCCACCAT | NLS- | pcDN | HindIII |
| NgAgo- | NLS- | | GGTGCCAAAAAAGAAGA | NgAgo- | A3.1/ | and |
| pcDNA | HindIII- | | GAAAGGTAGCC | red | Hygro | BamHI |
| 3.1 | F2 | | | | (+) | |
| | NgAgo- | 136 | CGGGATCCtcaGAGGAA | | | |
| | Bam- | | TCCGACATTAGACTCG | | | |
| | TGA-R | | | | | |
| SpCas9- | SpCas9- | 137 | CCCAAGCTTGCCACCAT | pX330-U6- | pcDN | HindIII |
| pcDNA | Hind-F | | GGACTATAAGGACCACG | Chimeric_ | A3.1/ | and |
| 3.1 | | | ACG | BB-CBh- | Hygro | BamHI |
| | SpCas9- | 138 | CGGGATCCTCACTTTTT | hSpCas9 | (+) | |
| | Bam-R | | CTTTTTTGCCTGGC | | | |
| pACYC | pACY600- | 139 | CCCAAGCTTAACGACCC | pACYCDuet- | | HindIII |
| Duet- | Hind-F | | TGCCCTGAAC | GFP | | and |
| GFP | pACY2683- | 140 | CGCGGATCCGGGCATGA | | | BamHI |
| | Bam-R | | CTAACATGAGAATTAC | | | |
| | GFP- | 141 | CCCAAGCTTGGGGGACT | EGFP-N1 | | |
| | Hind-R | | TGTACAGCTCGTCC | without | | |
| | CMV- | 142 | CGCGGATCCTAGTTATT | MCS | | |
| | Bam-F | | AATAGTAATCAATTACG | | | |
| sgRNA | sgRNA-F | 143 | CACCGGCCCAGTGGCTG | | phU6- | BvbII |
| (DYRK1A) | | | CTAATACCT | | gRNA | |
| | sgRNA-R | 144 | AAACAGGTATTAGCAGC | | | |
| | | | CACTGGGCC | | | |
| sgRNA | sgRNA1-F | 145 | CACCGGCTGGGCATAAT | | phU6- | BvbII |
| (CMV) | | | GCCAGGCGG | | gRNA | |
| | sgRNA1-R | 146 | AAACCCGCCTGGCATTA | | | |
| | | | TGCCCAGCC | | | |
| sgRNA | sgRNA2-F | 147 | CACCGGCTCGTGACCAC | | phU6- | BvbII |
| (GFP) | | | CCTGACCTA | | gRNA | |
| | sgRNA2-R | 148 | AAACTAGGTCAGGGTGG | | | |
| | | | TCACGAGCC | | | |
| sgRNA | sgRNA3-F | 149 | CACCGGGAGATGGCGCC | | phU6- | BvbII |
| (HBA2) | | | TTCCTCTCA | | gRNA | |
| | sgRNA3-R | 150 | AAACTGAGAGGAAGGCG | | | |
| | | | CCATCTCCC | | | |
| sgRNA | sgRNA4-F | 151 | CACCGGGGCGCCCGCGC | | phU6- | BvbII |
| (GATA4) | | | CGTGCATGA | | gRNA | |
| | sgRNA4-R | 152 | AAACTCATGCACGGCGC | | | |
| | | | GGGCGCCCC | | | |

### Example 8. Sequence Homology Among Argonautes

The Argonaute from N. gregoryi SP2 (NgAgo, protein identifier AFZ73749.1), PhAgo, and MiAgo were identified by performing Position-Specific Iterative Basic Local Alignment Search Tool (PSI-BLAST) against the US National Center for Biotechnology Information (NCBI) nonredundant protein sequence database using the amino acid sequences of Thermus thermophilus Argonaute (“TtAgo”) and Pyrococcus furiosus Argonaute (“PfAgo”). Other Argonautes described herein were identified similarly by performing PSI-BLAST using the amino acid sequences of NgAgo, PhAgo, and MiAgo.

Argonautes in Table 1 were all capable of modifying DNA at 10-60° C., especially at 37° C. These Argonautes are capable of using a 5′-phosphorylated single-stranded guide DNA to cleave both strands of a target DNA, thereby inducing double-strand breaks. In contrast, Argonautes from most other species use an RNA guide to cleavage target RNA.

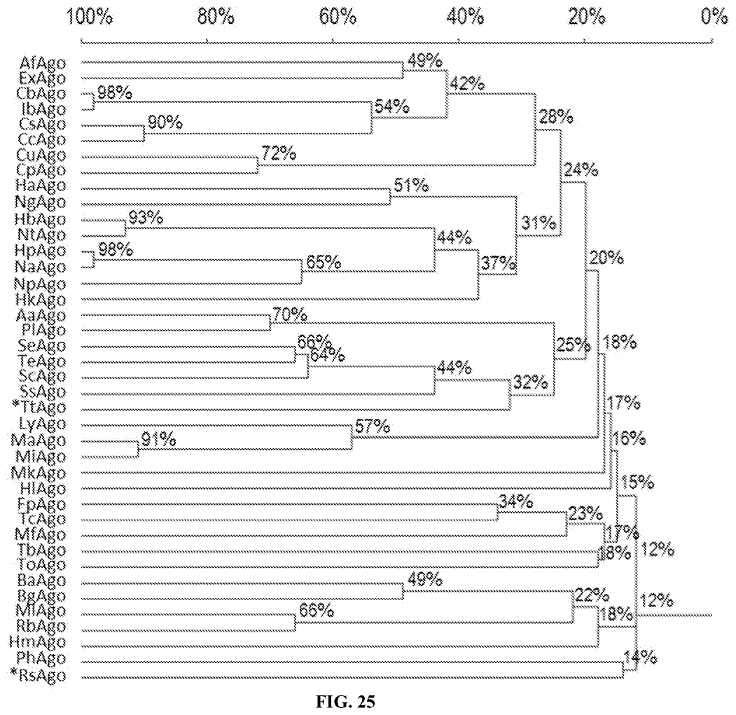

FIGS. 24A and 24B show sequence alignments of the Ago proteins surrounding the 5′ phosphate binding site and the nuclease active site. Most of the Ago proteins described herein comprise at least 2 of 3 conserved amino acids in the KQK motif of the 5′ phosphate binding site in the MID domain (highlighted in FIG. 24A); and at least 2 of 3 conserved amino acids in the DDE motif of the nuclease active site in the PIWI domain (highlighted in FIG. 24B). FIG. 25 shows a phylogenetic tree of the Ago proteins based on their sequence homology.

### Example 9. Degradation of Target DNA by NgAgo-gDNA Expressed in Bacteria

It has been reported that there are 5′ end phosphorylated short DNAs present in bacteria and archaea, although the mechanism of 5′ end phosphorylation is unclear. This example demonstrates the ability of NgAgo to bind invasive DNA and degrade target DNA genome in bacteria cells.

### In Vivo Genome Degradation by NgAgo-gDNA

A stable transgenic NgAgo-expressing E. coli strain (E. coli MG1655-NgAgo) was made by transforming E. coli MG1655 with a linearized PGEX-6P-1 plasmid (NgAgo-6P-1, FIG. 5), which contained an NgAgo expression cassette. The NgAgo cassette was under control of a lac operator, which could be induced by addition of IPTG in the culture.

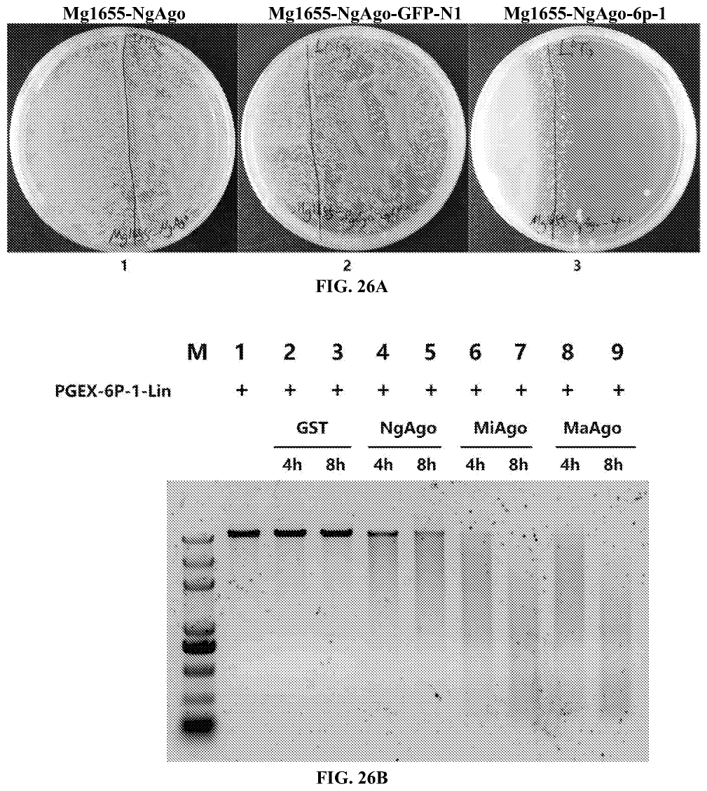

In panel 1 of FIG. 26A, E. coli MG1655-NgAgo bacteria were plated on an LB plate, which was supplemented with 0.1 mM IPTG on the right hand side of the plate. The transgenic bacteria were alive on both the left side and the right side of the plate. In panel 2 of FIG. 26A, E. coli MG1655-NgAgo bacteria were transformed with an unrelated GFP-N1 plasmid, which had no sequence homology to NgAgo-6P-1 plasmid, and plated on an LB plate having the right hand side supplemented with 0.1 mM IPTG. Again, the transgenic bacteria were alive on both the left side and the right side the plate. In panel 3 of FIG. 26A, E. coli MG1655-NgAgo bacteria were transformed with the PGEX-6P-1 plasmid, and plated on an LB plate. As the PGEX-6P-1 sequence was in the genome of the E. coli MG1655-NgAgo bacteria, the bacteria died after addition of 0.1 mM IPTG on the right side of plate. Without being bound by any theory or hypothesis, NgAgo expressed by the bacterial cells incorporated the gDNAs derived from the degraded PGEX-6P-1 plasmid, and the NgAgo-gDNA complex targeted and broke the genome of the bacterial cells.

### In Vitro Target DNA Degradation by Ago-gDNA

E. coli strain BL21 DE3 was transformed with a PGEX-6P-1 plasmid containing an NgAgo, MiAgo or MaAgo expression cassette fused with a GST tag. The expressed Ago proteins together with their bound guide DNAs were purified using GST, and incubated with a linearized empty vector of PGEX-6P-1, which was subsequently analyzed by agarose gel electrophoresis.

Briefly, using the NgAgo as an example, the NgAgo-6P-1 plasmid was transformed into E. coli strain BL21 DE3 to provide an NgAgo-6P-1 BL21 bacterial strain. 5 mL LB medium (supplemented with 1:1000 Ampicillin) was inoculated with the NgAgo-6P-1 BL21 bacteria at a 1:1000 ratio, and incubated overnight. The overnight bacteria culture was inoculated into a 50 mL culture medium at a 1:100 ratio, and incubated for 3 hours at 200 rpm and 37° C., until OD₆₀₀ reached 0.8-1.0. The bacteria culture and shaker were cooled to 21° C., and 0.1 mM IPTG and 20 mM MgCl2 were added to the culture to induce the bacteria with shaking at 100 rpm. The bacteria were harvested after 8 hours of induction, and bacteria pellet were stored at −20° C.

To purify the NgAgo with its associated gDNA from the bacteria pellet, the bacteria pellet was removed from the −20° C. freezer, thawed on ice, and resuspended in 20 mL of pre-chilled lysis buffer (20 mM Tris-HCl, pH 7.0, 20 mM MgCl₂, 100 mM NaCl). The mixture was then sonicated at 250 W with 3 seconds on and 8 seconds off for a total of 10 minutes. The sonicated mixture was subsequently sonicated at 4° C., 12000 rpm for 45 minutes. The supernatant was incubated with 20 μL GST Sepharose beads for 3 hours, and the supernatant was allowed to flow through. The GST beads were washed with 50 mL of wash buffer (20 mM Tris-HCl, pH 7.0, 20 mM MgCl₂, 100 mM NaCl) for 3 times. The NgAgo was then eluted with elution buffer (20 mM Tris-HCl, pH 7.0, 20 mM MgCl₂, 100 mM NaCl, 20 mM reduced glutathione). As prokaryotes have endogenous ssDNA production mechanisms, the purified NgAgo was pre-loaded with ssDNA.

In vitro enzyme cleavage reaction was set up as follows: 200 ng purified NgAgo was mixed with 100 ng linearized PGEX-6P-1, 1 μl 10× buffer (10 mM Tris-HCl, pH 7.0, 150 mM NaCl, 1 mM MgCl2, 0.4% glycerin, and 20 μg/ml BSA), and water to a total volume of 10 μl. The enzyme cleavage reaction mixtures were incubated at 37° C. for 4 hours or 8 hours, and then subjected to 1% agarose gel electrophoresis and silver staining.

FIG. 26B shows the electrophoresis results of the in vitro enzyme reaction mixtures. Since the Agos were bound to gDNAs derived from the PGEX-6P-1 plasmids in E. coli, the Ago-gDNA complexes could completely degrade the linearized PGEX-6P-1 empty vector in vitro.

1. A method of modifying a target nucleic acid, comprising contacting the target nucleic acid with an Argonaute (Ago) protein and a single-stranded guide DNA at a temperature of about 10° C. to about 60° C., wherein the Ago protein and the guide DNA form a complex that specifically recognizes a target locus in the target nucleic acid, and wherein the target locus comprises a sequence that is complementary to the sequence of the guide DNA.

2. The method of claim 1, wherein the Ago protein cleaves the target locus.

3. The method of claim 2, wherein the target locus is a double-stranded DNA, and wherein the Ago protein induces a double-strand break in the target locus.

4. The method of claim 1, wherein the temperature is about 37° C.

5. The method of claim 1, wherein the Ago protein and the guide DNA are present in a pre-formed complex.

6. (canceled)

7. The method of claim 1, wherein the sequence of the target locus comprises no more than about 3 mismatches to the sequence of the guide DNA.

8. The method of claim 1, wherein the target locus has a GC content of at least about 60%.

9. (canceled)

10. The method of claim 1, wherein the Ago protein is derived from Natronobacterium gregoryi.

11. The method of claim 1, wherein the Ago protein comprises an amino acid sequence having at least about 80% sequence homology to a sequence selected from the group consisting of SEQ ID NOs:1-42.

12. (canceled)

13. The method of claim 1, wherein the guide DNA is phosphorylated at the 5′ terminus.

14. The method of claim 1, wherein the target nucleic acid is an isolated DNA.

15. The method of claim 1, wherein the target nucleic acid is present in a cell.

16. The method of claim 15, wherein the method comprises transfecting the cell with the guide DNA and a nucleic acid encoding the Ago protein, wherein the guide DNA is transfected into the cell simultaneously with or prior to the nucleic acid encoding the Ago protein.

17-22. (canceled)

23. The method of claim 15, wherein the method comprises delivering a pre-formed complex comprising the Ago protein and the guide DNA into the cell, wherein the pre-formed complex is delivered into the cell via a vehicle selected from the group consisting of a cell-penetrating peptide, a virus-like particle, a nanocarrier, a liposome, a polymer, and a nanoparticle-stabilized nanocapsule.

24-33. (canceled)

34. The method of claim 1, wherein the modifying comprises site-specific cleavage of the target nucleic acid.

35. The method of claim 15, wherein the modifying comprises introducing a mutation at the target locus selected from an insertion, a deletion, and a frameshift mutation.

36. The method of claim 15, further comprising contacting the target nucleic acid with a donor DNA comprising a sequence homologous to the sequence of the target locus under a condition that allows integration of the donor DNA at the target locus.

37-41. (canceled)

42. The method of claim 15, wherein the modifying comprises one or more of: altering expression of the target nucleic acid, introducing a knockout mutation at the target locus, knocking in an exogenous sequence at the target locus, or introducing a substitution mutation at the target locus.

43-46. (canceled)

47. A composition comprising a complex comprising an Ago protein and a single-stranded guide DNA, wherein the complex is capable of specifically recognizing a target locus at a temperature of about 10° C. to about 60° C., and wherein the target locus comprises a sequence that is complementary to the sequence of the guide DNA.

48. A delivery system comprising a complex comprising an Ago protein and a single-stranded guide DNA, and a vehicle suitable for intracellular delivery of the complex, wherein the complex is capable of specifically recognizing a target locus at a temperature of about 10° C. to about 60° C., and wherein the target locus comprises a sequence that is complementary to the sequence of the guide DNA.

49-50. (canceled)


SEQUENCE LISTING (249 sequences)

SEQ ID NO: 1 - protein, 887 aa, Natronobacterium gregoryi SP2
MTVIDLDSTTTADELTSGHTYDISVTLTGVYDNTDEQHPRMSLAFEQDNGERRYITLWKNTTPKDVFTYDYATGSTYIFTNIDYEVKDGYENLTATYQTTVENATAQEVGTTDEDETFAGGEPLDHHLDDALNETPDDAETESDSGHVMTSFASRDQLPEWTLHTYTLTATDGAKTDTEYARRTLAYTVRQELYTDHDAAPVATDGLMLLTPEPLGETPLDLDCGVRVEADETRTLDYTTAKDRLLARELVEEGLKRSLWDDYLVRGIDEVLSKEPVLTCDEFDLHERYDLSVEVGHSGRAYLHINFRHRFVPKLTLADIDDDNIYPGLRVKTTYRPRRGHIVWGLRDECATDSLNTLGNQSVVAYHRNNQTPINTDLLDAIEAADRRVVETRRQGHGDDAVSFPQELLAVEPNTHQIKQFASDGFHQQARSKTRLSASRCSEKAQAFAERLDPVRLNGSTVEFSSEFFTGNNEQQLRLLYENGESVLTFRDGARGAHPDETFSKGIVNPPESFEVAVVLPEQQADTCKAQWDTMADLLNQAGAPPTRSETVQYDAFSSPESISLNVAGAIDPSEVDAAFVVLPPDQEGFADLASPTETYDELKKALANMGIYSQMAYFDRFRDAKIFYTRNVALGLLAAAGGVAFTTEHAMPGDADMFIGIDVSRSYPEDGASGQINIAATATAVYKDGTILGHSSTRPQLGEKLQSTDVRDIMKNAILGYQQVTGESPTHIVIHRDGFMNEDLDPATEFLNEQGVEYDIVEIRKQPQTRLLAVSDVQYDTPVKSIAAINQNEPRATVATFGAPEYLATRDGGGLPRPIQIERVAGETDIETLTRQVYLLSQSHIQVHNSTARLPITTAYADQASTHATKGYLVQTGAFESNVGFL

SEQ ID NO: 2 - protein, 747 aa, Microcystis aeruginosa NIES-843
MNYTAANTANSPIFLSEISSLTLKNSCLNCFQLNHQVTRKIGNRFSWQFSRKFPDVVVIFEDNCFWVLAKDEKSIPSLQQWKEALSDIQEVLREDIGDHYYSIHWLKDFQITALVTAQLAVRILKIFGKFSDPIVFPKDSQISENQVQVRREVNFWAEIINDTDPAICLTVDSSIVYSGDLEQFYENHPYRQDAVKLLVGLKVKDRETNGTAKIIRIAGRIGERREDLLTKATGSISRRKLEEAHLGQPVVAVQFGKNPQEYIYPLAALKPWVTDEDESLFQVNYGNLLKATKIFYAERQELLKLYKQEAQKALNNFGFQLREKSINSQEYPELFWTPSISIEQTPILFGQGERGEKREIIKGLSKGGVYKRHREYVDPARKIRLAILKPANLKVGDFREQLEKRLKLYKFETILPPENQINFSVEGLGFEKRARLEEAVDRLIGVEIPVDIALVFLPQEDRNADNTEEGSLYSWIKRKFLGRGVITQMIYEKTLNDKSNYKNILNQVVPGILAKLGNLPYVLAEPLEIADYFIGLDVGRMPKKNLPGSLNVCASVRLYGKQGEFVRCRVEDSLTEGEEIPQRILENCLPQAELKNQTVLIYRDGKFQGKEVENLLARARAINAKFILVECYKTGIPRLYNLQQKQINAPSKGLALALSNREVILITSQVSEQIGVPRPLRLKVHELGEQRNLKQLVDTTLKLTLLHYGSLKDPRLPIPLYGADIIAYRRLQGIYPSLLEDDCQFWL

SEQ ID NO: 3 - protein, 863 aa, Halogeometricum pallidum JCM 14848
MVKRYISFHLFPRIKLCGVYLCLRMNTKDDIAHKQPITIEVQVLKELDKPSPKMATRLLVADRAGNRFPLAIWKNNALSDYDWTIGQWYRLENARGNVFNGKQSLNGSSNMRATPLEASEEDETRADDVGRVDTILGNLSPNQAYLSLFPISRSFDTLSVYEYSIEAAEAFEDDPDTVTYQCAGRLRRITGAGVAYAGPMQIVSTRKLPDKLADPFSLSEPTERELKAADARDRHRIERLLKSLVKAAIDDSTYDPYQINRIRARTPAITAGDGLFEACYEFAARVDVMPSGDAFVGIEVRYHARSQVTADVYEDKTGELVGTIVEHDPERYNVSGTGRVVGFTDHYFTDALDELGGLSLADWYAQKDRVPEGVLEALREKNPRLVDIQYQEDEPAQIHVPELLRVAPRKEVVKELDPTFHRRWDREAKMLPDKRFRHAIEFVDHLGSLPDIDATVAPEPLGPSLSYMSTAVDREENLRFKDGRTATTPSSGIRSGVYQQPTSFDIAYVYPTESEQESKQFISNFENKLSRCHCEPTATRHVPYELGGELSYLAVINELESVDAVLAVVPPRNDDRIAAGDITDPYPEFKKGLGKQKVPSQMVVTENLDTRWVMNNTAMGLIAGAGGVPWRVDEMPGEADCFIGLDVTRDPETGQHLGASANVVYADGTVFASKTQTLQSGETFDEQSIIDVIKDVFQEFVRREGRSPEHIVIHRDGRLFEDADEIQAPFADSGVSIDILDIRKSGAPRIARYEDNSFKIDEKGRLFISQDDTHGFIATTGKPEFDDSDNLGTPKTLRVVRRAGDTPMLTLLKQVYWLSEAHIGSVSRSVRLPITTYYADRCAEHAREGYLLHGELIEGVPYL

SEQ ID NO: 4 - protein, 839 aa, Natrialba asiatica
MKTQDDIAHKQPITIEVQILKELDKPSPKMATRFLVADRDGNRFSLAIWKNNALSDYDWTIGQWYRLENARGNVFNGKQSLNGSSKMRATPLEASEEDETSTDDVGRVDTILGNMSPDQAYLSLFPISRSFDTLSVYEYSIEAAEAFEDAPDTVTYRCAGRLRRITGAGVAYAGSMRIVSTRKLPDKLADPFSLSEPTERELNATDARDRHRIERLLKSLVKAAIDDSTYDPYQINRIRARTPSITAGDGLFEACYEFAARVDVMPSGDAFVGIEVRYHTRSQVTADVYEDKTAELVGTIVEHDPERYNISGTGRVVGFTDHHFTDALDELGGLSLADWYAQKDRVPEGVLEALREKNPRLVDIQYQEDEPARIHVPDLLRVAPRKEVVKELDPAFHRRWDREAKMLPDKRFRHAIEFVDHLGSLPDIDATVAPEPLGPSLSYMSTAVDREKNLRFKDGRTATTPSSGIRSGVYQQPTSFDIAYVYPTESEQESKQFISNFENKLSQCQCEPTAARHVPYELGGELSYLAVINELESVDAVLAVVPPRDDDRITAGDITDPYPEFKKGLGKQKIPSQMIVTENLGTRWVMNNTAMGLIAGAGGVPWRVDEMPGEADCFIGLDVTRDPETGQHLGASANVVYADGTVFASKTQTLQSGETFDEQSIIDVIKDVFQEFVRREGRSPEHIVIHRDGRLFEDADEIQAPFADSGVSIDILDIRKSGAPRIAQYEDNSFKIDEKGRLFISQDDTHGFIATTGKPEFDDSDNLGTPKTLRVVRRAGDTPMLTLLKQVYWLSEAHVGSVSRSVRLPITTYYADRCAEHAREGYLLHGELIEGVPYL

SEQ ID NO: 5 - protein, 889 aa, Natronorubrum tibetense
MAVKTDIEDGKQIDISLRVTGTDEWDHDAIARKVQLEDVEGTPVELTVFHNNEIADFEWDDERWYVLENVVGNEYRGEMQLNPGYDLIVTPLDEPPAAAENGGAENTSATQSSESGDSGSSTEADQSAESESARESEVTSEPRPTADGGGELLHQQPLSEGNYLLQFELGDLPELTVHEYELRATGSGGINPDDFTNGIEGFTAKAANYYQSRINSPVTTADASRRRIYATEKLHGKISIHGYTVKPVHQGETTLEARSYTDDGPLQEFVKQDVKRAVAGRFEVSGIDSIIEPTPQRTANSGLFEAYRKYKCRIRVDADGTVICGVNVAYHLESTFSAADWVQRGHDIAEVTVEHDTDLYDSARTATVKEVIDMDYDDVLDGPGVPMSEYHEQHVEQDVINSMQAGDPIIADLQYGSDEDSIFPQLLEYCKVIPTFDQLGSVDDTFLDVIHNESRMEPEERFSVVTSFVDLLGPTPYFSFDPVPQPTNAGYREHKTPNTPNLRFGDGKTGFYGAGGLERKGYGIYKAPESFDIIALYPEDEEDDARPYVLSLLNKLADYDAGPTVFDQETYELGSEFHYSQHAQKASDYDAALIVVPDADKAAAADYDDPYPEFKRRLGQLGVPSQMISVDNLGNDNYRGNICSSLIGKAGGVPWRIDDVPGDVDAFVGLDVTYDHATKQHLGAAANVIMADGTILASEAVTKQAGETFDEDDVANVIKHVLEIFAEEEGRPPRHVVIHRDGKFYLDVENLVKRLDKARDLIQRFDLVEIRKSGNPRIAAYDESESRFDIADKGIAFHVHNGDHSYLTTTGGREGSPGTPRPLQIVKRHGSTDLDTLAEQTYWLSEAHVGSLSRSTRLPITTYYADKCADFAMKGYLTKGSVIRGVPYI

SEQ ID NO: 6 - protein, 847 aa, Natrinema pellirubrum
MPTQSDIEDGERIDIQVKVLSELDRPSEKMAKRLRVRDTDGNEFPLTIWKNNALCDFAWERGRWYELENARGNEFRGEKSLNGSSRLHADPVDNPIDSDRSQQSTTAESTDKQFDSLEDGLPYLSLFPIDREFETVDVYEYRIEADGPFDDDPMDATYTLAAYLRSCSDAAVTHAGIFSVIATNRLTNALPDPFELTDESRVTLRADDETDNECLVRLLQQVFKTAVDDETYETGRVDRIRTQDPVITGQDGLFEACLAYTARLEILPSGKAFVGIDISYHARSQVTVDKYVDRINASVDELIDTPVEHDPERYEKSGSGRLKGFADVTFTDPVDDFGNQSLADWYEQKGRISDDMLERLRSEDPQLVEIQYNPNSDETNLHVPQLLRVAPRKEIVKKLAPTFHRKWDRAAKMLPDDRFRKATRFVARLDSLSEVDAQIEPNPVGPNISFMSTEVDRSDNLRFGDDQTTTLPNNGLKRYGIYRRPSSLHLHYLVPERYTDEFASFREQLERQLATIGCSPDDISYDEYGLGNAINYNTTAAAVDDVDIVLAVVPAPDNDFIRNGTIDDPYPEFKKSLGKQTIPSQMVREDNLDDRWILRNTALGVIAGAGGVPWRVDEMPGDVDCFVGLDATRDPETGQFLGASANVVLSDGTVFVSKTQSLQSGETFDENAIVDVLKDVHREFVREEGKSPNNIVIHRDGRLFEDVDTILEPFDETDIDIDILDVRKSGAPRAAVYQDDQFQVDHKGRLFVAQSGDYGFLTTTGRPEFDEDDGLGTPRSLRIVRRAGETPMRTLLEQVYWLSESHVGSAQRSTRLPITTYYADRCAEHAREGYLVNGELIRGVPYL

SEQ ID NO: 7 - protein, 895 aa, Halogeometricum borinquense
MAVKADIEDGEEIDIALHVTGIDEWEHDAIARKIQLEDVDGAAIDLTVFHNNDVSDFEWEIGEWYLLENVVGNEFRGEMQLNPGYDLTVTLLNDPPAAAGNDKSPGSVPPEEPVDQSGESGSSGAAASTSGEPGDAEFVRGSEVDDGSRPTADGGGKLLHQQPLSDGNYLLQFELGSLPELPVHEYELRATGSGGIDPDDFTNGIEGFTAKAANYYQSRIGSPVTTADASRRRIYATEKLHGTISMHGYTVKPVHQGETTLEARSYTNDGPLQEFVKQDVKRAVAGRFEVSGIDSIIEPTPQRTANSGLFEAYRKYKCRIRVDADGTVICGVNVAYHLESTFSAAEWVQRGHDIADVTVEHDTDLYDSARTATVKEIIDMDYDDMLDGPGVPMSEYHEKHVEQDVIDSMRAGNPVIADLQYGSGEDSIFPQLLEYCKVIPTFDQLGRVDETFLNVIHNESRMKPEERFNVVTSFVDLLGPTPYFDFGPVPQPTNAGYREQKTPNIPNLRFGDGRTGYYGAGGLERKGYGVYKAPESFDIIALYPDSEQAAARPYVLSVLGKLAEYDGKPTKFDQETYELGSEFHYSQHAQKTSDYDAALIVVPDKDKAAAADYDDPYPEFKRRLGQLGVPSQMITIDNLGNDSYLGNISSSLIGKAGGVPWRIDDVPGDVDAFVGLDVTYDHATKQHLGAAANVIMADGTILASEAVTKQAGETFDEDDVANVIKHVLEIFAEEEGRPPRHVVIHRDGKFYLDIESLIKRLDKARDLIQRFDLVEIRKSGNPRIAEYDESKSRFDIADKGVAFHVHNGDHSYLTTTGGKEGSPGTPRPLQIVKRHGSTDLDTLAEQTYWLSEAHVGSLSRSTRLPITTYYADKCADFAMKGYLTKGSVIRGVPYI

SEQ ID NO: 8 - protein, 834 aa, Thermococcus barophilus
MSPKSKQATILEFLSVKGKDKQQKTIKSQPLMKSSEPEEESFPLVEVDSEWNIIRINPEIAKSLFAKTVKTYYGLGKNYVAENLLHDEEKVEFLRRVYELFPYNYLAKSENGIPYYEYYSTAENIDADIVDHLQLIKIGEYHWEELDPHIAVRLITAHIRSSFGEFLKNKLSSRELKDVWIRLPKGRERVSLEFVKKFELGTLGYSIEDGEIWMALRRRSSASAVLLADFPPKVLEYVMIKLKKLAEEAVKQDYRKVRIYSETQPGSGTAAVIDVIIGHDNVIRFLEEHPKGAFHVKKRLEELGKSLDDVNYLLIGVYDNSISLKRALEDENCQYYFTEHNAYLVLTTEVQKELFGRIVDDWRGEIVEEYREKLNEITTSGKLFSQVSGIKGITVNIRENLLVRTLKGEITVKDVKDIMRDESIFLNLLPISEKILNETKRHNLRLLIFYPKKFDERIRKSIFLDQIERLRNGLLSNSFSSVDEVDLIPVSRDRSLDYHKTMLEAGLDKVRDYTLATIIFPEQYNKRDLELREFYNWLKKEFYDETKPLVFQGARVESVFGMYKRYAVPNIVLQMAAKLGVYPYSLETSSGYDYIIGIDYTYWHERDAVSVGGGAVVVSPSGLIEGIYPIAIPSKRESLDMKEILQEWFLKTVQNNPEIMKQGRVTVLISRDGMVPKYERNRIQEFLCEYSDELDMTIEAVEVRKRILTRIWSLDGPNTFYSPVKVGNYTYYMVNAHAGYPLRRKERKIYYSKPYLIGSLYRFQSGKVQPIHGSASRHLVESLIRLQKINYTTTKMDNVRLPLPIDVTHKLINFIRDVNMRVKRVGIPNSLFMA

SEQ ID NO: 9 - protein, 756 aa, Thermosynechococcus elongates
MPRETCYDKRTTPSQYGWLPIDSLSVMPTQFQEVEVILNRFFVKKLSRPDLTFHEYQCQFTQVPEQGSEQKAISSVCYKLGVTAVRLGSCIITREPIDPERMRTKDWQLQLIGCRELSCQNYRERQALETFERKILEEKLKETFKKTIIEKDYELGLIWWISGEEGLEKTGHGWEVHRGRQIDLKIETDEKLYLEIDIHHRFYTPFKLEWWLSEYPNIQIKYVRNTYKDKKKWILENFADKSPNEIQIEALGISLAEYHRQEGATQQEIDESRVVIVKKISDYKAKPVYHLSQRLSPILTMETLAQIAEQGREKKEIQGVFDYIRKNIGTRLQESQKIAQVIFKNVYNLSSQPEIMKVNGFVMPRAKLLARNNKEVNQTARIKSFGCAKIGETKFGCLNLFDNKPEYPEEVHKCLLAIARSSGVQIKIDSYFTGSDYPKDDLAQQRFWQQWAAQGIKTVLVVMPWSPHEEKTRLRIQALKAGIATQFMIPTPQDNPYKALNVALGLLCKAKWQPVYLKPLDDPQAADLIIGFDTSTNRRLYYGTSAFAILANGQSLGWELPDIQRGETFSGQSIWQVVSKLVLKFQDNYDSYPKKILLMRDGLVQDGEFEQTIRELTHQGIDVDILSVRKSGSGRMGRELTSGNTAITYDDAEVGTVIFYSATDSFILQTTEVIKTKTGPLGSARPLRVVRHYGNTPLELLALQTYHLTQLHPASGFRSCRLPWVLHLADRSSKEFQRIGQISLLQNVDREKLIAV

SEQ ID NO: 10 - protein, 718 aa, Halorubrum lacusprofundi
MQAEIDDAFADVPESATEQTGHALTTFRSEKSLSEFAVHEYTLKAKDGYRPDDHQAALRDTYKARRDILGEFESSSPPVIAVRDALALATPSPLPVEDLELKNFEMVNDGPHTLDYTDYSDKAVTKGLIDASLRRILDGDYEVRGIDTVLSKRPVIQKQDFRLHERWNLSLSVTNAGVVYLSVDFRHKTISEYTLDKFDLDKLYRGLRVNVTYKQSGKGAFVDELMDKTVNESIDDMGNQSVVEYHEGAERIPDRVLKEIARADRRVVKVTKQGSHNTEYYPQRLLALQGHPQNVKAFAPRFNEATRGKTRLSAQRCLNRATTFVENLPPVIPLGGATLSFDATPVNGDDTWEMSRLFETDAHILQFAEEQTGDHPRRVKHNEVYEAPEEFSVCLVHPSAGPHAERWENLDQQLKDIGAGPEAVNRVQYDPFKTADEIYTDLLVDVPDDHEYSAACVILPKADFSMGESSASDIYHEMKKALRQRRVDSQMAHIDTLATSYALPNVALGLVAAAGGIPFTTEDAMPGETDLFIGIDVSHRYPRDTDERVHIAASTTSIYGDGTILGYTSAKPQTGEKVPPKELKNLTRQSIAGYKQEHGEYPDRIVIHRDGFMREDLDQVEEMLESMDINYDVVEIRKQSPARVLNLADGVAKIPDKGIAALNREENRAILATFGDPESQATSSNTGLPQPIQVERKAGDTDIKTLVVSQSPLEFSSN

SEQ ID NO: 11 - protein, 747 aa, Microcystis aeruginosa
MNYTETKTANSPIFLSEISSLTLNNNCLNCFKLNHQVTRKIGNRFSWQFSRKFPAVVVIFEDNCFWVLAKDEKLLPSPQQWKEALSDIQEVLREDIGDHYYSIHWLKDFQITALVTAQLAVRILKIFGKFSYPIVFPKDSQISENQVQVRREVNFWAEIINDTDPAICLTLESSIVYSGDLEQFYENHPYRQDAAKLLVGLKVKTIETNGTAKIIRIAGTIGERREELLTKATGSISRRKLEEAHLGQPVVAVQFGKNSQEYIYPLAALKPCMTDKDESLFQVNYGELLKETKIFYAERQERLKLYKQEAQNTLNNFGFRLGEKSINSREYPELFWNPSISLEQTPILFGKGERGEKIKTLKGLSKGGVYKRHREYLDPARKIRLAILKPANLKVGDFREQLEKRLELYKFETILPAENQINFSVEGVGFEKRARLEEAVDQLIRGEIPVDIALVFLPQEDRNADNTEEGSLYSWIKKKFLERRVMTQMIYEKTLNDKSNYKNILNQVVPGILAKLGNLPYVLAESLEIADYFIGLDVGRMPKKNLPGSLNVCASVRLYGKQGEFVRCRVEDSLTEGEEIPQRILENCLPQAELKNQTVLIYRDGKFQGKEVDNLLARARAINAKFILVECYKTGIPRLYNFEQKQINAPSKGLAFALSKREVILITSQVSEQIGVPRPLRLKVHELGDQVNLKQLVDTTLKLTLLHYGSLKEPRLPIPLYGADAIAYRRLQGICPSLLEDDCQFWL

SEQ ID NO: 12 - protein, 735 aa, Synechococcus sp. PCC7942
MDLLSNLRRSSIVLNRFYVKSLSQSDLTAYEYRCIFKKTPELGDEKRLLASICYKLGAIAVRIGSNIITKEAVRPEKLQGHDWQLVQMGTKQLDCRNDAHRCALETFERKFLERDLSASSQTEVRKAAEGGLIWWVVGAKGIEKSGNGWEVHRGRRIDVSLDAEGNLYLEIDIHHRFYTPWTVHQWLEQYPEIPLSYVRNNYLDERHGFINWQYGRFTQERPQDILLDCLGMSLAEYHLNKGATEEEVQQSYVVYVKPISWRKGKLTAHLSRRLSPSLTMEMLAKVAEDSTVCDREKREIRAVFKSIKQSINQRLQEAQKTASWILTKTYGISSPAIALSCDGYLLPAAKLLAANKQPVSKTADIRNKGCAKIGETSFGYLNLYNNQLQYPLEVHKCLLEIANKNNLQLSLDQRRVLSDYPQDDLDQQMFWQTWSSQGIKTVLVVMPWDSHHDKQKIRIQAIQAGIATQFMVPLPKADKYKALNVTLGLLCKAGWQPIQLESVDHPEVADLIIGFDTGTNRELYYGTSAFAVLADGQSLGWELPAVQRGETFSGQAIWQTVSKLIIKFYQICQRYPQKLLLMRDGLVQEGEFQQTIELLKERKIAVDVISVRKSGAGRMGQEIYENGQLVYRDAAIGSVILQPAERSFIMVTSQPVSKTIGSIRPLRIVHEYGSTDLELLALQTYHLTQLHPASGFRSCRLPWVLHLADRSSKEFQRIGQISVLQNISRDKLIAV

SEQ ID NO: 13 - protein, 735 aa, Clostridium bartlettii CAG1329
MVGLDREFNVITEFKNELKPEDIKIFLYSMPIKDINERHSENYAIVQELKKINENPNIVFNEYIIASFNPIINWGKYKDIDVKPDNRNIDLDNSTERKILERLLLCDIKNNINNTTWEQQNKYEIRGNANPAVYLRKPIYLNNNLIIRRKLNFDVNIDKKDIIIGFFLNHEFEYQKTLDEEIKCGNIQKGDKVKDFYNNITYEFLEMAPFSISQENKYMRSSIIEYYLNKGQSYIISGLDKNTKAVLVKNKEGSIFPYIPNRLKKICVFENLGNKRIIEGNKYIKMNPSQNMSESIKLAEDILKNSKYVKFNKANMIVEKMGYKKDIVKRPALKFGKNESNFSAMYGLNKSGSYEQKNIEIDYFIDPKILNNKRDYQIVYSFLNDIISKSKDLGVEINTDKSYINLTPINIKNENEFESDIMEIIKNYNNPVLVILEKENIDKYYETLKKIFGGRNSIATQFVDLDTIKRCDPKIDNKRGKESIFLNILLGIYCKSGIQPWVLANGLSADCYIGLDVCRENNMSTAGLIQVIGKDGRVLKSKTISSHQSGEKIQINIIKDIIFEAKQAYKNTYNKKLEHIVFHRDGINREDIDLLKEITNSLEIKFDYVEVTKNINRRMAMLEKSDENYNHRDKENKKWITEIGMCLKKENEAYLITTNPSENMGMARPLRIKKVYGNQNIDDIVKDIYKLSFMHIGSIMKSRLPITTHYADLSSIYSHRELMPKSVDNNILHFI

SEQ ID NO: 14 - protein, 745 aa, Clostridium perfringens
MSNLTVEAFEGIGSVNPMLFYQYKVTGKGKYDNVYKIIKSARYKMHSKNRFKPVFIKDDKLYTLEKLPDIEDLDFANINFVKSEVLSIEDNMSIYGEVVEYYINLKLKKVKVLGKYPKYRINYSKEILSNTLLTRELKDEFKKSNKGFNLKRKFRISPVVNKMGKVILYLSCSADFSTNKNIYEMLKEGLEVEGLAVKSEWSNISGNLVIESVLETKISEPTSLGQSLIDYYKNNNQGYRVKDFTDEDLNANIVNVRGNKKIYMYIPHALKPIITREYLAKNDPEFSKEIEQLIKMNMNYRYETLKSFVNDIGVIEELNNLSFKNKYYEDVKLLGYSSGKIDEPVLMGAKGIIKNKMQIFSNGFYKLPEGKVRFGVLYPKEFDGVSRKAIRAIYDFSKEGKYHGESNKYIAEHLINVEFNPKECIFEGYELGDITEYKKAALKLNNYNNVDFVIAIVPNMSDEEIENSYNPFKKIWAELNLPSQMISVKTAEIFANSRDNTALYYLHNIVLGILGKIGGIPWVVKDMKGDVDCFVGLDVGTREKGIHYPACSVVFDKYGKLINYYKPNIPQNGEKINTEILQEIFDKVLISYEEENGAYPKNIVIHRDGFSREDLDWYENYFGKKNIKFNIIEVKKSTPLKIASINEGNITNPEKGSYILRGNKAYMVTTDIKENLGSPKPLKIEKSYGDIDMLTALSQIYALTQIHVGATKSLRLPITTGYADKICKAIEFIPQGRVDNRLFFL

SEQ ID NO: 15 - protein, 705 aa, Clostridium sartagoforme
MKEFNVITEFKNGINSKSIEIYIYKMMVRDFEKRHNENYDVVKELINLNNNSTIVFYEQYIASFKEIEKWGNEQYINVEKRAINLESNEKKILERLLLKEIKNNIDNNKYKVVKDSIYINKPVYNEKGIKIDRYFNLDINVESNGDIIIGFDISHNFEYINTLEYEIKNNNIKIGDRVKDYFYNLTYEYVGIAPFTISEENEYMGCSIVDYYENKNQSYIVNKLPKDMKAILVKNNKNSIFPYIPSRLKKVCRFENLPQNVLRDFNTRVKQKTNEKMQFMVDEVINIVKNSEHIDVKKKNMMCDNIGYKIEDLQQPDLLFGNARAQRYPLYGLKNFGVYENKRIEIKYFIDPILAKSKMNLEKISKFCDELEQFSSKLGVGLNRVKLNNIVNFKEIRMDNEDIFSYEIRKIVSNYNETTIVILSEENLNKYYNIIKKTFSGGNEVPTQCIGFNTLSYTEKNKDSIFLNILLGVYAKSGIQPWILNEKLNSDCFIGLDVSRENKVNKAGVIQVVGKDGRVLKTKVISSSQSGEKIKLETLREIVFEAINSYENTYRCKPKHITFHRDGINREELENLKNTMTNLGVEFDYIEITKGINRRIATISEGEEWKTIMGRCYYKDNSAYVCTTKPYEGIGMAKPIRIRRVFGTLDIEKIVEDAYKLTFMHVGAINKIRLPITTYYADLSSTYGNRDLIPTNIDTNCLYFI

SEQ ID NO: 16 - protein, 705 aa, Clostridium sp. CAG265
MREFNVITEFKNSINANNIEIYIYKMRVRDFENKHNENYDIVKELIKLNNNPTIIFYEQYIASFIEIQNWGSEKYIDVEKRIITLDSNEKKILERLLLKEIKDSIDKEKYKVIKNSIYINKPVYYEKGIKIDRYFNFDININCNNEIIIGFDAKHIFEYINTLECEIKNDNVKIGDKVKDYFYNLTYEYVGIAPFTISEANEYMGCSIIDYYENKNQGYIVNKLPKDMKAVLVKNNKNSIFPYIPSRLKKVCRFENLPQNVLKDFNNRVKQKTNEKMQFMVSEVINIVKNSEHINVEKINMLCGNIGYKLKDLQHPDLLFGNSRAQKYPLYGLKNFGVYENKTIEIKYFIDPILAKNNINLKKVIKFCDELECFSNMLGVKLDRVKLKDKVNFKEIKIDNEDIFSYELKKIASNYNETTIVILSEENLSKYYNIIKKTFSGGNEIPTQCIGFDTLSYNEKNKDSIFFNILLGIYAKSGIQPWILKEKLNSDCFIGLDVSRENKVNKAGVIQVVGKDGKVLKTKVISASQSGEKIKLETLKEIVFEAVGSYENAYGCRPKHITFHRDGISREELENLKNTMNNLGIEFDYIEITKGINRRIATISDDKQWKTVMGRCYYKDDSAFICTTKPYEGMGMAQPIRIRRVFGSLDIEKIVEDAYKLTFMHVGAINKVRLPITTYYADLSSTYGNRDLIPSNIDTNCLYFI

SEQ ID NO: 17 - protein, 736 aa, Intestinibacter bartlettii
MVSLDREFNVITEFKNELKPEDIKIFLYSMPIKDINERHSENYAIVQELKKINENPNIVFNEYIIASFNPIINWGKYKDIDVKPDNRNINLDNSTERKILERLLLCDIKNNINNNTTWAQQNKYEIRGNANPAVYLRKPIYLNDNLIIRRKLNFDVNIDKKDIIIGFFLNHEFEYQKTLDEEIKCGNIQKGDKVKDFYNNITYEFLEMAPFSISQENKYMRSSIIEYYLNKGQSYIISGLDKNTKAVLVKNKEGSIFPYIPNRLKKICVFENLGNRRIIEGNKYIKMNPSQNMSESIKLAEDILKNSKYVKFNKANMIVEKIGYKKDIVKRPALKFGKNESNFSAMYGLNKSGSYEQKNIEIDYFIDPKILNNKRDYQIVYSFLNDIISKSKDLGVEINTDKSYINLTPINIKNENVFELNIIQIIENYNNPVLVILEKENIDKYYETLKKIFGGRNNIPTQFVDLDTIKKCDPKIDNKRGKESIFLNILLGIYCKSGIQPWVLANGLSADCYIGLDVCRENNMSTAGLIQVIGKDGRVLKSKTISSHQSGEKIQINILKDIIFEAKQAYKNTYNKKLEHIVFHRDGINREDIDLLKEITNSLEIKFDYVEVTKNINRRMAMLEKSDENYNHRDKENKKWITEIGMCLKKENEAYLITTNPSENMGMARPLRIKKVYGNQNMDDIVKDIYKLSFMHIGSIMKSRLPITTYYADLSSIYSHRELMPKSVDNNILHFI

SEQ ID NO: 18 - protein, 782 aa, Ferroglobus placidus
MMLNIFEVDKNRVEVPQDVYLYKVHLKTLEQRKRDMCIAVLRNSFGYLDVNSFVIYSYKEIRDLPRRIKKYCDLEPTGKVKMNEVDENVRNALVKTFLRSKIRKDIKKLLKKFKKFQQSVGRWTVALDLERIELVEHNGEILVSFNVKLNISSMINLWDIIERDVNRLKGLCWSPENLNDNRIWFRYIPHLIIEEVEDFEESAKSFILSDVHTGEEFGGWTTEDLKKYPENEYGLSEDAIKKIGNYTQFDSTQPIIKGVTWSGKEYPFLPQHCIPAYNPMLATAEEKKRIEEIKINLKNKKDEIIRKIIEQLPYLKQPDNIEIKKVQETARLRAKFVKVEVTKGKITKTLSQPYEKPVSSTLDLFGWISRIIDGGDGIIEICIPDYIPENLSRIKEIEAFLLIENDLNENERKVGDKLLNDAIYVYNFVRSVCLRCGINIPYLNYKGNRFYFENSKEGIRDIYKRITTSLSGEIGFALIFGKRDNYEDEEGEDSFDYYNPLKSALFRNNILSQNFDVTNYVRGDGKINKNTIKYAVSNIIYNIFGKLGVKFFVLEEDVPYDYILGIDVGYGEAYTGKVAGCTTVHDSEGRLRNLIPIEKQNYPSKETARIKALLEEIEQKKKIYNIDFENKSILILRDGRINKEEINQLMEFSEERNCRITYIEIRKNIVHQFLVNSSQACYVKIGDYYILKAHNPRIGFPRAIKIARKIVIEGDAWRESSLTEDDILLIYKLTALNYSTIGRDSNLRIPAPIYYADKLVKALKKGWKFDERFLRYGILYFL

SEQ ID NO: 19 - protein, 759 aa, Halobacterium sp. DL1
MQAEIDDAFADVPESATEQTGHALTTFRSEKSLSEFAVHEYTLKAKDGYRPDDHQAALRDTYKARRDILGEFESSSPPVIAVRDALALATPSPLPVEDLELKNFEMVNDGPHTLDYTDYSDKAVTKGLIDASLRRILDGDYEVRGIDTVLSKRPVIQKQDFRLHERWNLSLSVTNAGVVYLSVDFRHKTISEYTLDKFDLDKLYRGLRVNVTYKQSGKGAFVDELMDKTVNESIDDMGNQSVVEYHEGAERIPDRVLKEIARADRRVVKVTKQGSHNTEYYPQRLLALQGHPQNVKAFAPRFNEATRGKTRLSAQRCLNRATTFVENLPPVIPLGGATLSFDATPVNGDDTWEMSRLFETDAHILQFAEEQTGDHPRRVKHNEVYEAPEEFSVCLVHPSAGPHAERWENLDQQLKDIGAGPEAVNRVQYDPFKTADEIYTDLLVDVPDDHEYSAACVILPKADFSMGESSASDIYHEMKKALRQRRVDSQMAHIDTLATSYALPNVALGLVAAAGGIPFTTEDAMPGETDLFIGIDVSHRYPRDTDERVHIAASTTSIYGDGTILGYTSAKPQTGEKVPPKELKNLTRQSIAGYKQEHGEYPDRIVIHRDGFMREDLDQVEEMLESMDINYDVVEIRKQSPARVLNLADGVAKIPDKGIAALNREENRAILATFGDPESQATSSNTGLPQPIQVERKAGDTDIKTLTAQVYLLSQSHVGAMNSTARLPITTYYADRASEAAAEGYLPETSKLRQNIGFI

SEQ ID NO: 20 - protein, 706 aa, Methanocaldococcus fervens
MKNLRYKINAYRIKKDYIPKEVYRYRIRSFIENINIYRFVGFYGGVALNQSEFILPYPVENLVLEYDGKDVKLEHIDTLNLEDIENKDKEKAEKLVRGYLTSIYKLKPILYKILRDVRESKIINDIRVDPIPDFTVKRHNNEYYLVIDFNHTATVLKNLWDFVGRDKLKLEDYIGKKIIFKPNPKKRYTIKSIEKQNKKDIDDIVEHIIEYYKWTEEEIKSTFGEIDYTQPIIHCEGIPYPFAPQFCNIVFTMEDLDENTLKDLQSYWRLPNEIKGNIINQIAKKLRFVENEPIELEFIKFNNTPLIVKDENGKPTKIYTTNRLFRWNYDSKSKLYLPYDIPDIIKNKTLTTFVLIDENLKNVSGKIKRKVYQMFKNYNKIASKTELPKFDFANKWKYFSNNNIRDVIRKIKDEFNEELGFALIIGNRYYENDYYETLKMQLFNLNIISQNILWENWSKDDNNFMTNNLLIQIMGKLGIKYFALDAKVNYDYIMGLDSGLGAFKSNRVSGCTVIYDSEGKIRRIQPIDVPSPGERIPIHLVVEFLETKTDINMENKNILFLRDGFVQNSEREELKKLSKELNSNIEVISIRKNNKYKVFTSDYGIGSIFGNDGIFLPHKTTFGSNPVKLSTWLRFNSGNEEKLKINESIMQLLYDLTKMNYSALYGEGRNLRIPAPIHYADKFVKALGKNWKIDEELLKHGFLYFI

SEQ ID NO: 21 - protein, 743 aa, Pseudomonas luteola
MSKVGIAALQLERNLGSLPVYVYDLTFVSSPSDQEPIQLTRRGASQLSWVNRQTAITDFGHFKLVTLRPLSAYEANWQGVHCVVQGERELTLSAENELHRETIKRLVNQCLMQGTRQLANVSNGAMQAEYGQGYQVELIDCLASSRVAVRNEFLDVFQCLRLVPEVLPDGSTLVSFMVRHRLLPREHITLDWVIKTRPEWLESIKRVRHRYSSQGKAPGSANFIAVEKSRSAQSQISTAQGKISLFDYHDQRGNILPGEKSLAYDSSVVKVSYGRSKESFEHLATLLQPMFDFEALQGIDSKLLERIAKGLKWPIADRLEAAAQRIKNLKVGELACGLTTVRNLDERVQYLRPPIRLRFANDKIGDHEKLVTKHGAFRGMQRELVVPIVIGGTNEEQQAAKQHFYNVEKICQLWSNESPRWKQVPSANDELELDARLSSKTLPEALLFIALGRNANKQAIRNVAYRYGLATQFMRLDHPARIYQQTYYNNLAAGLFSKAGGLICGLDQMPGDTDLFIGLDLGGVGQRLPGTAFLFTRSGAQLGWQLAEAQKGERVENEVLHDLLERSLQAFVRANDGLLPQRITLHRDGHLYESLDVIKRFEQKHNVGIDVLEVIKSGCPPLFRRSQGPEGKAIYSNPEVGDAFELSGLDELIVATYSSQDLGQAWGDKVTVRPLRLRKRYGLTDLHTLAKQVILLSRIHGASLYRHPRLPVTTHHADRFASLRQECHLDDLSRMDRLCPVYL

SEQ ID NO: 22 - protein, 799 aa, Thermogladius cellulolyticus 1633
MFRVDLETIRGELEGSPSINVYGLAFKEPIREDDPCIQALENIGYYDGGSRRLYTTRPIGDLPEPCRDSVSLGSHKAVTLKEVPVDGIRSIIKVYVRDKVLRGSRAVLEESDEVGGILDEVLKGLTSKGYRNVRRSRSVFHVGRWNARLTVDSNDVYVFWFNDELFVALNVGLDIDYNGNLWDHMAERSLDKLRSLVEHVRCRYILDIARGRPGTYVVANVIDRSSYEEKSPPMGYEELVKYPVVKAREEGLETRFERLMKKLVGEVDPNQPVLEATRAGKMGRRPVYIPAQYCIPVYDPRLAEKHEDRALAKLRELVNSIGYKGKLINAIARSLGYLSEATHLNIERIEGEKPLKVELVEVEYNGGKPSIINRSKREVSFTADVMKPLRLISIVKEVEEGRRVKIEIPVPNYVPATLRELDELQLLVLVEDGLDPLSFEIMDVVVENVTRLYEIVREVYEPLKIKLPKLRVHRQPILFTHGDYSNVLKEVRKVTGGKLAFAFILGERPADDEEDYYRPLKKVLFKGNVISQNIDVRKYVSDGRVDKRILMPAISNMFYDMLGKLGVKYLTLSKSTDYDLIIGVDVGHGEVEGSRIVGSVVLFDKMGRLMNLVPVYGLGYPGRETARIGDLLEQIEYENYAELKGKRILVLRDGRLTKEEVTQLADISRTKDCTIEVINVKKRTPFQQLSDSRVAWFVDIGDAYLLKTHMLKRDMLPRPIKIDKVKYVFAKGTVREERVSVDDLRLLRGLSHLNYSTIEGETGLRLPAPVHYADKLLDALRSGWEIERRYLRDGLLYFL

SEQ ID NO: 23 - protein, 727 aa, Aromatoleum aromaticum
MPVYYYRLTFDDDHEDINPTQLTRRGARMLSGANGWCAVTDGEPLRVLSLRPLKVLRHEWNGMCCTASDETAGTLKSANPSEREAIQRLLNQCLMQGARKLAYSSDGGLEAEQGAGNLVRLTACQPSERVSAGGDYLDAFQSVTLIPDVLPDGTALVGFDVRHRLLPRPHLTLDWVIRKRPEWMEGIRRVRHRYVSNGQYGTAELIGIATGRTALSTFPTPKGEVSLLGYHESKGNLPEGEREAVAASHVVQVRYGRDTRAKPMEHLAALLQPMFGFDTLSQIDSRLLERIARSLKWPVGERLKAAIRLVKGLKVSELDACLQAVEDTQRFARDLKPEFRLLFRGAAVADSERAVLRHGAYQGMTRKLVVPLVVGGTALEQAVAVRHFEAVERVCRQWHSDLPSWKTAPPAGDAEALNQRLAQRKPENALLLIGLGRGADKRKIRNVAYRYGLATQFMRLDHPPRTYQSTYYNNLAAGVFSKGGGVLCAIDDMPGETDLFIGLDLGGVSQRAPGLAFLFTREGAQLGWQLAEAQRGERVEDAVLGDLLERSLQAYRQVHLGALPRRIALHRDGRLFESLDVIRNFERDYSVRVDVLEVVKSGCPPLYRRGVVAENKKAFRNPEVGDAFELPGLDELIIATYSGEELGSSWGDKVTVRPLRLRKRYGETDLHTLARQVVLLSRIHGASLYRHPRLPVTTHHADRFATLRQECNLDDLSKMDRLCPVYL

SEQ ID NO: 24 - protein, 615 aa, Synechococcus sp. PCC 7002
MICWQKTEADIRSDVSGWEIYRGFKLDIFVSSQGNVFLEVDEHYKLFSAWTLTQWLEWYPDFSAKILRNTYDGRTWKLQKITNEDPNNIDSGTGQTLATYHKNHQKPATDQEINSSKVIYVKPYNSKKEESYPHLSSRVKPCLFLDTLSELASQGDRKVKEVFNLIKPSIEDRFSRATEIAKQLAVEIYNIDEEISDDIKVVQVQAQQSSQDKSILLAHSSKRIDKVFKSLDQGCFRVGEIKFGCINLLDKNQDGWSKFILQKLQKLATIHNINIDANLVKAAVDIPEKDFEIDQFWESWVEQDIKTILVISEWLDNKYKTKLTRDALEHGITLQFMLPLKENVRRIESNKGVESKITCFTSDQYRANNILLGLLAKAGWQAVGLPLLNNEYAADLVIGFDAGRNETLSYGTSSFAVLADGQILGWELPEAQKGEILDPDHVRRTVRRIISQFQKMNGKQSPKRILLMRDGLIQQQEFSLITEALEEGEIKYDLISVRKSGAGRIGRRNSDGTYVDAPKGTVIFSGNDKFKIVTSEAKAGGSARPLFVVREQGDTPIEIIAEQFYRLTQLHPVSGSFTSRLPMPLNYADKLAKKIQSLGSVGILQNLDRKKLFFV

SEQ ID NO: 25 - protein, 766 aa, Thermococcus onnurineus
MEKQTFYQGNMYRLKDELIQDILSDIIVARVTNMPSNPEEAYSEIQKIGGIILNYDEMTNSAWVVGKESLLQNHYPDDMKEVRAFSFSELSKENKTKLVLNILNAEGYLRDIRGHREVVKSINSERSIIRKFLVTVEYDGQHFYLVTLPKYKIIENHTIMELLIEGKITVKELVHNLLKDPKWKIQTSRKDVPLPPGHRVVEIILKTKDPDRYQQELERINEYFTKKTELGPIDDSKYPDDYNIIFRSQTRGKYLSYHSARTKLIRPINKEILREIYRSNEFIKALNIAKKLVADIIYDSTKYPGRAIFPAFKIDERTISYKAVFLKNKTITEKTIQPYYNIKGTFNWLFTNTPFDDISELIIPIQSPEFLRDKTIGVYILYPAKYRENSESLKVIQNLIKSVDSTIKRLSEYFTFLRKVNEGLSLPSAIDIISRIPVNYENLIESAFTRIHSKKGVEYDYHLAITLIPDMRQEQFDKIKGFFFNNGILHKAININNLRDPSKDQKKLIESMILQALYAFGIYFYSLDNLNYDFIIGLDVTREMDKSGRYYGISGAAVVQNKNGQVLKIIPITSPQSSSETANINYLIGNIQQEAAAILNRKGYADILFLRDGKVPGGELEQFKEISRKYNYRFTIIEILKRPLVRFFWENYKEHTVKSPRHNYYFKIGDTYYLTAHYFTNYLKVPLKLGNTYFVARGKISKNVISREDIMTITKLTKLNYSQPENPDKMKLPAPVHLSHRLINYERRELKFNRYEFLKEGALYFL

SEQ ID NO: 26 - protein, 711 aa, Methanopyrus kandleri AV19
MPWRSNAVRVRMDFEVLRREDYHDRGAARGAVKDHLREREFAFQVDTVVYSAGDRGERVSWDDLSPTARAYAVRAALRLGLEGLGFELDTGYSLLCDRDFFEVCYGGRFDGERIPGVYALGNWREVRDRVLRTLGREYGPVEVRLGLEVRARALGDGTVLLSVDPRARAISRVRLKDLVRDRGVGWAERVLPGTYVIPTDGGLTGRRLKVVDAVPADEWEGEDGWDLERLREHWSGYGVEVDPEVVVTVKVGGGVLHYPDDVLRWQVPVHPVTEYFRLGVADRVSVGRYLLDRGLGEFRRRFPDVAVEVREHPGSTDTRRVRPPAVVAGGEKRGKRTELNDLLWNYGPYEGAGELDGEAVHLVADRELPGRAGISERDLERLLELVVGRLRRLLGHDVEAGDVRVADAVDLPDLVGDADGPVLVGVLSDDDRTYARVKRRDPLVQCFTERVLSDGKTVKYAATVLAVGIHCKHAGQPYAVRATSGPKDVAVVGVDVSRKVEGGRVVEGRACCSCFVVDEDGLIEHGRTFTVPVGERGETSGRTAEIVLETAREYSDRVVYLRDGTVPGEELEAVREVGRELGLDVTVVEVIKSDPTVYAIEGEGWRAPRGAYVRLDGSTVHLCCSPYTPRRRGDKKPGTPRPIALRRRDDKLDGDLIGLVHDLTASNWGNPSGTWSRLPAPVLYADRASRLARYGVSVGPGDPVSERPWPV

SEQ ID NO: 27 - protein, 735 aa, Synechococcus elongates
MDLLSNLRRSSIVLNRFYVKSLSQSDLTAYEYRCIFKKTPELGDEKRLLASICYKLGAIAVRIGSNIITKEAVRPEKLQGHDWQLVQMGTKQLDCRNDAHRCALETFERKFLERDLSASSQTEVRKAAEGGLIWWVVGAKGIEKSGNGWEVHRGRRIDVSLDAEGNLYLEIDIHHRFYTPWTVHQWLEQYPEIPLSYVRNNYLDERHGFINWQYGRFTQERPQDILLDCLGMSLAEYHLNKGATEEEVQQSYVVYVKPISWRKGKLTAHLSRRLSPSLTMEMLAKVAEDSTVCDREKREIRAVFKSIKQSINQRLQEAQKTASWILTKTYGISSPAIALSCDGYLLPAAKLLAANKQPVSKTADIRNKGCAKIGETSFGYLNLYNNQLQYPLEVHKCLLEIANKNNLQLSLDQRRVLSDYPQDDLDQQMFWQTWSSQGIKTVLVVMPWDSHHDKQKIRIQAIQAGIATQFMVPLPKADKYKALNVTLGLLCKAGWQPIQLESVDHPEVADLIIGFDTGTNRELYYGTSAFAVLADGQSLGWELPAVQGGETFSGQAIWQTVSKLIIKFYQICQRYPQKLLLMRDGLVQEGEFQQTIELLKERKIAVDVISVRKSGAGRMGQEIYENGQLVYRDAAIGSVILQPAERSFIMVTSQPVSKTIGSIRPLRIVHEYGSTDLELLALQTYHLTQLHPASGFRSCRLPWVLHLADRSSKEFQRIGQISVLQNISRDKLIAV

SEQ ID NO: 28 - protein, 731 aa, Synechococcus sp. JA-3-3Ab
MPMRMLQQEVPVILNRFLVKELTQEDLTFYEYDCRPNPPPELGEEQRAIARVCNRLDVIAARLGSRIITQERVSPSQLKTPEWELEERGLRVLTCANAQERSALESFERKRIGLRLKSQYKKTEVEWIGGGLLWWVTAQKGVELSGEGWEIHRGRMIDVAVEPDGKLYLEVDIHYRFYTPWTLHEWLESYPDVPIEIVRNTYFDANGKRLTWKYLGILSNQSPREIRLPEQNISLAEYHLQKYNALVEEVESSWVVEVASKERRYPHLSRRLSPALTLEMLASLEDNRSPGGKVSAAVIECIRKSLKERFEESEETARTIIKEVYKLSPEEIKPLKTQGYILPKPKLLGSGRRPVENPARVRYRGCAKVGETKFACLNLYDDKREYPSEVLNCLLEVAQKSGAEIEVDFYATQQDLPKGDLARKRFWQTWAEQGIKTVLVVMPWSPNERKQRIRMEALEAGIATQFMIPGADPYKALNVVLGLLCKAAWQPVLLEPLDDPVGPELIIGFDVGTNRRLYYGASAFAVLADGQSLGWELPAVQRGETFSGDAIYQTVSKLVDRFYDKLSRYPSKVLLMRDGLVQGGEFSRTIEELEKEKIAVDIVGIRKSGTGRMGIEEGKGKYKDAKIGTVVFDYSRKSFTLITSQPIRKGGNSLGSARPLRAIHEHGKTPLEVLALQTYHLSQLHPASGFQACRLPWVLHFADKSGKEFQRLGDNIFSILQNIDRQKLIAV

SEQ ID NO: 29 - protein, 707 aa, Anoxybacillus flavithermus
MNQYYITECMANENANNIPLHIYTVSLLNPNQKYSFAHHIVYELRKRNPNVTIAVHGQYIASFQEIAHWGDHEYIKHEYRAIECFNATERTVLERLLKQELIERCKSDYKIYKDLFCLKTEQSIEMNEIIVYPAIYLSFTVEENGNILIGFEYQHRFEYKKTLEDLLRDRSPLIKEGVEVVDSYNKKSYYYTFVEVAPYTAGEKSPYLQQSVIDYYITKNEKWKLKGVHNKTPIVHVRSQTGDIFPYLPHLLRLTCSYEQLMPSTAKQVNRIIKLPPNKKMPKLYQEIFRLLQYQDMLTFRKENVRAANLNYNIFKLSPPLMKFGRNYTTTKVSDGLGQAGVYEPKSVTVSYFVDPELNYDQRKKDEILDFTKKLQDLSGKLGVKLNVSKKPRQLFGVLPVDFFKSERLSFELKSITNAFEGTIVVIGTEENIERAYMAIKKEFGAKADIMTQFVIFDSSIVNNSFYYYNVLLGIYAKSGVQPWILLDHMNSDCFIGLDVSHEQGKHASGIIQVIGKDGRIIKQKSVMATEAGETIARCTMEEIINESIFSYEKMYGMKPSHITFHRDGVCREDLAHLTQYMQSFQIPFDFVEIIKKPRRRMAVYKNKSWFTEQGLCYAKERMAYLCATDPRESVGMAQVVKIVQKTNCLSIRDIVSDAYKLSFMHIHSMLKTRLPITIHYADLSSTFHNRGLIHPRSVHERALPFV

SEQ ID NO: 30 - protein, 605 aa, Exiguobacterium sp. AT1b
MKKQLERLAHPNYMMNRGKFRSKQAHPKSTSGLLIYPALDLNVNVSETAEIVFGFDLTHQFEYRENLLQQIKRDPKSVSAGMRVIDSTHPKSYEYEFVEVAPYRANEVSPIMRCSIIDYFAKKDPKRVIEPDALVVHVKDRNNQILIYLPEQLKQSCSFETIPARHLGAVSRIIKLSPDARMSKLMPEALALIGRLPMLQFERQDVRAARLGYSIQTLPSPRLRFGKGRTTSYAKTGLKQGGVYETGEATVSFFVDPKLRDHQKLQVLEFINKLKTTSERFGVTLNVSHKPKGLSQKLPSDLLQTEDILYQLKNIPQHFEGVVVVIAEEASLQHSYQAIKRQFGGKQDVVTQCVELHDRVLNSEDTLYNILLGIYVKAGLQPWILGEPLHSDCFVGLDVSHENGKHAAGIIQIIGKDGAMIKQKALSTSEAGEKISSETMREIVYDTLHAFEEQYGHAPKHITFHRDGFGREDLTLIDSILSPREIQFDYVEILKNINRRMAIHEDEWKTSQGLSYTKERMGYLLSTNPHARVGMAKPLKVVQQTTTLPFEAILTDVYRLSFMHVHSLLKTRLPITTHYADLSSTFHNRGLLNANTEHEEALPFV

SEQ ID NO: 31 - protein, 735 aa, Synechococcus elongatus PCC 6301; identical to SEQ ID NO: 27

SEQ ID NO: 32 - protein, 735 aa, Synechococcus elongatus PCC 7942; identical to SEQ ID NO: 12

SEQ ID NO: 33 - protein, 764 aa, Lyngbya sp. PCC 8106
MNTTSQAPQNSTSSSIYLSEIFPLTILKPNLICFRLTPEVDREVGNRLSWRFSQKFAEIVVIWENKYFWVLAKPTQKMPSPEQWRQALGEILEQLKEDIGDHYYSIQWVRDPQVTASTLAQLAVRVLKIRKPFSPDIIFSENQVQVQSEVDFWPETIELANTLTPAITLTLKSRFLYRGTLAEFYANHPYRNKPKELLVGLKVRDRETNSSATIVEIAAIDEDRRKELIEKATGAVSRQALEEAPDDQPLVSVRFGKNQKLFDYPMEALCPSITKETASKFDVNYGDLIKQTKPPYQERQNHLTQSKQKAEESLAVYGFQIDKSINNLDYPSLFQTLQFNLEDTELLFGKDSSGKHFVSKRGSVLKGLSQGGVYRRHQDYENYSTPIRISLLNLCNSKVGKFVSQVEERLKQYKFETIRFEKDSLDRRKEIKVDNLDSAEARVAVEKALDELMVIPTNIVLTFLPESDRHTDDTEDGSFYSFVSSRLLRRGISSQVIYEDTLKNPNNYSYILNQVIPGILAKLGNLPFILAKPLEIADYFIGLDISRTPKKRKSGSLNVCASVRLYGKYGEFIRYRLEDALTQGEEIDKRTLERFLPAADLSGKTVLIYRDGRFCGDEIKHLRERAKAMGSKFILVECIKSGIPRLYEVQELTVKDKKKPILKAPPKGLALRLSSHEVMLVTTEVKSEKMGLPNPLRLKVIPEEGQQVSLESLVEATLKLTLLHHGSLKEPRLPIPLYGSDIIAYRRLQGISPGELDGDRQFWL

SEQ ID NO: 34 - protein, 746 aa, Synechococcus sp. JA-2-3B′a(2-13)
MNLLQKKVEVTLNRFFVKELTQEELTFYEYSCRPNPLPDLGEEQRALSKICNQLGVMAVRLGNRIITREKVPASKLKTEEWELEELGSKALDCTDPTERQALETFERKKLDWSLRSKYKRTEIERSTGGGLIWWVTGLEGVERIEDSEGVKPLCGDGWEVHRGRELDVVIRQDKKLYLEIDIRYHFYSPWTLHEWLENYPDVSIEIVRNTYRTKNNKYPTWEYRGIISNQSPREVWLAELNTSLADYHVQKYGATPEEVDGSLVVQVVSRERNFRQKAEPILHLSRRLSPVLTLEMLASIEDQRWTGKKVSSLVSEHIRKSLEERLKESKETAKTIIENVYNISDFQADPLKAEGYIMPSPRLLGFGHRQVQNPARVRYQGCARVGETKFGLLNLDDNKREYPTEVRKCLLEVAKKSGARVEIDFYATRQDLPEGDLAQQRFWQSWADKGIKTVLVVMPLSPNEHKQKIRLQALRAGIATQFMIPGADPYKALNVVLGLLCKAAWQPVLLEPLDHPECSELTIGFDVGTNRELYYGTSAFAVLANGQSLGWELPDIQRGETFSGEAIYQTVSKLVNRFYNKFHRYPRKVLLMRDGLVQRGEFDKTIEELRKEKIAVDIVGVRKSGTVRMGREEKGSYRDAAMGTVIFDHERRSFMLVTSQPIKKSSVPIGSARPLRVVHEHGNTPLEVLAIQTYHLSQLHPASGFQACRLPWVLHFADKSSKEFQRIGNSIFSVLQNINREKLIAV

SEQ ID NO: 35 - protein, 748 aa, Clostridium butyricum
MNNLTFEAFEGIGQLNELNFYKYRLIGKGQIDNVHQAIWSVKYKLQANNFFKPVFVKGEILYSLDELKVIPEFENVEVILDGNIILSISENTDIYKDVIVFYINNALKNIKDITNYRKYITKNTDEIICKSILTTNLKYQYMKSEKGFKLQRKFKISPVVFRNGKVILYLNCSSDFSTDKSIYEMLNNGLDVVGLQVKNRWTNSNGNIFIEEVLDKSISEPGTSGKLGQSLIDYYINGNQKYRVEKFTDEDKKAKVIKAKIKNKTYNYIPQALTPVITREYLSHTDKKFSKQIENVIKMDMNYRYQTLKSFVEDIGVIKELNNLHFKNQYYTNFDFMGFESGILEEPVLMGANGKIKDKKQIFINGFFKNPKENVKFGVLYPEGCMENAQSIARSILDFATAGKYNKQENKYISKNLMNIGFKPSECIFESYKLGDITEYKATARKLKEHEKVGFVIAVIPDMNESEVENPYNPFKKVWAKLNIPSQMITLKTTEKFKNIVDKSGLYYLHNIALNILGKIGGIPWIIKDMPGNIDCFIGLDVGTREKGIHFPACSVLFDKYGKLINYYKPTIPQSGEKIAETILQEIFDNVLISYKEENGEYPKNIVIHRDGFSRENIDWYKEYFDKKGIKFNIIEVKKNIPVKIAKVVGSNICNPIKGSYVLKNDKAFIVTTDIKDGVASPNPLKIEKTYGDVEMKSILEQIYSLSQIHVGSTKSLRLPITTGYADKICKAIEYIPQGVVDNRLFFL

SEQ ID NO: 36 - protein, 745 aa, Halorubrum kocurii
MSQDSRSTEVERQAEIQPGTYLLNGRGEIQLDEVDAFQYDLKVSGGVEQYWDREQFTSSAAYYLDQEHGSPVAEIGKMNVLSKTDLSRSVRVWQRNVTPINRQSVTLTAAQPEDREKIKSFVQSCFKRAVPTEKYSFRFLNKIVRDEPEFTTGSEGFSAHPKHDVKIQVTADGNVLVHVDSGFSIRSNSTLDEIYSEQDNPYGKRVAHDPERYGTQGQGTLRGWSDYRYTDHISDAGSSVNEMHKGVADEEWRQRLAEENPRLLKVEYGNKTRRQAPHFLRLSPRIEQVQDQDREFYSRFNSRSAMMPDERFELSKEFLQNVSRLPVLDMELEPGPVNSSYELLEMREENRLVFGGKQRARDPGSGLRENGVYQSPSQYRLGVLTPERWGEKASELIPLIVSGLNDLSASAGVRAYGYELGDVSNYTPVVQDLHEETDAVLAVVPNKGVAEDFGIDDPYKELKRTLLRKGIPTQMMQKSTVDEIVGQKAGIGNDKFLNALSAVVAKVGGTPWQIDSLPGKTDAFMGLDVTYDESSEQHAGASASVVLADGTTFAAESTTQQGGEKFSARHVEQFVRDLVFDFAGEQGRDIDRLCIMRDGKISEDIDAVREGLSGIEAEIDIVGIRKSGQPRIAEFDGTRFRIAEKGVGFVDADRSQSIIHAFGKPEIHDDNPVGTPRTFRLTKDSGPTDVETLTRQAYWLSEIHFGSPVRSPRLPVPIEYADMAAEYVREEYVSPGTVIEGPAYI

SEQ ID NO: 37 - protein, 1048 aa, Burkholderia ambifaria
MSDVVEKVQWAAIPQMSIDAFVRSIAVNQNRPVCLLLGAGASITSGMPSAQRCIWEWKRDIFITKNPTLRDSLDELSLPGTRRIIQSWLDLQARYPVEGSPDEYSFYAEECYPTSLDRRTFFHRFISEAKPHIGYKLTALLAEAGCVRTIWTTNFDGLVARACTAADVVCVEVGIDTAHRASRAQNDNELRLVSLHGDFRYDALKNTADELREQDAALRKEFLHELKDYDLIVIGYSGRDESLMRVLSAAYADRSSCRLYWCGYGAEPGTEVQRLISSIDPSRESAFYINTKGFDDVISRLAVRRLSGKQLAFAHELIETMAPTVGQRMAFAVPPLSPSALVKGNAYRLSYPGNALKLDIELPELGSWREWLAERMPPTLGQSVVFENGALCLADTAVASRVFDEALRRPPRRIEISDENIVTDGRITSLFRRAIVKAAAKTLNVRTDFRRRIWEPIHYQTRELDNVRYLIHRALSMNIVGIDGIPHVVLTPEIVATMEDGGVAPFEPQKALRVAIYGFQHNDKFDGDLSYWTRQLVEKALDADGGGAFTISKIPLYAGLAQKGKPPLPPTLAKHAKQSGIIVPDAPLVFSAKVGTSEVRNPNPLHGLVLNRPWDHSLTATGLCPSTETAVICPADASTRFERFLRGLQEVARPEQSERDYLHDFPGFPAAFGLPLKIPVRGDSTWMTIDDSVSTDALTGAKQLAHRICEGLDHLRRARPSDTVVIFVPKRWEPFKVVDTQHERFNLHDYIKAYAARHSQSTQFVREETVLNSYTCRVRWWLSLALYVKAMRTPWRLDALDENTAFVGIGYSLDSEAERGNHVLLGCSHIYSARGEGLQFRLGRIENPVMRGRNPFMSEDDARRTGDTIRQLFYDSKMHLPTRVVIHKRTHFTDEERRGLVQGLDGVKNIELIEINVEDSLRYLSSKFKDGKLDIDTFPVYRGTTIVESDDTALLWVHGATPSAQNKYWRYYQGKRRIPAPLRIRRFLGQSDVVQCATEILGLSKMNWNTLDYYSRMPATLDSASSIAKFGTYLDGFSSAPYDYRLLI

SEQ ID NO: 38 - protein, 1063 aa, Burkholderia graminis
MHNRAALQTGSSVRRMGVDALVRSLAVSQDRPLMLFLGAGASMTSGMPSANQCIWEWKRDIFLSNNPGIEEQFSELSLPSVRDRIQTWLDRQRCYPVAGHPDEYGAYIEACFSRSDDRRRYFERWVKQSTPHTGYRLLAELAASGLIQTVWTTNFDGLIARAAAATNLTPIEIGVDSQQRLYRAPGKGELACVSMHGDYRYDRLKNSSGELAQVEVQLRDSLIEALRTHTVVVAGYSGRDESVMQAFHQYAISGPVRTDLPLFWTQYGEAPPLDTVNALLSTNHGEPSRFLVPGVSFDDLMRRLALYLSKGPARDRVNKILDEHATTPVNQLTAFGLPPLPPTGLIKSNAIPLTPPQELLEFDLLQWPASGTVWATLRELGDRHNFVAAPFRSKIYAIAMAESLRVAFGENLKGEIKRVPLNDDDLRYEDGVINQLVRRATVLALSAKANCPSDGESLIWTSEKVEDLRLDRVDWKVHQAVLVQIRPLGSELALVLKPTLYVTDRSGAIAPKDTERLVKQRVLGYQHNKEFNGATEAWRRRLVPQRDFRVRFPDHENGIDLTFSGRPLFARITDERERTVSLSAAQESAARQAGLQLAEPRLKFARKSAAGLAFDTHPVRGLINNRPFDSSLTTTGIASSIRVGIIAPARDATRVHQYLSQLHVAAQPGKDADYLPPFPGFASAYQCPIEIPSVGEQSFVQLDEPDSMTPSSARALAGAITRSIASLSASQRPDVTIIYVPDRWAPLRNYMIDDEEFDLHDFVKAAAIPKGCATQFVEEDTLRNTQQQCRVRWWLSLALYVKSMRTPWTLEGLSEKSAYVGLGFSVKRKTTQNAGAHVVLGCSHLYSPNGIGLQFRLSKIEDPIMRNKNPFMSFDDARRLGEGIRELFFAAHLRLPERVVIHKQTPFLREERSGLQAGLEGVACVELLQIFVDDTLRYVASHPTSDGKFEIDNYPIRRGTTVVIDDHTALLWVHGASTALNPGRHYFQGKRRIPAPLVIRRHAGTTDLMTIADEVLGLSKMNFNSFDLYGQLPATIETSRRVARIGALLDRFSDHSYDYRLFM

SEQ ID NO: 39 - protein, 472 aa, Haloarcula marismortui
MTPQDTPFTLRHLEEPEIQFEGGTETSPKRGLIRYGPRLYEEGHHTIKLGIIGDRDSIRRLTELLHDMEVGIHPGTSDNPWQVPYPGLGKSSPLNLSINAKKGWRRQIRRRDIQSVTSKSTPRDRMERFLKLVQKDIEIIERDSVQPNAIIVCIPQEVMDACTPENQDHARIQSEGSDLRNRIKLIGMEARIPTQLIKPSTLAIRTDRQRASRAWNLTVGLLYKSQRGHPWKTRQIEDGHCYAGLSFYRERDEGDDVIRAALAHVFHGRDHIILQSDPLPDITEDENGSPHLSYEAARQVGEQILEYYEAQKGTRPSRFVLHKPSVFWEEEREGLLDATDGVRDLDLVWVRRRPKVRLFPPTDYPAMRGTLLSVPDDDVHYLYTSGYVPEETTYQGSGVPSPIEIRPDEICETPSLEICKEILFFTKLDWNTSDYAIRMPATVSVAKRVGTILSEVDTESISEVRPQYFYYM

SEQ ID NO: 40 - protein, 484 aa, Mesorhizobium loti
MKFETKIFDEPLLEFGNQHYHPDPRLGLFEAGPLQTPLGDVINIAVVGSAKTVKDSRDFLQAAAVGFAGKSEKHPNLHPPFPGLGNQNPFRCRFEIPDGAVTAIPQARIERIRKEPHHGKAVEMAVDEIIEQLQTIDEGSSRPDVAIIALPVELIERVWNAKVDSDATLEEEDSSGSDAPNFRGMLKAKAMHFRFPIQIVWEDVLDERAVIPLKIKESSARQIQDQASRTWNLLTTLYYKGSGRVPWRRAPQEGEFSACYIGVSFYREAGGQQLFTSAAQMFDERGRGFILKGKGAQTESRGRHPYLTQDDAKTLIADALAAYKKHHMNYPARVIVLKTSRFRDEEADGIFEALDEVGTELRDLVWVQESSFVKVFRDGNYPVMRGTFVKLDGKGLLYTNGSIPYYGTYPGMYDPKPLLICPYKTSDSTVAQIANEIFGLTKINWNSTQMNQKLPIPIRAARKVGEVLKYITDEKVSSDYRRYI

SEQ ID NO: 41 - protein, 626 aa, Pedobacter heparinus
MRNILLNFLKFENQDFGTTVFRKEAEAGVFKDGFSYYDFEVDGRNVKYEISDTALEGYTSFTLPSYLNVGLVSKKLYEKIIDASNQVNGRFILHPEKEYNRRIHFEIEPHPKGRKCVWVEPYFLKSKQVWGLLIGFQFIVSQNVLSGGYKVDRDIQIASGSLNARGLSNLDFYLFKYNHITTFIKNILPGINLNLNNAINSSLFPVESYLLDAKQYMFKDNRIDNSSYFGLSKYAPLQPVSKETTFYFIYRKSDRGIAVNLLKGLRGESHPNTFSGIQKFFKIPFTNDHIKGFALDDYNEPNISKVVEDIKAEVNNVLPVIITNSKKEESDDKLYFSLKHRFTNEGIPCQVVTKDLIINDNALKFSLGNIALQMFAKAGGIPWKMKPATTEYLIIGIGQSYNIEITEDGNKVEKNITYSVLTDSSGIFKDLQVLSEGVATDDSYYTQLVNNIAGIINNGKYKKIAVHTPFRLSKDKVLDKVVKLIDHNIELSVLVINDKTDFFGFDASNNGLVPFESTFLKLSSQEYLVWFEGIQPSNPKITKRFGNPLSIKFWYTNNPQFFQDIDYKESLLQDCINLSGANWRGFKAKQLPVSVFYCQRISEFIGKFRQYELSHIDINNLKPWFL

SEQ ID NO: 42 - protein, 491 aa, Rhodobacterales bacterium
MSDFKTKIFPEPELEFGDQHHHPDPRLGLLQAGPLQTNLGDTIKVGVVGSALTVEKSGEFLNAIEDGFEGKTEKHPNLHPDFPGLRNQNPYRCRFEMVAAEDGVLTKGQIEKIAKEPSDARAVEIAVDAVMAQLEKLEAHHERPDVVMVSLPVKLIERVWRNERARDDEGIEGEAADAKAGRETSPNFRGLLKARAMDLRFSIQIVWEDVINPDAKIPRKIKENSDRQTQDRADLAWNLMTTLYYKGSGKVPWRRLPEEGEFTACYIGISFFKDAETDEIWTSAAQMFDERGRGFILRGGPAQSESRGRHPFLTIDEAHKLTESALAAYKSVHRTMPARVIVMKTSRFREDEAEGVGKALDEAGVELRDLVWIHESYSVKVLRDGDFPVLRGTFVELDGNGLLYTNGSIPYYGTYPGLYVPNPLLLCPHPQSESTIEQIAKEVFSLTKVNWNSTQMNQRLPIPIRAARKVGDVLKYVPSGQKVSSDYRKYI

SEQ ID NO: 43 - protein, 685 aa, Thermus thermophilus
MNHLGKTEVFLNRFALRPLNPEELRPWRLEVVLDPPPGREEVYPLLAQVARRAGGVTVRMGDGLASWSPPEVLVLEGTLARMGQTYAYRLYPKGRRPLDPKDPGERSVLSALARRLLQERLRRLEGVWVEGLAVYRREHARGPGWRVLGGAVLDLWVSDSGAFLLEVDPAYRILCEMSLEAWLAQGHPLPKRVRNAYDRRTWELLRLGEEDPKELPLPGGLSLLDYHASKGRLQGREGGRVAWVADPKDPRKPIPHLTGLLVPVLTLEDLHEEEGSLALSLPWEERRRRTREIASWIGRRLGLGTPEAVRAQAYRLSIPKLMGRRAVSKPADALRVGFYRAQETALALLRLDGAQGWPEFLRRALLRAFGASGASLRLHTLHAHPSQGLAFREALRKAKEEGVQAVLVLTPPMAWEDRNRLKALLLREGLPSQILNVPLREEERHRWENALLGLLAKAGLQVVALSGAYPAELAVGFDAGGRESFRFGGAACAVGGDGGHLLWTLPEAQAGERIPQEVVWDLLEETLWAFRRKAGRLPSRVLLLRDGRVPQDEFALALEALAREGIAYDLVSVRKSGGGRVYPVQGRLADGLYVPLEDKTFLLLTVHRDFRGTPRPLKLVHEAGDTPLEALAHQIFHLTRLYPASGFAFPRLPAPLHLADRLVKEVGRLGIRHLKEVDREKLFFV

SEQ ID NO: 44 - protein, 777 aa, Rhodobacter sphaeroides
MAPVQAADEMYDSNPHPDRRQLVSNGFEVNLPDQVEVIVRDLPDPSKVKEERTRLMGYWFVHWFDGKLFHLRIKAGGPNVDGEHRAIRTAEHPWLLRARLDDALEEALPKYAAVKKRPFTFLAQKDELIDAAATAAGLSHRLLNSFKVIPRFALSPKIYEPVDGTTRVGVFVTIGMRYDIEASLRDLLEAGIDLRGMYVVRRKRQPGERGLLGRVRAISDDMVQLFEETDLASVNVNDAKLEGSKENFTRCLSALLGHNYKKLLNALDDQEAGYRTGPRFDDAVRRMGEFLAKKPIRLADNINAQVGDRIVFSNEGQARNVRLAPKVEYVFDRTGAKSAEYAWRGLSQFGPFDRPSFANRSPRILVVYPSSTQGKVENFLSAFRDGMGSNYSGFSKGFVDLMGLTKVEFVMCPVEVSSADRNGAHTKYNSAIEDKLAGAGEVHAGIVVLFEDHARLPDDRNPYIHTKSLLLTLGVPTQQVRMPTVLLEPKSLQYTLQNFSIATYAKLNGTPWTVNHDKAINDELVVGMGLAELSGSRTEKRQRFVGITTVFAGDGSYLLGNVSKECEYEGYSDAIRESMTGILRELKKRNNWRPGDTVRVVFHAHRPLKRVDVASIVFECTREIGSDQNIQMAFVTVSHDHPFVLIDRSERGLEAYKGSTARKGVFAPPRGAISRVGRLTRLLAVNSPQLIKRANTPLPTPLLVSLHPDSTFKDVDYLAEQALKFTSLSWRSTLPAATPVTIFYSERIAELLGRLKSIPNWSSANLNIKLKWSRWFL

SEQ ID NO: 45 - DNA, 24 bp, Artificial Sequence
  Synthetic Construct
tgcttcagccgctaccccgaccac

SEQ ID NO: 46 - DNA, 24 bp, Artificial Sequence
  Synthetic Construct
gtggtcggggtagcggctgaagca

SEQ ID NO: 47 - DNA, 24 bp, Artificial Sequence
  Synthetic Construct
ccgccccgagttcaaggtggagcg

SEQ ID NO: 48 - DNA, 24 bp, Artificial Sequence
  Synthetic Construct
cggtaaactgcccacttggcagta

SEQ ID NO: 49 - DNA, 24 bp, Artificial Sequence
  Synthetic Construct
ccaagtaggaaagtcccataaggt

SEQ ID NO: 50 - DNA, 24 bp, Artificial Sequence
  Synthetic Construct
aagggcgaggagctgttcaccggg

SEQ ID NO: 51 - DNA, 24 bp, Artificial Sequence
  Synthetic Construct
gtggtcggggtagcggctgaagca

SEQ ID NO: 52 - DNA, 24 bp, Artificial Sequence
  Synthetic Construct
cctaccagaatcgcccagtggctg

SEQ ID NO: 53 - DNA, 24 bp, Artificial Sequence
  Synthetic Construct
cagccactgggcgattctggtagg

SEQ ID NO: 54 - DNA, 24 bp, Artificial Sequence
  Synthetic Construct
atcgcccagtggctgctaatacct

SEQ ID NO: 55 - DNA, 24 bp, Artificial Sequence
  Synthetic Construct
aggtattagcagccactgggcgat

SEQ ID NO: 56 - DNA, 24 bp, Artificial Sequence
  Synthetic Construct
ggctgctaataccttggactttgg

SEQ ID NO: 57 - DNA, 24 bp, Artificial Sequence
  Synthetic Construct
ccaaagtccaaggtattagcagcc

SEQ ID NO: 58 - DNA, 24 bp, Artificial Sequence
  Synthetic Construct
ataccttggactttggacagaatg

SEQ ID NO: 59 - DNA, 24 bp, Artificial Sequence
  Synthetic Construct
cattctgtccaaagtccaaggtat

SEQ ID NO: 60 - DNA, 24 bp, Artificial Sequence
  Synthetic Construct
gctccattctgtccaaagtccaag

SEQ ID NO: 61 - DNA, 24 bp, Artificial Sequence
  Synthetic Construct
agacggtcaaattaacgtccatag

SEQ ID NO: 62 - DNA, 24 bp, Artificial Sequence
  Synthetic Construct
ctgctcctcttggttggtcaggca

SEQ ID NO: 63 - DNA, 24 bp, Artificial Sequence
  Synthetic Construct
tgcctgaccaaccaagaggagcag

SEQ ID NO: 64 - DNA, 24 bp, Artificial Sequence
  Synthetic Construct
ctcctcttggttggtcaggcactg

SEQ ID NO: 65 - DNA, 24 bp, Artificial Sequence
  Synthetic Construct
cagtgcctgaccaaccaagaggag

SEQ ID NO: 66 - DNA, 24 bp, Artificial Sequence
  Synthetic Construct
cgctgtccaccttccagcagatgt

SEQ ID NO: 67 - DNA, 24 bp, Artificial Sequence
  Synthetic Construct
acatctgctggaaggtggacagcg

SEQ ID NO: 68 - DNA, 24 bp, Artificial Sequence
  Synthetic Construct
cagcaagcaggagtatgacgagtc

SEQ ID NO: 69 - DNA, 24 bp, Artificial Sequence
  Synthetic Construct
gactcgtcatactcctgcttgctg

SEQ ID NO: 70 - DNA, 24 bp, Artificial Sequence
  Synthetic Construct
gtccggcccctccatcgtccaccg

SEQ ID NO: 71 - DNA, 24 bp, Artificial Sequence
  Synthetic Construct
cggtggacgatggaggggccggac

SEQ ID NO: 72 - DNA, 24 bp, Artificial Sequence
  Synthetic Construct
ccctccatcgtccaccgcaaatgc

SEQ ID NO: 73 - DNA, 24 bp, Artificial Sequence
  Synthetic Construct
agcatttgcggtggacgatggagg

SEQ ID NO: 74 - DNA, 24 bp, Artificial Sequence
  Synthetic Construct
cccacgagggcagagtgctgcttg

SEQ ID NO: 75 - DNA, 24 bp, Artificial Sequence
  Synthetic Construct
caagcagcactctgccctcgtggg

SEQ ID NO: 76 - DNA, 24 bp, Artificial Sequence
  Synthetic Construct
gccaatggggaggacatcgatgtc

SEQ ID NO: 77 - DNA, 24 bp, Artificial Sequence
  Synthetic Construct
gacatcgatgtcctccccattggc

SEQ ID NO: 78 - DNA, 24 bp, Artificial Sequence
  Synthetic Construct
tgtcacctccaatgactagggtgg

SEQ ID NO: 79 - DNA, 24 bp, Artificial Sequence
  Synthetic Construct
ccaccctagtcattggaggtgaca

SEQ ID NO: 80 - DNA, 24 bp, Artificial Sequence
  Synthetic Construct
gcaaccacaaacccacgagggcag

SEQ ID NO: 81 - DNA, 24 bp, Artificial Sequence
  Synthetic Construct
ctgccctcgtgggtttgtggttgc

SEQ ID NO: 82 - DNA, 24 bp, Artificial Sequence
  Synthetic Construct
tgctggccaggcccctgcgtgggc

SEQ ID NO: 83 - DNA, 24 bp, Artificial Sequence
  Synthetic Construct
gcccacgcaggggcctggccagca

SEQ ID NO: 84 - DNA, 24 bp, Artificial Sequence
  Synthetic Construct
gagatggcgccttcctctcagggc

SEQ ID NO: 85 - DNA, 24 bp, Artificial Sequence
  Synthetic Construct
gcgccttcctctcagggcagagga

SEQ ID NO: 86 - DNA, 24 bp, Artificial Sequence
  Synthetic Construct
ctcttctctgcacagctcctaagc

SEQ ID NO: 87 - DNA, 24 bp, Artificial Sequence
  Synthetic Construct
ggcgcccgcgccgtgcatgaaggc

SEQ ID NO: 88 - DNA, 24 bp, Artificial Sequence
  Synthetic Construct
agctccggtggggccgcgtctggt

SEQ ID NO: 89 - DNA, 24 bp, Artificial Sequence
  Synthetic Construct
ggtccctggcggccgccgccgccg

SEQ ID NO: 90 - DNA, 24 bp, Artificial Sequence
  Synthetic Construct
gataaggtccttgaattgcagtat

SEQ ID NO: 91 - DNA, 24 bp, Artificial Sequence
  Synthetic Construct
ttgcagggagtcgacgagttgaag

SEQ ID NO: 92 - DNA, 24 bp, Artificial Sequence
  Synthetic Construct
atgaatgagaccgacccaaagagc

SEQ ID NO: 93 - DNA, 24 bp, Artificial Sequence
  Synthetic Construct
gcggggccgggcctgggctgcggg

SEQ ID NO: 94 - DNA, 24 bp, Artificial Sequence
  Synthetic Construct
accgtaggtttcggacatggccgt

SEQ ID NO: 95 - DNA, 24 bp, Artificial Sequence
  Synthetic Construct
ctccaccctccgtccggccgcgac

SEQ ID NO: 96 - DNA, 24 bp, Artificial Sequence
  Synthetic Construct
cgcgtgcgggccgccactgtgggc

SEQ ID NO: 97 - DNA, 24 bp, Artificial Sequence
  Synthetic Construct
catggcctgcacctcgccgcggta

SEQ ID NO: 98 - DNA, 24 bp, Artificial Sequence
  Synthetic Construct
gcctcaagagctggttcgagcccc

SEQ ID NO: 99 - DNA, 24 bp, Artificial Sequence
  Synthetic Construct
agaaggtatacacgtcggaagaat

SEQ ID NO: 100 - DNA, 18 bp, Artificial Sequence
  Synthetic Construct
gttctttcaggtgcgtca

SEQ ID NO: 101 - DNA, 20 bp, Artificial Sequence
  Synthetic Construct
gggactcttctctatcagcc

SEQ ID NO: 102 - DNA, 18 bp, Artificial Sequence
  Synthetic Construct
acggctctgcccaggtta

SEQ ID NO: 103 - DNA, 18 bp, Artificial Sequence
  Synthetic Construct
cattgttggcacattccg

SEQ ID NO: 104 - DNA, 22 bp, Artificial Sequence
  Synthetic Construct
cccctttgatttttgatcttcg

SEQ ID NO: 105 - DNA, 18 bp, Artificial Sequence
  Synthetic Construct
tgtgcaggaccgggctgt

SEQ ID NO: 106 - DNA, 20 bp, Artificial Sequence
  Synthetic Construct
caggagggccaggagatttg

SEQ ID NO: 107 - DNA, 20 bp, Artificial Sequence
  Synthetic Construct
tgaaatcgaggatctgggcg

SEQ ID NO: 108 - DNA, 20 bp, Artificial Sequence
  Synthetic Construct
ccatccccttctgtgaatgt

SEQ ID NO: 109 - DNA, 20 bp, Artificial Sequence
  Synthetic Construct
ggagattggagacacggaga

SEQ ID NO: 110 - DNA, 21 bp, Artificial Sequence
  Synthetic Construct
ggaactggaggaacaactgac

SEQ ID NO: 111 - DNA, 18 bp, Artificial Sequence
  Synthetic Construct
tcggcgttcagtgattgt

SEQ ID NO: 112 - DNA, 18 bp, Artificial Sequence
  Synthetic Construct
atgcgctgtgcacagcgc

SEQ ID NO: 113 - DNA, 21 bp, Artificial Sequence
  Synthetic Construct
tcagggaaatcgggactcagc

SEQ ID NO: 114 - DNA, 21 bp, Artificial Sequence
  Synthetic Construct
cacgaaactaccttcaactcc

SEQ ID NO: 115 - DNA, 21 bp, Artificial Sequence
  Synthetic Construct
gacttcctgtaacaacgcatc

SEQ ID NO: 116 - DNA, 21 bp, Artificial Sequence
  Synthetic Construct
ggtcactgttgaaactcatcc

SEQ ID NO: 117 - DNA, 19 bp, Artificial Sequence
  Synthetic Construct
cttgtagatgaaggtgccg

SEQ ID NO: 118 - DNA, 21 bp, Artificial Sequence
  Synthetic Construct
ctaactgaaacacggaaggag

SEQ ID NO: 119 - DNA, 20 bp, Artificial Sequence
  Synthetic Construct
cttgtagcggttcagtgtgt

SEQ ID NO: 120 - DNA, 16 bp, Artificial Sequence
  Synthetic Construct
aagttcagcgtgtccg

SEQ ID NO: 121 - DNA, 17 bp, Artificial Sequence
  Synthetic Construct
gccgtttcttctgcttg

SEQ ID NO: 122 - DNA, 20 bp, Artificial Sequence
  Synthetic Construct
gcccttgaatgtatttggga

SEQ ID NO: 123 - DNA, 20 bp, Artificial Sequence
  Synthetic Construct
gtgcgtcagcaatttcctgc

SEQ ID NO: 124 - DNA, 20 bp, Artificial Sequence
  Synthetic Construct
atgcggttcaccagggtgtc

SEQ ID NO: 125 - DNA, 22 bp, Artificial Sequence
  Synthetic Construct
gggtcttgtagttgccgtcgtc

SEQ ID NO: 126 - DNA, 25 bp, Artificial Sequence
  Synthetic Construct
ggaattcgtgagcaagggcgaggag

SEQ ID NO: 127 - DNA, 28 bp, Artificial Sequence
  Synthetic Construct
ccgcttaaggccgatttcggcctattgg

SEQ ID NO: 128 - DNA, 29 bp, Artificial Sequence
  Synthetic Construct
gaagatctacagtgattgacctcgattcg

SEQ ID NO: 129 - DNA, 34 bp, Artificial Sequence
  Synthetic Construct
ccgctcgagctagaggaatccgacattagactcg

SEQ ID NO: 130 - DNA, 63 bp, Artificial Sequence
  Synthetic Construct
cccaagcttgccaccatggattacaaggatgacgacgataagacagtgattgacctcgattcg

SEQ ID NO: 131 - DNA, 60 bp, Artificial Sequence
  Synthetic Construct
cgggatccttaagcgtaatctggaacatcgtatgggtagaggaatccgacattagactcg

SEQ ID NO: 132 - DNA, 45 bp, Artificial Sequence
  Synthetic Construct
ccaaaaaagaagagaaaggtagccacagtgattgacctcgattcg

SEQ ID NO: 133 - DNA, 45 bp, Artificial Sequence
  Synthetic Construct
cccaagcttgccaccatggtgccaaaaaagaagagaaaggtagcc

SEQ ID NO: 134 - DNA, 32 bp, Artificial Sequence
  Synthetic Construct
cgggatcccggaggaatccgacattagactcg

SEQ ID NO: 135 - DNA, 45 bp, Artificial Sequence; identical to SEQ ID NO: 133
  Synthetic Construct

SEQ ID NO: 136 - DNA, 33 bp, Artificial Sequence
  Synthetic Construct
cgggatcctcagaggaatccgacattagactcg

SEQ ID NO: 137 - DNA, 37 bp, Artificial Sequence
  Synthetic Construct
cccaagcttgccaccatggactataaggaccacgacg

SEQ ID NO: 138 - DNA, 31 bp, Artificial Sequence
  Synthetic Construct
cgggatcctcactttttcttttttgcctggc

SEQ ID NO: 139 - DNA, 27 bp, Artificial Sequence
  Synthetic Construct
cccaagcttaacgaccctgccctgaac

SEQ ID NO: 140 - DNA, 33 bp, Artificial Sequence
  Synthetic Construct
cgcggatccgggcatgactaacatgagaattac

SEQ ID NO: 141 - DNA, 31 bp, Artificial Sequence
  Synthetic Construct
cccaagcttgggggacttgtacagctcgtcc

SEQ ID NO: 142 - DNA, 34 bp, Artificial Sequence
  Synthetic Construct
cgcggatcctagttattaatagtaatcaattacg

SEQ ID NO: 143 - DNA, 26 bp, Artificial Sequence
  Synthetic Construct
caccggcccagtggctgctaatacct

SEQ ID NO: 144 - DNA, 26 bp, Artificial Sequence
  Synthetic Construct
aaacaggtattagcagccactgggcc

SEQ ID NO: 145 - DNA, 26 bp, Artificial Sequence
  Synthetic Construct
caccggctgggcataatgccaggcgg

SEQ ID NO: 146 - DNA, 26 bp, Artificial Sequence
  Synthetic Construct
aaacccgcctggcattatgcccagcc

SEQ ID NO: 147 - DNA, 26 bp, Artificial Sequence
  Synthetic Construct
caccggctcgtgaccaccctgaccta

SEQ ID NO: 148 - DNA, 26 bp, Artificial Sequence
  Synthetic Construct
aaactaggtcagggtggtcacgagcc

SEQ ID NO: 149 - DNA, 26 bp, Artificial Sequence
  Synthetic Construct
caccgggagatggcgccttcctctca

SEQ ID NO: 150 - DNA, 26 bp, Artificial Sequence
  Synthetic Construct
aaactgagaggaaggcgccatctccc

SEQ ID NO: 151 - DNA, 26 bp, Artificial Sequence
  Synthetic Construct
caccggggcgcccgcgccgtgcatga

SEQ ID NO: 152 - DNA, 26 bp, Artificial Sequence
  Synthetic Construct
aaactcatgcacggcgcgggcgcccc

SEQ ID NO: 153 - DNA, 960 bp, Artificial Sequence
  Synthetic Construct
gcttttgatggaagattaaagcttggaaacagtcctctgaaaaatccttttaaaaatctgttctttcaggtgcgtcagcaatttcctgctcctcttggttggtcaggcactgaagctcctacacaggtcactgttgaaactcatcctgttcaagaaacaacctttcatgtagcccctcaacagaatgcattgcatcatcaccatggtaacagttcccatcaccatcaccaccaccaccaccatcaccaccaccatggacaacaagccttgggtaaccggaccaggccaagggtctacaattctccaacgaatagctcctctacccaagattctatggaggttggccacagtcaccactccatgacatccctgtcttcctcaacgacttcttcctcgacatcttcctcctctactggtaaccaaggcaatcaggcctaccagaatcgcccagtggctgctaataccttggactttggacagaatggagctatggacgttaatttgaccgtctactccaatccccgccaagagactggcatagctggacatccaacataccaattttctgctaatacaggtcctgcacattacatgactgaaggacatctgacaatgaggcaaggggctgatagagaagagtcccccatgacaggagtttgtgtgcaacagagtcctgtagctagctcgtgactacattgaaacttgagtttgtttcttgtgtgtttttatagaagtggtgttttttttccaaaaacaaagtgcaaagctgcttgaatcaggaggagattaacacactgaaccgctacaagagggcaaagctgattttttttttaacttgaaaagattgcaaagggacattgaagtgtttaaaagagccatgtccaaacccatcttcatggatagctcagaggtatcctctttttgctcccccattttaacttgccacatcccagtcacagt

SEQ ID NO: 154 - DNA, 960 bp, Artificial Sequence
  Synthetic Construct
actgtgactgggatgtggcaagttaaaatgggggagcaaaaagaggatacctctgagctatccatgaagatgggtttggacatggctcttttaaacacttcaatgtccctttgcaatcttttcaagttaaaaaaaaaatcagctttgccctcttgtagcggttcagtgtgttaatctcctcctgattcaagcagctttgcactttgtttttggaaaaaaaacaccacttctataaaaacacacaagaaacaaactcaagtttcaatgtagtcacgagctagctacaggactctgttgcacacaaactcctgtcatgggggactcttctctatcagccccttgcctcattgtcagatgtccttcagtcatgtaatgtgcaggacctgtattagcagaaaattggtatgttggatgtccagctatgccagtctcttggcggggattggagtagacggtcaaattaacgtccatagctccattctgtccaaagtccaaggtattagcagccactgggcgattctggtaggcctgattgccttggttaccagtagaggaggaagatgtcgaggaagaagtcgttgaggaagacagggatgtcatggagtggtgactgtggccaacctccatagaatcttgggtagaggagctattcgttggagaattgtagacccttggcctggtccggttacccaaggcttgttgtccatggtggtggtgatggtggtggtggtggtgatggtgatgggaactgttaccatggtgatgatgcaatgcattctgttgaggggctacatgaaaggttgtttcttgaacaggatgagtttcaacagtgacctgtgtaggagcttcagtgcctgaccaaccaagaggagcaggaaattgctgacgcacctgaaagaacagatttttaaaaggatttttcagaggactgtttccaagctttaatcttccatcaaaagc

SEQ ID NO: 155 - DNA, 1069 bp, Artificial Sequence
  Synthetic Construct
ggaattcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggccttaagcgg

SEQ ID NO: 156 - DNA, 960 bp, Artificial Sequence; identical to SEQ ID NO: 153
  Synthetic Construct

SEQ ID NO: 157 - protein, 4 aa, Artificial Sequence
  Synthetic Construct
  VARIANT 4: Xaa = Any Amino Acid
DEDX

SEQ ID NO: 158 - DNA, 63 bp, Artificial Sequence
  Synthetic Construct
gtcgtgctgcttcatgtggtcggggtagcggctgaagcactgcacgccgtaggtcagggtggt

SEQ ID NO: 159 - DNA, 63 bp, Artificial Sequence
  Synthetic Construct
accaccctgacctacggcgtgcagtgcttcagccgctaccccgaccacatgaagcagcacgac

SEQ ID NO: 160 - DNA, 59 bp, Artificial Sequence
  Synthetic Construct
cctgacctacggcgtgcagtgcttcagccgctaccccgaccacatgaagcagcacgact

SEQ ID NO: 161 - DNA, 49 bp, Artificial Sequence
  Synthetic Construct
cctgacctacggcgtgcagtgcttcaggaccacatgaagcagcacgact

SEQ ID NO: 162 - DNA, 44 bp, Artificial Sequence
  Synthetic Construct
cctgacctacggcgtgcagtgcttcaacatgaagcagcacgact

SEQ ID NO: 163 - DNA, 42 bp, Artificial Sequence
  Synthetic Construct
cctgacctacggcgtgcagtgaccacatgaagcagcacgact

SEQ ID NO: 164 - DNA, 40 bp, Artificial Sequence
  Synthetic Construct
cctgacctacggcgtgcagtccacatgaagcagcacgact

SEQ ID NO: 165 - DNA, 33 bp, Artificial Sequence
  Synthetic Construct
ccgcggcaattcgattctgaagcactgcacgcc

SEQ ID NO: 166 - DNA, 30 bp, Artificial Sequence
  Synthetic Construct
attcactagtgattgaccacatgaagcagc

SEQ ID NO: 167 - DNA, 86 bp, Artificial Sequence
  Synthetic Construct
gcatggcggacttgaagaagtcgtgctgcttcatgtggtcggggtagcggctgaagcactgcacgccgtaggtcagggtggtcacg

SEQ ID NO: 168 - DNA, 20 bp, Artificial Sequence
  Synthetic Construct
aagggcgaggagctgttcac

SEQ ID NO: 169 - DNA, 21 bp, Artificial Sequence
  Synthetic Construct
aagggcgaggagctgttcacc

SEQ ID NO: 170 - DNA, 22 bp, Artificial Sequence
  Synthetic Construct
aagggcgaggagctgttcaccg

SEQ ID NO: 171 - DNA, 23 bp, Artificial Sequence
  Synthetic Construct
aagggcgaggagctgttcaccgg

SEQ ID NO: 172 - DNA, 25 bp, Artificial Sequence
  Synthetic Construct
aagggcgaggagctgttcaccgggg

SEQ ID NO: 173 - DNA, 26 bp, Artificial Sequence
  Synthetic Construct
aagggcgaggagctgttcaccggggt

SEQ ID NO: 174 - DNA, 27 bp, Artificial Sequence
  Synthetic Construct
aagggcgaggagctgttcaccggggtg

SEQ ID NO: 175 - DNA, 74 bp, Artificial Sequence
  Synthetic Construct
ccaaggcaatcaggcctaccagaatcgcccagtggctgctaataccttggactttggacagaatggagctatgg

SEQ ID NO: 176 - DNA, 64 bp, Artificial Sequence
  Synthetic Construct
ccaaggcaatcaggcctaccagaatcgcccagtggctgctaatacctacagaatggagctatgg

SEQ ID NO: 177 - DNA, 74 bp, Artificial Sequence
  Synthetic Construct
ccaaggcaatcaggcctaccagaatcgcccagtggctgctaataccttggacttcggacagaatggagctatgg

SEQ ID NO: 178 - DNA, 57 bp, Artificial Sequence
  Synthetic Construct
ccaaggcaatcaggcctaccagaatcgcccaggactttggacagaatggagctatgg

SEQ ID NO: 179 - DNA, 49 bp, Artificial Sequence
  Synthetic Construct
ccaaggcaatcaggcctaccagaatcgcccggacagaatggagctatgg

SEQ ID NO: 180 - DNA, 75 bp, Artificial Sequence
  Synthetic Construct
ccaaggcaatcaggcctaccagaatcgcccagtggctgctaatacgcttggactttggacagaatggagctatgg

SEQ ID NO: 181 - DNA, 76 bp, Artificial Sequence
  Synthetic Construct
ccaaggcaatcaggcctaccagaatcgcccagtggctgctaataccttggacaatttggacagaatggagctatgg

SEQ ID NO: 182 - DNA, 42 bp, Artificial Sequence
  Synthetic Construct
ccaaggcaatcaggcctaccagaatacagaatggagctatgg

SEQ ID NO: 183 - DNA, 24 bp, Artificial Sequence
  Synthetic Construct
tcaaagtccaaggtattagcagcc

SEQ ID NO: 184 - DNA, 24 bp, Artificial Sequence
  Synthetic Construct
ctaaagtccaaggtattagcagcc

SEQ ID NO: 185 - DNA, 24 bp, Artificial Sequence
  Synthetic Construct
ccgaagtccaaggtattagcagcc

SEQ ID NO: 186 - DNA, 24 bp, Artificial Sequence
  Synthetic Construct
ccagagtccaaggtattagcagcc

SEQ ID NO: 187 - DNA, 24 bp, Artificial Sequence
  Synthetic Construct
ccaaggtccaaggtattagcagcc

SEQ ID NO: 188 - DNA, 24 bp, Artificial Sequence
  Synthetic Construct
ccaaaatccaaggtattagcagcc

SEQ ID NO: 189 - DNA, 24 bp, Artificial Sequence
  Synthetic Construct
ccaaagcccaaggtattagcagcc

SEQ ID NO: 190 - DNA, 24 bp, Artificial Sequence
  Synthetic Construct
ccaaagttcaaggtattagcagcc

SEQ ID NO: 191 - DNA, 24 bp, Artificial Sequence
  Synthetic Construct
ccaaagtctaaggtattagcagcc

SEQ ID NO: 192 - DNA, 24 bp, Artificial Sequence
  Synthetic Construct
ccaaagtccgaggtattagcagcc

SEQ ID NO: 193 - DNA, 24 bp, Artificial Sequence
  Synthetic Construct
ccaaagtccagggtattagcagcc

SEQ ID NO: 194 - DNA, 24 bp, Artificial Sequence
  Synthetic Construct
ccaaagtccaaagtattagcagcc

SEQ ID NO: 195 - DNA, 24 bp, Artificial Sequence
  Synthetic Construct
ccaaagtccaagatattagcagcc

SEQ ID NO: 196 - DNA, 24 bp, Artificial Sequence
  Synthetic Construct
ccaaagtccaaggcattagcagcc

SEQ ID NO: 197 - DNA, 24 bp, Artificial Sequence
  Synthetic Construct
ccaaagtccaaggtgttagcagcc

SEQ ID NO: 198 - DNA, 24 bp, Artificial Sequence
  Synthetic Construct
ccaaagtccaaggtactagcagcc

SEQ ID NO: 199 - DNA, 24 bp, Artificial Sequence
  Synthetic Construct
ccaaagtccaaggtatcagcagcc

SEQ ID NO: 200 - DNA, 24 bp, Artificial Sequence
  Synthetic Construct
ccaaagtccaaggtattggcagcc

SEQ ID NO: 201 - DNA, 24 bp, Artificial Sequence
  Synthetic Construct
ccaaagtccaaggtattaacagcc

SEQ ID NO: 202 - DNA, 24 bp, Artificial Sequence
  Synthetic Construct
ccaaagtccaaggtattagtagcc

SEQ ID NO: 203 - DNA, 24 bp, Artificial Sequence
  Synthetic Construct
ccaaagtccaaggtattagcggcc

SEQ ID NO: 204 - DNA, 24 bp, Artificial Sequence
  Synthetic Construct
ccaaagtccaaggtattagcaacc

SEQ ID NO: 205 - DNA, 24 bp, Artificial Sequence
  Synthetic Construct
ccaaagtccaaggtattagcagtc

SEQ ID NO: 206 - DNA, 24 bp, Artificial Sequence
  Synthetic Construct
ccaaagtccaaggtattagcagct

SEQ ID NO: 207 - DNA, 24 bp, Artificial Sequence
  Synthetic Construct
ttgaagtccaaggtattagcagcc

SEQ ID NO: 208 - DNA, 24 bp, Artificial Sequence
  Synthetic Construct
ccaaagtccaaaacattagcagcc

SEQ ID NO: 209 - DNA, 24 bp, Artificial Sequence
  Synthetic Construct
ccaaagtccaaggtattagcaatt

SEQ ID NO: 210 - DNA, 21 bp, Artificial Sequence
  Synthetic Construct
tcaaagtccaaggtattagca

SEQ ID NO: 211 - DNA, 21 bp, Artificial Sequence
  Synthetic Construct
ctaaagtccaaggtattagca

SEQ ID NO: 212 - DNA, 21 bp, Artificial Sequence
  Synthetic Construct
ccgaagtccaaggtattagca

SEQ ID NO: 213 - DNA, 21 bp, Artificial Sequence
  Synthetic Construct
ccagagtccaaggtattagca

SEQ ID NO: 214 - DNA, 21 bp, Artificial Sequence
  Synthetic Construct
ccaaggtccaaggtattagca

SEQ ID NO: 215 - DNA, 21 bp, Artificial Sequence
  Synthetic Construct
ccaaaatccaaggtattagca

SEQ ID NO: 216 - DNA, 21 bp, Artificial Sequence
  Synthetic Construct
ccaaagcccaaggtattagca

SEQ ID NO: 217 - DNA, 21 bp, Artificial Sequence
  Synthetic Construct
ccaaagttcaaggtattagca

SEQ ID NO: 218 - DNA, 21 bp, Artificial Sequence
  Synthetic Construct
ccaaagtctaaggtattagca

SEQ ID NO: 219 - DNA, 21 bp, Artificial Sequence
  Synthetic Construct
ccaaagtccgaggtattagca

SEQ ID NO: 220 - DNA, 21 bp, Artificial Sequence
  Synthetic Construct
ccaaagtccagggtattagca

SEQ ID NO: 221 - DNA, 21 bp, Artificial Sequence
  Synthetic Construct
ccaaagtccaaagtattagca

SEQ ID NO: 222 - DNA, 21 bp, Artificial Sequence
  Synthetic Construct
ccaaagtccaagatattagca

SEQ ID NO: 223 - DNA, 21 bp, Artificial Sequence
  Synthetic Construct
ccaaagtccaaggcattagca

SEQ ID NO: 224 - DNA, 21 bp, Artificial Sequence
  Synthetic Construct
ccaaagtccaaggtgttagca

SEQ ID NO: 225 - DNA, 21 bp, Artificial Sequence
  Synthetic Construct
ccaaagtccaaggtactagca

SEQ ID NO: 226 - DNA, 21 bp, Artificial Sequence
  Synthetic Construct
ccaaagtccaaggtatcagca

SEQ ID NO: 227 - DNA, 21 bp, Artificial Sequence
  Synthetic Construct
ccaaagtccaaggtattggca

SEQ ID NO: 228 - DNA, 21 bp, Artificial Sequence
  Synthetic Construct
ccaaagtccaaggtattaaca

SEQ ID NO: 229 - DNA, 21 bp, Artificial Sequence
  Synthetic Construct
ccaaagtccaaggtattagta

SEQ ID NO: 230 - DNA, 21 bp, Artificial Sequence
  Synthetic Construct
ccaaagtccaaggtattagcg

SEQ ID NO: 231 - DNA, 21 bp, Artificial Sequence
  Synthetic Construct
ttgaagtccaaggtattagca

SEQ ID NO: 232 - DNA, 21 bp, Artificial Sequence
  Synthetic Construct
ccaaagtccaaaacattagca

SEQ ID NO: 233 - DNA, 21 bp, Artificial Sequence
  Synthetic Construct
ccaaagtccaaggtattaatg

SEQ ID NO: 234 - RNA, 26 nt, Artificial Sequence
  Synthetic Construct
cccaguggcugcuaauaccuguuuua

SEQ ID NO: 235 - DNA, 50 bp, Artificial Sequence
  Synthetic Construct
gatctgggtcgaggggcgagatggcgccttcctctcagggcagaggatca

SEQ ID NO: 236 - DNA, 50 bp, Artificial Sequence
  Synthetic Construct
tgatcctctgccctgagaggaaggcgccatctcgcccctcgacccagatc

SEQ ID NO: 237 - RNA, 26 nt, Artificial Sequence
  Synthetic Construct
gagauggcgccuuccucucaguuuua

SEQ ID NO: 238 - DNA, 52 bp, Artificial Sequence
  Synthetic Construct
tacgaggcgggcggccccggcgccttcatgcacggcgcgggcgccgcgtcct

SEQ ID NO: 239 - DNA, 52 bp, Artificial Sequence
  Synthetic Construct
aggacgcggcgcccgcgccgtgcatgaaggcgccggggccgcccgcctcgta

SEQ ID NO: 240 - RNA, 26 nt, Artificial Sequence
  Synthetic Construct
ggcgcccgcgccgugcaugaguuuua

SEQ ID NO: 241 - DNA, 265 bp, Artificial Sequence
  Synthetic Construct
cgcccagtggctgctgacaacaccgaggaccactccggctcccagctgcagggcggaggaggcagcggcggaggaggcagcggcggaggaggcagcagaaggtatacacgtcggaagaatctgtagggatccaccggtcgccaccgtgagcaagggcgacgagctgtacaagtaaagcggccgcgactctgataaatgcttcaataatattagttattaatagtaatcaaacgtcggggcggcaggccctaataccttggacttt

SEQ ID NO: 242 - DNA, 42 bp, Artificial Sequence
  Synthetic Construct
cctaccagaatcgcccagtggctgctgacaacaccgaggacg

SEQ ID NO: 243 - DNA, 35 bp, Artificial Sequence
  Synthetic Construct
gcggcaggccctaataccttggactttggacagaa

SEQ ID NO: 244 - DNA, 58 bp, Artificial Sequence
  Synthetic Construct
ttatcttgtttcgtattcgcgtgtcgccctttgcgtcagcaatttcctgctcctcttg

SEQ ID NO: 245 - DNA, 24 bp, Artificial Sequence
  Synthetic Construct
ccaaagtccaaggtattagcagcc

SEQ ID NO: 246 - DNA, 59 bp, Artificial Sequence
  Synthetic Construct
ccagaatcgcccagtggctgctaataccatggtgagcaagggcgaggagctgttcaccg

SEQ ID NO: 247 - DNA, 63 bp, Artificial Sequence
  Synthetic Construct
caggagcgcgccatcttcttcaaggacgacggcaactacaagacccaagggcgacacgcgaag

SEQ ID NO: 248 - DNA, 61 bp, Artificial Sequence
  Synthetic Construct
tgtggatcagcaagcaggagggcggaggaggcagcatggtgagcaagggcgaggagctgtt

SEQ ID NO: 249 - DNA, 21 bp, Artificial Sequence
  Synthetic Construct
ccaaagtccaaggtattagca